(19) United States
(12) Patent Application Publication
(10) Pub. No.: US 2026/0137780 A1
(43) Pub. Date: May 21, 2026

(54) MATERIALS AND METHODS FOR IMPROVED EXPANSION AND USES OF IMMUNE CELLS

(71) Applicant: Janssen Biotech, Inc., Horsham, PA (US)

(72) Inventors: Renata GORDON, Huntingdon Valley, PA (US); Iqbal S. GREWAL, Newtown, PA (US); Sandra HAYES, Doylestown, PA (US); Dries DE MAEYER, Antwerp (BE); Marco GOTTARDIS, Princeton, NJ (US)

(21) Appl. No.: 18/706,501

(22) PCT Filed: Nov. 11, 2022
(86) PCT No.: PCT/US2022/079759

§ 371 Date: May 1, 2024

Related U.S. Application Data
(60) Provisional application No. 63/279,042, filed on Nov. 12, 2021.
(60) Provisional application No. 63/323,040, filed on Mar. 23, 2022.

(51) Int. Cl. A61K 40/11; A61K 35/17; A61K 38/00; A61K 40/31; A61K 40/42; C07K 16/28; C07K 16/30; C12N 5/0783; C12N 5/10

(52) U.S. Cl. CPC A61K 40/11; A61K 35/17; A61K 38/00; A61K 40/31; A61K 40/42; C07K 16/2809; C07K 16/30; C12N 5/0636; C12N 5/10; C07K 2317/31; C07K 2319/03; C12N 2500/02; C12N 2501/2302; C12N 2501/2315; C12N 2501/40; C12N 2510/00

(57) ABSTRACT

Provided herein are, inter alia, improved materials and methods for obtainment and use of immune cells (e.g., T cells). In particular, the present disclosure provides improved materials and methods of ex vivo immune cell activation, immune cell expansion, and/or enrichment of immune cells, immune cell subsets, and more specifically Vγ9Vδ2 cells. The present disclosure further provides isolated or purified populations of such Vγ9Vδ2 cells, and the ex vivo and in vivo uses thereof. Also provided herein, in various embodiments, are compositions comprising such improved cells, including the isolated or purified populations of such produced Vγ9Vδ2 cells. CAR cells, and the like, and uses of such compositions and cells in treating a subject in need thereof having, for example, a disease or a disorder.

### CROSS-REFERENCE TO RELATED APPLICATIONS

This application claims the benefit of priority to U.S. Provisional Application No. 63/323,040, filed Mar. 23, 2022 and U.S. Provisional Application No. 63/279,042, filed Nov. 12, 2021, the content of each of which is incorporated herein by reference in its entirety.

### 1. FIELD

Provided herein are, inter alia, improved materials and methods for obtainment and use of immune cells (e.g., T cells). In particular, the present disclosure provides improved materials and methods of ex vivo immune cell activation, immune cell expansion, and/or enrichment of immune cells, immune cell subsets, and more specifically Vγ9Vδ2 cells. The present disclosure further provides isolated or purified populations of such Vγ9Vδ2 cells, and ex vivo and in vivo uses thereof. Also provided herein, in various embodiments, are compositions comprising such cells, including the isolated or purified populations of such produced Vγ9Vδ2 cells, CAR cells, and the like, and uses of such compositions and cells in treating a subject in need thereof having, for example, a disease or a disorder.

### 2. SUMMARY

Provided herein are, inter alia, improved materials and methods for obtainment and use of immune cells (e.g., T cells). In one aspect, provided herein are methods for ex vivo activation and expansion of Vγ9Vδ2 T cells, comprising: (a) contacting a population of cells comprising T cells with a culture system comprising IL-2, IL-15, and a bisphosphonate or a mevalonate pathway intermediate; and (b) culturing the population of cells ex vivo in the culture system under a hypoxic condition to activate and expand Vγ9Vδ2 T cells.

In some embodiments, the method further comprises obtaining the population of cells from a subject. In certain embodiments, the subject is healthy. In certain embodiments, the subject is unhealthy.

In some embodiments, the population of cells is a population of mammalian cells. In certain embodiments, the mammalian cells are human cells. In certain embodiments, the human cells are engineered cells. In certain embodiments, the human cells are non-engineered cells.

In some embodiments, the population of cells is a population of peripheral blood mononuclear cells (PBMCs). In certain embodiments, the PBMCs are freshly obtained PBMCs. In certain embodiments, the PBMCs are frozen PBMCs.

In some embodiments, the population of cells is derived from a human tissue. In certain embodiments, the human tissue is fresh. In certain embodiments, the human tissue is frozen.

In some embodiments, the population of cells comprises tumor-infiltrating lymphocytes (TILs). In certain embodiments, the TILs are freshly obtained TILs. In certain embodiments, the TILs are frozen TILs.

In some embodiments, the population of cells is cultured in the culture system under the hypoxic condition for at least 3 days, or at least 5 days, or at least 7 days, or at least 9 days, or at least 11 days, or at least 13 days, or at least 15 days, or at least 17 days, or at least 19 days, or at least 21 days. In certain embodiments, the population of cells is cultured in the culture system under the hypoxic condition for from 3 days to 25 days, or from 4 days to 23 days, or from 5 days to 21 days, or from 6 days to 19 days, or from 7 days to 17 days, or from 8 days to 15 days, or from 9 days to 14 days, or from 10 days to 14 days, or from 11 days to 14 days, or from 12 days to 14 days. In certain embodiments, the population of cells is cultured in the culture system under the hypoxic condition for about 15 days. In certain embodiments, the population of cells is cultured in the culture system under the hypoxic condition for 14 days.

In some embodiments, the oxygen concentration of the hypoxic condition is less than 15%, or less than 13%, or less than 11%, or less than 9%, or less than 7%, or less than 5%, or less than 3%, or less than 1%, or less than 0.5%. In certain embodiments, the oxygen concentration of the hypoxic condition is from 0.1% to 15%, or from 0.5% to 13%, or from 1% to 13%, or from 1% to 11%, or from 1% to 9%, or from 1% to 7%, or from 2% to 5%. In certain embodiments, the oxygen concentration of the hypoxic condition is or is about 2%, or 5%, or 12%. In certain embodiments, the oxygen concentration of the hypoxic condition is or is about 5%.

In some embodiments, the method further comprises culturing the population of cells in the culture system under a normoxic condition to activate and expand Vγ9Vδ2 T cells. In certain embodiments, the population of cells is cultured under the normoxic condition for at least 1 hour prior to being cultured under the hypoxic condition. In certain embodiments, the population of cells is cultured under the normoxic condition for from 0.5 days to 7 days, or from 1 days to 6 days, or from 1 days to 5 days, or from 1 days to 4 days, or from 1 days to 3 days, or from 1 days to 2 days prior to being cultured under the hypoxic condition.

In certain embodiments, the oxygen concentration of the normoxic condition is or is about 18.2% or 18.6%.

In some embodiments, the IL-2 concentration within the culture system is 10 IU/mL to 1200 IU/mL. In some embodiments, the IL-2 concentration within the culture system is or is about 10 IU/ml. In certain embodiments, the IL-2 concentration within the culture system is adjusted to gradually decreased during the culturing process. In certain embodiments, the IL-2 concentration within the culture system is 1000 IU/mL or no more than 1000 IU/mL on days 0 and 1, 800 IU/mL or no more than 800 IU/mL on days 2, 3 and 4, and 100 IU/mL or no more than 100 IU/mL on day 5 and thereafter.

In some embodiments, the IL-15 concentration within the culture system is from 5 ng/mL to 25 ng/mL, or from 50 ng/mL to 300 ng/ml. In some embodiments, the IL-15 concentration within the culture system is or is about 100 ng/ml, or is or is about 200 ng/mL. In certain embodiments, the IL-15 concentration within the culture system is adjusted during the culturing process. In certain embodiments, the IL-15 concentration within the culture system is 10 ng/mL or no more than 10 ng/mL on days 0 and 1, 20 ng/ml or no more than 20 ng/mL on days 2, 3, and 4, and 10 ng/ml or no more than 10 ng/mL on day 5 and thereafter.

In some embodiments, the bisphosphonate is selected from the group consisting of zoledronic acid, risedronic acid, ibandronic acid, alendronic acid, pamidronic acid, tiludronic acid, etidronic acid, and clodronic acid. In some embodiments, the mevalonate pathway intermediate is selected from the group consisting of HMBPP, BrHPP, and isopentenyl pyrophosphate.

In some embodiments, the method increases the percentage of Vγ9Vδ2 T cells in the population of cells to more than 10%, or more than 15%, or more than 20%, or more than 25%, or more than 30%, or more than 35%, or more than 40%, or more than 45%, or more than 50%, or more than 55%, or more than 60%. In certain embodiments, the method increases the percentage of Vγ9Vδ2 T cells in the population of cells to from 10% to 99%, or from 20% to 95%, or from 30% to 95%, or from 35% to 95%, or from 40% to 95%, or from 45% to 95%, or from 50% to 95%, or from 55% to 95%, or from 60% to 95%, or from 65% to 95%.

In some embodiments, the method increases the total number of Vγ9Vδ2 T cells in the population of cells by at least 10-fold, or at least 30-fold, or at least 50-fold, or at least 100-fold, or at least 150-fold, or at least 200-fold, or at least 250-fold, or at least 300-fold, or at least 350-fold, or at least 400-fold, or at least 450-fold, or at least 500-fold, or at least 550-fold, or at least 600-fold as compared to the total number of Vγ9Vδ2 T cells in the population of cells before the contacting. In certain embodiments, the method increases the total number of Vγ9Vδ2 T cells in the population of cells by from 10-fold to 700-fold, or from 30-fold to 650-fold to fold, or from 50-fold to 600-fold, or from 100-fold to 600-fold, or from 150-fold to 600-fold, or from 200-fold to 600-fold, or from 250-fold to 600-fold, or from 300-fold to 600-fold, or from 350-fold to 600-fold, or from 400-fold to 600-fold, or from 450-fold to 600-fold, or from 500-fold to 600-fold as compared to the total number of Vγ9Vδ2 T cells in the population of cells before the contacting.

In some embodiments, the method further comprises enriching the ex vivo expanded Vγ9Vδ2 T cells from the population of cells.

In another aspect, provided herein are methods for ex vivo activation and expansion of Vγ9Vδ2 T cells, comprising: a) contacting a population of cells comprising T cells with a culture system comprising IL-2, IL-15, and zoledronic acid, b) culturing the population of cells ex vivo in the culture system under a hypoxic condition for about 14 days to activate and expand Vγ9Vδ2 T cells, and wherein (i) the IL-2 concentration in the culture system is (i) 1000 IU/mL or no more than 1000 IU/mL on days 0 and 1, 800 IU/mL or no more than 800 IU/mL on days 2, 3 and 4, and 100 IU/mL or no more than 100 IU/mL on day 5 and thereafter, or (2) is or is about 10; (ii) the IL-15 concentration within the culture system is (1) 10 ng/ml or no more than 10 ng/mL on days 0 and 1, 20 ng/ml or no more than 20 ng/ml on days 2, 3, and 4, and 10 ng/ml or no more than 10 ng/ml on day 5 and thereafter, or (2) is or is about 100 ng/ml, or is or is about 200 ng/ml; (iii) the oxygen concentration of the hypoxic condition is or is about 5%. In certain embodiments, (iv) the zoledronic acid concentration in the culture system is or is about 350 nM.

In another aspect, provided herein are methods for ex vivo activation and expansion of Vγ9Vδ2 T cells, comprising: a) contacting a population of cells comprising T cells with a culture system comprising IL-2, IL-15, and zoledronic acid, b) culturing the population of cells under a normoxic condition to activate and expand Vγ9Vδ2 T cells for at least 1 hour prior to being cultured under a hypoxic condition; and c) culturing the population of cells under the hypoxic condition for about 14 days to further activate and expand Vγ9Vδ2 T cells, wherein (i) the IL-2 concentration in the culture system is (i) 1000 IU/mL or no more than 1000 IU/mL on days 0 and 1, 800 IU/mL or no more than 800 IU/mL on days 2, 3 and 4, and 100 IU/mL or no more than 100 IU/mL on day 5 and thereafter, or (2) is or is about 10; (ii) the IL-15 concentration within the culture system is (1) 10 ng/ml or no more than 10 ng/ml on days 0 and 1, 20 ng/mL or no more than 20 ng/ml on days 2, 3, and 4, and 10 ng/ml or no more than 10 ng/mL on day 5 and thereafter, or (2) is or is about 100 ng/ml, or is or is about 200 ng/ml; (iii) the oxygen concentration of the hypoxic condition is or is about 5%; (iv) the oxygen concentration of the normoxic condition is or is about 18.2% (e.g., about 18.2% or 18.6%); optionally (v) the zoledronic acid concentration in the culture system is or is about 350 nM.

In another aspect, provided herein are isolated populations of Vγ9Vδ2 T cells produced by a presently disclosed method.

In another aspect, provided herein are isolated populations of cells, wherein the percentage of Vγ9Vδ2 T cells in the isolated population of cells is (a) more than 10%, or more than 15%, or more than 20%, or more than 25%, or more than 30%, or more than 35%, or more than 40%, or more than 45%, or more than 50%, or more than 55%, or more than 60%; or (b) from 10% to 99%, or from 20% to 95%, or from 30% to 95%, or from 35% to 95%, or from 40% to 95%, or from 45% to 95%, or from 50% to 95%, or from 55% to 95%, or from 60% to 95%, or from 65% to 95%.

In yet another aspect, provided herein are pharmaceutical compositions comprising the isolated population of Vγ9Vδ2 T cells disclosed herein and a pharmaceutically acceptable excipient.

In yet another aspect, provided herein are methods for treating a disease or disorder in a subject using the presently disclosed cells or compositions. In certain embodiments, the method comprises comprising administering to the subject: (i) a therapeutically effective amount of the Vγ9Vδ2 T cells or pharmaceutical composition disclosed herein, and (ii) a therapeutically effective amount of one or more multispecific antibodies. In some embodiments, each of the multispecific antibodies comprises: a first binding domain that binds to an antigen expressed on a Vγ9Vδ2 T cell; and a second binding domain that binds to an antigen expressed on an unhealthy cell. In certain embodiments, the antigen expressed on the Vγ9Vδ2 T cell is T Cell Receptor Gamma Variable 9 (TRGV9) or CD3. In certain embodiments, the unhealthy cell is a cancer cell, and wherein the antigen expressed on the unhealthy cell is a tumor-associated antigen (TAA). In certain embodiments, the cancer cell is a blood cancer cell or a solid tumor cancer cell. In some embodiments, the method comprises administering to the subject: (i) a therapeutically effective amount of the Vγ9Vδ2 T cells or pharmaceutical composition disclosed herein, and (ii) a therapeutically effective amount of Vγ9×TAA and/or CD3×TAA bispecific antibodies. In some embodiments, the disease or disorder is cancer. In certain embodiments, the cancer is a blood cancer. In certain embodiments, the cancer is a solid tumor cancer. In some embodiments, the subject is a human subject in need thereof.

In yet another aspect, provided herein are processes for making a chimeric antigen receptor (CAR) T cell product, comprising: (i) a step of performing a function of obtaining the isolated population of Vγ9Vδ2 T cells disclosed herein; and (ii) a step of performing a function of expressing a CAR in the Vγ9Vδ2 T cells. In some embodiments, the CAR comprises an extracellular domain, a transmembrane domain, and an intracellular domain. In certain embodiments, the extracellular domain binds to an antigen expressed on an unhealthy cell. In certain embodiments, the unhealthy cell is a cancer cell. In certain embodiments, the cancer cell is a blood cancer cell or a solid tumor cancer cell.

In yet another aspect, provided herein are methods for making a chimeric antigen receptor (CAR) T cell product, comprising: (i) obtaining a population of cells comprising the Vγ9Vδ2 T cells disclosed herein; and (ii) introducing a nucleic acid encoding a CAR into the population of the cells. In some embodiments, the CAR comprises an extracellular domain, a transmembrane domain, and an intracellular domain. In certain embodiments, the extracellular domain binds to an antigen expressed on an unhealthy cell. In certain embodiments, the unhealthy cell is a cancer cell. In certain embodiments, the cancer cell is a blood cancer cell or a solid tumor cancer cell.

In yet another aspect, provided herein are CAR T cell products produced by a method disclosed herein.

In yet another aspect, provided herein are CAR T cells comprising a CAR comprising an extracellular domain, a transmembrane domain, and an intracellular domain, wherein the CAR T cell is a Vγ9Vδ2 T cell.

In yet another aspect, provided herein are pharmaceutical compositions comprising the CAR T cell products or the CAR T cells disclosed herein, and a pharmaceutically acceptable excipient.

In yet another aspect, provided herein are methods for treating a disease or disorder in a subject, comprising administering to the subject a therapeutically effective amount of the CAR T cells disclosed herein or the pharmaceutical composition comprising the CAR T cell products or the CAR T cells disclosed herein.

In yet another aspect, provided herein are methods for treating a disease or disorder in a subject, comprising administering to the subject a therapeutically effective amount of a CAR T cell, wherein the CAR T cell comprises a CAR comprising an extracellular domain, a transmembrane domain, and an intracellular domain, and wherein the CAR T cell is a Vγ9Vδ2 T cell.

In yet another aspect, provided herein are methods for establishing an in vivo engraftment of Vγ9Vδ2 T cells in a receiving subject. In certain embodiments, the method comprises (i) obtaining a population of cells comprising Vγ9Vδ2 T cells disclosed herein; and (ii) adoptively transferring the population of cells to the receiving subject.

In some embodiments, the population of cells comprising Vγ9Vδ2 T cells is produced by ex vivo activation and expansion of the population of cells comprising T cells obtained from the receiving subject.

In some embodiments, the population of cells comprising Vγ9Vδ2 T cells comprises more than 10%, or more than 15%, or more than 20%, or more than 25%, or more than 30%, or more than 35%, or more than 40%, or more than 45%, or more than 50%, or more than 55%, or more than 60% Vγ9Vδ2 T cells in the population of cells.

In some embodiments, the population of cells comprising Vγ9Vδ2 T cells comprises from 10% to 99%, or from 20% to 95%, or from 30% to 95%, or from 35% to 95%, or from 40% to 95%, or from 45% to 95%, or from 50% to 95%, or from 55% to 95%, or from 60% to 95%, or from 65% to 95% Vγ9Vδ2 T cells in the population of cells.

In some embodiments, the population of cells comprising Vγ9Vδ2 T cells comprises less than 95%, or less than 90%, or less than 85%, or less than 80%, or less than 75%, or less than 70%, or less than 65%, or less than 60%, or less than 55%, or less than 50%, or less than 45%, or less than 40%, or less than 35%, or less than 30%, or less than 25%, or less than 20%, or less than 15%, or less than 10%, or less than 5%, or less than 1%, or is devoid of αβ T cells in the population of cells.

In some embodiments, the population of cells comprises at least about or about 5×10⁶ cells, at least about or about 1×10⁷ cells, at least about or about 5×10⁷ cells, at least about or about 1×10⁸ cells, at least about or about ×10⁸ cells, at least about or about 3×10⁸ cells, at least about or about 4×10⁸ cells, at least about or about 5×10⁸ cells, at least about or about 6×10⁸ cells, at least about or about 7×10⁸ cells, at least about or about 8×10⁸ cells, at least about or about 9×10⁸ cells, at least about or about 1×10⁹ cells, at least about or about 2×10⁹ cells, at least about or about 3×10⁹ cells, at least about or about 4×10⁹ cells, at least about or about 5×10⁹ cells, at least about or about 6×10⁹ cells, at least about or about 7×10⁹ cells, at least about or about 8×10⁹ cells, at least about or about 9×10⁹ cells, or at least about or about 1×10¹⁰ cells.

In some embodiments, the population of cells comprising Vγ9Vδ2 T cells is a purified population of Vγ9Vδ2 T cells.

In some embodiments, the population of cells comprising Vγ9Vδ2 T cells is enriched to comprise more than 95% Vγ9Vδ2 T cells one day before the adoptive transfer of the population of cells to the receiving subject.

In some embodiments, the adoptively transferring comprises administering to the receiving subject the population of cells. In some embodiments, the method further comprises (iii) administering an effective amount of a composition comprising IL-2, IL-15, a bisphosphonate or a mevalonate pathway intermediate, or a combination thereof, concurrently or sequentially with the adoptive transfer of the population of cells. In some embodiments, the bisphosphonate is selected from the group consisting of zoledronic acid, risedronic acid, ibandronic acid, alendronic acid, pamidronic acid, tiludronic acid, etidronic acid, and clodronic acid. In some embodiments, the mevalonate pathway intermediate is selected from the group consisting of HMBPP, BrHPP, and isopentenyl pyrophosphate. In some embodiments, the bisphosphonate is zoledronic acid. In some embodiments, the zoledronic acid is administered at the dosage of 2.5 mg/kg of body weight. In some embodiments, the administration of the population of the cells and/or administration of the effective amount of the composition are intravenous administration or intraperitoneal administration. In some embodiments, the IL-2 is administered at the dosage of 2×10⁴ IU/kg of body weight. In some embodiments, the receiving subject expresses IL-15. In some embodiments, the receiving subject is a model animal.

In some embodiments of the method for establishing an in vivo engraftment of Vγ9Vδ2 T cells in a receiving subject as described herein, the adoptively transferred Vγ9Vδ2 T cells produce progeny cells in the subject. In some embodiments, the progeny cells are CD45⁺ cells. In some embodiments, the progeny cells are CD56⁺ cells. In some embodiments, the progeny cells are CD69⁺ cells. In some embodiments, the adoptively transferred Vγ9Vδ2 T cells are present in the blood of the receiving subject at least 7 days, at least 14 days, at least 21 days, or at least 28 days after adoptive transfer of the population of cells to the receiving subject. In some embodiments, the adoptively transferred Vγ9Vδ2 T cells infiltrate into a tissue in the receiving subject. In some embodiments, the tissue is a spleen tissue, a liver tissue, a lung tissue, an intestine tissue, a skin tissue, or a combination thereof. In some embodiments, the tissue comprises an unhealthy cell. In some embodiments, the unhealthy cell is a cancer cell. In some embodiments, the cancer cell is a blood cancer cell or a solid tumor cancer cell. In some embodiments, the receiving subject does not develop any symptom of a Graft Versus Host Disease (GvHD) at least 7 days, at least 14 days, at least 21 days, or at least 28 days after adoptive transfer of the population of cells to the receiving subject.

In some embodiments of the method for establishing an in vivo engraftment of Vγ9Vδ2 T cells in a receiving subject as described herein, the Vγ9Vδ2 T cells are chimeric antigen receptor (CAR) T cells comprising an extracellular domain, a transmembrane domain, and an intracellular domain. In some embodiments, the extracellular domain binds to an antigen expressed on an unhealthy cell. In some embodiments, the unhealthy cell is a cancer cell. In some embodiments, the cancer cell is a blood cancer cell or a solid tumor cancer cell.

In yet another aspect, provided herein are methods for treating a disease or disorder in a subject, comprising administering to the subject: (i) a therapeutically effective amount of a population of Vγ9Vδ2 T cells, and (ii) a therapeutically effective amount of one or more multispecific antibodies. In some embodiments, each of the multispecific antibodies comprising: a first binding domain that binds to an antigen expressed on a Vγ9Vδ2 T cell; and a second binding domain that binds to an antigen expressed on an unhealthy cell. In certain embodiments, the antigen expressed on the Vγ9Vδ2 T cell is T Cell Receptor Gamma Variable 9 (TRGV9) or CD3. In certain embodiments, the unhealthy cell is a cancer cell, and wherein the antigen expressed on the unhealthy cell is a tumor-associated antigen (TAA). In certain embodiments, the cancer cell is a blood cancer cell or a solid tumor cancer cell. In some embodiments, the method comprises administering to the subject: (i) a therapeutically effective amount of the Vγ9Vδ2 T cells or pharmaceutical composition disclosed herein, and (ii) a therapeutically effective amount of Vγ9×TAA and/or CD3×TAA bispecific antibodies. In some embodiments, the disease or disorder is cancer. In certain embodiments, the cancer is a blood cancer. In certain embodiments, the cancer is a solid tumor cancer. In some embodiments, the subject is a human subject in need thereof.

In yet another aspect, the present disclosure provides the pharmaceutical composition disclosed herein, the isolated population of Vγ9Vδ2 T cells disclosed herein, or the isolated population of cells disclosed herein, for use in the treatment of a disease or disorder in a subject. In certain embodiments, the treatment comprises administering to the subject: (i) a therapeutically effective amount of the isolated population of Vγ9Vδ2 T cells, the isolated population of cells, or the pharmaceutical composition, and (ii) a therapeutically effective amount of at least one multispecific antibody.

In some embodiments, the at least one multispecific antibody comprises (a) a first binding domain that binds to an antigen expressed on a Vγ9Vδ2 T cell; and (b) a second binding domain that binds to an antigen expressed on an unhealthy cell.

In some embodiments, (a) the antigen expressed on the Vγ9Vδ2 T cell is T Cell Receptor Gamma Variable 9 (TRGV9) or CD3; or (b) the unhealthy cell is a cancer cell, and wherein the antigen expressed on the unhealthy cell is a tumor-associated antigen (TAA). In certain embodiments, the cancer cell is a blood cancer cell or a solid tumor cancer cell.

In some embodiments, the at least one multispecific antibody comprises a Vγ9×TAA and/or CD3×TAA bispecific antibody.

In some embodiments, the disease or disorder is cancer. In certain embodiments,, the cancer is a blood cancer or a solid tumor cancer. In certain embodiments, the subject is a human subject in need thereof.

### 3. BRIEF DESCRIPTION OF THE FIGURES

FIG. 1 is an exemplary schematic diagram of a process of the present disclosure, selective in vitro expansion of Vγ9Vδ2 T cells. Briefly, peripheral blood mononuclear cells (PBMCs) from a healthy donor were cultured in complete growth medium containing IL-2, IL-15, and zoledronic acid (zol). Monocytes selectively uptook zol, and zol and was subsequently metabolized into IPP (isoprenyl diphosphate) and DMAPP (dimethylallyl diphosphate). Generated phosphates bound the cytoplasmic tail of CD277, resulting in its conformational change on the surface of monocytes. Upon conformational change, CD277 was recognized by the Vγ9Vδ2 T cell receptor (TCR), which subsequently triggered their activation and expansion, while other immune cell types underwent apoptosis.

FIGS. 2A and 2B depict the starting Vγ9Vδ2 T cell populations in PBMC prior to in vitro expansion. FIG. 2A shows the identification of Vγ9Vδ2 T cell populations by flow cytometry gating on single viable CD3⁺ cells on PBMC from one healthy donor at day 0 prior to in vitro expansion. FIG. 2B shows the distribution of CD3⁺Vγ9Vδ2 T cells prior to in vitro expansion across a cohort of 68 healthy donors.

FIGS. 3A to 3C depict the expansion of Vγ9Vδ2 T cells after 14-day in vitro expansion under the normoxic condition (18.2% oxygen) from 68 healthy donors. FIG. 3A shows Vγ9Vδ2 T cell expansion values (black line) plotted against frequency of starting CD3⁺Vγ9Vδ2 T cell populations (grey bars). The left Y axis illustrates a range of frequency of starting CD3⁺Vγ9Vδ2 T cell, and the right Y axis illustrates expansion efficiency in fold changes. Each individual bar/line point represents a single donor. FIG. 3B shows Vγ9Vδ2 T cell expansion (black bars) plotted against total number of Vγ9Vδ2 T cells obtained after the expansion (grey line). The left Y axis illustrates expansion efficiency in fold changes, and the right Y axis illustrates total number of Vγ9Vδ2 T cells obtained after the expansion and normalized to starting amount of 50×10⁶ PBMC. Each individual bar/line point represents a single donor. FIG. 3C shows frequency of Vγ9Vδ2 T cell populations at day 0 (grey line) and day 14 (black line). The left Y axis illustrates frequency of Vγ9Vδ2 T cell populations at day 0, and the right Y axis illustrates frequency of Vγ9Vδ2 T cell populations at day 14.

FIGS. 4A to 4C depict the features of low expanders and high expanders regarding the expansion of Vγ9Vδ2 T cells after 14-day in vitro expansion under the normoxic condition. FIG. 4A shows immune cell clusters within PBMC at day 0 from 5 low expanders (left panel) and 5 high expanders (right panel). FIG. 4B compares the presence of αβ T cells, Vγ9Vδ2 T cells, NK cells and monocytes throughout the 14-day expansion period between high expanders (solid line, n=10) and low expanders (dashed lane, n=10). FIG. 4C compares the proliferative capacities of high expanders (n=10) and low expanders (n=10) by measuring the frequency of Vγ9Vδ2 T cells expressing the proliferation marker Ki-67 at day 14.

FIGS. 5A to 5C show that differences in Vγ9Vδ2 T cell expansion were independent of demographic parameters. FIG. 5A shows linear regression analysis of Vγ9Vδ2 T cells expansion efficiency plotted against donor age. FIG. 5B compares the Vγ9Vδ2 T cell expansion efficiency between genders. FIG. 5C compares the Vγ9Vδ2 T cell expansion efficiency across ethnical identity.

FIGS. 6A to 6C depict immune cell clusters identified based on transcriptomes of cells compiled from high and low expanders under different experimental conditions. FIG. 6A shows Uniform Manifold Approximation and Projection “UMAP” plot of immune cell clusters representing compiled 358370 sequenced cells from 10 donors (5 high expanders and 5 low expanders) across 3 experimental conditions (whole blood PBMC at day 0, expanded non-enriched Vγ9Vδ2 T cell at day 14, and non-expanded enriched γδ T cells at day 0). FIG. 6B shows UMAP plots of immune cell clusters representing compiled sequenced cells from PBMC of 10 donors at day 0. FIG. 6C shows UMAP plots of immune cell clusters representing compiled sequenced cells from expanded non-enriched Vγ9Vδ2 T cell of 10 donors at day 14.

FIGS. 7A to 7F depict purity of γδ T cells in clusters of expanded non-enriched Vγ9Vδ2 T cells at day 14 (“expanded cohort”) and non-expanded enriched Vγ9Vδ2 T cells at day 0 (“enriched cohort”). FIG. 7A shows UMAP plot illustrating prevalence of γδ T cells corresponding to TRGD signatures (dark shade) in the expanded cohort. FIG. 7B shows UMAP plot illustrating presence of αβ T cells corresponding to TRAB signatures (dark shade) in the expanded cohort. FIG. 7C shows UMAP plot illustrating cells corresponding to TRDG signatures (light shade) and TRAB signatures (dark shade) in the expanded cohort. FIG. 7D shows UMAP plot illustrating prevalence of γδ T cells corresponding to TRGD signatures (dark shade) in the purified cohort. FIG. 7E shows UMAP plot illustrating presence of αβ T cells corresponding to TRAB signatures (dark shade) in the purified cohort. FIG. 7F shows UMAP plot illustrating cells corresponding to TRDG signatures (light shade) and TRAB signatures (dark shade) in the purified cohort.

FIGS. 8A and 8B depict differential population distribution between high and low expanders in clusters of expanded non-enriched Vγ9Vδ2 T cells at day 14 (“expanded cohort”) and non-expanded enriched Vγ9Vδ2 T cells at day 0 (“enriched cohort”). FIG. 8A shows UMAP plots illustrating compiled data of γδ T cell populations from 5 low expanders (left panel) and 5 high expanders (right panel) collected from the expanded cohort. High cell density is indicated by solid black. FIG. 8B shows UMAP plots illustrating compiled data of γδ T cell populations from 5 low expanders (left panel) and 5 high expanders (right panel) collected from the enriched cohort. High cell density is indicated by solid black.

FIGS. 9A and 9B depict differentially expressed gene (DEG) analysis of high and low expanders in clusters of expanded non-enriched Vγ9Vδ2 T cells at day 14 (“expanded cohort”). FIG. 9A is a volcano plot representing differential expression of genes between high and low expanders. FIG. 9B is a table listing 20 top DEGs with highest significance and/or highest fold changes. Gene description highlighted in grey are hypoxia-related genes. The gene list was adapted from the Database for Annotation, Visualization, and Integrated Discovery (DAVID) (Dennis et al., Genome Biology volume 4, Article number: R60 (2003)).

FIGS. 10A to 10C depict the hypoxia-related genes that were co-localized with the unique γδ T cluster in high expanders identified in FIG. 8A. FIG. 10A shows HIF1A-AS3 expression plotted on the compiled cell density plots from high and low expanders in the expanded cohort. FIG. 10B shows BNIP 3L expression plotted on the compiled cell density plots from high and low expanders in the expanded cohort. FIG. 10C shows MIF expression plotted on the compiled cell density plots from high and low expanders in the expanded cohort.

FIGS. 11A and 11B show the DEG analysis of high and low expanders in clusters of non-expanded enriched Vγ9Vδ2 T cells at day 0 (“enriched cohort”). FIG. 11A is a volcano plot illustrating differential expression of genes between high and low expanders. FIG. 11B is a table listing 20 top DEGs with highest significance and/or highest fold changes. Gene description highlighted in grey are hypoxia-related genes.

FIGS. 12A to 12E show increased purity of Vγ9Vδ2 T cells from a single donor that were cultured under hypoxic conditions. FIG. 12A shows flow cytometry plots (gated on single viable CD3⁺ Vγ9⁺ cells) indicating increased purity of Vγ9Vδ2 T cell populations in one donor following in vitro expansion for 14 days under different culture conditions (normoxia control, 18.2% oxygen; hypoxia: 12% oxygen, 5% oxygen, 2% oxygen). FIG. 12B shows histogram overlap of plots shown in FIG. 12A, indicating the increase of Vγ9Vδ2 T cell populations. FIG. 12C shows Vγ9Vδ2 T cell increased purity in fold changes observed under different culture conditions (normoxia control, 18.2% oxygen; hypoxia: 12% oxygen, 5% oxygen, 2% oxygen). Each line represents a single donor. FIG. 12D shows total Vγ9Vδ2 T cell frequency following in vitro expansion for 14 days under different culture conditions (normoxia control, 18.2% oxygen; hypoxia: 12% oxygen, 5% oxygen, 2% oxygen). Each line represents a single donor. FIG. 12E shows total αβ T cell frequency following in vitro expansion for 14 days under different culture conditions (normoxia control, 18.2% oxygen; hypoxia: 12% oxygen, 5% oxygen, 2% oxygen). Each line represents a single donor

FIG. 13 is a schematic diagram of a process of the present disclosure of redirecting Vγ9Vδ2 T cells to tumor-associated antigen (TAA)-expressing tumor cells using bispecific antibodies such as Vγ9×TAA or CD3×TAA.

FIG. 14 shows that Vγ9Vδ2 T cells expanded under normoxic condition (18.2% oxygen, top panel) and hypoxic condition (5% oxygen, bottom panel) maintained robust effector profile in the presence of Vγ9×HER2 or CD3×HER2 bispecific antibodies.

FIG. 15A depicts the expansion of Vγ9Vδ2 T cells among different donors from fresh peripheral blood mononuclear cells (PBMCs). FIG. 15B shows the total number of Vγ9Vδ2 T cells obtained from post-expansion at day 14.

FIG. 16 shows the percentage of Vγ9⁺ CD3⁺ cells during enrichment from day 0 to day 14 and that the cells could be further enriched using negative selection.

FIGS. 17A and 17B shows the gating strategies for Vγ9Vδ2 T cells. FIG. 17A depicts a gating strategy for determining the number of Vγ9Vδ2 T cells. FIG. 17B depicts a gating strategy for determining the number of Vγ9Vδ2 T cells using both Vγ9 and Vδ2 stains.

FIGS. 18A and 18B show the study design and purity of Vγ9Vδ2 T cells of Example 4 disclosed herein. FIG. 18A depicts the protocol and timeline of the adoptive transfer process for NSG, NSG-IL15, and NOG-IL15 mice as disclosed in the Example 4 of the present disclosure. FIG. 18B shows the purity of non-enriched and enriched Vγ9Vδ2 T cells.

FIG. 19 shows the flow cytometry analysis of mice 7 days post the adoptive transfer.

FIG. 20 shows the flow cytometry analysis of Vγ9Vδ2 T cell engraftment among the NSG, NSG-IL15, and NOG-IL15 mouse strains and treatment groups.

FIG. 21 shows enriched Vγ9Vδ2 T cell engraftment kinetics among the NSG, NSG-IL15, and NOG-IL15 mouse strains.

FIG. 22 shows engraftment kinetics of human cells (CD45⁺) in mouse peripheral blood.

FIG. 23 shows the percentage of Vγ9Vδ2 T cells in mouse circulation normalized to the percentage of CD45⁺CD3⁺ cells.

FIG. 24 shows the purity of Vγ9Vδ2 T cells in the CD45⁺CD3⁺ population.

FIG. 25 shows the cell counts of Vγ9Vδ2 T cells per microliter of mouse blood.

FIG. 26 shows the presence of αβ T cells in NOG-IL15 mice who received non-enriched Vγ9Vδ2 T cells.

FIG. 27 shows the body weight of NSG, NSG-IL15, and NOG-IL15 mice following administration of purified Vγ9Vδ2 T cells.

FIG. 28 shows the CD56 expression on adoptively transferred Vγ9Vδ2 T cells.

FIG. 29 shows the CD69 expression on adoptively transferred Vγ9Vδ2 T cells.

FIG. 30 depicts the protocol and timeline of the adoptive transfer process in NOG-IL15 mice using fresh or frozen Vγ9Vδ2 T cells as disclosed in Example 5 of the present disclosure.

FIG. 31 shows the Vγ9Vδ2 T cell purity at the day of the adoptive transfer.

FIG. 32 shows the Vγ9Vδ2 T cell engraftment among various donor groups and fresh or frozen cells.

FIG. 33 shows the Vγ9Vδ2 T cell engraftment kinetics among the various donor groups.

FIG. 34 shows the purity of negatively enriched Vγ9Vδ2 T cells.

FIG. 35 shows the cell counts per microliter of donor Vγ9Vδ2 T cells in mouse blood.

FIG. 36 shows the body weight of NOG-IL 15 mice following the administration of donor Vγ9Vδ2 T cells.

FIG. 37 shows the infiltrating γδ T cells (dark stain) in mouse liver tissues.

FIGS. 38A and 38B are schematic showing of the study design of Example 6 of the present disclosure. FIG. 38A depicts the three study groups and models used in Example 6. FIG. 38B is a schematic showing of the treatment schedule of Example 6.

FIG. 39 is a table showing the design of the three study groups of Example 6.

FIGS. 40A and 40B show the gating strategy and the characterization of the starting Vγ9Vδ2 T cells. FIG. 40A shows the gating strategy used for the detection of the Vγ9Vδ2 T cells in mouse peripheral blood. FIG. 40B shows the purity and phenotypes of Vγ9Vδ2 T cells prior to the adoptive transfer and subcutaneous implantation.

FIGS. 41A and 41B show the persistence of Vγ9Vδ2 T cells in mouse peripheral blood. FIG. 41A shows representative flowcytometry analysis of percentages of Vγ9Vδ2 T cells in mouse peripheral blood. FIG. 41B shows the percentages of Vγ9Vδ2 T cells in mouse peripheral blood in three study groups throughout the study period.

FIGS. 42A and 42B show the kinetic changes in T cell memory phenotypes of Vγ9Vδ2 T cells. FIG. 42A shows representative flowcytometry analysis of percentages of memory phenotypes of Vγ9Vδ2 T cells. FIG. 42B shows the percentages of central memory Vγ9Vδ2 T cells and effector memory T cells in mouse peripheral blood throughout the study period.

FIG. 43 shows the kinetic changes in phenotypes of Vγ9Vδ2 T cells.

FIG. 44 shows the tumor volume measurements throughout the study period in three study groups.

FIGS. 45A to 45C show the tumor volume measurements of each individual mouse throughout the study period in Group 1 (FIG. 45A), Group 2 (FIG. 45B), and Group 3 (FIG. 45C). Each line represents the tumor volume measurements of one mouse.

FIG. 46 shows the body weight measurements throughout the study period in three study groups.

FIGS. 47A to 47C show the body weight measurements of each individual mouse throughout the study period in Group 1 (FIG. 47A), Group 2 (FIG. 47B), and Group 3 (FIG. 47C). Each line represents the body weight measurements of one mouse.

### 4. DETAILED DESCRIPTION

Adoptive transfer of immune cells genetically modified to recognize malignancy-associated antigens is a promising cancer treatment (see, e.g., Brenner et al, Current Opinion in Immunology, 22 (2): 251-257 (2010); Rosenberg et al., Nature Reviews Cancer, 8 (4): 299-308 (2008)). In particular, genetically engineered immune cells (e.g., T cells) expressing chimeric antigen receptors (CARs) is a powerful promising cancer treatment.

One known approach to generate clinical grade autologous CAR T cells is to collect T cells from patients by leukapheresis (or peripheral blood), activate, transduce with CAR constructs using viral vectors, expand, and then reinfuse to the same patients after lymphodepleting chemotherapy as a single time treatment (see Ruella et al., BioDrugs, 31 (6): 473-481 (2017)).

Despite certain unprecedented treatment outcomes of CAR T therapy, this approach has challenges, for example, being limited by its autologous feature. For example, T cells cannot be harvested and/or expanded from some patients, and the quality of T cells may not meet the manufacturing standards. In addition, high costs are unavoidable for such highly personalized procedures. Although allogeneic cell therapy products are being actively explored and developed, one of the biggest hurdles of traditional T cell therapy lies in the inability of T cells to infiltrate and persist in the immunosuppressive tumor microenvironment or act effectively against non-solid or semi-solid cancers. Moreover, CAR T therapy has intrinsic manufacturing challenges, such as manufacturing failures, time delays, insufficient cell expansion, or heterogeneous products which can be detrimental to the recipient patients.

Against this background, the present disclosure addresses various challenges in immune cell therapies.

The present disclosure is based, in part, on novel methods or processes for producing immune cells, such as Vγ9Vδ2 T cells, their improved and advantageous properties, and uses thereof for making cellular therapies for treating a subject in need thereof, such as a subject having a disease or a disorder.

### 4.1. DEFINITIONS

Techniques and procedures described or referenced herein include those that are generally well understood and/or commonly employed using conventional methodology by those skilled in the art in view of the teachings in the specification, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009);

Monoclonal Antibodies: Methods and Protocols (Albitar ed. 2010); and Antibody Engineering Vols 1 and 2 (Kontermann and Dübel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies, antibody compositions with polyepitopic or monoepitopic specificity, polyclonal or monovalent antibodies, multivalent antibodies, multispecific antibodies (e.g., bispecific antibodies so long as they exhibit the desired biological activity), single chain antibodies, and fragments thereof (e.g., domain antibodies). An antibody can be human, humanized, chimeric and/or affinity matured, as well as an antibody from other species, for example, mouse, rabbit, llama, etc.

An “antigen” is a structure to which an antibody can selectively bind. In some embodiments, the target antigen is a polypeptide. In certain embodiments, an antigen is associated with a cell, for example, is present on or in a cell.

“Chimeric antigen receptor” or “CAR” as used herein refers to genetically engineered receptors, which can be used to graft one or more antigen specificity onto immune cells, such as T cells. CARs are also known as “artificial T cell receptors,” “chimeric T cell receptors,” or “chimeric immune receptors.” In some embodiments, the CAR comprises an extracellular antigen binding domain specific for one or more antigens (such as tumor antigens), a transmembrane domain, and an intracellular signaling domain of a T cell and/or other receptors. “CAR T cell” refers to a T cell that expresses a CAR.

An “isolated nucleic acid” is a nucleic acid, for example, an RNA, DNA, or a mixed nucleic acid, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome.

The term “host cell” as used herein refers to a particular subject cell that may be transfected with a nucleic acid molecule and the progeny or potential progeny of such a cell.

Progeny of such a cell may not be identical to the parent cell transfected with the nucleic acid molecule due to mutations or environmental influences that may occur in succeeding generations or integration of the nucleic acid molecule into the host cell genome.

As used herein, the term “autologous” is meant to refer to any material derived from the same individual to whom it is later to be re-introduced into the individual.

As used herein, the term “allogeneic” refers to a graft derived from a different individual of the same species.

As used herein, the term “normoxic” refers to a condition of normal oxygen concentration.

As used herein, the term “hypoxic” refers to a condition of oxygen concentration lower than the normal level.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, the term “isolation” or “isolating” refers to a process of increasing the percentage of a certain substance in a composition. For example, isolating a type of cells from a population of cells refers to a process of creating a population of cells in which the percentage of this type of cells increases as compared to the percentage of this type of cells in the original population of cells. Therefore, the term “isolated” when used in the context of a type of cells does not mean that the isolated population of cells comprises 100% of this type of cells, rather it means the percentage of this type of cells increases in a population of cells after the isolation process.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

The term “effective amount” or “therapeutically effective amount” as used herein refers to the amount of a single domain antibody or a therapeutic molecule comprising an agent and the single domain antibody or pharmaceutical composition provided herein which is sufficient to result in the desired outcome.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., diabetes or a cancer).

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 4.2. Methods for Ex Vivo Activation and Expansion of Vγ9Vδ2 T cells

Provided herein, in one aspect, are methods for ex vivo activation and expansion of Vγ9Vδ2 T cells under hypoxic conditions. In some embodiments, the methods provided herein comprise contacting a population of cells comprising T cells with a culture system comprising IL-2, IL-15, and a bisphosphonate or a mevalonate pathway intermediate, and culturing the population of cells comprising T cells ex vivo in the culture system under a hypoxic condition for enhanced ex vivo activation and expansion of Vγ9Vδ2 T cells in the population of cells comprising T cells.

### 4.2.1. Obtaining a Population of Cells Comprising T cells

Exemplary T cells include effector T cells, accessory T cells, cytotoxic T cells, helper T cells, regulatory T cells, and natural killer T cells. It is known in the art that T cells can be obtained from a number of sources. In some embodiments, the population of cells comprising T cells are obtained from a cultured T cell line. In some embodiments, the population of cells comprising T cells are collected, isolated, purified or induced from a body fluid, a tissue or an organ including but not limited to peripheral blood, umbilical cord blood, bone marrow, lymph node, spleen, or other tissues or fluids of a subject. In certain embodiments, the population of cells comprising T cells are peripheral blood lymphocytes, precursor cells of T cells (such as hematopoietic stem cells, lymphocyte precursor cells, etc.) or a cell population comprising them. Immature T cells may be found in the thymus.

In certain embodiments, the population of cells comprising T cells are peripheral blood mononuclear cells (PBMCs). In certain embodiments, the PBMCs are freshly obtained PBMCs. In certain embodiments, the PBMCs are frozen PBMCs. Various methods of collecting and preparing PBMCs are known in the art.

In certain embodiments, the population of cells comprising T cells are derived from a human tissue. In specific embodiments, the human tissue is fresh. In certain embodiments, the human tissue is frozen. Various methods of collecting and preparing human tissue are known in the art.

In certain embodiments, the population of cells comprising T cells are tumor-infiltrating lymphocytes (TILs). In certain embodiments, the TILs are freshly obtained TILs. In certain embodiments, the TILs are frozen TILs. Various methods of collecting and preparing TILs are known in the art.

In some embodiments, the population of cells comprising T cells are mammalian cells. In certain embodiments, the mammalian cells are human cells. In certain embodiments, the human cells are engineered cells. In certain embodiments, the human cells are non-engineered cells. In certain embodiments, the mammalian cells are non-human cells.

In specific embodiments, the non-human cells are engineered cells or non-engineered cells.

In some embodiments, the population of cells comprising T cells are obtained from a subject. In certain embodiments, the population of cells comprising T cells are obtained from a healthy subject. In certain embodiments, the population of cells comprising T cells are obtained from an unhealthy subject. In certain embodiments, the unhealthy subject has a solid tumor cancer. In certain embodiments, the unhealthy subject has a blood cancer. In certain embodiments, the unhealthy subject has both a solid tumor cancer and a blood cancer. In certain embodiments, the unhealthy subject has an autoimmune or an inflammatory disease. In certain embodiments, the unhealthy subject has a neurological disease.

### 4.2.2. Activation and Expansion Conditions

The methods provided herein comprise a step of ex vivo activation and expansion of Vγ9Vδ2 T cells in a culture system.

In some embodiments, the activation and expansion conditions comprise cytokines. Non-limiting examples of cytokines that can be used with the presently disclosed subject matter include lectin, hepatic growth factor, prostaglandin, fibroblast growth factor, prolactin, placental lactogen, OB protein, tumor necrosis factor-a, tumor necrosis factor-β, mullerian-inhibiting substance, mouse gonadotropin-associated peptide, inhibin, activin, vascular endothelial growth factor, integrin, thrombopoietin (TPO), a nerve growth factor (NGF), platelet-growth factor, TGF-α, TGF-β, insulin-like growth factor-I, insulin-like growth factor-II, erythropoietin (EPO), an osteoinductive factor, interferon-α, interferon-β, interferon-2, macrophage-CSF (M-CSF), granulocyte-macrophage-CSF (GM-CSF), granulocyte-CSF (G-CSF), interleukin-1 (IL-1), IL-1a, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, LIF, kit-ligand, FLT-3, angiostatin, thrombospondin, endostatin, tumor necrosis factor, and LT (lymphotoxin).

In some embodiments, the activation and expansion conditions comprise substances other than cytokines. In some embodiments, the activation and expansion conditions comprise an anti-Vγ9 mAb. In some embodiments, the activation and expansion conditions comprise a bisphosphonate. In some embodiments, the activation and expansion conditions comprise a mevalonate pathway intermediate. In certain embodiments, the bisphosphonate is selected from the group consisting of zoledronic acid, risedronic acid, ibandronic acid, alendronic acid, pamidronic acid, tiludronic acid, etidronic acid, and clodronic acid. In certain embodiments, the mevalonate pathway intermediate is selected from the group consisting of HMBPP, BrHPP, and isopentenyl pyrophosphate. In certain embodiments, the bisphosphonate is zoledronic acid. In certain embodiments, the bisphosphonate is risedronic acid. In certain embodiments, the bisphosphonate is ibandronic acid. In certain embodiments, the bisphosphonate is alendronic acid. In certain embodiments, the bisphosphonate is pamidronic acid. In certain embodiments, the bisphosphonate is tiludronic acid. In certain embodiments, the bisphosphonate is etidronic acid. In certain embodiments, the bisphosphonate is clodronic acid. In certain embodiments, the mevalonate pathway intermediate is HMBPP. In certain embodiments, the mevalonate pathway intermediate is BrHPP. In certain embodiments, the mevalonate pathway intermediate is isopentenyl pyrophosphate.

In some embodiments, the activation and expansion conditions comprise both cytokines and a bisphosphonate. In some embodiments, the activation and expansion conditions comprise both cytokines and a mevalonate pathway intermediate.

In certain embodiments, the activation and expansion conditions comprise IL-2, IL-15, and zoledronic acid.

In some embodiments, the IL-2 concentration within the culture system is from 10 IU/mL to 1200 IU/mL. In some embodiments, the IL-2 concentration within the culture system is from 50 IU/mL to 1200 IU/mL. In some embodiments, the IL-2 concentration within the culture system is from 100 IU/mL to 1200 IU/mL. In some embodiments, the IL-2 concentration within the culture system is 100 IU/mL to 1100 IU/mL. In some embodiments, the IL-2 concentration within the culture system is from 100 IU/mL to 1000 IU/mL. In some embodiments, the IL-2 concentration within the culture system is from 100 IU/mL to 1000 IU/mL. In certain embodiments, the IL-2 concentration within the culture system is 10 IU/mL. In certain embodiments, the IL-2 concentration within the culture system is 50 IU/mL. In certain embodiments, the IL-2 concentration within the culture system is 100 IU/mL. In certain embodiments, the IL-2 concentration within the culture system is 200 IU/mL. In certain embodiments, the IL-2 concentration within the culture system is 300 IU/mL. In certain embodiments, the IL-2 concentration within the culture system is 400 IU/mL. In certain embodiments, the IL-2 concentration within the culture system is 500 IU/mL. In certain embodiments, the IL-2 concentration within the culture system is 600 IU/mL. In certain embodiments, the IL-2 concentration within the culture system is 700 IU/mL. In certain embodiments, the IL-2 concentration within the culture system is 800 IU/mL. In certain embodiments, the IL-2 concentration within the culture system is 900 IU/mL. In certain embodiments, the IL-2 concentration within the culture system is 1000 IU/mL.

In some embodiments, the IL-2 concentration within the culture system is from 1 IU/mL to 100 IU/mL, from 1 IU/mL to 50 IU/mL, from 1 IU/mL to 40 IU/mL, from 1 IU/mL to 30 IU/mL, from 1 IU/mL to 20 IU/mL, from 5 IU/mL to 50 IU/mL, from 5 IU/mL to 30 IU/mL, from 5 IU/mL to 20 IU/mL, or from 5 IU/mL to 15 IU/mL. In certain embodiments, the IL-2 concentration within the culture system is about 10 IU/mL. In certain embodiments, the IL-2 concentration within the culture system is 10 IU/ml.

In some embodiments, the IL-2 concentration within the culture system remains the same during the culturing process. In some embodiments, the IL-2 concentration within the culture system is adjusted during the culturing process. In certain embodiments, the IL-2 concentration within the culture system is adjusted to gradually decrease along the culturing process. In certain embodiments, the IL-2 concentration within the culture system is the highest on days 0 and 1, is lower on days 2, 3 and 4, and is the lowest on day 5 and thereafter. In certain embodiments, the IL-2 concentration within the culture system is 1000 IU/mL or no more than 1000 IU/mL on days 0 and 1, 800 IU/mL or no more than 800 IU/mL on days 2, 3 and 4, and 100 IU/mL or no more than 100 IU/ml on day 5 and thereafter.

In some embodiments, the IL-15 concentration within the culture system is from 5 ng/mL to 25 ng/mL. In some embodiments, the IL-15 concentration within the culture system is from 5 ng/mL to 20 ng/mL. In some embodiments, the IL-15 concentration within the culture system is from 10 ng/ml to 20 ng/ml. In certain embodiments, the IL-15 concentration within the culture system is 5 ng/mL. In certain embodiments, the IL-15 concentration within the culture system is 8 ng/mL. In certain embodiments, the IL-15 concentration within the culture system is 10 ng/mL. In certain embodiments, the IL-15 concentration within the culture system is 13 ng/mL. In certain embodiments, the IL-15 concentration within the culture system is 15 ng/mL. In certain embodiments, the IL-15 concentration within the culture system is 18 ng/mL. In certain embodiments, the IL-15 concentration within the culture system is 20 ng/mL. In certain embodiments, the IL-15 concentration within the culture system is 23 ng/mL. In certain embodiments, the IL-15 concentration within the culture system is 25 ng/mL.

In some embodiments, the IL-15 concentration within the culture system is at least 50 ng/ml, at least 100 ng/ml, at least 150 ng/ml, at least 200 ng/ml, at least 250 ng/ml, at least 300 ng/ml, at least 400 ng/ml, at least 500 ng/ml, up to 50 ng/ml, up to 100 ng/ml, up to 150 ng/ml, up to 200 ng/ml, up to 250 ng/ml, up to 300 ng/ml, up to 400 ng/ml, and/or up to 500 ng/ml. In some embodiments, the IL-15 concentration within the culture system is from 50 ng/ml to 500 ng/ml, from 50 ng/ml to 400 ng/ml, from 300 ng/ml to 250 ng/ml, from 50 ng/ml to 200 ng/ml, from 50 ng/ml to 150 ng/ml, from 100 ng/ml to 500 ng/ml, from 100 ng/ml to 400 ng/ml, from 100 ng/ml to 300 ng/ml, from 100 ng/ml to 250 ng/ml, from 100 ng/ml to 200 ng/ml, from 150 ng/ml to 500 ng/ml, from 150 ng/ml to 400 ng/ml, from 150 ng/ml to 300 ng/ml, from 150 ng/ml to 250 ng/ml, from 150 ng/ml to 200 ng/ml, from 200 ng/ml to 500 ng/ml, from 200 ng/ml to 400 ng/ml, from 200 ng/ml to 300 ng/ml, from 250 ng/ml to 500 ng/ml, from 250 ng/ml to 400 ng/ml, from 250 ng/ml to 300 ng/ml, from 300 ng/ml to 500 ng/ml, from 300 ng/ml to 400 ng/ml, or from 400 ng/ml to 500 ng/ml. In some embodiments, the IL-15 concentration within the culture system is or is about 50 ng/ml, is or is about 100 ng/ml, is or is about 150 ng/ml, is or is about 200 ng/ml, is or is about 250 ng/ml, is or is about 300 ng/ml, is or is about 400 ng/ml, or is or is about 500 ng/ml.

In some embodiments, the IL-15 concentration within the culture system is from 50 ng/ml to 150 ng/ml, or from 150 ng/ml to 250 ng/ml. In some embodiments, the IL-15 concentration within the culture system at least 100 ng/ml. In some embodiments, the IL-15 concentration within the culture system at least 200 ng/ml. In some embodiments, the IL-15 concentration within the culture system is up to 100 ng/ml. In some embodiments, the IL-15 concentration within the culture system is up to 200 ng/ml. In some embodiments, the IL-15 concentration within the culture system is or is about 100 ng/ml. In some embodiments, the IL-15 concentration within the culture system is or is about 200 ng/ml.

In some embodiments, the IL-15 concentration within the culture system remains the same during the culturing process. In some embodiments, the IL-15 concentration within the culture system is adjusted during the culturing process. In certain embodiments, the IL-15 concentration within the culture system is the highest on days 2, 3 and 4, and is lower on days 0, 1, 5, and thereafter. In specific embodiments, the IL-15 concentration within the culture system is 10 ng/ml or no more than 10 ng/mL on days 0 and 1, 20 ng/ml or no more than 20 ng/mL on days 2, 3 and 4, and 10 ng/ml or no more than 10 ng/mL on day 5 and thereafter.

In some embodiments, the zoledronic acid concentration within the culture system is from 100 nM to 1000 nM. In some embodiments, the zoledronic acid concentration within the culture system is from 200 nM to 500 nM. In some embodiments, the zoledronic acid concentration within the culture system is from 300 nM to 400 nM. In certain embodiments, the zoledronic acid concentration within the culture system is 100 nM. In certain embodiments, the zoledronic acid concentration within the culture system is 150 nM. In certain embodiments, the zoledronic acid concentration within the culture system is 200 nM. In certain embodiments, the zoledronic acid concentration within the culture system is 250 nM. In certain embodiments, the zoledronic acid concentration within the culture system is 300 nM. In certain embodiments, the zoledronic acid concentration within the culture system is 350 nM. In certain embodiments, the zoledronic acid concentration within the culture system is 400 nM. In certain embodiments, the zoledronic acid concentration within the culture system is 450 nM. In certain embodiments, the zoledronic acid concentration within the culture system is 500 nM. In certain embodiments, the zoledronic acid concentration within the culture system is 550 nM. In certain embodiments, the zoledronic acid concentration within the culture system is 600 nM. In certain embodiments, the zoledronic acid concentration within the culture system is 650 nM. In certain embodiments, the zoledronic acid concentration within the culture system is 700 nM. In certain embodiments, the zoledronic acid concentration within the culture system is 750 nM. In certain embodiments, the zoledronic acid concentration within the culture system is 800 nM.

### 4.2.3. Expansion of Vγ9Vδ2 T cells under Hypoxia Conditions

The methods provided herein comprise a step of culturing the population of cells comprising T cells ex vivo in the culture system under a hypoxic condition for enhanced ex vivo activation and expansion of Vγ9Vδ2 T cells in the population of cells comprising T cells.

In some embodiments, the population of cells comprising T cells are ex vivo activated and expanded in the culture system under the hypoxic condition for at least 3 days, at least 5 days, at least 7 days, at least 9 days, at least 11 days, at least 13 days, at least 15 days, at least 17 days, at least 19 days, or at least 21 days. In some embodiments, the population of cells comprising T cells are ex vivo activated and expanded in the culture system under the hypoxic condition for at least 3 days. In some embodiments, the population of cells comprising T cells are ex vivo activated and expanded in the culture system under the hypoxic condition for at least 5 days. In some embodiments, the population of cells comprising T cells are ex vivo activated and expanded in the culture system under the hypoxic condition for at least 7 days. In some embodiments, the population of cells comprising T cells are ex vivo activated and expanded in the culture system under the hypoxic condition for at least 9 days. In some embodiments, the population of cells comprising T cells are ex vivo activated and expanded in the culture system under the hypoxic condition for at least 11 days. In some embodiments, the population of cells comprising T cells are ex vivo activated and expanded in the culture system under the hypoxic condition for at least 13 days. In some embodiments, the population of cells comprising T cells are ex vivo activated and expanded in the culture system under the hypoxic condition for at least 15 days. In some embodiments, the population of cells comprising T cells are ex vivo activated and expanded in the culture system under the hypoxic condition for at least 17 days. In some embodiments, the population of cells comprising T cells are ex vivo activated and expanded in the culture system under the hypoxic condition for at least 19 days. In some embodiments, the population of cells comprising T cells are ex vivo activated and expanded in the culture system under the hypoxic condition for at least 21 days. In certain embodiments, the population of cells comprising T cells are ex vivo activated and expanded in the culture system under the hypoxic condition for from 3 days to 28 days. In certain embodiments, the population of cells comprising T cells are ex vivo activated and expanded in the culture system under the hypoxic condition for from 3 days to 25 days. In certain embodiments, the population of cells comprising T cells are ex vivo activated and expanded in the culture system under the hypoxic condition for from 4 days to 23 days. In certain embodiments, the population of cells comprising T cells are ex vivo activated and expanded in the culture system under the hypoxic condition for from 5 days to 21 days. In certain embodiments, the population of cells comprising T cells are ex vivo activated and expanded in the culture system under the hypoxic condition for from 6 days to 19 days. In certain embodiments, the population of cells comprising T cells are ex vivo activated and expanded in the culture system under the hypoxic condition for from 7 days to 17 days. In certain embodiments, the population of cells comprising T cells are ex vivo activated and expanded in the culture system under the hypoxic condition for from 8 days to 15 days. In certain embodiments, the population of cells comprising T cells are ex vivo activated and expanded in the culture system under the hypoxic condition for from 9 days to 14 days. In certain embodiments, the population of cells comprising T cells are ex vivo activated and expanded in the culture system under the hypoxic condition for from 10 days to 14 days. In certain embodiments, the population of cells comprising T cells are ex vivo activated and expanded in the culture system under the hypoxic condition for from 11 days to 14 days. In certain embodiments, the population of cells comprising T cells are ex vivo activated and expanded in the culture system under the hypoxic condition for from 12 days to 14 days. In certain embodiments, the population of cells comprising T cells are ex vivo activated and expanded in the culture system under the hypoxic condition for about 10 days. In certain embodiments, the population of cells comprising T cells are ex vivo activated and expanded in the culture system under the hypoxic condition for about 11 days. In certain embodiments, the population of cells comprising T cells are ex vivo activated and expanded in the culture system under the hypoxic condition for about 12 days. In certain embodiments, the population of cells comprising T cells are ex vivo activated and expanded in the culture system under the hypoxic condition for about 13 days. In certain embodiments, the population of cells comprising T cells are ex vivo activated and expanded in the culture system under the hypoxic condition for about 14 days. In certain embodiments, the population of cells comprising T cells are ex vivo activated and expanded in the culture system under the hypoxic condition for about 15 days. In certain embodiments, the population of cells comprising T cells are ex vivo activated and expanded in the culture system under the hypoxic condition for about 16 days. In certain embodiments, the population of cells comprising T cells are ex vivo activated and expanded in the culture system under the hypoxic condition for about 17 days. In certain embodiments, the population of cells comprising T cells are ex vivo activated and expanded in the culture system under the hypoxic condition for about 18 days. In certain embodiments, the population of cells comprising T cells are ex vivo activated and expanded in the culture system under the hypoxic condition for about 19 days. In certain embodiments, the population of cells comprising T cells are ex vivo activated and expanded in the culture system under the hypoxic condition for about 20 days. In certain embodiments, the population of cells comprising T cells are ex vivo activated and expanded in the culture system under the hypoxic condition for about 21 days.

In some embodiments, the oxygen concentration of the hypoxic condition is less than 17%, less than 15%, less than 13%, less than 11%, less than 9%, less than 7%, less than 5%, less than 3%, less than 1%, or less than 0.5%. In some embodiments, the oxygen concentration of the hypoxic condition is less than 17%. In some embodiments, the oxygen concentration of the hypoxic condition is less than 15%. In some embodiments, the oxygen concentration of the hypoxic condition is less than 13%. In some embodiments, the oxygen concentration of the hypoxic condition is less than 11%. In some embodiments, the oxygen concentration of the hypoxic condition is less than 9%. In some embodiments, the oxygen concentration of the hypoxic condition is less than 7%. In some embodiments, the oxygen concentration of the hypoxic condition is less than 5%. In some embodiments, the oxygen concentration of the hypoxic condition is less than 3%. In some embodiments, the oxygen concentration of the hypoxic condition is less than 1%. In some embodiments, the oxygen concentration of the hypoxic condition is less than 0.5%. In certain embodiments, the oxygen concentration of the hypoxic condition is from 0.1% to 17%. In certain embodiments, the oxygen concentration of the hypoxic condition is from 0.1% to 15%. In certain embodiments, the oxygen concentration of the hypoxic condition is from 0.5% to 13%. In certain embodiments, the oxygen concentration of the hypoxic condition is from 1% to 13%. In certain embodiments, the oxygen concentration of the hypoxic condition is from 1% to 11%. In certain embodiments, the oxygen concentration of the hypoxic condition is from 1% to 9%. In certain embodiments, the oxygen concentration of the hypoxic condition is from 1% to 7%. In certain embodiments, the oxygen concentration of the hypoxic condition is from 2% to 5%. In certain embodiments, the oxygen concentration of the hypoxic condition is about 15%. In certain embodiments, the oxygen concentration of the hypoxic condition is about 14%. In certain embodiments, the oxygen concentration of the hypoxic condition is about 13%. In certain embodiments, the oxygen concentration of the hypoxic condition is about 12%. In certain embodiments, the oxygen concentration of the hypoxic condition is about 11%. In certain embodiments, the oxygen concentration of the hypoxic condition is about 10%. In certain embodiments, the oxygen concentration of the hypoxic condition is about 9%. In certain embodiments, the oxygen concentration of the hypoxic condition is about 8%. In certain embodiments, the oxygen concentration of the hypoxic condition is about 7%. In certain embodiments, the oxygen concentration of the hypoxic condition is about 6%. In certain embodiments, the oxygen concentration of the hypoxic condition is about 5%. In certain embodiments, the oxygen concentration of the hypoxic condition is about 4%. In certain embodiments, the oxygen concentration of the hypoxic condition is about 3%. In certain embodiments, the oxygen concentration of the hypoxic condition is about 2%. In certain embodiments, the oxygen concentration of the hypoxic condition is about 1%.

In some embodiments, the methods provided herein further comprise culturing the population of cells comprising T cells ex vivo in the culture system under a normoxic condition to activate and expand Vγ9Vδ2 T cells in the population of cells comprising T cells prior to culturing the population of cells comprising T cells ex vivo in the culture system under a hypoxic condition. In certain embodiments, the population of cells comprising T cells are ex vivo activated and expanded under the normoxic condition for at least 1 hour. In certain embodiments, the population of cells comprising T cells are ex vivo activated and expanded under the normoxic condition for at least 6 hours. In certain embodiments, the population of cells comprising T cells are ex vivo activated and expanded under the normoxic condition for at least 0.5 day. In certain embodiments, the population of cells comprising T cells are ex vivo activated and expanded under the normoxic condition for at least 1 day. In certain embodiments, the population of cells comprising T cells are ex vivo activated and expanded under the normoxic condition for at least 2 days. In certain embodiments, the population of cells comprising T cells are ex vivo activated and expanded under the normoxic condition for at least 3 days. In certain embodiments, the population of cells comprising T cells are ex vivo activated and expanded under the normoxic condition for at least 4 days. In certain embodiments, the population of cells comprising T cells are ex vivo activated and expanded under the normoxic condition for at least 5 days. In certain embodiments, the population of cells comprising T cells are ex vivo activated and expanded under the normoxic condition for from 1 hour to 7 days. In certain embodiments, the population of cells comprising T cells are ex vivo activated and expanded under the normoxic condition for from 6 hours to 7 days. In certain embodiments, the population of cells comprising T cells are ex vivo activated and expanded under the normoxic condition for from 0.5 day to 7 days. In certain embodiments, the population of cells comprising T cells are ex vivo activated and expanded under the normoxic condition for from 1 day to 7 days. In certain embodiments, the population of cells comprising T cells are ex vivo activated and expanded under the normoxic condition for from 1 day to 6 days. In certain embodiments, the population of cells comprising T cells are ex vivo activated and expanded under the normoxic condition for from 1 day to 5 days. In certain embodiments, the population of cells comprising T cells are ex vivo activated and expanded under the normoxic condition for from 1 day to 4 days. In certain embodiments, the population of cells comprising T cells are ex vivo activated and expanded under the normoxic condition for from 1 day to 3 days. In certain embodiments, the population of cells comprising T cells are ex vivo activated and expanded under the normoxic condition for from 1 day to 2 days.

In some embodiments, the oxygen concentration of the normoxic condition is at least 18%. In some embodiments, the oxygen concentration of the normoxic condition is at least 19%. In some embodiments, the oxygen concentration of the normoxic condition is at least 20%. In certain embodiments, the oxygen concentration of the normoxic condition is from 18% to 22%. In certain embodiments, the oxygen concentration of the normoxic condition is from 18% to 21%. In certain embodiments, the oxygen concentration of the normoxic condition is from 18% to 20%. In certain embodiments, the oxygen concentration of the normoxic condition is or is about 18%. In certain embodiments, the oxygen concentration of the normoxic condition is or is about 18.2%. In certain embodiments, the oxygen concentration of the normoxic condition is or is about 18.6%. In certain embodiments, the oxygen concentration of the normoxic condition is or is about 19%. In certain embodiments, the oxygen concentration of the normoxic condition is or is about 19.5%. In certain embodiments, the oxygen concentration of the normoxic condition is or is about 20%. In certain embodiments, the oxygen concentration of the normoxic condition is or is about 20.5%. In certain embodiments, the oxygen concentration of the normoxic condition is or is about 21%.

In some embodiments, the amount of a specific type of cells is measured by methods well known to those skilled in the art. In some embodiments, the amount of a specific type of cells is measured by flow cytometric analysis.

As disclosed herein, the total percentage of Vγ9Vδ2 T cells in the population of cells is calculated by dividing the total number of Vγ9Vδ2 T cell as measured by the methods known in the art (e.g., flow cytometric analysis, e.g., methods disclosed in Section 6.1.8 of the present disclosure) by the total number of cells in the population (i.e., total number of Vγ9Vδ2 T cell/total number of cells in the population).

In some embodiments, the methods provided herein increase the total percentage of Vγ9Vδ2 T cells in the population of cells comprising T cells to more than 10%, more than 15%, more than 20%, more than 25%, more than 30%, more than 35%, more than 40%, more than 45%, more than 50%, more than 55%, or more than 60%. In some embodiments, the methods provided herein increase the total percentage of Vγ9Vδ2 T cells in the population of cells comprising T cells to more than 10%. In some embodiments, the methods provided herein increase the total percentage of Vγ9Vδ2 T cells in the population of cells comprising T cells to more than 15%. In some embodiments, the methods provided herein increase the total percentage of Vγ9Vδ2 T cells in the population of cells comprising T cells to more than 20%. In some embodiments, the methods provided herein increase the total percentage of Vγ9Vδ2 T cells in the population of cells comprising T cells to more than 25%. In some embodiments, the methods provided herein increase the total percentage of Vγ9Vδ2 T cells in the population of cells comprising T cells to more than 30%. In some embodiments, the methods provided herein increase the total percentage of Vγ9Vδ2 T cells in the population of cells comprising T cells to more than 35%. In some embodiments, the methods provided herein increase the total percentage of Vγ9Vδ2 T cells in the population of cells comprising T cells to more than 40%. In some embodiments, the methods provided herein increase the total percentage of Vγ9Vδ2 T cells in the population of cells comprising T cells to more than 45%. In some embodiments, the methods provided herein increase the total percentage of Vγ9Vδ2 T cells in the population of cells comprising T cells to more than 50%. In some embodiments, the methods provided herein increase the total percentage of Vγ9Vδ2 T cells in the population of cells comprising T cells to more than 55%. In some embodiments, the methods provided herein increase the total percentage of Vγ9Vδ2 T cells in the population of cells comprising T cells to more than 60%. In certain embodiments, the methods provided herein increase the total percentage of Vγ9Vδ2 T cells in the population of cells comprising T cells to from 10% to 99%. In certain embodiments, the methods provided herein increase the total percentage of Vγ9Vδ2 T cells in the population of cells comprising T cells to from 20% to 95%. In certain embodiments, the methods provided herein increase the total percentage of Vγ9Vδ2 T cells in the population of cells comprising T cells to from 30% to 95%. In certain embodiments, the methods provided herein increase the total percentage of Vγ9Vδ2 T cells in the population of cells comprising T cells to from 35% to 95%. In certain embodiments, the methods provided herein increase the total percentage of Vγ9Vδ2 T cells in the population of cells comprising T cells to from 40% to 95%. In certain embodiments, the methods provided herein increase the total percentage of Vγ9Vδ2 T cells in the population of cells comprising T cells to from 45% to 95%. In certain embodiments, the methods provided herein increase the total percentage of Vγ9Vδ2 T cells in the population of cells comprising T cells to from 50% to 95%. In certain embodiments, the methods provided herein increase the total percentage of Vγ9Vδ2 T cells in the population of cells comprising T cells to from 60% to 95%. In certain embodiments, the methods provided herein increase the total percentage of Vγ9Vδ2 T cells in the population of cells comprising T cells to from 65% to 95%. In certain embodiments, the methods provided herein increase the total percentage of Vγ9Vδ2 T cells in the population of cells comprising T cells to about 50%. In certain embodiments, the methods provided herein increase the total percentage of Vγ9Vδ2 T cells in the population of cells comprising T cells to about 55%. In certain embodiments, the methods provided herein increase the total percentage of Vγ9Vδ2 T cells in the population of cells comprising T cells to about 60%. In certain embodiments, the methods provided herein increase the total percentage of Vγ9Vδ2 T cells in the population of cells comprising T cells to about 65%. In certain embodiments, the methods provided herein increase the total percentage of Vγ9Vδ2 T cells in the population of cells comprising T cells to about 70%. In certain embodiments, the methods provided herein increase the total percentage of Vγ9Vδ2 T cells in the population of cells comprising T cells to about 75%. In certain embodiments, the methods provided herein increase the total percentage of Vγ9Vδ2 T cells in the population of cells comprising T cells to about 80%. In certain embodiments, the methods provided herein increase the total percentage of Vγ9Vδ2 T cells in the population of cells comprising T cells to about 85%. In certain embodiments, the methods provided herein increase the total percentage of Vγ9Vδ2 T cells in the population of cells comprising T cells to about 90%. In certain embodiments, the methods provided herein increase the total percentage of Vγ9Vδ2 T cells in the population of cells comprising T cells to about 95%.

In some embodiments, the methods provided herein increase the total number of Vγ9Vδ2 T cells in the population of cells comprising T cells by at least 10-fold, at least 30-fold, at least 50-fold, at least 100-fold, at least 150-fold, at least 200-fold, at least 250-fold, at least 300-fold, at least 350-fold, at least 400-fold, at least 450-fold, at least 500-fold, at least 550-fold, or at least 600-fold as compared to the total number of Vγ9Vδ2 T cells in the population of cells comprising T cells before the expansion. In some embodiments, the methods provided herein increase the total number of Vγ9Vδ2 T cells in the population of cells comprising T cells by at least 10-fold. In some embodiments, the methods provided herein increase the total number of Vγ9Vδ2 T cells in the population of cells comprising T cells by at least 30-fold. In some embodiments, the methods provided herein increase the total number of Vγ9Vδ2 T cells in the population of cells comprising T cells by at least 50-fold. In some embodiments, the methods provided herein increase the total number of Vγ9Vδ2 T cells in the population of cells comprising T cells by at least 100-fold. In some embodiments, the methods provided herein increase the total number of Vγ9Vδ2 T cells in the population of cells comprising T cells by at least 150-fold. In some embodiments, the methods provided herein increase the total number of Vγ9Vδ2 T cells in the population of cells comprising T cells by at least 200-fold. In some embodiments, the methods provided herein increase the total number of Vγ9Vδ2 T cells in the population of cells comprising T cells by at least 250-fold. In some embodiments, the methods provided herein increase the total number of Vγ9Vδ2 T cells in the population of cells comprising T cells by at least 300-fold. In some embodiments, the methods provided herein increase the total number of Vγ9Vδ2 T cells in the population of cells comprising T cells by at least 350-fold. In some embodiments, the methods provided herein increase the total number of Vγ9Vδ2 T cells in the population of cells comprising T cells by at least 400-fold. In some embodiments, the methods provided herein increase the total number of Vγ9Vδ2 T cells in the population of cells comprising T cells by at least 450-fold. In some embodiments, the methods provided herein increase the total number of Vγ9Vδ2 T cells in the population of cells comprising T cells by at least 500-fold. In some embodiments, the methods provided herein increase the total number of Vγ9Vδ2 T cells in the population of cells comprising T cells by at least 550-fold. In some embodiments, the methods provided herein increase the total number of Vγ9Vδ2 T cells in the population of cells comprising T cells by at least 600-fold. In certain embodiments, the methods provided herein increase the total number of Vγ9Vδ2 T cells in the population of cells comprising T cells by from 10-fold to 900-fold. In certain embodiments, the methods provided herein increase the total number of Vγ9Vδ2 T cells in the population of cells comprising T cells by from 10-fold to 800-fold. In certain embodiments, the methods provided herein increase the total number of Vγ9Vδ2 T cells in the population of cells comprising T cells by from 30-fold to 700-fold. In certain embodiments, the methods provided herein increase the total number of Vγ9Vδ2 T cells in the population of cells comprising T cells by from 30-fold to 650-fold. In certain embodiments, the methods provided herein increase the total number of Vγ9Vδ2 T cells in the population of cells comprising T cells by from 50-fold to 600-fold. In certain embodiments, the methods provided herein increase the total number of Vγ9Vδ2 T cells in the population of cells comprising T cells by from 100-fold and 600-fold. In certain embodiments, the methods provided herein increase the total number of Vγ9Vδ2 T cells in the population of cells comprising T cells by from 150-fold to 600-fold. In certain embodiments, the methods provided herein increase the total number of Vγ9Vδ2 T cells in the population of cells comprising T cells by from 200-fold to 600-fold. In certain embodiments, the methods provided herein increase the total number of Vγ9Vδ2 T cells in the population of cells comprising T cells by from 250-fold to 600-fold. In certain embodiments, the methods provided herein increase the total number of Vγ9Vδ2 T cells in the population of cells comprising T cells by from 300-fold to 600-fold. In certain embodiments, the methods provided herein increase the total number of Vγ9Vδ2 T cells in the population of cells comprising T cells by from 350-fold to 600-fold. In certain embodiments, the methods provided herein increase the total number of Vγ9Vδ2 T cells in the population of cells comprising T cells by from 400-fold to 600-fold. In certain embodiments, the methods provided herein increase the total number of Vγ9Vδ2 T cells in the population of cells comprising T cells by from 450-fold to 600-fold. In certain embodiments, the methods provided herein increase the total number of Vγ9Vδ2 T cells in the population of cells comprising T cells by from 500-fold to 600-fold.

### 4.2.4. Enrichment of Ex Vivo Expanded Vγ9Vδ2 T Cells

In some embodiments, the methods provided here further comprise isolating or enriching Vγ9Vδ2 T cells from the population of cells comprising T cells after culturing the population of cells comprising T cells ex vivo in the culture system under a hypoxic condition for enhanced ex vivo activation and expansion of Vγ9Vδ2 T cells in the population of cells comprising T cells.

In certain embodiments, the methods comprise isolating Vγ9⁺ cells, and subsequently isolating Vδ2⁺ cells. In certain embodiments, the methods comprise isolating Vδ2⁺ cells, and subsequently isolating Vγ9⁺ cells. In certain embodiments, the method comprises isolating Vγ9⁺ cells and Vδ2⁺ cells simultaneously. In certain embodiments, the method for isolating or enriching Vγ9Vδ2 T cells is as described in Section 6.1.5 below.

The methods of isolating or enriching Vγ9⁺ cells are known to those skilled in the art. In some embodiments, the method of enriching Vγ9⁺ cells comprises a positive selection. In some embodiments, the method of enriching Vγ9⁺ cells comprises a negative selection.

In some embodiments, the method of enriching Vγ9⁺ cells comprises a negative selection comprising incubating the cell mixture with reagents that bind to undesired cells. In some embodiments, the method of enriching Vγ9⁺ cells comprises density gradient centrifugation, using, for example albumin, dextran, Ficoll, metrizamid, Percoll, and/or the like, to remove undesired cells.

In some embodiments, the method of enriching Vγ9⁺ cells comprises a positive selection comprising selecting or sorting cells having a cell surface expression of Vγ9. In some embodiments, the method of enriching Vγ9⁺ cells comprises FACS sorting of Vγ9⁺ cells. In some embodiments, the method of enriching Vγ9⁺ cells comprises using an anti-Vγ9 antibody that targets any one of Vγ9 chains. In some embodiments, the method of enriching Vγ9⁺ cells comprises an affinity column immobilized with a binding agent to Vγ9. In some embodiments, the method of enriching Vγ9⁺ cells comprises a combination of two or more of the above methods.

In certain embodiments, the method of enriching Vγ9⁺ cells comprises using magnetic beads coated with an anti-Vγ9 antibody. In certain embodiments, the anti-Vγ9 antibody-coated magnetic beads are coated with a secondary reagent that binds to the anti-Vγ9 antibody. In certain embodiments, the method of enriching Vγ9⁺ cells comprises using magnetic microparticles coated with an anti-Vγ9 antibody. In certain embodiments, the anti-Vγ9 antibody-coated magnetic microparticles are coated with a secondary reagent that binds to the anti-Vγ9 antibody. In certain embodiments, the method of enriching Vγ9⁺ cells comprises using magnetic nanoparticles coated with an anti-Vγ9 antibody. In certain embodiments, the anti-Vγ9 antibody-coated magnetic nanoparticles are coated with a secondary reagent that binds to the anti-Vγ9 antibody.

The methods of isolating or enriching Vδ2⁺ cells are known to those skilled in the art. In some embodiments, the method of enriching Vδ2⁺ cells comprises a positive selection. In some embodiments, the method of enriching Vδ2⁺ cells comprises a negative selection.

In some embodiments, the method of enriching Vδ2⁺ cells comprises a negative selection comprising incubating the cell mixture with reagents that bind to undesired cells. In some embodiments, the method of enriching Vδ2⁺ cells comprises density gradient centrifugation, using, for example albumin, dextran, Ficoll, metrizamid, Percoll, and/or the like, to remove undesired cells. In some embodiments, the method of enriching Vδ2⁺ cells comprises a positive selection comprising selecting or sorting cells having a cell surface expression of Vδ2. In some embodiments, the method of enriching Vδ2⁺ cells comprises FACS sorting of Vδ2⁺ cells. In some embodiments, the method of enriching Vδ2⁺ cells comprises using an anti-Vδ2 antibody which targets any one of Vδ2 chains. In some embodiments, the method of enriching Vδ2⁺ cells comprises an affinity column immobilized with a binding agent to Vδ2. In some embodiments, the method of enriching Vδ2⁺ cells comprises a combination of two or more of the above methods.

In certain embodiments, the method of enriching Vδ2⁺ cells comprises using magnetic beads coated with an anti-Vδ2 antibody. In certain embodiments, the anti-Vδ2 antibody-coated magnetic beads are coated with a secondary reagent that binds to the anti-Vδ2 antibody. In certain embodiments, the method of enriching Vδ2⁺ cells comprises using magnetic microparticles coated with an anti-Vδ2 antibody, In certain embodiments, the anti-Vδ2 antibody-coated magnetic microparticles are coated with a secondary reagent that binds to the anti-Vδ2 antibody. In certain embodiments, the method of enriching Vδ2⁺ cells comprises using magnetic nanoparticles coated with an anti-Vδ2 antibody. In certain embodiments, the anti-Vδ2 antibody-coated magnetic nanoparticles are coated with a secondary reagent that binds to the anti-Vδ2 antibody.

In certain embodiments, after enriching Vγ9Vδ2 T cells from the population of cells comprising T cells, the percentage of Vγ9Vδ2 T cells in the population of cells is increased as compared to an unenriched population. In some embodiments, the percentage of Vγ9Vδ2 T cells in the enriched population of cells is greater than about 65%. In some embodiments, the percentage of Vγ9Vδ2 T cells in the enriched population of cells is greater than about 70%. In some embodiments, the percentage of Vγ9Vδ2 T cells in the enriched population of cells is greater than about 75%. In some embodiments, the percentage of Vγ9Vδ2 T cells in the enriched population of cells is greater than about 80%. In some embodiments, the percentage of Vγ9Vδ2 T cells in the enriched population of cells is greater than about 85%. In some embodiments, the percentage of Vγ9Vδ2 T cells in the enriched population of cells is greater than about 90%. In some embodiments, the percentage of Vγ9Vδ2 T cells in the enriched population of cells is greater than about 95%. In some embodiments, the percentage of Vγ9Vδ2 T cells in the enriched population of cells is greater than about 99%.

### 4.3. An Isolated Population of Vγ9Vδ2 T Cells

In another aspect, provided herein are isolated populations of Vγ9Vδ2 T cells produced by the methods provided in Section 4.2 above.

In some embodiments, the percentage of the Vγ9Vδ2 T cells in the isolated population of Vγ9Vδ2 T cells is more than 10%, more than 15%, more than 20%, more than 25%, more than 30%, more than 35%, more than 40%, more than 45%, more than 50%, more than 55%, or more than 60%. In some embodiments, the percentage of the Vγ9Vδ2 T cells in the isolated population of Vγ9Vδ2 T cells is more than 10%. In some embodiments, the percentage of the Vγ9Vδ2 T cells in the isolated population of Vγ9Vδ2 T cells is more than 15%. In some embodiments, the percentage of the Vγ9Vδ2 T cells in the isolated population of Vγ9Vδ2 T cells is more than 20%. In some embodiments, the percentage of the Vγ9Vδ2 T cells in the isolated population of Vγ9Vδ2 T cells is more than 25%. In some embodiments, the percentage of the Vγ9Vδ2 T cells in the isolated population of Vγ9Vδ2 T cells is more than 30%. In some embodiments, the percentage of the Vγ9Vδ2 T cells in the isolated population of Vγ9Vδ2 T cells is more than 35%. In some embodiments, the percentage of the Vγ9Vδ2 T cells in the isolated population of Vγ9Vδ2 T cells is more than 40%. In some embodiments, the percentage of the Vγ9Vδ2 T cells in the isolated population of Vγ9Vδ2 T cells is more than 45%. In some embodiments, the percentage of the Vγ9Vδ2 T cells in the isolated population of Vγ9Vδ2 T cells is more than 50%. In some embodiments, the percentage of the Vγ9Vδ2 T cells in the isolated population of Vγ9Vδ2 T cells is more than 55%. In some embodiments, the percentage of the Vγ9Vδ2 T cells in the isolated population of Vγ9Vδ2 T cells is more than 60%. In certain embodiments, the percentage of the Vγ9Vδ2 T cells in the isolated population of Vγ9Vδ2 T cells is from 10% to 99%. In certain embodiments, the percentage of the Vγ9Vδ2 T cells in the isolated population of Vγ9Vδ2 T cells is from 20% to 95%. In certain embodiments, the percentage of the Vγ9Vδ2 T cells in the isolated population of Vγ9Vδ2 T cells is from 30% to 95%. In certain embodiments, the percentage of the Vγ9Vδ2 T cells in the isolated population of Vγ9Vδ2 T cells is from 35% to 95%. In certain embodiments, the percentage of the Vγ9Vδ2 T cells in the isolated population of Vγ9Vδ2 T cells is from 40% to 95%. In certain embodiments, the percentage of the Vγ9Vδ2 T cells in the isolated population of Vγ9Vδ2 T cells is from 45% to 95%. In certain embodiments, the percentage of the Vγ9Vδ2 T cells in the isolated population of Vγ9Vδ2 T cells is from 50% to 95%. In certain embodiments, the percentage of the Vγ9Vδ2 T cells in the isolated population of Vγ9Vδ2 T cells is from 60% to 95%. In certain embodiments, the percentage of the Vγ9Vδ2 T cells in the isolated population of Vγ9Vδ2 T cells is from 65% to 95%. In certain embodiments, the percentage of the Vγ9Vδ2 T cells in the isolated population of Vγ9Vδ2 T cells is about 50%. In certain embodiments, the percentage of the Vγ9Vδ2 T cells in the isolated population of Vγ9Vδ2 T cells is about 55%. In certain embodiments, the percentage of the Vγ9Vδ2 T cells in the isolated population of Vγ9Vδ2 T cells is about 60%. In certain embodiments, the percentage of the Vγ9Vδ2 T cells in the isolated population of Vγ9Vδ2 T cells is about 65%. In certain embodiments, the percentage of the Vγ9Vδ2 T cells in the isolated population of Vγ9Vδ2 T cells is about 70%. In certain embodiments, the percentage of the Vγ9Vδ2 T cells in the isolated population of Vγ9Vδ2 T cells is about 75%. In certain embodiments, the percentage of the Vγ9Vδ2 T cells in the isolated population of Vγ9Vδ2 T cells is about 80%. In certain embodiments, the percentage of the Vγ9Vδ2 T cells in the isolated population of Vγ9Vδ2 T cells is about 85%. In certain embodiments, the percentage of the Vγ9Vδ2 T cells in the isolated population of Vγ9Vδ2 T cells is about 90%. In certain embodiments, the percentage of the Vγ9Vδ2 T cells in the isolated population of Vγ9Vδ2 T cells is about 95%.

In yet another aspect, provided herein are methods for using the Vγ9Vδ2 T cells provided herein in combination with multispecific antibodies that are described in more details below in Sections 4.4, 4.7, 4.8, and 4.9.

In yet another aspect, provided herein are methods for using the Vγ9Vδ2 T cells provided herein, for example, for allogenic CAR T cell therapies that are described in more details below in Section 4.5 to Section 4.9.

### 4.4. Multispecific Antibodies for T Cells Redirection

In another aspect, provided herein are methods for using the Vγ9Vδ2 T cells provided herein in combination with multispecific antibodies so that the Vγ9Vδ2 T cells are directed to target cells.

In some embodiments, the multispecific antibodies are trispecific. In some embodiments, the multispecific antibodies are bispecific. In some embodiments, the multispecific antibody comprises a first binding domain that binds to an antigen expressed on a Vγ9Vδ2 T cell and a second binding domain that binds to an antigen expressed on an unhealthy cell.

Antigens expressed on Vγ9Vδ2 T cells are well known in the art. In some embodiments, the antigen expressed on the Vγ9Vδ2 T cell is T Cell Receptor Gamma Variable 9. In some embodiments, the antigen expressed on the Vγ9Vδ2 T cell is CD3.

In some embodiments, the unhealthy cell is a cancer cell. In certain embodiments, the cancer cell is a blood cancer cell or a solid tumor cancer cell. In some embodiments, the unhealthy cell is from a subject having an autoimmune and inflammatory disease. In some embodiments, the unhealthy cell is from a subject having a neurological disease.

In some embodiments, the antigen expressed on the unhealthy cell is a tumor antigen. Exemplary tumor antigens include, but are not limited to, a glioma-associated antigen, carcinoembryonic antigen (CEA), β-human chorionic gonadotropin, alphafetoprotein

(AFP), lectin-reactive AFP, thyroglobulin, RAGE-1, MN-CAIX, human telomerase reverse transcriptase, RUI, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, and mesothelin.

In some embodiments, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and gp 100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA).

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that induce a state of immunologic tolerance to the antigen. The expression of the antigen on the tumor may occur under conditions that enable the immune system to respond to the antigen. TAAs may be antigens that are expressed on normal cells during fetal development, when the immune system is immature and unable to respond, or they may be antigens that are normally present at extremely low levels on normal cells but are expressed at much higher levels on tumor cells.

Non-limiting examples of TSA or TAA antigens include: differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7.

Other large, protein-based antigens include TSP-180, MAGE-4, MAGE-5, MAGE-6, RAGE, NY-ESO, pl85erbB2, p180erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

### 4.5. Methods for Producing Vγ9Vδ2 T Cells Expressing Chimeric Antigen Receptors

### 4.5.1. Vγ9Vδ2 T Cells

In another aspect, provided herein are methods for making a chimeric antigen receptor (CAR) T cell product. In certain embodiments, the method comprises obtaining a population of cells comprising Vγ9Vδ2 T cells, and introducing a nucleic acid encoding a CAR into the Vγ9Vδ2 T cells. In some embodiments, the Vγ9Vδ2 T cells are produced according to the methods described in Section 4.2 above. In some embodiments, the Vγ9Vδ2 T cells are the cells described in Section 4.3 above.

More specifically, in some embodiments, provided herein are methods for making a CAR T cell. I In certain embodiments, the methodcomprises contacting a population of cells comprising T cells with a culture system comprising IL-2, IL-15, and a bisphosphonate or a mevalonate pathway intermediate, and culturing the population of cells comprising T cells ex vivo in the culture system under a hypoxic condition for enhanced ex vivo activation and expansion of Vγ9Vδ2 T cells in the population of cells comprising T cells.

T cells can be obtained from a number of sources. In some embodiments, the population of cells comprising T cells are obtained from a cultured T cell line. In some embodiments, the population of cells comprising T cells are collected, isolated, purified or induced from a body fluid, a tissue or an organ including but not limited to peripheral blood, umbilical cord blood, bone marrow, lymph node, spleen, or other tissues or fluids of a subject. In certain embodiments, the population of cells comprising T cells are peripheral blood lymphocytes, precursor cells of T cells (such as hematopoietic stem cells, lymphocyte precursor cells, etc.) or a cell population comprising them. Immature T cells may be found in the thymus.

In certain embodiments, the population of cells comprising T cells are peripheral blood mononuclear cells (PBMCs). In certain embodiments, the PBMCs are freshly obtained PBMCs. In certain embodiments, the PBMCs are frozen PBMCs. Various methods of collecting and preparing PBMCs are known in the art.

In certain embodiments, the population of cells comprising T cells are derived from a human tissue. In specific embodiments, the human tissue is fresh. In certain embodiments, the human tissue is frozen. Various methods of collecting and preparing human tissue are known in the art.

In certain embodiments, the population of cells comprising T cells are tumor-infiltrating lymphocytes (TILs). In certain embodiments, the TILs are freshly obtained TILs. In certain embodiments, the TILs are frozen TILs. Various methods of collecting and preparing TILs are known in the art.

In some embodiments, the population of cells comprising T cells are mammalian cells. In certain embodiments, the mammalian cells are human cells. In specific embodiments, the human cells are engineered cells. In specific embodiments, the human cells are non-engineered cells. In certain embodiments, the mammalian cells are non-human cells. In specific embodiments, the non-human cells are engineered cells. In specific embodiments, the non-human cells are non-engineered cells.

In some embodiments, the population of cells comprising T cells are obtained from a subject. In certain embodiments, the population of cells comprising T cells are obtained from a healthy subject. In certain embodiments, the population of cells comprising T cells are obtained from an unhealthy subject. In certain embodiments, the unhealthy subject has a solid tumor cancer. In certain embodiments, the unhealthy subject has a blood cancer. In certain embodiments, the unhealthy subject has both a solid tumor cancer and a blood cancer. In certain embodiments, the unhealthy subject has an autoimmune and inflammatory disease. In certain embodiments, the unhealthy subject has a neurological disease.

The population of cells comprising T cells are cultured ex vivo in a culture system for activation and expansion of Vγ9Vδ2 T cells.

In some embodiments, the activation and expansion conditions comprise cytokines. Non-limiting examples of cytokines include lectin, hepatic growth factor, prostaglandin, fibroblast growth factor, prolactin, placental lactogen, OB protein, tumor necrosis factor-a, tumor necrosis factor-β, mullerian-inhibiting substance, mouse gonadotropin-associated peptide, inhibin, activin, vascular endothelial growth factor, integrin, thrombopoietin (TPO), a nerve growth factor (NGF), platelet-growth factor, TGF-α, TGF-β, insulin-like growth factor-I, insulin-like growth factor-II, erythropoietin (EPO), an osteoinductive factor, interferon-α, interferon-β, interferon-2, macrophage-CSF (M-CSF), granulocyte-macrophage-CSF (GM-CSF), granulocyte-CSF (G-CSF), interleukin-1 (IL-1), IL-1a, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, LIF, kit-ligand, FLT-3, angiostatin, thrombospondin, endostatin, tumor necrosis factor, and LT (lymphotoxin).

In some embodiments, the activation and expansion conditions comprise substances other than cytokines. In some embodiments, the activation and expansion conditions comprise one or more anti-Vγ9 antibodies. In some embodiments, the activation and expansion conditions comprise a bisphosphonate. In some embodiments, the activation and expansion conditions comprise a mevalonate pathway intermediate. In certain embodiments, the bisphosphonate is selected from the group consisting of zoledronic acid, risedronic acid, ibandronic acid, alendronic acid, pamidronic acid, tiludronic acid, etidronic acid, and clodronic acid. In certain embodiments, the mevalonate pathway intermediate is selected from the group consisting of HMBPP, BrHPP, and isopentenyl pyrophosphate. In certain embodiments, the bisphosphonate is zoledronic acid. In certain embodiments, the bisphosphonate is risedronic acid. In certain embodiments, the bisphosphonate is ibandronic acid. In certain embodiments, the bisphosphonate is alendronic acid. In certain embodiments, the bisphosphonate is pamidronic acid. In certain embodiments, the bisphosphonate is tiludronic acid. In certain embodiments, the bisphosphonate is etidronic acid. In certain embodiments, the bisphosphonate is clodronic acid. In certain embodiments, the mevalonate pathway intermediate is HMBPP. In certain embodiments, the mevalonate pathway intermediate is BrHPP. In certain embodiments, the mevalonate pathway intermediate is isopentenyl pyrophosphate.

In some embodiments, the activation and expansion conditions comprise both cytokines and a bisphosphonate. In some embodiments, the activation and expansion conditions comprise both cytokines and a mevalonate pathway intermediate. In certain embodiments, the activation and expansion conditions comprise IL-2, IL-15, and zoledronic acid.

In some embodiments, the IL-2 concentration within the culture system is from 10 IU/mL to 1200 IU/mL. In some embodiments, the IL-2 concentration within the culture system is from 50 IU/mL to 1200 IU/mL. In some embodiments, the IL-2 concentration within the culture system is from 100 IU/mL to 1200 IU/mL. In some embodiments, the IL-2 concentration within the culture system is from 100 IU/mL to 1100 IU/mL. In some embodiments, the IL-2 concentration within the culture system is from 100 IU/mL to 1000 IU/mL. In certain embodiments, the IL-2 concentration within the culture system is 10 IU/mL. In certain embodiments, the IL-2 concentration within the culture system is 50

IU/mL. In certain embodiments, the IL-2 concentration within the culture system is 100 IU/mL. In certain embodiments, the IL-2 concentration within the culture system is 200 IU/mL. In certain embodiments, the IL-2 concentration within the culture system is 300 IU/mL. In certain embodiments, the IL-2 concentration within the culture system is 400 IU/mL. In certain embodiments, the IL-2 concentration within the culture system is 500 IU/mL. In certain embodiments, the IL-2 concentration within the culture system is 600 IU/mL. In certain embodiments, the IL-2 concentration within the culture system is 700 IU/mL. In certain embodiments, the IL-2 concentration within the culture system is 800 IU/mL. In certain embodiments, the IL-2 concentration within the culture system is 900 IU/mL. In certain embodiments, the IL-2 concentration within the culture system is 1000 IU/mL.

In some embodiments, the IL-2 concentration within the culture system is from 1 IU/mL to 100 IU/mL, from 1 IU/mL to 50 IU/mL, from 1 IU/mL to 40 IU/mL, from 1 IU/mL to 30 IU/mL, from 1 IU/mL to 20 IU/mL, from 5 IU/mL to 50 IU/mL, from 5 IU/mL to 30 IU/mL, from 5 IU/mL to 20 IU/mL, or from 5 IU/mL to 15 IU/mL. In certain embodiments, the IL-2 concentration within the culture system is about 10 IU/mL. In certain embodiments, the IL-2 concentration within the culture system is 10 IU/ml.

In some embodiments, the IL-2 concentration within the culture system remains the same during the culturing process. In some embodiments, the IL-2 concentration within the culture system is adjusted during the culturing process. In certain embodiments, the IL-2 concentration within the culture system is adjusted to gradually decrease during the culturing process. In certain embodiments, the IL-2 concentration within the culture system is the highest on days 0 and 1, is lower on days 2, 3 and 4, and is the lowest on day 5 and thereafter. In certain embodiments, the IL-2 concentration within the culture system is 1000 IU/mL or no more than 1000 IU/mL on days 0 and 1, 800 IU/mL or no more than 800 IU/mL on days 2, 3 and 4, and 100 IU/mL or no more than 100 IU/ml on day 5 and thereafter.

In some embodiments, the IL-15 concentration within the culture system is from 5 ng/ml to 25 ng/mL. In some embodiments, the IL-15 concentration within the culture system is from 5 ng/mL to 20 ng/mL. In some embodiments, the IL-15 concentration within the culture system is from 10 ng/ml to 20 ng/mL. In certain embodiments, the IL-15 concentration within the culture system is 5 ng/mL. In certain embodiments, the IL-15 concentration within the culture system is 8 ng/mL. In certain embodiments, the IL-15 concentration within the culture system is 10 ng/mL. In certain embodiments, the IL-15 concentration within the culture system is 13 ng/mL. In certain embodiments, the IL-15 concentration within the culture system is 15 ng/ml. In certain embodiments, the IL-15 concentration within the culture system is 18 ng/mL. In certain embodiments, the IL-15 concentration within the culture system is 20 ng/mL. In certain embodiments, the IL-15 concentration within the culture system is 23 ng/mL. In certain embodiments, the IL-15 concentration within the culture system is 25 ng/ml.

In some embodiments, the IL-15 concentration within the culture system is at least 50 ng/ml, at least 100 ng/ml, at least 150 ng/ml, at least 200 ng/ml, at least 250 ng/ml, at least 300 ng/ml, at least 400 ng/ml, at least 500 ng/ml, up to 50 ng/ml, up to 100 ng/ml, up to 150 ng/ml, up to 200 ng/ml, up to 250 ng/ml, up to 300 ng/ml, up to 400 ng/ml, or up to 500 ng/ml. In some embodiments, the IL-15 concentration within the culture system is from 50 ng/ml to 500 ng/ml, from 50 ng/ml to 400 ng/ml, from 300 ng/ml to 250 ng/ml, from 50 ng/ml to 200 ng/ml, from 50 ng/ml to 150 ng/ml, from 100 ng/ml to 500 ng/ml, from 100 ng/ml to 400 ng/ml, from 100 ng/ml to 300 ng/ml, from 100 ng/ml to 250 ng/ml, from 100 ng/ml to 200 ng/ml, from 150 ng/ml to 500 ng/ml, from 150 ng/ml to 400 ng/ml, from 150 ng/ml to 300 ng/ml, from 150 ng/ml to 250 ng/ml, from 150 ng/ml to 200 ng/ml, from 200 ng/ml to 500 ng/ml, from 200 ng/ml to 400 ng/ml, from 200 ng/ml to 300 ng/ml, from 250 ng/ml to 500 ng/ml, from 250 ng/ml to 400 ng/ml, from 250 ng/ml to 300 ng/ml, from 300 ng/ml to 500 ng/ml, from 300 ng/ml to 400 ng/ml, or from 400 ng/ml to 500 ng/ml. In some embodiments, the IL-15 concentration within the culture system is or is about 50 ng/ml, is or is about 100 ng/ml, is or is about 150 ng/ml, is or is about 200 ng/ml, is or is about 250 ng/ml, is or is about 300 ng/ml, is or is about 400 ng/ml, is or is about 500 ng/ml.

In some embodiments, the IL-15 concentration within the culture system is from 50 ng/ml to 150 ng/ml or from 150 ng/ml to 250 ng/ml. In some embodiments, the IL-15 concentration within the culture system at least 100 ng/ml. In some embodiments, the IL-15 concentration within the culture system at least 200 ng/ml. In some embodiments, the IL-15 concentration within the culture system up to 100 ng/ml. In some embodiments, the IL-15 concentration within the culture system up to 200 ng/ml. In some embodiments, the IL-15 concentration within the culture system is or is about 100 ng/ml. In some embodiments, the IL-15 concentration within the culture system is or is about 200 ng/ml.

In some embodiments, the IL-15 concentration within the culture system remains the same during the culturing process. In some embodiments, the IL-15 concentration within the culture system is adjusted during the culturing process. In certain embodiments, the IL-15 concentration within the culture system is the highest on days 2, 3 and 4, and is lower on days 0 and 1 and day 5 and thereafter. In specific embodiments, the IL-15 concentration within the culture system is 10 ng/ml or no more than 10 ng/ml on days 0 and 1, 20 ng/ml or no more than 20 ng/mL on days 2, 3 and 4, and 10 ng/ml or no more than 10 ng/ml on day 5 and thereafter.

In some embodiments, the zoledronic acid concentration within the culture system is from 100 nM to 1000 nM. In some embodiments, the zoledronic acid concentration within the culture system is from 200 nM to 500 nM. In some embodiments, the zoledronic acid concentration within the culture system is from 300 nM to 400 nM. In certain embodiments, the zoledronic acid concentration within the culture system is 100 nM. In certain embodiments, the zoledronic acid concentration within the culture system is 150 nM. In certain embodiments, the zoledronic acid concentration within the culture system is 200 nM.

In certain embodiments, the zoledronic acid concentration within the culture system is 250 nM. In certain embodiments, the zoledronic acid concentration within the culture system is 300 nM. In certain embodiments, the zoledronic acid concentration within the culture system is 350 nM. In certain embodiments, the zoledronic acid concentration within the culture system is 400 nM. In certain embodiments, the zoledronic acid concentration within the culture system is 450 nM. In certain embodiments, the zoledronic acid concentration within the culture system is 500 nM. In certain embodiments, the zoledronic acid concentration within the culture system is 550 nM. In certain embodiments, the zoledronic acid concentration within the culture system is 600 nM. In certain embodiments, the zoledronic acid concentration within the culture system is 650 nM. In certain embodiments, the zoledronic acid concentration within the culture system is 700 nM. In certain embodiments, the zoledronic acid concentration within the culture system is 750 nM. In certain embodiments, the zoledronic acid concentration within the culture system is 800 nM.

The population of cells comprising T cells are ex vivo activated and expanded in the culture system under a hypoxic condition for enhanced ex vivo activation and expansion of Vγ9Vδ2 T cells in the population of cells comprising T cells.

In some embodiments, the population of cells comprising T cells are ex vivo activated and expanded in the culture system under the hypoxic condition for at least 3 days, at least 5 days, at least 7 days, at least 9 days, at least 11 days, at least 13 days, at least 15 days, at least 17 days, at least 19 days, or at least 21 days. In some embodiments, the population of cells comprising T cells are ex vivo activated and expanded in the culture system under the hypoxic condition for at least 3 days. In some embodiments, the population of cells comprising T cells are ex vivo activated and expanded in the culture system under the hypoxic condition for at least 5 days. In some embodiments, the population of cells comprising T cells are ex vivo activated and expanded in the culture system under the hypoxic condition for at least 7 days. In some embodiments, the population of cells comprising T cells are ex vivo activated and expanded in the culture system under the hypoxic condition for at least 9 days. In some embodiments, the population of cells comprising T cells are ex vivo activated and expanded in the culture system under the hypoxic condition for at least 11 days. In some embodiments, the population of cells comprising T cells are ex vivo activated and expanded in the culture system under the hypoxic condition for at least 13 days. In some embodiments, the population of cells comprising T cells are ex vivo activated and expanded in the culture system under the hypoxic condition for at least 15 days. In some embodiments, the population of cells comprising T cells are ex vivo activated and expanded in the culture system under the hypoxic condition for at least 17 days. In some embodiments, the population of cells comprising T cells are ex vivo activated and expanded in the culture system under the hypoxic condition for at least 19 days. In some embodiments, the population of cells comprising T cells are ex vivo activated and expanded in the culture system under the hypoxic condition for at least 21 days. In certain embodiments, the population of cells comprising T cells are ex vivo activated and expanded in the culture system under the hypoxic condition for from 3 days to 28 days. In certain embodiments, the population of cells comprising T cells are ex vivo activated and expanded in the culture system under the hypoxic condition for from 3 days to 25 days. In certain embodiments, the population of cells comprising T cells are ex vivo activated and expanded in the culture system under the hypoxic condition for from 4 days to 23 days. In certain embodiments, the population of cells comprising T cells are ex vivo activated and expanded in the culture system under the hypoxic condition for from 5 to 21 days. In certain embodiments, the population of cells comprising T cells are ex vivo activated and expanded in the culture system under the hypoxic condition for from 6 days to 19 days. In certain embodiments, the population of cells comprising T cells are ex vivo activated and expanded in the culture system under the hypoxic condition from 7 days to 17 days. In certain embodiments, the population of cells comprising T cells are ex vivo activated and expanded in the culture system under the hypoxic condition from 8 days to 15 days. In certain embodiments, the population of cells comprising T cells are ex vivo activated and expanded in the culture system under the hypoxic condition for from 9 days to 14 days. In certain embodiments, the population of cells comprising T cells are ex vivo activated and expanded in the culture system under the hypoxic condition for from 10 days to 14 days. In certain embodiments, the population of cells comprising T cells are ex vivo activated and expanded in the culture system under the hypoxic condition for from 11 days to 14 days. In certain embodiments, the population of cells comprising T cells are ex vivo activated and expanded in the culture system under the hypoxic condition for from 12 days to 14 days. In certain embodiments, the population of cells comprising T cells are ex vivo activated and expanded in the culture system under the hypoxic condition for about 10 days. In certain embodiments, the population of cells comprising T cells are ex vivo activated and expanded in the culture system under the hypoxic condition for about 11 days. In certain embodiments, the population of cells comprising T cells are ex vivo activated and expanded in the culture system under the hypoxic condition for about 12 days. In certain embodiments, the population of cells comprising T cells are ex vivo activated and expanded in the culture system under the hypoxic condition for about 13 days. In certain embodiments, the population of cells comprising T cells are ex vivo activated and expanded in the culture system under the hypoxic condition for about 14 days. In certain embodiments, the population of cells comprising T cells are ex vivo activated and expanded in the culture system under the hypoxic condition for about 15 days. In certain embodiments, the population of cells comprising T cells are ex vivo activated and expanded in the culture system under the hypoxic condition for about 16 days. In certain embodiments, the population of cells comprising T cells are ex vivo activated and expanded in the culture system under the hypoxic condition for about 17 days. In certain embodiments, the population of cells comprising T cells are ex vivo activated and expanded in the culture system under the hypoxic condition for about 18 days. In certain embodiments, the population of cells comprising T cells are ex vivo activated and expanded in the culture system under the hypoxic condition for about 19 days. In certain embodiments, the population of cells comprising T cells are ex vivo activated and expanded in the culture system under the hypoxic condition for about 20 days. In certain embodiments, the population of cells comprising T cells are ex vivo activated and expanded in the culture system under the hypoxic condition for about 21 days.

In some embodiments, the oxygen concentration of the hypoxic condition is less than 17%, 15%, less than 13%, less than 11%, less than 9%, less than 7%, less than 5%, less than 3%, less than 1%, or less than 0.5%. In some embodiments, the oxygen concentration of the hypoxic condition is less than 17%. In some embodiments, the oxygen concentration of the hypoxic condition is less than 15%. In some embodiments, the oxygen concentration of the hypoxic condition is less than 13%. In some embodiments, the oxygen concentration of the hypoxic condition is less than 11%. In some embodiments, the oxygen concentration of the hypoxic condition is less than 9%. In some embodiments, the oxygen concentration of the hypoxic condition is less than 7%. In some embodiments, the oxygen concentration of the hypoxic condition is less than 5%. In some embodiments, the oxygen concentration of the hypoxic condition is less than 3%. In some embodiments, the oxygen concentration of the hypoxic condition is less than 1%. In some embodiments, the oxygen concentration of the hypoxic condition is less than 0.5%. In certain embodiments, the oxygen concentration of the hypoxic condition is from 0.1% to 17%. In certain embodiments, the oxygen concentration of the hypoxic condition is from 0.1% to 15%. In certain embodiments, the oxygen concentration of the hypoxic condition is from 0.5% to 13%. In certain embodiments, the oxygen concentration of the hypoxic condition is from 1% to 13%. In certain embodiments, the oxygen concentration of the hypoxic condition is from 1% to 11%. In certain embodiments, the oxygen concentration of the hypoxic condition is from 1% to 9%. In certain embodiments, the oxygen concentration of the hypoxic condition is from 1% to 7%. In certain embodiments, the oxygen concentration of the hypoxic condition is from 2% to 5%. In certain embodiments, the oxygen concentration of the hypoxic condition is about 15%. In certain embodiments, the oxygen concentration of the hypoxic condition is about 14%. In certain embodiments, the oxygen concentration of the hypoxic condition is about 13%. In certain embodiments, the oxygen concentration of the hypoxic condition is about 12%. In certain embodiments, the oxygen concentration of the hypoxic condition is about 11%. In certain embodiments, the oxygen concentration of the hypoxic condition is about 10%. In certain embodiments, the oxygen concentration of the hypoxic condition is about 9%. In certain embodiments, the oxygen concentration of the hypoxic condition is about 8%. In certain embodiments, the oxygen concentration of the hypoxic condition is about 7%. In certain embodiments, the oxygen concentration of the hypoxic condition is about 6%. In certain embodiments, the oxygen concentration of the hypoxic condition is about 5%. In certain embodiments, the oxygen concentration of the hypoxic condition is about 4%. In certain embodiments, the oxygen concentration of the hypoxic condition is about 3%. In certain embodiments, the oxygen concentration of the hypoxic condition is about 2%. In certain embodiments, the oxygen concentration of the hypoxic condition is about 1%.

In some embodiments, the population of cells comprising T cells are further cultured ex vivo in the culture system under a normoxic condition to activate and expand Vγ9Vδ2 T cells in the population of cells comprising T cells prior to being cultured ex vivo in the culture system under a hypoxic condition. In certain embodiments, the population of cells comprising T cells are ex vivo activated and expanded under the normoxic condition for at least 1 hour. In certain embodiments, the population of cells comprising T cells are ex vivo activated and expanded under the normoxic condition for at least 6 hours. In certain embodiments, the population of cells comprising T cells are ex vivo activated and expanded under the normoxic condition for at least 0.5 day. In certain embodiments, the population of cells comprising T cells are ex vivo activated and expanded under the normoxic condition for at least 1 day. In certain embodiments, the population of cells comprising T cells are ex vivo activated and expanded under the normoxic condition for at least 2 days. In certain embodiments, the population of cells comprising T cells are ex vivo activated and expanded under the normoxic condition for at least 3 days. In certain embodiments, the population of cells comprising T cells are ex vivo activated and expanded under the normoxic condition for at least 4 days. In certain embodiments, the population of cells comprising T cells are ex vivo activated and expanded under the normoxic condition for at least 5 days. In certain embodiments, the population of cells comprising T cells are ex vivo activated and expanded under the normoxic condition for from 1 hour to 7 days. In certain embodiments, the population of cells comprising T cells are ex vivo activated and expanded under the normoxic condition for from 6 hours to 7 days. In certain embodiments, the population of cells comprising T cells are ex vivo activated and expanded under the normoxic condition for from 0.5 day to 7 days. In certain embodiments, the population of cells comprising T cells are ex vivo activated and expanded under the normoxic condition for from 1 day to 7 days. In certain embodiments, the population of cells comprising T cells are ex vivo activated and expanded under the normoxic condition for from 1 day to 6 days. In certain embodiments, the population of cells comprising T cells are ex vivo activated and expanded under the normoxic condition for from 1 day to 5 days. In certain embodiments, the population of cells comprising T cells are ex vivo activated and expanded under the normoxic condition for from 1 day to 4 days. In certain embodiments, the population of cells comprising T cells are ex vivo activated and expanded under the normoxic condition for from 1 day to 3 days. In certain embodiments, the population of cells comprising T cells are ex vivo activated and expanded under the normoxic condition for from 1 day to 2 days.

In some embodiments, the oxygen concentration of the normoxic condition is at least 18%. In some embodiments, the oxygen concentration of the normoxic condition is at least 19%. In some embodiments, the oxygen concentration of the normoxic condition is at least 20%. In certain embodiments, the oxygen concentration of the normoxic condition is from 18% to 22%. In certain embodiments, the oxygen concentration of the normoxic condition is from 18% to 21%. In certain embodiments, the oxygen concentration of the normoxic condition is from 18% to 20%. In certain embodiments, the oxygen concentration of the normoxic condition is or is about 18.2%. In certain embodiments, the oxygen concentration of the normoxic condition is or is about 18.6%. In certain embodiments, the oxygen concentration of the normoxic condition is or is about 19%. In certain embodiments, the oxygen concentration of the normoxic condition is or is about 19.5%. In certain embodiments, the oxygen concentration of the normoxic condition is or is about 20%. In certain embodiments, the oxygen concentration of the normoxic condition is or is about 20.5%. In certain embodiments, the oxygen concentration of the normoxic condition is or is about 21%.

In some embodiments, the amount of a specific type of cells is measured by methods well known to those skilled in the art. In some embodiments, the amount of a specific type of cells is measured by flow cytometric analysis.

In some embodiments, the total percentage of Vγ9Vδ2 T cells in the population of cells comprising T cells is increased to more than 10%, more than 15%, more than 20%, more than 25%, more than 30%, more than 35%, more than 40%, more than 45%, more than 50%, more than 55%, or more than 60%. In some embodiments, the total percentage of Vγ9Vδ2 T cells in the population of cells comprising T cells is increased to more than 10%. In some embodiments, the total percentage of Vγ9Vδ2 T cells in the population of cells comprising T cells is increased to more than 15%. In some embodiments, the total percentage of Vγ9Vδ2 T cells in the population of cells comprising T cells is increased to more than 20%. In some embodiments, the total percentage of Vγ9Vδ2 T cells in the population of cells comprising T cells is increased to more than 25%. In some embodiments, the total percentage of Vγ9Vδ2 T cells in the population of cells comprising T cells is increased to more than 30%. In some embodiments, the total percentage of Vγ9Vδ2 T cells in the population of cells comprising T cells is increased to more than 35%. In some embodiments, the total percentage of Vγ9Vδ2 T cells in the population of cells comprising T cells is increased to more than 40%. In some embodiments, the total percentage of Vγ9Vδ2 T cells in the population of cells comprising T cells is increased to more than 45%. In some embodiments, the total percentage of Vγ9Vδ2 T cells in the population of cells comprising T cells is increased to more than 50%. In some embodiments, the total percentage of Vγ9Vδ2 T cells in the population of cells comprising T cells is increased to more than 55%. In some embodiments, the total percentage of Vγ9Vδ2 T cells in the population of cells comprising T cells is increased to more than 60%. In certain embodiments, the total percentage of Vγ9Vδ2 T cells in the population of cells comprising T cells is increased to from 10%-99%. In certain embodiments, the total percentage of Vγ9Vδ2 T cells in the population of cells comprising T cells is increased to from 20% to 95%. In certain embodiments, the total percentage of Vγ9Vδ2 T cells in the population of cells comprising T cells is increased to from 30% to 95%. In certain embodiments, the total percentage of Vγ9Vδ2 T cells in the population of cells comprising T cells is increased to from 35% to 95%. In certain embodiments, the total percentage of Vγ9Vδ2 T cells in the population of cells comprising T cells is increased to from 40% to 95%. In certain embodiments, the total percentage of Vγ9Vδ2 T cells in the population of cells comprising T cells is increased to from 45% to 95%. In certain embodiments, the total percentage of Vγ9Vδ2 T cells in the population of cells comprising T cells is increased to from 50% to 95%. In certain embodiments, the total percentage of Vγ9Vδ2 T cells in the population of cells comprising T cells is increased to from 60% to 95%. In certain embodiments, the total percentage of Vγ9Vδ2 T cells in the population of cells comprising T cells is increased to from 65% to 95%. In certain embodiments, the total percentage of Vγ9Vδ2 T cells in the population of cells comprising T cells is increased to about 50%. In certain embodiments, the total percentage of Vγ9Vδ2 T cells in the population of cells comprising T cells is increased to about 55%. In certain embodiments, the total percentage of Vγ9Vδ2 T cells in the population of cells comprising T cells is increased to about 60%. In certain embodiments, the total percentage of Vγ9Vδ2 T cells in the population of cells comprising T cells is increased to about 65%. In certain embodiments, the total percentage of Vγ9Vδ2 T cells in the population of cells comprising T cells is increased to about 70%. In certain embodiments, the total percentage of Vγ9Vδ2 T cells in the population of cells comprising T cells is increased to about 75%. In certain embodiments, the total percentage of Vγ9Vδ2 T cells in the population of cells comprising T cells is increased to about 80%. In certain embodiments, the total percentage of Vγ9Vδ2 T cells in the population of cells comprising T cells is increased to about 85%. In certain embodiments, the total percentage of Vγ9Vδ2 T cells in the population of cells comprising T cells is increased to about 90%. In certain embodiments, the total percentage of Vγ9Vδ2 T cells in the population of cells comprising T cells is increased to about 95%.

In some embodiments, the total number of Vγ9Vδ2 T cells in the population of cells comprising T cells is increased by at least 10-fold, at least 30-fold, at least 50-fold, at least 100-fold, at least 150-fold, at least 200-fold, at least 250-fold, at least 300-fold, at least 350-fold, at least 400-fold, at least 450-fold, at least 500-fold, at least 550-fold, or at least 600-fold as compared to the total number of Vγ9Vδ2 T cells in the population of cells comprising T cells before the expansion. In some embodiments, the total number of Vγ9Vδ2 T cells in the population of cells comprising T cells is increased by at least 10-fold. In some embodiments, the total number of Vγ9Vδ2 T cells in the population of cells comprising T cells is increased by at least 30-fold. In some embodiments, the total number of Vγ9Vδ2 T cells in the population of cells comprising T cells is increased by at least 50-fold. In some embodiments, the total number of Vγ9Vδ2 T cells in the population of cells comprising T cells is increased by at least 100-fold. In some embodiments, the total number of Vγ9Vδ2 T cells in the population of cells comprising T cells is increased by at least 150-fold. In some embodiments, the total number of Vγ9Vδ2 T cells in the population of cells comprising T cells is increased by at least 200-fold. In some embodiments, the total number of Vγ9Vδ2 T cells in the population of cells comprising T cells is increased by at least 250-fold. In some embodiments, the total number of Vγ9Vδ2 T cells in the population of cells comprising T cells is increased by at least 300-fold. In some embodiments, the total number of Vγ9Vδ2 T cells in the population of cells comprising T cells is increased by at least 350-fold. In some embodiments, the total number of Vγ9Vδ2 T cells in the population of cells comprising T cells is increased by at least 400-fold. In some embodiments, the total number of Vγ9Vδ2 T cells in the population of cells comprising T cells is increased by at least 450-fold. In some embodiments, the total number of Vγ9Vδ2 T cells in the population of cells comprising T cells is increased by at least 500-fold. In some embodiments, the total number of Vγ9Vδ2 T cells in the population of cells comprising T cells is increased by at least 550-fold. In some embodiments, the total number of Vγ9Vδ2 T cells in the population of cells comprising T cells are increased by at least 600-fold. In certain embodiments, the total number of Vγ9Vδ2 T cells in the population of cells comprising T cells is increased by from 10-fold to 900-fold. In certain embodiments, the total number of Vγ9Vδ2 T cells in the population of cells comprising T cells is increased by from 10-fold to 800-fold. In certain embodiments, the total number of Vγ9Vδ2 T cells in the population of cells comprising T cells is increased by from 30-fold to 700-fold. In certain embodiments, the total number of Vγ9Vδ2 T cells in the population of cells comprising T cells is increased by from 30-fold to 650-fold. In certain embodiments, the total number of Vγ9Vδ2 T cells in the population of cells comprising T cells is increased by from 50-fold to 600-fold. In certain embodiments, the total number of Vγ9Vδ2 T cells in the population of cells comprising T cells is increased by from 100-fold to 600-fold. In certain embodiments, the total number of Vγ9Vδ2 T cells in the population of cells comprising T cells are increased by from 150-fold to 600-fold. In certain embodiments, the total number of Vγ9Vδ2 T cells in the population of cells comprising T cells is increased by from 200-fold to 600-fold. In certain embodiments, the total number of Vγ9Vδ2 T cells in the population of cells comprising T cells is increased by from 250-fold to 600-fold. In certain embodiments, the total number of Vγ9Vδ2 T cells in the population of cells comprising T cells is increased by from 300-fold to 600-fold. In certain embodiments, the total number of Vγ9Vδ2 T cells in the population of cells comprising T cells is increased by from 350-fold to 600-fold. In certain embodiments, the total number of Vγ9Vδ2 T cells in the population of cells comprising T cells is increased by from 400-fold to 600-fold. In certain embodiments, the total number of Vγ9Vδ2 T cells in the population of cells comprising T cells is increased by from 450-fold to 600-fold. In certain embodiments, the total number of Vγ9Vδ2 T cells in the population of cells comprising T cells is increased by from 500-fold to 600-fold.

In some embodiments, the Vγ9Vδ2 T cells are isolated or enriched from the population of cells comprising T cells after culturing the population of cells comprising T cells ex vivo in the culture system under a hypoxic condition for enhanced ex vivo activation and expansion of Vγ9Vδ2 T cells in the population of cells comprising T cells.

In certain embodiments, Vγ9⁺ cells are isolated first and Vδ2⁺ cells are isolated second. In certain embodiments, Vδ2⁺ cells are isolated first and Vγ9⁺ cells are isolated second. In certain embodiments, Vγ9⁺ cells and Vδ2⁺ cells are isolated simultaneously. In certain embodiments, Vγ9Vδ2 T cells are isolated or enriched as described in Section 6.1.5 below.

The methods of isolating or enriching Vγ9⁺ cells are known to those skilled in the art. In some embodiments, the method of enriching Vγ9⁺ cells comprises a positive selection. In some embodiments, the method of enriching Vγ9⁺ cells comprises a negative selection.

In some embodiments, the method of enriching Vγ9⁺ cells comprises a negative selection comprising incubating the cell mixture with reagents that bind to undesired cells. In some embodiments, the method of enriching Vγ9⁺ cells comprises density gradient centrifugation, using, for example albumin, dextran, Ficoll, metrizamid, Percoll, and/or the like, to remove undesired cells. In some embodiments, the method of enriching Vγ9⁺ cells comprises a positive selection comprising selecting or sorting cells having a cell surface expression of Vγ9. In some embodiments, the method of enriching Vγ9⁺ cells comprises FACS sorting of Vγ9⁺ cells. In some embodiments, the method of enriching Vγ9⁺ cells comprises using an anti-Vγ9 antibody which targets any one of Vγ9 chains. In some embodiments, the method of enriching Vγ9⁺ cells comprises an affinity column immobilized with a binding agent to Vγ9. In some embodiments, the method of enriching Vγ9⁺ cells comprises a combination of two or more of the above methods.

In certain embodiments, the method of enriching Vγ9⁺ cells comprises using magnetic beads coated with an anti-Vγ9 antibody. In certain embodiments, the anti-Vγ9 antibody-coated magnetic beads are coated with a secondary reagent that binds to the anti-Vγ9 antibody. In certain embodiments, the method of enriching Vγ9⁺ cells comprises using magnetic microparticles coated with an anti-Vγ9 antibody. In certain embodiments, the anti-Vγ9 antibody-coated magnetic microparticles are coated with a secondary reagent that binds to the anti-Vγ9 antibody. In certain embodiments, the method of enriching Vγ9⁺ cells comprises using magnetic nanoparticles coated with an anti-Vγ9 antibody. In certain embodiments, the anti-Vγ9 antibody-coated magnetic nanoparticles are coated with a secondary reagent that binds to the anti-Vγ9 antibody.

The method of isolating or enriching Vδ2⁺ cells are known to those skilled in the art. In some embodiments, the method of enriching Vδ2⁺ cells comprises a positive selection. In some embodiments, the method of enriching Vδ2⁺ cells comprises a negative selection.

In some embodiments, the method of enriching Vδ2⁺ cells comprises a negative selection comprising incubating the cell mixture with reagents that bind to undesired cells. In some embodiments, the method of enriching Vδ2⁺ cells comprises density gradient centrifugation, using, for example albumin, dextran, Ficoll, metrizamid, Percoll and/or the like, to remove undesired cells. In some embodiments, the method of enriching Vδ2⁺ cells comprises a positive selection comprising sorting or selecting cells having a cell surface expression of Vδ2. In some embodiments, the method of enriching Vδ2⁺ cells comprises FACS sorting of Vδ2⁺ cells. In some embodiments, the method of enriching Vδ2⁺ cells comprises using an anti-Vδ2 antibody which targets any one of Vδ2 chains. In some embodiments, the method of enriching Vδ2⁺ cells comprises an affinity column immobilized with a binding agent to Vδ2. In some embodiments, the method of enriching Vδ2⁺ cells comprises a combination of two or more of the above methods.

In certain embodiments, the method of enriching Vδ2⁺ cells comprises using magnetic beads coated with an anti-Vδ2 antibody. In certain embodiments, the anti-Vδ2 antibody-coated magnetic beads are coated with a secondary reagent that binds to the anti-Vδ2 antibody. In certain embodiments, the method of enriching Vδ2⁺ cells comprises using magnetic microparticles coated with an anti-Vδ2 antibody. In certain embodiments, the anti-Vδ2 antibody-coated magnetic microparticles are coated with a secondary reagent that binds to the anti-Vδ2 antibody. In certain embodiments, the method of enriching Vδ2⁺ cells comprises using magnetic nanoparticles coated with an anti-Vδ2 antibody. In certain embodiments, the anti-Vδ2 antibody-coated magnetic nanoparticles are coated with a secondary reagent that binds to the anti-Vδ2 antibody.

### 4.5.2. Chimeric Antigen Receptors

The method provided herein for generating a CAR T cell comprises introducing a nucleic acid encoding a chimeric antigen receptor (CAR) into a Vγ9Vδ2 T cell.

In some embodiments, the CAR provided herein comprises a polypeptide comprising: (a) an extracellular antigen binding domain; (b) a transmembrane domain; and (c) an intracellular signaling domain.

### Signal Peptide

In certain embodiments, the CARs provided herein may comprise a signal peptide (also known as a signal sequence) at the N-terminus of the polypeptide. In general, signal peptides are peptide sequences that target a polypeptide to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the CARs described herein will be evident to one skilled in the art. In some embodiments, the signal peptide is derived from a molecule selected from the group consisting of CD8«, GM-CSF receptor «, and IgGI heavy chain.

### Extracellular Antigen Binding Domain

The extracellular antigen binding domain of the CARs described herein comprises one or more antigen binding domains. In some embodiments, the extracellular antigen binding domain of the CAR provided herein is mono-specific. In other embodiments, the extracellular antigen binding domain of the CAR provided herein is multispecific. In some embodiments, the extracellular antigen binding domain comprises two or more antigen binding domains which are fused to each other directly via peptide bonds, or via peptide linkers.

In some embodiments, the extracellular antigen binding domain comprises an antibody or a fragment thereof. For example, the binding domain may be derived from monoclonal antibodies (including agonist, antagonist, neutralizing antibodies, full length or intact monoclonal antibodies), antibody with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies). An antibody can be human, humanized, chimeric and/or affinity matured, as well as an antibody from other species, for example, mouse, rabbit, llama, etc. In some embodiments, the antibody include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

In a specific embodiment, the extracellular antigen binding domain of the present CARs comprise a single-chain Fv (sFv or scFv). ScFvs are antibody fragments that comprise the VH and VL antibody domains connected into a single polypeptide chain. Preferably, the scFv polypeptide further comprises a polypeptide linker between the VH and VL domains which enables the sFv to form the desired structure for antigen binding. See Pluckthun in The Pharmacology of Monoclonal Antibodies, vol. 113, Rosenburg and Moore eds., Springer-Verlag, New York, pp. 269-315 (1994).

In another specific embodiment, the extracellular antigen binding domain of the present CARs comprises one or more single domain antibodies (sdAbs). The sdAbs may be of the same or different origins, and of the same or different sizes. Exemplary sdAbs include, but are not limited to, heavy chain variable domains from heavy-chain only antibodies (e.g., VHH or VNAR), binding molecules naturally devoid of light chains, single domains (such as VH or VL) derived from conventional 4-chain antibodies, humanized heavy-chain only antibodies, human single domain antibodies produced by transgenic mice or rats expressing human heavy chain segments, and engineered domains and single domain scaffolds other than those derived from antibodies. Any sdAbs known in the art or developed by the present disclosure, including the single domain antibodies described above in the present disclosure, may be used to construct the CARs described herein. The sdAbs may be derived from any species including, but not limited to mouse, rat, human, camel, llama, lamprey, fish, shark, goat, rabbit, and bovine. Single domain antibodies contemplated herein also include naturally occurring single domain antibody molecules from species other than Camelidae and sharks.

In some embodiments, the sdAb is derived from a naturally occurring single domain antigen binding molecule known as heavy chain antibody devoid of light chains (also referred herein as “heavy chain only antibodies”). Such single domain molecules are disclosed in WO 94/04678 and Hamers-Casterman, C. et al., Nature 363:446-448 (1993), for example. For clarity reasons, the variable domain derived from a heavy chain molecule naturally devoid of light chain is known herein as a VHH to distinguish it from the conventional V_{H} of four chain immunoglobulins. Such a VHH molecule can be derived from antibodies raised in (′amelidae species, for example, camel, llama, vicuna, dromedary, alpaca and guanaco. Other species besides Camelidae may produce heavy chain molecules naturally devoid of light chain, and such VHHs are within the scope of the present disclosure. In addition, humanized versions of VHHs as well as other modifications and variants are also contemplated and within the scope of the present disclosure. In some embodiments, the sdAb is derived from a variable region of the immunoglobulin found in cartilaginous fish. For example, the sdAb can be derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov, Protein Sci. 14:2901-2909 (2005).

In some embodiments, naturally occurring VHH domains against a particular antigen or target, can be obtained from (naïve or immune) libraries of Camelid VHH sequences. Such methods may or may not involve screening such a library using said antigen or target, or at least one part, fragment, antigenic determinant or epitope thereof using one or more screening techniques known in the field. Such libraries and techniques are for example described in WO 99/37681, WO 01/90190, WO 03/025020 and WO 03/035694. Alternatively, improved synthetic or semi-synthetic libraries derived from (naïve or immune) VHH libraries may be used, such as VHH libraries obtained from (naïve or immune) VHH libraries by techniques such as random mutagenesis and/or CDR shuffling, as for example described in WO 00/43507.

In some embodiments, the sdAb is recombinant, CDR-grafted, humanized, camelized, de-immunized and/or in vitro generated (e.g., selected by phage display). In some embodiments, the amino acid sequence of the framework regions may be altered by “camelization” of specific amino acid residues in the framework regions. Camelization refers to the replacing or substitution of one or more amino acid residues in the amino acid sequence of a (naturally occurring) VH domain from a conventional 4-chain antibody by one or more of the amino acid residues that occur at the corresponding position(s) in a VHH domain of a heavy chain antibody. This can be performed in a manner known in the field, which will be clear to the skilled person. Such “camelizing” substitutions are preferably inserted at amino acid positions that form and/or are present at the VH-VL interface, and/or at the so-called Camelidae hallmark residues, as defined herein (see for example WO 94/04678, Davies and Riechmann FEBS Letters 339:285-290 (1994); Davies and Riechmann, Protein Engineering 9 (6): 531-537 (1996); Riechmann, J. Mol. Biol. 259:957-969 (1996); and Riechmann and Muyldermans, J. Immunol. Meth. 231:25-38 (1999)).

In some embodiments, the sdAb is a human single domain antibody produced by transgenic mice or rats expressing human heavy chain segments. See, e.g., US20090307787, U.S. Pat. No. 8,754,287, US20150289489, US20100122358, and WO2004049794.

In some embodiments, the single domain antibodies are generated from conventional four-chain antibodies. See, for example, EP 0 368 684; Ward et al., Nature, 341 (6242): 544-6 (1989); Holt et al., Trends Biotechnol., 21 (11): 484-490 (2003); WO 06/030220; and WO 06/003388.

In some embodiments, the extracellular antigen binding domain comprises humanized antibodies or fragment thereof. A humanized antibody can comprise human framework region and human constant region sequences.

Humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592, 106 and EP 519,596; Padlan, 1991, Molecular Immunology 28 (4/5): 489-498; Studnicka et al., 1994, Protein

Engineering 7 (6): 805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, WO 93/17105, Tan et al., J. Immunol. 169:1119 25 (2002), Caldas et al., Protein Eng. 13 (5): 353-60 (2000), Morea et al., Methods 20 (3): 267 79 (2000), Baca et al., J. Biol. Chem. 272 (16): 10678-84 (1997), Roguska et al., Protein Eng. 9 (10): 895 904 (1996), Couto et al., Cancer Res. 55 (23 Supp): 5973s-5977s (1995), Couto et al., Cancer Res. 55 (8): 1717-22 (1995), Sandhu J S, Gene 150 (2): 409-10 (1994), and Pedersen et al., J. Mol. Biol. 235 (3): 959-73 (1994). See also U.S. Patent Pub. No. US 2005/0042664 A1 (Feb. 24, 2005), each of which is incorporated by reference herein in its entirety.

Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, VL6 subgroup I (VL6I) and VH subgroup III (VHIII). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., 2002, J. Immunol. 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the FR library approach, a collection of residue variants is introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In FR shuffling, whole FRs are combined with the non-human CDRs instead of creating combinatorial libraries of selected residue variants (see, e.g., Dall'Acqua et al., 2005, Methods 36:43-60). The libraries may be screened for binding in a two-step process, first humanizing VL, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder et al., 2007, Mol. Immunol. 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence.

The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody. Such antibodies can comprise human constant region sequences, e.g., human light chain and/or heavy chain constant regions.

A deimmunized antibody is an antibody in which T cell epitopes have been removed. Methods for making deimmunized antibodies have been described. See, e.g., Jones et al., Methods Mol Biol. 2009; 525:405-23, xiv, and De Groot et al., Cell. Immunol. 244:148-153 (2006)). Deimmunized antibodies comprise T cell epitope-depleted variable regions and human constant regions. Briefly, VH and VL of an antibody are cloned and T cell epitopes are subsequently identified by testing overlapping peptides derived from the VH and VL of the antibody in a T cell proliferation assay. T cell epitopes are identified via in silico methods to identify peptide binding to human MHC class II. Mutations are introduced in the VH and VL to abrogate binding to human MHC class II. Mutated VH and VL are then utilized to generate the deimmunized antibody.

In certain embodiments, the extracellular antigen binding domain comprises multiple binding domains. In some embodiments, the extracellular antigen binding domain comprises multispecific antibodies or fragments thereof. In other embodiments, the extracellular antigen binding domain comprises multivalent antibodies or fragments thereof. The term “specificity” refers to selective recognition of an antigen binding protein for a particular epitope of an antigen. The term “multispecific” as used herein denotes that an antigen binding protein has two or more antigen-binding sites of which at least two bind different antigens. The term “valent” as used herein denotes the presence of a specified number of binding sites in an antigen binding protein. A full-length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

Multispecific antibodies such as bispecific antibodies are antibodies that have binding specificities for at least two different antigens. Methods for making multispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

The antibodies of the present disclosure can be multivalent antibodies with two or more antigen binding sites (e.g., tetravalent antibodies), which can be readily produced by recombinant expression of nucleic acid encoding the polypeptide chains of the antibody. In certain embodiments, a multivalent antibody comprises (or consists of) three to about eight antigen binding sites. In one such embodiment, a multivalent antibody comprises (or consists of) four antigen binding sites. The multivalent antibody comprises at least one polypeptide chain (e.g., two polypeptide chains), wherein the polypeptide chain(s) comprise two or more variable domains. For instance, the polypeptide chain(s) may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH—CH1-Fc region chain; or VH—CH1-VH-CH1-Fc region chain. The multivalent antibody herein may further comprise at least two (e.g., four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

In case there are multiple binding domains in the extracellular antigen binding domain of the present CARs. The various domains may be fused to each other via peptide linkers. In some embodiments, the domains are directly fused to each other without any peptide linkers. The peptide linkers may be the same or different. Each peptide linker may have the same or different length and/or sequence depending on the structural and/or functional features of the various domains. Each peptide linker may be selected and optimized independently. The length, the degree of flexibility and/or other properties of the peptide linker(s) used in the CARs may have some influence on properties, including but not limited to the affinity, specificity or avidity for one or more particular antigens or epitopes. In some embodiment, a peptide linker comprises flexible residues (such as glycine and serine) so that the adjacent domains are free to move relative to each other. For example, a glycine-serine doublet can be a suitable peptide linker.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. Exemplary flexible linkers include but not limited to glycine polymers (G)ₙ, glycine-serine polymers (including, for example, (GS)ₙ, (GSGGS)ₙ, (GGGS)ₙ, and (GGGGS)ₙ, where n is an integer of at least one), glycine-alanine polymers, alanine-serine polymers, and other flexible linkers known in the art. Other linkers known in the art, for example, as described in WO2016014789, WO2015158671, WO2016102965, US20150299317, WO2018067992, U.S. Pat. No. 7,741,465, Colcher et al., J. Nat. Cancer Inst. 82:1191-1197 (1990), and Bird et al., Science 242:423-426 (1988) may also be included in the CARs provided herein, the disclosure of each of which is incorporated herein by reference.

In some embodiments, the extracellular antigen binding domain provided in the present CARs recognizes an antigen that acts as a cell surface marker on target cells associated with a special disease state. The antigens targeted by the CAR may be antigens on a single diseased cell or antigens that are expressed on different cells that each contribute to the disease. The antigens targeted by the CAR may be directly or indirectly involved in the diseases. In some embodiments, the extracellular antigen binding domain binds to an antigen expressed on an unhealthy cell. In some embodiments, the unhealthy cell is a cancer cell. In certain embodiments, the cancer cell is a blood cancer cell or a solid tumor cancer cell. In some embodiments, the unhealthy cell is from a subject having an autoimmune and inflammatory disease. In some embodiments, the unhealthy cell is from a subject having a neurological disease.

In some embodiments, the extracellular antigen binding domain binds to a tumor antigen. Exemplary tumor antigens include, but are not limited to, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RUI, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, and mesothelin.

In some embodiments, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and gp 100 in melanoma and prostatic acid phosphatase (PAP) and prostate-specific antigen (PSA) in prostate cancer.

Other target molecules belong to the group of transformation-related molecules such as the oncogene HER2/Neu/ErbB-2. Yet another group of target antigens are onco-fetal antigens such as carcinoembryonic antigen (CEA).

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that induce a state of immunologic tolerance to the antigen. The expression of the antigen on the tumor may occur under conditions that enable the immune system to respond to the antigen. TAAs may be antigens that are expressed on normal cells during fetal development, when the immune system is immature and unable to respond, or they may be antigens that are normally present at extremely low levels on normal cells but are expressed at much higher levels on tumor cells.

Non-limiting examples of TSA or TAA antigens include: differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7.

Other large, protein-based antigens include TSP-180, MAGE-4, MAGE-5, MAGE-6, RAGE, NY-ESO, pl85erbB2, p180erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

### Hinge Region

In some embodiments, the CARs provided herein comprise a hinge domain that is located between the extracellular antigen binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular antigen binding domain relative to the transmembrane domain of the effector molecule can be used.

Hinge domains of antibodies, such as an IgG, IgA, IgM, IgE, or IgD antibodies, are also compatible for use in the pH-dependent chimeric receptor systems described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgGI antibody.

Non-naturally occurring peptides may also be used as hinge domains for the chimeric receptors described herein. In some embodiments, the hinge domain between the C-terminus of the extracellular ligand-binding domain of an Fc receptor and the N-terminus of the transmembrane domain is a peptide linker, such as a (G×S)n linker, wherein x and n, independently can be an integer between 3 and 12, including 3, 4, 5, 6, 7, 8, 9, 10, 11, 12, or more.

The hinge domain may comprise about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor. In some embodiments, the hinge domain is derived from CD8a. In some embodiments, the hinge domain is a portion of the hinge domain of CD8a, e.g., a fragment containing about 15-100 (e.g., 20, 25, 30, 35, or 40) consecutive amino acids of the hinge domain of CD8a.

### Transmembrane Domain

The CARs of the present disclosure comprise a transmembrane domain that can be directly or indirectly fused to the extracellular antigen binding domain. The transmembrane domain may be derived either from a natural or from a synthetic source. As used herein, a “transmembrane domain” refers to any protein structure that is thermodynamically stable in a cell membrane, preferably a eukaryotic cell membrane. Transmembrane domains compatible for use in the CARs described herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three-dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the CAR described herein is derived from a Type I single-pass membrane protein. In some embodiments, transmembrane domains from multi-pass membrane proteins may also be compatible for use in the CARs described herein. Multi-pass membrane proteins may comprise a complex (at least 2, 3, 4, 5, 6, 7 or more) alpha helices or a beta sheet structure. In some embodiments, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

Transmembrane domains for use in the CARs described herein can also comprise at least a portion of a synthetic, non-naturally occurring protein segment. In some embodiments, the transmembrane domain is a synthetic, non-naturally occurring alpha helix or beta sheet. In some embodiments, the protein segment has a length of about 15-100 amino acids. In some embodiments, the protein segment has a length of at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776, the relevant disclosures of which are incorporated by reference herein.

The transmembrane domain provided herein may comprise a transmembrane region and a cytoplasmic region located at the C-terminus of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain of the CAR provided herein comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

In some embodiments, the transmembrane domain of the CAR comprises a transmembrane domain chosen from the transmembrane domain of an alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD1 1a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7Ra, ITGA1, VLA1, CD49a, ITGA4, IA4, CD49D, ITGA6, VLA-6, CD49f, ITGAD, CD1 1d, ITGAE, CD103, ITGAL, CD1 1a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

### Intracellular Signaling Domain(s)

The intracellular signaling domain(s) of the CARs provided herein are responsible for activation of at least one of the normal effector functions of the immune effector cell expressing the CARs. The term “effector function” refers to a specialized function of a cell. Effector function of a T cell, for example, may be cytolytic activity or helper activity including the secretion of cytokines. Thus, the term “cytoplasmic signaling domain” refers to the portion of a protein which transduces the effector function signal and directs the cell to perform a specialized function. While usually the entire cytoplasmic signaling domain can be employed, in many cases it is not necessary to use the entire chain. To the extent that a truncated portion of the cytoplasmic signaling domain is used, such truncated portion may be used in place of the intact chain as long as it transduces the effector function signal. The term cytoplasmic signaling domain is thus meant to include any truncated portion of the cytoplasmic signaling domain sufficient to transduce the effector function signal.

In some embodiments, the intracellular signaling domain comprises a primary intracellular signaling domain of an immune effector cell. In some embodiments, the CAR comprises an intracellular signaling domain consisting essentially of a primary intracellular signaling domain of an immune effector cell. “Primary intracellular signaling domain” refers to cytoplasmic signaling sequence that acts in a stimulatory manner to induce immune effector functions. In some embodiments, the primary intracellular signaling domain comprises a signaling motif known as immunoreceptor tyrosine-based activation motif, or ITAM. An “ITAM,” as used herein, is a conserved protein motif that is generally present in the tail portion of signaling molecules expressed in many immune cells. The motif may comprise two repeats of the amino acid sequence YxxL/I separated by 6-8 amino acids, wherein each x is independently any amino acid, producing the conserved motif YxxL/Ix (6-8) YxxL/I. ITAMs within signaling molecules are important for signal transduction within the cell, which is mediated at least in part by phosphorylation of tyrosine residues in the ITAM following TCR or CAR engagement. ITAMs may also function as docking sites for other proteins involved in signaling pathways. Exemplary ITAM-containing primary cytoplasmic signaling sequences include those derived from CD3 zeta, FcR gamma (FCER1G), FcR beta (FCER1B), CD3 gamma, CD3 delta, CD3 epsilon, CD5, CD22, CD79a, CD79b, and CD66d.

In some embodiments, the primary intracellular signaling domain is derived from CD3 zeta. In some embodiments, the intracellular signaling domain consists of a cytoplasmic signaling domain of CD3 zeta. In some embodiments, the primary intracellular signaling domain is a cytoplasmic signaling domain of wild-type CD3 zeta.

### Co-stimulatory Signaling Domain

In some embodiments, the CAR comprises at least one co-stimulatory signaling domain. The term “co-stimulatory signaling domain,” as used herein, refers to at least a portion of a protein that mediates signal transduction within a cell to induce an immune response such as an effector function. Many immune effector cells require co-stimulation, in addition to stimulation of an antigen-specific signal, to promote cell proliferation, differentiation and survival, as well as to activate effector functions of the cell.

The co-stimulatory signaling domain of the CAR described herein can be a cytoplasmic signaling domain from a co-stimulatory protein, which transduces a signal and modulates responses mediated by immune cells, such as T cells, NK cells, macrophages, neutrophils, or eosinophils. “Co-stimulatory signaling domain” can be the cytoplasmic portion, or one or more domains thereof, of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival.

In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains. In some embodiments, the intracellular signaling domain comprises two or more co-stimulatory signaling domains from different co-stimulatory proteins, such as any two or more co-stimulatory proteins described herein. In some embodiments, the intracellular signaling domain comprises a primary intracellular signaling domain (such as cytoplasmic signaling domain of CD3 zeta) and one or more co-stimulatory signaling domains. In some embodiments, the one or more co-stimulatory signaling domains and the primary intracellular signaling domain (such as cytoplasmic signaling domain of CD3 zeta) are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as cytoplasmic signaling domain of CD3 zeta). Multiple co-stimulatory signaling domains may provide additive or synergistic stimulatory effects.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the CARs described herein. The type(s) of co-stimulatory signaling domain is selected based on factors such as the type of the immune effector cells in which the effector molecules would be expressed (e.g., T cells, NK cells, macrophages, neutrophils, or eosinophils) and the desired immune effector function (e.g., ADCC effect, proliferation, cytokine release, and cytotoxicity). Examples of co-stimulatory signaling domains for use in the CARs can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BlyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD7, CD53, CD82/Kai-1, CD90/Thyl, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the one or more co-stimulatory signaling domains are selected from the group consisting of CD27, CD28, CD137, OX40, CD30, CD40, lymphocyte function-associated antigen-1 (LFA-1), CD2, CD7, LIGHT, NKG2C, B7-H3 and ligands that bind to CD83. In some embodiments, the co-stimulatory signaling domains for use in the CARs comprise intracellular signaling domains from cytokine receptors.

In some embodiments, the intracellular signaling domain of the CAR of the present disclosure comprises a co-stimulatory signaling domain derived from CD137 (i.e., 4-1BB). In some embodiments, the intracellular signaling domain comprises a cytoplasmic signaling domain of CD3 zeta and a co-stimulatory signaling domain of CD137.

In some embodiments, the co-stimulatory signaling domains are variants of any of the co-stimulatory signaling domains described herein, such that the co-stimulatory signaling domain is capable of modulating an immune response of an immune cell. In some embodiments, the co-stimulatory signaling domains comprise up to 10 amino acid residue variations (e.g., 1, 2, 3, 4, 5, or 8) as compared to a wild-type counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

### 4.5.3. Polynucleotides

In certain embodiments, the present disclosure provides polynucleotides that encode the CARs provided herein. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide.

The present disclosure further relates to variants of the polynucleotides described herein, wherein the variant encodes, for example, fragments, analogs, and/or derivatives of the antibody or CAR of the disclosure. In certain embodiments, the present disclosure provides a polynucleotide comprising a polynucleotide comprising a nucleotide sequence that is at least about 75% identical, at least about 80% identical, at least about 85% identical, at least about 90% identical, at least about 95% identical, and in some embodiments, at least about 96%, 97%, 98% or 99% identical to a polynucleotide encoding the CAR of the disclosure. As used herein, the phrase “a polynucleotide comprising a nucleotide sequence at least, for example, 95% “identical” to a reference nucleotide sequence” is intended to mean that the nucleotide sequence of the polynucleotide is identical to the reference sequence except that the polynucleotide sequence can include up to five point-mutations per each 100 nucleotides of the reference nucleotide sequence. In other words, to obtain a polynucleotide comprising a nucleotide sequence that is at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

### 4.5.4. Vectors

Also provided are vectors comprising the nucleic acids described herein. In an embodiment, the nucleic acids can be incorporated into a recombinant expression vector.

The present disclosure provides vectors for cloning and expressing any one of the CARs described herein. In some embodiments, the vector is suitable for replication and integration in eukaryotic cells, such as mammalian cells. In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the immunomodulator (such as immune checkpoint inhibitor) coding sequence and/or self-inactivating lentiviral vectors carrying chimeric antigen receptors can be packaged with protocols known in the art. The resulting lentiviral vectors can be used to transduce a mammalian cell (such as primary human T cells) using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector comprises any one of the nucleic acids encoding a CAR described herein. The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in mammalian cells, and any of the promoters known in the art may be used in the present disclosure. Promoters may be roughly categorized as constitutive promoters or regulated promoters, such as inducible promoters.

In some embodiments, the nucleic acid encoding the CAR is operably linked to a constitutive promoter. Constitutive promoters allow heterologous genes (also referred to as transgenes) to be expressed constitutively in a host cells. Exemplary constitutive promoters contemplated herein include, but are not limited to, Cytomegalovirus (CMV) promoters, human elongation factors-1 alpha (hEF1a), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. For example, Michael C. Milone et al compared the efficiencies of CMV, hEF1a, UbiC and PGK to drive chimeric antigen receptor expression in primary human T cells, and concluded that hEF1a promoter not only induced the highest level of transgene expression, but was also optimally maintained in the CD4 and CD8 human T cells (Molecular Therapy, 17 (8): 1453-1464 (2009)). In some embodiments, the nucleic acid encoding the CAR is operably linked to a hEF1a promoter.

In some embodiments, the nucleic acid encoding the CAR is operably linked to an inducible promoter. Inducible promoters belong to the category of regulated promoters. The inducible promoter can be induced by one or more conditions, such as a physical condition, microenvironment of the engineered immune effector cell, or the physiological state of the engineered immune effector cell, an inducer (i.e., an inducing agent), or a combination thereof.

In some embodiments, the inducing condition does not induce the expression of endogenous genes in the engineered mammalian cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered mammalian cell.

In some embodiments, the vector also comprises a selectable marker gene or a reporter gene to select cells expressing the CAR from the population of host cells transfected through lentiviral vectors. Both selectable markers and reporter genes may be flanked by appropriate regulatory sequences to enable expression in the host cells. For example, the vector may comprise transcription and translation terminators, initiation sequences, and promoters useful for regulation of the expression of the nucleic acid sequences.

### 4.6. Vγ9Vδ2 T Cells Expressing CARs

In yet another aspect, provided herein is a CAR T cell product produced according to the methods provided herein, e.g., as described in Section 4.5 above.

More specifically, in some embodiments, the CAR T cell expressing a CAR, wherein the CAR T cell is a Vγ9Vδ2 T cell.

In some embodiments, the CAR in the present CAR T cells comprises an extracellular antigen binding domain; a transmembrane domain; and an intracellular signaling domain. In some embodiments, the CAR further comprises one or more additional regions/domains such as a signal peptide, hinge region, co-stimulatory signaling domain, linkers, etc., each of which is as described in Section 4.5.2 above.

Specifically, in certain embodiments, the CARs provided herein may comprise a signal peptide at the N-terminus of the polypeptide. In some embodiments, the signal peptide targets the effector molecule to the secretory pathway of the cell and allows for integration and anchoring of an effector molecule into a lipid bilayer. Signal peptides including signal sequences of naturally occurring proteins or synthetic, non-naturally occurring signal sequences, which are compatible for use in the CARs described herein are evident to one skilled in the art. In some embodiments, the signal peptide is derived from a molecule selected from the group consisting of CD8α, GM-CSF receptor a, and IgGI heavy chain.

The extracellular antigen binding domain of the CARs described herein comprises one or more antigen binding domains. In some embodiments, the extracellular antigen binding domain comprises an antibody or a fragment thereof. In certain embodiment, the extracellular antigen binding domain of the present CARs comprises a single-chain Fv (sFv or scFv). In some embodiments, the extracellular antigen binding domain comprises a humanized antibody or a fragment thereof.

In certain embodiments, the extracellular antigen binding domain comprises multiple binding domains. In some embodiments, the extracellular antigen binding domain comprises multispecific antibodies or fragments thereof. In other embodiments, the extracellular antigen binding domain comprises multivalent antibodies or fragments thereof.

In case there are multiple binding domains in the extracellular antigen binding domain of the present CARs, the various domains may be fused to each other via peptide linkers. In some embodiments, the domains are directly fused to each other without any peptide linkers. The peptide linkers may be the same or different. Each peptide linker may have the same or different length and/or sequence depending on the structural and/or functional features of the various domains. Each peptide linker may be selected and optimized independently.

In some embodiments, the extracellular antigen binding domain provided in the present CARs recognizes an antigen that acts as a cell surface marker on target cells associated with a special disease state. The antigens targeted by the CAR may be antigens on a single diseased cell or antigens that are expressed on different cells that each contribute to the disease. The antigens targeted by the CAR may be directly or indirectly involved in the diseases. In some embodiments, the extracellular antigen binding domain binds to an antigen expressed on an unhealthy cell. In some embodiments, the unhealthy cell is a cancer cell. In certain embodiments, the cancer cell is a blood cancer cell or a solid tumor cancer cell. In some embodiments, the unhealthy cell is from a subject having an autoimmune and inflammatory disease. In some embodiments, the unhealthy cell is from a subject having a neurological disease.

In some embodiments, the extracellular antigen binding domain binds to a tumor antigen. Exemplary tumor antigens include, but not limited to, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RUI, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, and mesothelin.

In some embodiments, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and gp 100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA).

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include: differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, pl5; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7.

Other large, protein-based antigens include TSP-180, MAGE-4, MAGE-5, MAGE-6, RAGE, NY-ESO, pl85erbB2, pl80erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

In some embodiments, the CARs provided herein comprise a hinge domain that is located between the extracellular antigen binding domain and the transmembrane domain. In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor. In some embodiments, the hinge domain is derived from CD8a. In some embodiments, the hinge domain is a portion of the hinge domain of CD8a, e.g., a fragment containing about 15-100 (e.g., 20, 25, 30, 35, or 40) consecutive amino acids of the hinge domain of CD8a.

The CARs of the present disclosure comprise a transmembrane domain that can be directly or indirectly fused to the extracellular antigen binding domain. The transmembrane domain may be derived either from a natural or from a synthetic source. Transmembrane domains compatible for use in the CARs described herein may be obtained from a naturally occurring protein. Alternatively, it can be a synthetic, non-naturally occurring protein segment, e.g., a hydrophobic protein segment that is thermodynamically stable in a cell membrane. In some embodiments, the transmembrane domains are derived from membrane proteins of Type I, Type II or Type III. In some embodiments, the transmembrane domain of the CAR described herein is derived from a Type I single-pass membrane protein. In some embodiments, transmembrane domains from multi-pass membrane proteins may also be compatible for use in the CARs described herein. Transmembrane domains for use in the CARs described herein can also comprise at least a portion of a synthetic, non-naturally occurring protein segment. In some embodiments, the protein segment has a length of about 15-100 amino acids. In some embodiments, the transmembrane domain is a synthetic, non-naturally occurring alpha helix or beta sheet. In some embodiments, the protein segment has a length of at least approximately 20 amino acids, e.g., at least 18, 19, 20, 21, 22, 23, 24, 25, 26, 27, 28, 29, 30, or more amino acids.

The transmembrane domain provided herein may comprise a transmembrane region and a cytoplasmic region located at the C-terminal side of the transmembrane domain. In some embodiments, the transmembrane region of the transmembrane domain comprises hydrophobic amino acid residues.

In some embodiments, the transmembrane domain of the CAR comprises a transmembrane domain chosen from the transmembrane domain of an alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD1 1a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7Ra, ITGA1, VLA1, CD49a, ITGA4, IA4, CD49D, ITGA6, VLA-6, CD49f, ITGAD, CD11d, ITGAE, CD103, ITGAL, CD1 1a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

The intracellular signaling domain in the CARs provided herein is responsible for activation of at least one of the normal effector functions of the immune effector cell expressing the CARs. In some embodiments, the intracellular signaling domain comprises a primary intracellular signaling domain of an immune effector cell. In some embodiments, the CAR comprises an intracellular signaling domain consisting essentially of a primary intracellular signaling domain of an immune effector cell. In some embodiments, the primary intracellular signaling domain comprises a signaling motif known as immunoreceptor tyrosine-based activation motif, or ITAM. Exemplary ITAM-containing primary cytoplasmic signaling sequences include those derived from CD3 zeta, FcR gamma (FCER1G), FcR beta (FCER1B), CD3 gamma, CD3 delta, CD3 epsilon, CD5, CD22, CD79a, CD79b, and CD66d. In some embodiments, the primary intracellular signaling domain is derived from CD3 zeta. In some embodiments, the intracellular signaling domain consists of a cytoplasmic signaling domain of CD3 zeta. In some embodiments, the primary intracellular signaling domain is a cytoplasmic signaling domain of wild-type CD3 zeta.

In some embodiments, the CAR comprises at least one co-stimulatory signaling domain. The co-stimulatory signaling domain of the chimeric receptor described herein can be a cytoplasmic signaling domain from a co-stimulatory protein, which transduces a signal and modulates responses mediated by immune cells. In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains. In some embodiments, the intracellular signaling domain comprises two or more co-stimulatory signaling domains from different co-stimulatory proteins, such as any two or more co-stimulatory proteins described herein. In some embodiments, the intracellular signaling domain comprises a primary intracellular signaling domain (such as cytoplasmic signaling domain of CD3 zeta) and one or more co-stimulatory signaling domains. In some embodiments, the one or more co-stimulatory signaling domains and the primary intracellular signaling domain (such as cytoplasmic signaling domain of CD3 zeta) are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as cytoplasmic signaling domain of CD3 zeta). Multiple co-stimulatory signaling domains may provide additive or synergistic stimulatory effects.

The co-stimulatory signaling domain of any co-stimulatory molecule may be compatible for use in the CARs described herein. Examples of co-stimulatory signaling domains for use in the CARs can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BlyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thyl, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the one or more co-stimulatory signaling domains are selected from the group consisting of CD27, CD28, CD137, OX40, CD30, CD40, lymphocyte function-associated antigen-1 (LFA-1), CD2, CD7, LIGHT, NKG2C, B7-H3 and ligands that s bind to CD83. In some embodiments, co-stimulatory signaling domains for use in the CARs comprise intracellular signaling domains from cytokine receptors.

In some embodiments, the intracellular signaling domain in the CAR of the present disclosure comprises a co-stimulatory signaling domain derived from CD137 (i.e., 4-1BB). In some embodiments, the intracellular signaling domain comprises a cytoplasmic signaling domain of CD3 zeta and a co-stimulatory signaling domain of CD137.

### 4.7. Pharmaceutical Compositions

In one aspect, the present disclosure further provides pharmaceutical compositions comprising the activated and expanded Vγ9Vδ2 T cells or the engineered Vγ9Vδ2 T cells of the present disclosure. In some embodiments, a pharmaceutical composition comprises a therapeutically effective amount of the activated and expanded Vγ9Vδ2 T cells or the engineered Vγ9Vδ2 T cells of the present disclosure and a pharmaceutically acceptable excipient.

In a specific embodiment, the term “excipient” can also refer to a diluent, adjuvant (e.g., Freunds' adjuvant (complete or incomplete), carrier or vehicle. Pharmaceutical excipients can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid excipients. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical excipients are described in Remington's Pharmaceutical Sciences (1990) Mack Publishing Co., Easton, PA. Such compositions will contain a prophylactically or therapeutically effective amount of the active ingredient provided herein, such as in purified form, together with a suitable amount of excipient so as to provide the form for proper administration to the patient. The formulation should suit the mode of administration.

In some embodiments, the choice of excipient is determined in part by the particular cell, and/or by the method of administration. Accordingly, there are a variety of suitable formulations.

Typically, acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g., Zn-protein complexes); chelating agents such as EDTA and/or non-ionic surfactants.

Buffers may be used to control the pH in a range which optimizes therapeutic effectiveness, especially if stability is pH dependent. Suitable buffering agents for use with the present disclosure include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris. Preservatives may be added to retard microbial growth. Suitable preservatives for

use with the present disclosure include octadecyldimethylbenzyl ammonium chloride;

hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol. Tonicity agents, sometimes known as “stabilizers” can be present to adjust or

maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Exemplary tonicity agents include polyhydric sugar alcohols, trihydric or higher sugar alcohols, such as glycerin, erythritol, arabitol, xylitol, sorbitol and mannitol.

Additional exemplary excipients include: (1) bulking agents, (2) solubility enhancers, (3) stabilizers and (4) agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone;
monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) may be present to help solubilize the therapeutic agent as well as to protect the therapeutic protein against agitation-induced aggregation, which also permits the formulation to be exposed to shear surface stress without causing denaturation of the active therapeutic protein or antibody. Suitable non-ionic surfactants include, e.g., polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they are preferably sterile. The pharmaceutical composition may be rendered sterile by filtration through sterile filtration membranes. The pharmaceutical compositions herein generally can be placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

In another embodiment, a pharmaceutical composition can be provided as a controlled release or sustained release system. In one embodiment, a pump may be used to achieve controlled or sustained release (see, e.g., Sefton, Crit. Ref. Biomed. Eng. 14:201-40 (1987); Buchwald et al., Surgery 88:507-16 (1980); and Saudek et al., N. Engl. J. Med. 321:569-74 (1989)). In another embodiment, polymeric materials can be used to achieve controlled or sustained release of a prophylactic or therapeutic agent (e.g., a fusion protein as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61-126 (1983); Levy et al., Science 228:190-92 (1985); During et al., Ann. Neurol. 25:351-56 (1989); Howard et al., J. Neurosurg. 71:105-12 (1989); U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, Science 249:1527-33 (1990). Any technique known to one of skill in the art can be used to produce sustained release formulations comprising one or more agents as described herein (see, e.g., U.S. Pat. No. 4,526,938, PCT publication Nos. WO 91/05548 and WO 96/20698, Ning et al., Radiotherapy & Oncology 39:179-89 (1996); Song et al., PDA J. of Pharma. Sci. & Tech. 50:372-97 (1995); Cleek et al., Pro. Int'l. Symp. Control. Rel. Bioact. Mater. 24:853-54 (1997); and Lam et al., Proc. Int'l. Symp. Control Rel. Bioact. Mater. 24:759-60 (1997)).

The pharmaceutical compositions described herein may also contain more than one active compound or agent as necessary for the particular indication being treated. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 18th edition.

Various compositions and delivery systems are known and can be used with therapeutic agents provided herein, including, but not limited to, encapsulation in liposomes, microparticles, microcapsules, recombinant cells capable of expressing the single domain antibody or therapeutic molecule provided herein, construction of a nucleic acid as part of a retroviral or other vector, etc.

In some embodiments, the pharmaceutical composition provided herein contains the binding molecules and/or cells in amounts effective to treat or prevent the disease or disorder, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined.

### 4.8. Methods and Uses

In another aspect, provided herein are methods for using and uses of the activated and expanded Vγ9Vδ2 T cells, such as those described in Sections 4.3 and 4.4 above.

In yet another aspect, provided herein are methods for using and uses of the engineered Vγ9Vδ2 T cells expressing the recombinant receptors, such as those described in Section 4.6 above, including a CAR T cell, wherein the CAR T cell is a Vγ9Vδ2 T cell.

In some embodiments, provided herein are methods for treating a disease or disorder in a subject, comprising administering to the subject: (i) a therapeutically effective amount of the activated and expanded Vγ9Vδ2 T cells or a pharmaceutical composition comprising the activated and expanded Vγ9Vδ2 T cells and (ii) a therapeutically effective amount of one or more multispecific antibodies so that the activated and expanded Vγ9Vδ2 T cells are directed to target cells. In certain embodiments, provided herein are methods for treating a disease or disorder in a subject, comprising administering to the subject: (i) a therapeutically effective amount of the activated and expanded Vγ9Vδ2 T cells or a pharmaceutical composition comprising the activated and expanded Vγ9Vδ2 T cells and (ii) a therapeutically effective amount of Vγ9×TAA bispecific antibodies. In certain embodiments, provided herein are methods for treating a disease or disorder in a subject comprising administering to the subject: (i) a therapeutically effective amount of the activated and expanded Vγ9Vδ2 T cells or a pharmaceutical composition comprising the activated and expanded Vγ9Vδ2 T cells and (ii) a therapeutically effective amount of CD3×TAA bispecific antibodies. In certain embodiments, provided herein are methods for treating a disease or disorder in a subject, comprising administering to the subject: (i) a therapeutically effective amount of the activated and expanded Vγ9Vδ2 T cells or a pharmaceutical composition comprising the activated and expanded Vγ9Vδ2 T cells and (ii) a therapeutically effective amount of Vγ9×TAA and CD3×TAA bispecific antibodies.

In some embodiments, provided herein are methods for treating a disease or disorder in a subject, comprising administering to the subject a therapeutically effective amount of CAR T cells, wherein the CAR T cells are produced by a method comprising: (i) obtaining a population of cells comprising Vγ9Vδ2 T cells; and (ii) introducing a nucleic acid encoding a chimeric antigen receptor (CAR) into the population of cells.

In some embodiments, the activated and expanded Vγ9Vδ2 T cells or the engineered Vγ9Vδ2 T cells provided herein are useful as allogenic CAR T cell therapies. In some embodiments, the activated and expanded Vγ9Vδ2 T cells or the engineered Vγ9Vδ2 T cells provided herein have more safety features that are absent from the traditional autologous T cell therapy, for example, no or low cytokine storm, no stimulation of regulatory T cells, reduced self-tissue damage, reduced induction of autoimmunity, reduced graft-versus-host disease, etc.

Such methods and uses include therapeutic methods and uses, for example, involving administration of the cells, or compositions containing the same, to a subject having a disease or disorder. In some embodiments, the cell is administered in an effective amount to effectively treat the disease or disorder. Uses include uses of the cells in such methods and treatments, and in the preparation of a medicament in order to carry out such therapeutic methods. In some embodiments, the methods are carried out by administering the cells, or compositions comprising the same, to the subject having or suspected of having the disease or condition. In some embodiments, the methods thereby treat the disease or disorder in the subject.

In some embodiments, the treatment provided herein cause complete or partial amelioration or reduction of a disease or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms include, but do not imply, complete curing of a disease or complete elimination of any symptom or effect(s) on all symptoms or outcomes.

As used herein, in some embodiments, the treatment provided herein delay development of a disease or disorder, e.g., defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease or disorder. For example, a late stage cancer, such as development of metastasis, may be delayed. In other embodiments, the method or the use provided herein prevents a disease or disorder.

In some embodiments, the present T cell therapies are used for treating solid tumor cancer. In some embodiments, the present T cell therapies are used for treating blood cancer. In some embodiments, the present T cell therapies are used for treating an autoimmune and inflammatory disease. In some embodiments, the present T cell therapies are used for treating a neurological disease.

In some embodiments, the methods include adoptive cell therapy, whereby genetically engineered cells are administered to a subject. Such administration can promote activation of the cells (e.g., T cell activation), such that the cells of the disease or disorder are targeted for destruction.

In some embodiments, the methods include administration of the cells or a composition comprising the cells to a subject, tissue, or cell, such as one having, at risk for, or suspected of having the disease or disorder. In some embodiments, the cells, populations, and compositions are administered to a subject having the particular disease or disorder to be treated, e.g., via adoptive cell therapy, such as adoptive T cell therapy. In some embodiments, the cells or compositions are administered to a subject, such as a subject having or at risk for the disease or disorder. In some embodiments, the methods thereby treat, e.g., ameliorate one or more symptom of the disease or disorder.

Methods for administration of cells for adoptive cell therapy are known, as described, e.g., in US Patent Application Publication No. 2003/0170238; U.S. Pat. No. 4,690,915; Rosenberg, Nat Rev Clin Oncol. 8 (10): 577-85 (2011); Themeli et al., Nat Biotechnol. 31 (10): 928-933 (2013); Tsukahara et al., Biochem Biophys Res Commun 438 (1): 84-9 (2013); and Davila et al., PloS ONE 8 (4): e61338 (2013). These methods may be used in connection with the methods and compositions provided herein.

In some embodiments, the cell therapy (e.g., adoptive T cell therapy) is carried out by autologous transfer, in which the cells are isolated and/or otherwise prepared from the subject who is to receive the cell therapy, or from a sample derived from such a subject. Thus, in some aspects, the cells are derived from a subject in need of a treatment and the cells, following isolation and processing are administered to the same subject. In other embodiments, the cell therapy (e.g., adoptive T cell therapy) is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. The subject can be male or female and can be any suitable age, including infant, juvenile, adolescent, adult, and geriatric subjects. In some examples, the subject is a validated animal model for disease, adoptive cell therapy, and/or for assessing toxic outcomes.

The composition provided herein can be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjunctival injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration.

The amount of a prophylactic or therapeutic agent provided herein that will be effective in the prevention and/or treatment of a disease or condition can be determined by standard clinical techniques. Effective doses may be extrapolated from dose-response curves derived from in vitro or animal model test systems. For the prevention or treatment of disease, the appropriate dosage of the binding molecule or cell may depend on the type of disease or disorder to be treated, the type of binding molecule, the severity and course of the disease or disorder, whether therapeutic agent is administered for preventive or therapeutic purposes, previous therapy, the patient's clinical history and response to the agent, and the discretion of the attending physician. The compositions, molecules and cells are in some embodiments suitably administered to the patient at one time or over a series of treatments. Multiple doses may be administered intermittently. An initial higher loading dose, followed by one or more lower doses may be administered.

In the context of genetically engineered cells, in some embodiments, a subject may be administered the range of about one million to about 100 billion cells and/or that amount of cells per kilogram of body weight. In some embodiments, wherein the pharmaceutical composition comprises any one of the engineered immune cells described herein, the pharmaceutical composition is administered at a dosage of at least about any of 104, 105, 106, 107, 108, or 10⁹ cells/kg of body weight of the individual. Dosages may vary depending on attributes particular to the disease or disorder and/or patient and/or other treatments.

In some embodiments, the pharmaceutical composition is administered for a single time. In some embodiments, the pharmaceutical composition is administered for multiple times (such as any of 2, 3, 4, 5, 6, or more times). In some embodiments, the pharmaceutical composition is administered once or multiple times during a dosing cycle. A dosing cycle can be, e.g., 1, 2, 3, 4, 5 or more week(s), or 1, 2, 3, 4, 5, or more month(s). The optimal dosage and treatment regime for a particular patient can be determined by one skilled in the art of medicine by monitoring the patient for signs of disease and adjusting the treatment accordingly.

In some embodiments, the compositions provided herein are administered as part of a combination treatment, such as simultaneously with or sequentially with, in any order, another therapeutic intervention, such as another antibody or engineered cell or receptor or agent, such as a cytotoxic or therapeutic agent.

In some embodiments, the compositions provided herein are co-administered with one or more additional therapeutic agents or in connection with another therapeutic intervention, either simultaneously or sequentially in any order. In some embodiments, the cells are co-administered with another therapy sufficiently close in time such that the cell populations enhance the effect of one or more additional therapeutic agents, or vice versa. In some embodiments, the compositions provided herein are administered prior to the one or more additional therapeutic agents. In some embodiments, the compositions provided herein are administered after to the one or more additional therapeutic agents.

In certain embodiments, once the cells are administered to a mammal (e.g., a human), the biological activity of the engineered cell populations is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32 (7): 689-702 (2009), and Herman et al. J. Immunological Methods, 285 (1): 25-40 (2004). In certain embodiments, the biological activity of the cells also can be measured by assaying expression and/or secretion of certain cytokines, such as CD107a, IFNγ, IL-2, and TNF. In some aspects, the biological activity is measured by assessing clinical outcome, such as reduction in tumor burden or load.

In yet another aspect, provided herein are methods for establishing an in vivo engraftment of Vγ9Vδ2 T cells as described herein in a receiving subject. In some embodiments, the method comprises (i) obtaining a population of cells comprising Vγ9Vδ2 T cells disclosed herein; and (ii) adoptively transferring the population of cells to the receiving subject. In some embodiments, the population of cells comprising Vγ9Vδ2 T cells for the adoptive transfer are obtained using the methods for ex vivo activation and expansion of Vγ9Vδ2 T cells as described herein.

In some embodiments, the population of cells comprising Vγ9Vδ2 T cells for the adoptive transfer is produced by ex vivo activation and expression of a population of cells comprising T cells that are obtained from the receiving subject. In some embodiments, the population of cells comprising Vγ9Vδ2 T cells for the adoptive transfer is produced by ex vivo activation and expression of a population of cells comprising T cells that are obtained from a subject other than the receiving subject. In some embodiments, the subject from which the population of cells comprising T cells are obtained is a donor subject. In some embodiments, the donor subject and the receiving subject are of the same species. In some embodiments, the donor subject and the receiving subject from the same species are genetically related. In some embodiments, the donor subject and the receiving subject are of different species. In some embodiments, the receiving subject is a human in need thereof. In some embodiments, the receiving subject is a model animal. In some embodiments, the donor subject is a human. In some embodiments, the donor subject is a human individual, and the receiving subject is a different human individual. In some embodiments, the donor subject is a human individual, and the receiving subject is the same human individual. In some embodiments, the donor subject is a human individual, and the receiving subject is a non-human mammal. In some embodiments, the non-human animal is a model animal. In some embodiments, the non-human individual is a mouse, a pig, a monkey, a chimpanzee, a cow, or a sheep. In some embodiments, the receiving subject is a patient suffering from a disease or condition. In some embodiments, the receiving subject has cancer. In some embodiments, the receiving subject has a blood cancer. In some embodiments, the receiving subject has a solid tumor.

In some embodiments, the population of cells comprising Vγ9Vδ2 T cells for the adoptive transfer comprises more than 10%, more than 15%, more than 20%, more than 25%, more than 30%, more than 35%, more than 40%, more than 45%, more than 50%, more than 55%, or more than 60% Vγ9Vδ2 T cells in the population of cells. In some embodiments, the population of cells comprising Vγ9Vδ2 T cells for the adoptive transfer comprises more than 10% Vγ9Vδ2 T cells in the population of cells. In some embodiments, the population of cells comprising Vγ9Vδ2 T cells for the adoptive transfer comprises more than 15% Vγ9Vδ2 T cells in the population of cells. In some embodiments, the population of cells comprising Vγ9Vδ2 T cells for the adoptive transfer comprises more than 20% Vγ9Vδ2 T cells in the population of cells. In some embodiments, the population of cells comprising Vγ9Vδ2 T cells for the adoptive transfer comprises more than 25% Vγ9Vδ2 T cells in the population of cells.

In some embodiments, the population of cells comprising Vγ9Vδ2 T cells for the adoptive transfer comprises more than 30% Vγ9Vδ2 T cells in the population of cells. In some embodiments, the population of cells comprising Vγ9Vδ2 T cells for the adoptive transfer comprises more than 35% Vγ9Vδ2 T cells in the population of cells. In some embodiments, the population of cells comprising Vγ9Vδ2 T cells for the adoptive transfer comprises more than 40% Vγ9Vδ2 T cells in the population of cells. In some embodiments, the population of cells comprising Vγ9Vδ2 T cells for the adoptive transfer comprises more than 45% Vγ9Vδ2 T cells in the population of cells. In some embodiments, the population of cells comprising Vγ9Vδ2 T cells for the adoptive transfer comprises more than 50% Vγ9Vδ2 T cells in the population of cells. In some embodiments, the population of cells comprising Vγ9Vδ2 T cells for the adoptive transfer comprises more than 55% Vγ9Vδ2 T cells in the population of cells. In some embodiments, the population of cells comprising Vγ9Vδ2 T cells for the adoptive transfer comprises more than 60% Vγ9Vδ2 T cells in the population of cells.

In some embodiments, the population of cells comprising Vγ9Vδ2 T cells for the adoptive transfer comprises 10%-99%, 20%-95%, 30%-95%, 35%-95%, 40%-95%, 45%-95%, 50%-95%, 55%-95%, 60%-95%, or 65%-95% Vγ9Vδ2 T cells in the population of cells. In some embodiments, the population of cells comprising Vγ9Vδ2 T cells for the adoptive transfer comprises 10%-99% Vγ9Vδ2 T cells in the population of cells. In some embodiments, the population of cells comprising Vγ9Vδ2 T cells for the adoptive transfer comprises 20%-95% Vγ9Vδ2 T cells in the population of cells. In some embodiments, the population of cells comprising Vγ9Vδ2 T cells for the adoptive transfer comprises 30%-95% Vγ9Vδ2 T cells in the population of cells. In some embodiments, the population of cells comprising Vγ9Vδ2 T cells for the adoptive transfer comprises 35%-95% Vγ9Vδ2 T cells in the population of cells. In some embodiments, the population of cells comprising Vγ9Vδ2 T cells for the adoptive transfer comprises 40%-95% Vγ9Vδ2 T cells in the population of cells. In some embodiments, the population of cells comprising Vγ9Vδ2 T cells for the adoptive transfer comprises 45%-95% Vγ9Vδ2 T cells in the population of cells. In some embodiments, the population of cells comprising Vγ9Vδ2 T cells for the adoptive transfer comprises 50%-95% Vγ9Vδ2 T cells in the population of cells. In some embodiments, the population of cells comprising Vγ9Vδ2 T cells for the adoptive transfer comprises 55%-95% Vγ9Vδ2 T cells in the population of cells. In some embodiments, the population of cells comprising Vγ9Vδ2 T cells for the adoptive transfer comprises 60%-95% Vγ9Vδ2 T cells in the population of cells. In some embodiments, the population of cells comprising Vγ9Vδ2 T cells for the adoptive transfer comprises 65%-95% Vγ9Vδ2 T cells in the population of cells.

In some embodiments, the population of cells comprising Vγ9Vδ2 T cells for the adoptive transfer is enriched for Vγ9Vδ2 T cells. In some embodiments, the enrichment for the Vγ9Vδ2 T cells is using any method for enriching Vγ9Vδ2 T cells described herein. In some embodiments, the enriched population of cells comprising Vγ9Vδ2 T cells for the adoptive transfer comprises more than 65%, more than 70%, more than 75%, more than 80%, more than 85%, more than 90%, more than 95%, more than 99% Vγ9Vδ2 T cells in the population of cells. In some embodiments, the enriched population of cells comprising Vγ9Vδ2 T cells for the adoptive transfer comprises more than 65% Vγ9Vδ2 T cells in the population of cells. In some embodiments, the enriched population of cells comprising Vγ9Vδ2 T cells for the adoptive transfer comprises more than 70% Vγ9Vδ2 T cells in the population of cells. In some embodiments, the enriched population of cells comprising Vγ9Vδ2 T cells for the adoptive transfer comprises more than 75% Vγ9Vδ2 T cells in the population of cells. In some embodiments, the enriched population of cells comprising Vγ9Vδ2 T cells for the adoptive transfer comprises more than 80% Vγ9Vδ2 T cells in the population of cells. In some embodiments, the enriched population of cells comprising Vγ9Vδ2 T cells for the adoptive transfer comprises more than 85% Vγ9Vδ2 T cells in the population of cells. In some embodiments, the enriched population of cells comprising Vγ9Vδ2 T cells for the adoptive transfer comprises more than 90% Vγ9Vδ2 T cells in the population of cells. In some embodiments, the enriched population of cells comprising Vγ9Vδ2 T cells for the adoptive transfer comprises more than 95% Vγ9Vδ2 T cells in the population of cells. In some embodiments, the enriched population of cells comprising Vγ9Vδ2 T cells for the adoptive transfer comprises more than 99% Vγ9Vδ2 T cells in the population of cells.

In some embodiments, the population of cells comprising Vγ9Vδ2 T cells for the adoptive transfer is a purified population of Vγ9Vδ2 T cells. In some embodiments, the purified population of Vγ9Vδ2 T cells for the adoptive transfer comprises more than 65%, more than 70%, more than 75%, more than 80%, more than 85%, more than 90%, more than 95%, more than 99% Vγ9Vδ2 T cells in the population of cells. In some embodiments, the purified population of Vγ9Vδ2 T cells for the adoptive transfer comprises more than 65%

Vγ9Vδ2 T cells in the population of cells. In some embodiments, the purified population of Vγ9Vδ2 T cells for the adoptive transfer comprises more than 65% Vγ9Vδ2 T cells in the population of cells. In some embodiments, the purified population of Vγ9Vδ2 T cells for the adoptive transfer comprises more than 70% Vγ9Vδ2 T cells in the population of cells. In some embodiments, the purified population of Vγ9Vδ2 T cells for the adoptive transfer comprises more than 75% Vγ9Vδ2 T cells in the population of cells. In some embodiments, the purified population of Vγ9Vδ2 T cells for the adoptive transfer comprises more than 80% Vγ9Vδ2 T cells in the population of cells. In some embodiments, the purified population of Vγ9Vδ2 T cells for the adoptive transfer comprises more than 85% Vγ9Vδ2 T cells in the population of cells. In some embodiments, the purified population of Vγ9Vδ2 T cells for the adoptive transfer comprises more than 90% Vγ9Vδ2 T cells in the population of cells. In some embodiments, the purified population of Vγ9Vδ2 T cells for the adoptive transfer comprises more than 95% Vγ9Vδ2 T cells in the population of cells. In some embodiments, the purified population of Vγ9Vδ2 T cells for the adoptive transfer comprises more than 99% Vγ9Vδ2 T cells in the population of cells. In some embodiments, the purified population of Vγ9Vδ2 T cells for the adoptive transfer comprises 100% Vγ9Vδ2 T cells in the population of cells.

In some embodiments, the population of cells comprising Vγ9Vδ2 T cells for the adoptive transfer comprises less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1%, or is devoid of αβ T cells in the population of cells. In some embodiments, the population of cells comprising Vγ9Vδ2 T cells for the adoptive transfer comprises less than 95% αβ T cells in the population of cells. In some embodiments, the population of cells comprising Vγ9Vδ2 T cells for the adoptive transfer comprises less than 90% αβ T cells in the population of cells. In some embodiments, the population of cells comprising Vγ9Vδ2 T cells for the adoptive transfer comprises less than 85% αβ T cells in the population of cells. In some embodiments, the population of cells comprising Vγ9Vδ2 T cells for the adoptive transfer comprises less than 80% αβ T cells in the population of cells. In some embodiments, the population of cells comprising Vγ9Vδ2 T cells for the adoptive transfer comprises less than 75% αβ T cells in the population of cells. In some embodiments, the population of cells comprising Vγ9Vδ2 T cells for the adoptive transfer comprises less than 70% αβ T cells in the population of cells. In some embodiments, the population of cells comprising Vγ9Vδ2 T cells for the adoptive transfer comprises less than 65% αβ T cells in the population of cells. In some embodiments, the population of cells comprising Vγ9Vδ2 T cells for the adoptive transfer comprises less than 60% αβ T cells in the population of cells. In some embodiments, the population of cells comprising Vγ9Vδ2 T cells for the adoptive transfer comprises less than 55% αβ T cells in the population of cells. In some embodiments, the population of cells comprising Vγ9Vδ2 T cells for the adoptive transfer comprises less than 50% αβ T cells in the population of cells. In some embodiments, the population of cells comprising Vγ9Vδ2 T cells for the adoptive transfer comprises less than 45% αβ T cells in the population of cells. In some embodiments, the population of cells comprising Vγ9Vδ2 T cells for the adoptive transfer comprises less than 40% αβ T cells in the population of cells. In some embodiments, the population of cells comprising Vγ9Vδ2 T cells for the adoptive transfer comprises less than 35% αβ T cells in the population of cells. In some embodiments, the population of cells comprising Vγ9Vδ2 T cells for the adoptive transfer comprises less than 30% αβ T cells in the population of cells. In some embodiments, the population of cells comprising Vγ9Vδ2 T cells for the adoptive transfer comprises less than 25% αβ T cells in the population of cells. In some embodiments, the population of cells comprising Vγ9Vδ2 T cells for the adoptive transfer comprises less than 20% αβ T cells in the population of cells. In some embodiments, the population of cells comprising Vγ9Vδ2 T cells for the adoptive transfer comprises less than 15% αβ T cells in the population of cells. In some embodiments, the population of cells comprising Vγ9Vδ2 T cells for the adoptive transfer comprises less than 10% αβ T cells in the population of cells. In some embodiments, the population of cells comprising Vγ9Vδ2 T cells for the adoptive transfer comprises less than 5% αβ T cells in the population of cells. In some embodiments, the population of cells comprising Vγ9Vδ2 T cells for the adoptive transfer comprises less than 1% αβ T cells in the population of cells. In some embodiments, the population of cells comprising Vγ9Vδ2 T cells for the adoptive transfer is devoid of αβ T cells in the population of cells.

In some embodiments, the population of cells comprising Vγ9Vδ2 T cells for the adoptive transfer comprises at least about or about 5×10⁶ cells, at least about or about 1×10⁷ cells, at least about or about 10×10⁷ cells, at least about or about 20×10⁷ cells, at least about or about 30×10⁷ cells, at least about or about 40×10⁷ cells, at least about or about 50×10⁷ cells, at least about or about 60×10⁷ cells, at least about or about 70×10⁷ cells, at least about or about 80×10⁷ cells, at least about or about 90×10⁷ cells, at least about or about 1×10⁸ cells, at least about or about 10×10⁸ cells, at least about or about 20×10⁸ cells, at least about or about 30×10⁸ cells, at least about or about 40×10⁸ cells, at least about or about 50×10⁸ cells, at least about or about 60×10⁸ cells, at least about or about 70×10⁸ cells, at least about or about 80×10⁸ cells, at least about or about 90×10⁸ cells, or at least about or about 1×10⁹ cells. In some embodiments, the population of cells comprising Vγ9Vδ2 T cells for the adoptive transfer comprises about 5×10⁶ cells. In some embodiments, the population of cells comprising Vγ9Vδ2 T cells for the adoptive transfer comprises about 1×10⁷ cells. In some embodiments, the population of cells comprising Vγ9Vδ2 T cells for the adoptive transfer comprises about 10×10⁷ cells. In some embodiments, the population of cells comprising Vγ9Vδ2 T cells for the adoptive transfer comprises about 20×10⁷ cells. In some embodiments, the population of cells comprising Vγ9Vδ2 T cells for the adoptive transfer comprises about 30×10⁷ cells. In some embodiments, the population of cells comprising Vγ9Vδ2 T cells for the adoptive transfer comprises about 40×10⁷ cells. In some embodiments, the population of cells comprising Vγ9Vδ2 T cells for the adoptive transfer comprises about 50×10⁷ cells. In some embodiments, the population of cells comprising Vγ9Vδ2 T cells for the adoptive transfer comprises about 60×10⁷ cells. In some embodiments, the population of cells comprising Vγ9Vδ2 T cells for the adoptive transfer comprises about 70×10⁷ cells. In some embodiments, the population of cells comprising Vγ9Vδ2 T cells for the adoptive transfer comprises about 80×10⁷ cells. In some embodiments, the population of cells comprising Vγ9Vδ2 T cells for the adoptive transfer comprises about 90×10⁷ cells. In some embodiments, the population of cells comprising Vγ9Vδ2 T cells for the adoptive transfer comprises about 1×10⁸ cells. In some embodiments, the population of cells comprising Vγ9Vδ2 T cells for the adoptive transfer comprises about 10×10⁸ cells. In some embodiments, the population of cells comprising Vγ9Vδ2 T cells for the adoptive transfer comprises about 20×10⁸ cells. In some embodiments, the population of cells comprising Vγ9Vδ2 T cells for the adoptive transfer comprises about 30×10⁸ cells. In some embodiments, the population of cells comprising Vγ9Vδ2 T cells for the adoptive transfer comprises about 40×10⁸ cells. In some embodiments, the population of cells comprising Vγ9Vδ2 T cells for the adoptive transfer comprises about 50×10⁸ cells. In some embodiments, the population of cells comprising Vγ9Vδ2 T cells for the adoptive transfer comprises about 60×10⁸ cells. In some embodiments, the population of cells comprising Vγ9Vδ2 T cells for the adoptive transfer comprises about 70×10⁸ cells. In some embodiments, the population of cells comprising Vγ9Vδ2 T cells for the adoptive transfer comprises about 80×10⁸ cells. In some embodiments, the population of cells comprising Vγ9Vδ2 T cells for the adoptive transfer comprises about 90×10⁸ cells. In some embodiments, the population of cells comprising Vγ9Vδ2 T cells for the adoptive transfer comprises about 1×10⁹ cells. In any of the embodiments described herein, the population of cells comprising Vγ9Vδ2 T cells for the adoptive transfer comprises more than 10%, more than 15%, more than 20%, more than 25%, more than 30%, more than 35%, more than 40%, more than 45%, more than 50%, more than 55%, or more than 60% Vγ9Vδ2 T cells in the population of cells. In any of the embodiments described herein, the population of cells comprising Vγ9Vδ2 T cells for the adoptive transfer comprises 10%-99%, 20%-95%, 30%-95%, 35%-95%, 40%-95%, 45%-95%, 50%-95%, 55%-95%, 60%-95%, or 65%-95% Vγ9Vδ2 T cells in the population of cells. In any of the embodiments described herein, the population of cells comprising Vγ9Vδ2 T cells for the adoptive transfer is enriched for Vγ9Vδ2 T cells. In some embodiments, the enriched population of cells comprising Vγ9Vδ2 T cells for the adoptive transfer comprises more than 65%, more than 70%, more than 75%, more than 80%, more than 85%, more than 90%, more than 95%, more than 99% Vγ9Vδ2 T cells in the population of cells. In any of the embodiments descried herein, the population of cells comprising Vγ9Vδ2 T cells for the adoptive transfer is a purified population of Vγ9Vδ2 T cells. In some embodiments, the purified population of Vγ9Vδ2 T cells for the adoptive transfer comprises more than 65%, more than 70%, more than 75%, more than 80%, more than 85%, more than 90%, more than 95%, more than 99% Vγ9Vδ2 T cells in the population of cells. In some embodiments, the purified population of Vγ9Vδ2 T cells for the adoptive transfer comprises 100% Vγ9Vδ2 T cells. In any of the embodiments descried herein, the population of cells comprising Vγ9Vδ2 T cells for the adoptive transfer comprises less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1%, or is devoid of αβ T cells in the population of cells.

In some embodiments, the enrichment for Vγ9Vδ2 T cells in the population of cells for adoptive transfer is produced by any of the method for enriching Vγ9Vδ2 T cells as described herein. In some embodiments, the enrichment for Vγ9Vδ2 T cells in the population of cells for adoptive transfer is obtained at least 1 day, at least 2 days, at least 3 days, at least 4 days, at least 5 days, at least 6 days, or at least 7 days before adoptively transferring the population of cells comprising Vγ9Vδ2 T cells to the receiving subject. In some embodiments, the enrichment for Vγ9Vδ2 T cells in the population of cells for adoptive transfer is obtained at least 1 day before adoptively transferring the population of cells comprising Vγ9Vδ2 T cells to the receiving subject. In some embodiments, the enrichment for Vγ9Vδ2 T cells in the population of cells for adoptive transfer is obtained at least 2 days before adoptively transferring the population of cells comprising Vγ9Vδ2 T cells to the receiving subject. In some embodiments, the enrichment for Vγ9Vδ2 T cells in the population of cells for adoptive transfer is obtained at least 3 days before adoptively transferring the population of cells comprising Vγ9Vδ2 T cells to the receiving subject. In some embodiments, the enrichment for Vγ9Vδ2 T cells in the population of cells for adoptive transfer is obtained at least 4 days before adoptively transferring the population of cells comprising Vγ9Vδ2 T cells to the receiving subject. In some embodiments, the enrichment for Vγ9Vδ2 T cells in the population of cells for adoptive transfer is obtained at least 5 days before adoptively transferring the population of cells comprising Vγ9Vδ2 T cells to the receiving subject. In some embodiments, the enrichment for Vγ9Vδ2 T cells in the population of cells for adoptive transfer is obtained at least 6 days before adoptively transferring the population of cells comprising Vγ9Vδ2 T cells to the receiving subject. In some embodiments, the enrichment for Vγ9Vδ2 T cells in the population of cells for adoptive transfer is obtained at least 7 days before adoptively transferring the population of cells comprising Vγ9Vδ2 T cells to the receiving subject.

In some embodiments, the adoptively transferring the population of cells comprising Vγ9Vδ2 T cells comprises administering the population of cells to the receiving subject. In some embodiments, the method further comprises (iii) administering, concurrently and/or sequentially with the adoptively transferring, an effective amount of a second composition. In some embodiments, the method further comprises administering, concurrently with the adoptively transferring, an effective amount of the second composition. In some embodiments, the method further comprises administering, sequentially with the adoptively transferring, an effective amount of a second composition. In some embodiments, the method further comprises administering, both concurrently and sequentially with the adoptively transferring, an effective amount of a second composition.

In some embodiments, administering the population of cells comprising Vγ9Vδ2 T cells to the receiving subject is by intravenous administration. In some embodiments, administering the population of cells comprising Vγ9Vδ2 T cells to the receiving subject is by intraperitoneal administration. In some embodiments, administration of the second composition to the receiving subject is by intravenous administration. In some embodiments, administration of the second composition to the receiving subject is by intraperitoneal administration. In some embodiments, administering the population of cells comprising Vγ9Vδ2 T cells to the receiving subject is by intravenous administration, and administration of the second composition to the receiving subject is by intravenous administration. In some embodiments, administering the population of cells comprising Vγ9Vδ2 T cells to the receiving subject is by intravenous administration, and administration of the second composition to the receiving subject is by intraperitoneal administration. In some embodiments, administering the population of cells comprising Vγ9Vδ2 T cells to the receiving subject is by intraperitoneal administration, and administration of the second composition to the receiving subject is by intravenous administration. In some embodiments, administering the population of cells comprising Vγ9Vδ2 T cells to the receiving subject is by intraperitoneal administration, and administration of the second composition to the receiving subject is by intraperitoneal administration.

In some embodiments, the second composition administered, concurrently and/or sequentially with the adoptively transferring the population of cells comprising Vγ9Vδ2 T cells to the receiving subject is a composition comprising IL-2, IL-15, a bisphosphonate or a mevalonate pathway intermediate, or a combination thereof. In some embodiments, the second composition comprises an effective amount of IL-2. In some embodiments, the second composition comprises an effective amount of IL-15. In some embodiments, the second composition comprises an effective amount of bisphosphonate. In some embodiments, the second composition comprises an effective amount of mevalonate pathway intermediate. In some embodiments, the second composition comprises an effective amount of IL-2 and IL-15. In some embodiments, the second composition comprises an effective amount of IL-2 and bisphosphonate. In some embodiments, the second composition comprises an effective amount of IL-2 and mevalonate pathway intermediate. In some embodiments, the second composition comprises an effective amount of IL-15 and bisphosphonate. In some embodiments, the second composition comprises an effective amount of IL-15 and mevalonate pathway intermediate. In some embodiments, the second composition comprises an effective amount of IL-2, bisphosphonate, and mevalonate pathway intermediate. In some embodiments, the second composition comprises an effective amount of IL-15, bisphosphonate, and mevalonate pathway intermediate.

In some embodiments, the bisphosphonate is selected from the group consisting of zoledronic acid, risedronic acid, ibandronic acid, alendronic acid, pamidronic acid, tiludronic acid, etidronic acid, and clodronic acid. In some embodiments, the bisphosphonate is zoledronic acid. In some embodiments, the bisphosphonate is risedronic acid. In some embodiments, the bisphosphonate is ibandronic acid. In some embodiments, the bisphosphonate is alendronic acid. In some embodiments, the bisphosphonate is pamidronic acid. In some embodiments, the bisphosphonate is tiludronic acid. In some embodiments, the bisphosphonate is etidronic acid. In some embodiments, the bisphosphonate is clodronic acid.

In some embodiments, the mevalonate pathway intermediate is selected from the group consisting of HMBPP, BrHPP, and isopentenyl pyrophosphate. In some embodiments, the mevalonate pathway intermediate is HMBPP. In some embodiments, the mevalonate pathway intermediate is BrHPP. In some embodiments, the mevalonate pathway intermediate is isopentenyl pyrophosphate.

In some embodiments, IL-2 is administered at a dosage of about 1×10³ IU/kg of body weight to about 1×10⁵ IU/kg of body weight. In some embodiments, IL-2 is administered at a dosage of about 2×10³ IU/kg of body weight. In some embodiments, IL-2 is administered at a dosage of about 3×10³ IU/kg of body weight. In some embodiments, IL-2 is administered at a dosage of about 4×103 IU/kg of body weight. In some embodiments, IL-2 is administered at a dosage of about 5×103 IU/kg of body weight. In some embodiments, IL-2 is administered at a dosage of about 6×10³ IU/kg of body weight. In some embodiments, IL-2 is administered at a dosage of about 7×10³ IU/kg of body weight. In some embodiments, IL-2 is administered at a dosage of about 8×10³ IU/kg of body weight.

In some embodiments, IL-2 is administered at a dosage of about 9×10³ IU/kg of body weight. In some embodiments, IL-2 is administered at a dosage of about 1×10⁴ IU/kg of body weight. In some embodiments, IL-2 is administered at a dosage of about 2×10⁴ IU/kg of body weight. In some embodiments, IL-2 is administered at a dosage of about 3×10⁴ IU/kg of body weight. In some embodiments, IL-2 is administered at a dosage of about 4×10⁴ IU/kg of body weight.

In some embodiments, IL-2 is administered at a dosage of about 5×10⁴ IU/kg of body weight. In some embodiments, IL-2 is administered at a dosage of about 6×10⁴ IU/kg of body weight. In some embodiments, IL-2 is administered at a dosage of about 7×10⁴ IU/kg of body weight. In some embodiments, IL-2 is administered at a dosage of about 8×10⁴ IU/kg of body weight. In some embodiments, IL-2 is administered at a dosage of about 9×10⁴ IU/kg of body weight. In some embodiments, IL-2 is administered at a dosage of about 1×10⁵ IU/kg of body weight.

In some embodiments, the bisphosphonate is administered at a dosage of about 10 ug/kg of body weight to about 100 mg/kg of body weight. In some embodiments, the bisphosphonate is administered at a dosage of about 20 ug/kg of body weight. In some embodiments, the bisphosphonate is administered at a dosage of about 30 ug/kg of body weight. In some embodiments, the bisphosphonate is administered at a dosage of about 40 ug/kg of body weight. In some embodiments, the bisphosphonate is administered at a dosage of about 50 ug/kg of body weight. In some embodiments, the bisphosphonate is administered at a dosage of about 60 ug/kg of body weight. In some embodiments, the bisphosphonate is administered at a dosage of about 70 ug/kg of body weight. In some embodiments, the bisphosphonate is administered at a dosage of about 80 ug/kg of body weight. In some embodiments, the bisphosphonate is administered at a dosage of about 90 ug/kg of body weight. In some embodiments, the bisphosphonate is administered at a dosage of about 100 μg/kg of body weight. In some embodiments, the bisphosphonate is administered at a dosage of about 150 ug/kg of body weight. In some embodiments, the bisphosphonate is administered at a dosage of about 200 μg/kg of body weight. In some embodiments, the bisphosphonate is administered at a dosage of about 300 μg/kg of body weight. In some embodiments, the bisphosphonate is administered at a dosage of about 400 μg/kg of body weight. In some embodiments, the bisphosphonate is administered at a dosage of about 500 μg/kg of body weight. In some embodiments, the bisphosphonate is administered at a dosage of about 750 ug/kg of body weight. In some embodiments, the bisphosphonate is administered at a dosage of about 1 mg/kg of body weight. In some embodiments, the bisphosphonate is administered at a dosage of about 1.5 mg/kg of body weight. In some embodiments, the bisphosphonate is administered at a dosage of about 2 mg/kg of body weight. In some embodiments, the bisphosphonate is administered at a dosage of about 2.5 mg/kg of body weight. In some embodiments, the bisphosphonate is administered at a dosage of about 3 mg/kg of body weight. In some embodiments, the bisphosphonate is administered at a dosage of about 3.5 mg/kg of body weight. In some embodiments, the bisphosphonate is administered at a dosage of about 4 mg/kg of body weight. In some embodiments, the bisphosphonate is administered at a dosage of about 4.5 mg/kg of body weight. In some embodiments, the bisphosphonate is administered at a dosage of about 5 mg/kg of body weight. In some embodiments, the bisphosphonate is administered at a dosage of about 10 mg/kg of body weight. In some embodiments, the bisphosphonate is administered at a dosage of about 20 mg/kg of body weight. In some embodiments, the bisphosphonate is administered at a dosage of about 30 mg/kg of body weight. In some embodiments, the bisphosphonate is administered at a dosage of about 40 mg/kg of body weight. In some embodiments, the bisphosphonate is administered at a dosage of about 50 mg/kg of body weight. In some embodiments, the bisphosphonate is administered at a dosage of about 60 mg/kg of body weight. In some embodiments, the bisphosphonate is administered at a dosage of about 70 mg/kg of body weight. In some embodiments, the bisphosphonate is administered at a dosage of about 80 mg/kg of body weight. In some embodiments, the bisphosphonate is administered at a dosage of about 90 mg/kg of body weight. In some embodiments, the bisphosphonate is administered at a dosage of about 100 mg/kg of body weight.

In some embodiments of the method for establishing an in vivo engraftment of Vγ9Vδ2 T cells in a receiving subject as described herein, the adoptively transferred Vγ9Vδ2 T cells produce progeny cells in the subject. In some embodiments, the progeny cells are

CD45⁺ cells. In some embodiments, the progeny cells are CD56+ cells. In some embodiments, the progeny cells are CD69⁺ cells. In some embodiments, the progeny cells are CD45⁺CD56⁺ cells. In some embodiments, the progeny cells are CD45-CD69+ cells. In some embodiments, the progeny cells are CD45⁺ CD56 CD69 cells.

In some embodiments, the adoptively transferred Vγ9Vδ2 T cells are present in the blood of the receiving subject at least 7 days, at least 14 days, at least 21 days, or at least 28 days after the adoptive transfer of the population of cells to the receiving subject. In some embodiments, the adoptively transferred Vγ9Vδ2 T cells are present in the blood of the receiving subject at least 7 days after the adoptive transfer of the population of cells to the receiving subject. In some embodiments, the adoptively transferred Vγ9Vδ2 T cells are present in the blood of the receiving subject at least 14 days after the adoptive transfer of the population of cells to the receiving subject. In some embodiments, the adoptively transferred Vγ9Vδ2 T cells are present in the blood of the receiving subject at least 21 days after the adoptive transfer of the population of cells to the receiving subject. In some embodiments, the adoptively transferred Vγ9Vδ2 T cells are present in the blood of the receiving subject at least 28 days after the adoptive transfer of the population of cells to the receiving subject.

In some embodiments, the adoptively transferred Vγ9Vδ2 T cells infiltrate into a tissue in the receiving subject. In some embodiments, the tissue is a spleen tissue, a liver tissue, a lung tissue, an intestine tissue, a skin tissue, or a combination thereof. In some embodiments, the adoptively transferred Vγ9Vδ2 T cells infiltrate into a spleen tissue in the receiving subject. In some embodiments, the adoptively transferred Vγ9Vδ2 T cells infiltrate into a liver tissue in the receiving subject. In some embodiments, the adoptively transferred Vγ9Vδ2 T cells infiltrate into a lung tissue in the receiving subject. In some embodiments, the adoptively transferred Vγ9Vδ2 T cells infiltrate into a intestine tissue in the receiving subject. In some embodiments, the adoptively transferred Vγ9Vδ2 T cells infiltrate into a skin tissue in the receiving subject.

In some embodiments, the tissue comprises an unhealthy cell. In some embodiments, the unhealthy cell is a cancer cell. In some embodiments, the cancer cell is a blood cancer cell or a solid tumor cancer cell.

In some embodiments, the receiving subject does not develop any symptom of a Graft Versus Host Disease (GvHD) at least 7 days, at least 14 days, at least 21 days, or at least 28 days after the adoptive transfer of the population of cells to the receiving subject. In some embodiments, the receiving subject does not develop any symptom of a Graft Versus Host Disease (GvHD) at least 7 days after the adoptive transfer of the population of cells to the receiving subject. In some embodiments, the receiving subject does not develop any symptom of a Graft Versus Host Disease (GvHD) at least 14 days after the adoptive transfer of the population of cells to the receiving subject. In some embodiments, the receiving subject does not develop any symptom of a Graft Versus Host Disease (GvHD) at least 21 days after the adoptive transfer of the population of cells to the receiving subject. In some embodiments, the receiving subject does not develop any symptom of a Graft Versus Host Disease (GvHD) at least 28 days after the adoptive transfer of the population of cells to the receiving subject.

In some embodiments of the method for establishing an in vivo engraftment of Vγ9Vδ2 T cells in a receiving subject as described herein, the Vγ9Vδ2 T cells are chimeric antigen receptor (CAR) T cells comprising an extracellular domain, a transmembrane domain, and an intracellular domain. In some embodiments, the extracellular domain binds to an antigen expressed on an unhealthy cell. In some embodiments, the unhealthy cell is a cancer cell. In some embodiments, the cancer cell is a blood cancer cell or a solid tumor cancer cell. In some embodiments, the CAR T cells is obtained using the method for producing a population of CAR T cells described herein.

### 4.9. Kits and Articles of Manufacture

Further provided are kits, unit dosages, and articles of manufacture comprising any of the activated and expanded Vγ9Vδ2 T cells or the engineered Vγ9Vδ2 T cells of the present disclosure. In some embodiments, a kit is provided which comprises any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The disclosure is generally disclosed herein using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

### 5. EMBODIMENTS

This invention provides the following non-limiting embodiments.

In one set of embodiments (embodiment set A), provided are:
A1. A method for ex vivo activation and expansion of Vγ9Vδ2 T cells, comprising:
   a) contacting a population of cells comprising T cells with a culture system; wherein the activation and expansion conditions comprise IL-2, IL-15, and a bisphosphonate or a mevalonate pathway intermediate; and
   b) culturing the population of cells comprising T cells ex vivo in the culture system under a hypoxic condition for enhanced ex vivo activation and expansion of Vγ9Vδ2 T cells in the population of cells comprising T cells.
A2. The method of embodiment A1, wherein the method further comprises obtaining the population of cells comprising T cells from a subject.
A3. The method of embodiment A2, wherein the subject is healthy.
A4. The method of embodiment A2, wherein the subject is unhealthy.
A5. The method of any one of embodiments A1 to A4, wherein the population of cells comprising T cells are mammalian cells.
A6. The method of embodiment A5, wherein the mammalian cells are human cells.
A7. The method of embodiment A6, wherein the human cells are engineered cells.
A8. The method of embodiment A6, wherein the human cells are non-engineered cells.
A9. The method of any one of embodiments A1 to A8, wherein the population of cells comprising T cells are peripheral blood mononuclear cells (PBMCs).
A10. The method of embodiment A9, wherein the PBMCs are freshly obtained PBMCs.
A11. The method of embodiment A9, wherein the PBMCs are frozen PBMCs.
A12. The method of any one of embodiments A1 to A8, wherein the population of cells comprising T cells are derived from a human tissue.
A13. The method of embodiment A12, wherein the human tissue is fresh.
A14. The method of embodiment A12, wherein the human tissue is frozen.
A15. The method of any one of embodiments A1 to A8, wherein the population of cells comprising T cells are tumor-infiltrating lymphocytes (TILs).
A16. The method of embodiment A15, wherein the TILs are freshly obtained TILs.
A17. The method of embodiment A15, wherein the TILs are frozen TILs.
A18. The method of any one of embodiments A1 to A17, wherein the population of cells comprising T cells are ex vivo activated and expanded in the culture system under the hypoxic condition for at least 3 days, at least 5 days, at least 7 days, at least 9 days, at least 11 days, at least 13 days, at least 15 days, at least 17 days, at least 19 days, or at least 21 days.
A19. The method of embodiment A18, wherein the population of cells comprising T cells are ex vivo activated and expanded in the culture system under the hypoxic condition for 3-25, 4-23, 5-21, 6-19, 7-17, 8-15, 9-14, 10-14, 11-14, or 12-14 days.
A20. The method of embodiment A19, wherein the population of cells comprising T cells are ex vivo activated and expanded in the culture system under the hypoxic condition for about 14 days.
A21. The method of any one of embodiments A1 to A20, wherein the oxygen concentration of the hypoxic condition is less than 15%, less than 13%, less than 11%, less than 9%, less than 7%, less than 5%, less than 3%, less than 1%, or less than 0.5%.
A22. The method of embodiment A21, wherein the oxygen concentration of the hypoxic condition is 0.1%-15%, 0.5%-13%, 1%-13%, 1%-11%, 1%-9%, 1%-7%, or 2%-5%.
A23. The method of embodiment A21, wherein the oxygen concentration of the hypoxic condition is or is about 2%, 5%, or 12%.
A24. The method of embodiment A21, wherein the oxygen concentration of the hypoxic condition is or is about 5%.
A25. The method of any one of embodiments A1 to A24, wherein the method further comprises culturing the population of cells comprising T cells ex vivo in the culture system under a normoxic condition to activate and expand Vγ9Vδ2 T cells in the population of cells comprising T cells prior to step b).
A26. The method of embodiment A25, wherein the population of cells comprising T cells are ex vivo activated and expanded under the normoxic condition for at least 1 hour prior to step b).
A27. The method of embodiment A26, wherein the population of cells comprising T cells are ex vivo activated and expanded under the normoxic condition for 0.5-7, 1-6, 1-5, 1-4, 1-3, or 1-2 days prior to step b).
A28. The method of any one of embodiments A25 to A27, wherein the oxygen concentration of the normoxic condition is or is about 18.2% or 18.6%.
A29. The method of any one of embodiments A1 to A28, wherein the IL-2 concentration within the culture system is 10 IU/mL to 1200 IU/mL.
A30. The method of embodiment A29, wherein the IL-2 concentration within the culture system is adjusted to gradually decrease along the culturing process.
A31. The method of embodiment A30, wherein the IL-2 concentration within the culture system is 1000 IU/mL on days 0 and 1, 800 IU/mL on days 2, 3 and 4, and 100 IU/mL on day and thereafter.
A32. The method of any one of embodiments A1 to A31, wherein the IL-15 concentration within the culture system is 5 ng/ml to 25 ng/ml.
A33. The method of embodiment A32, wherein the IL-15 concentration within the culture system is adjusted during the culturing process.
A34. The method of embodiment A33, wherein the IL-15 concentration within the culture system is 10 ng/ml on days 0 and 1, 20 ng/mL on days 2, 3, and 4, and 10 ng/mL on day 5 and thereafter.
A35. The method of any one of embodiments A1 to A34, wherein the bisphosphonate is selected from the group consisting of zoledronic acid, risedronic acid, ibandronic acid, alendronic acid, pamidronic acid, tiludronic acid, etidronic acid, and clodronic acid.
A36. The method of any one of embodiments A1 to A34, wherein the mevalonate pathway intermediate is selected from the group consisting of HMBPP, BrHPP, and isopentenyl pyrophosphate.
A37. The method of any one of embodiments A1 to A36, wherein the method increases the total percentage of Vγ9Vδ2 T cells in the population of cells comprising T cells to more than 10%, more than 15%, more than 20%, more than 25%, more than 30%, more than 35%, more than 40%, more than 45%, more than 50%, more than 55%, or more than 60%.
A38. The method of embodiment A37, wherein the method increases the total percentage of Vγ9Vδ2 T cells in the population of cells comprising T cells to 10%-99%, 20%-95%, 30%-95%, 35%-95%, 40%-95%, 45%-95%, 50%-95%, 55%-95%, 60%-95%, or 65%-95%.
A39. The method of any one of embodiments A1 to A38, wherein the method increases the total number of Vγ9Vδ2 T cells in the population of cells comprising T cells by at least 10-fold, at least 30-fold, at least 50-fold, at least 100-fold, at least 150-fold, at least 200-fold, at least 250-fold, at least 300-fold, at least 350-fold, at least 400-fold, at least 450-fold, at least 500-fold, at least 550-fold, or at least 600-fold as compared to the total number of Vγ9Vδ2 T cells in the population of cells comprising T cells before the expansion.
A40. The method of embodiment A39, wherein the method increases the total number of Vγ9Vδ2 T cells in the population of cells comprising T cells by 10-700-fold, 30-650-fold, 50-600-fold, 100-600-fold, 150-600-fold, 200-600-fold, 250-600-fold, 300-600-fold, 350-600-fold, 400-600-fold, 450-600-fold, or 500-600-fold as compared to the total number of Vγ9Vδ2 T cells in the population of cells comprising T cells before the expansion.
A41. The method of any one of embodiments A1 to A40, further comprising enriching the ex vivo expanded Vγ9Vδ2 T cells from the population of cells comprising T cells after step b).

In another set of embodiments (embodiment set B), provided are:
B1. An isolated population of Vγ9Vδ2 T cells produced by a method comprising:
   a) contacting a population of cells comprising T cells with a culture system; wherein the activation and expansion conditions comprise IL-2, IL-15, and a bisphosphonate or a mevalonate pathway intermediate; and
   b) culturing the population of cells comprising T cells in the culture system under a hypoxic condition for enhanced ex vivo activation and expansion of Vγ9Vδ2 T cells in the population of cells comprising T cells.
B2. The isolated population of Vγ9Vδ2 T cells of embodiment B1, wherein the method further comprises obtaining the population of cells comprising T cells from a subject.
B3. The isolated population of Vγ9Vδ2 T cells of embodiment B2, wherein the subject is healthy.
B4. The isolated population of Vγ9Vδ2 T cells of embodiment B2, wherein the subject is unhealthy.
B5. The isolated population of Vγ9Vδ2 T cells of any one of embodiments B1 to B4, wherein the population of cells comprising T cells are mammalian cells.
B6. The isolated population of Vγ9Vδ2 T cells of embodiment B5, wherein the mammalian cells are human cells.
B7 The isolated population of Vγ9Vδ2 T cells of embodiment B6, wherein the human cells are engineered cells.
B8. The isolated population of Vγ9Vδ2 T cells of embodiment B6, wherein the human cells are non-engineered cells.
B9. The isolated population of Vγ9Vδ2 T cells of any one of embodiments B1 to B8, wherein the population of cells comprising T cells are peripheral blood mononuclear cells (PBMCs).
B10. The isolated population of Vγ9Vδ2 T cells of embodiment B9, wherein the PBMCs are freshly obtained PBMCs.
B11. The isolated population of Vγ9Vδ2 T cells of embodiment B9, wherein the PBMCs are frozen PBMCs.
B12. The isolated population of Vγ9Vδ2 T cells of any one of embodiments B1 to B8, wherein the population of cells comprising T cells are derived from a human tissue.
B13. The isolated population of Vγ9Vδ2 T cells of embodiment B12, wherein the human tissue is fresh.
B14. The isolated population of Vγ9Vδ2 T cells of embodiment B12, wherein the human tissue is frozen.
B15. The isolated population of Vγ9Vδ2 T cells of any one of embodiments B1 to B8, wherein the population of cells comprising T cells are tumor-infiltrating lymphocytes (TILs).
B16. The isolated population of Vγ9Vδ2 T cells of embodiment B15, wherein the TILs are freshly obtained TILs.
B17. The isolated population of Vγ9Vδ2 T cells of embodiment B15, wherein the TILs are frozen TILs.
B18. The isolated population of Vγ9Vδ2 T cells of any one of embodiments B1 to B17, wherein the population of cells comprising T cells are ex vivo activated and expanded in the culture system under the hypoxic condition for at least 3 days, at least 5 days, at least 7 days, at least 9 days, at least 11 days, at least 13 days, at least 15 days, at least 17 days, at least 19 days, or at least 21 days.
B19. The isolated population of Vγ9Vδ2 T cells of embodiment B18, wherein the population of cells comprising T cells are ex vivo activated and expanded in the culture system under the hypoxic condition for 3-25, 4-23, 5-21, 6-19, 7-17, 8-15, 9-14, 10-14, 11-14, or 12-14 days.
B20. The isolated population of Vγ9Vδ2 T cells of embodiment B19, wherein the population of cells comprising T cells are ex vivo activated and expanded in the culture system under the hypoxic condition for 14 days.
B21. The isolated population of Vγ9Vδ2 T cells of any one of embodiments B1 to B20, wherein the oxygen concentration of the hypoxic condition is less than 15%, less than 13%, less than 11%, less than 9%, less than 7%, less than 5%, less than 3%, less than 1%, or less than 0.5%.
B22. The isolated population of Vγ9Vδ2 T cells of embodiment B21, wherein the oxygen concentration of the hypoxic condition is 0.1%-15%, 0.5%-13%, 1%-13%, 1%-11%, 1%-9%, 1%-7%, or 2%-5%.
B23. The isolated population of Vγ9Vδ2 T cells of embodiment B21, wherein the oxygen concentration of the hypoxic condition is or is about 2%, 5%, or 12%.
B24. The isolated population of Vγ9Vδ2 T cells of embodiment B21, wherein the oxygen concentration of the hypoxic condition is or is about 5%.
B25. The isolated population of Vγ9Vδ2 T cells of any one of embodiments B1 to B24, wherein the method further comprises culturing the population of cells comprising T cells ex vivo in the culture system under a normoxic condition to activate and expand Vγ9Vδ2 T cells in the population of cells comprising T cells prior to step b).
B26. The isolated population of Vγ9Vδ2 T cells of embodiment B25, wherein the population of cells comprising T cells are ex vivo activated and expanded under the normoxic condition for at least 1 hour prior to step b).
B27. The isolated population of Vγ9Vδ2 T cells of embodiment B26, wherein the population of cells comprising T cells are ex vivo activated and expanded under the normoxic condition for 0.5-7, 1-6, 1-5, 1-4, 1-3, or 1-2 days prior to step b).
B28. The isolated population of Vγ9Vδ2 T cells of any one of embodiments B25 to B27, wherein the oxygen concentration of the normoxic condition is or is about 18.2% or 18.6%.
B29. The isolated population of Vγ9Vδ2 T cells of any one of embodiments B1 to B28, wherein the IL-2 concentration within the culture system is 10 IU/mL to 1200 IU/mL. B30. The isolated population of Vγ9Vδ2 T cells of embodiment B29, wherein the IL-2 concentration within the culture system is adjusted to gradually decrease along the culturing process.
B31. The isolated population of Vγ9Vδ2 T cells of embodiment B30, wherein the IL-2 concentration within the culture system is 1000 IU/mL on days 0 and 1, 800 IU/mL on days 2, 3 and 4, and 100 IU/mL on day 5 and thereafter.
B32. The isolated population of Vγ9Vδ2 T cells of any one of embodiments B1 to B31, wherein the IL-15 concentration within the culture system is 5 ng/ml to 25 ng/ml.
B33. The isolated population of Vγ9Vδ2 T cells of embodiment B32, wherein the IL-15 concentration within the culture system is adjusted along the culturing process.
B34. The isolated population of Vγ9Vδ2 T cells of embodiment B33, wherein the IL-15 concentration within the culture system is 10 ng/mL on days 0 and 1, 20 ng/ml on days 2, 3, and 4, and 10 ng/mL on day 5 and thereafter.
B35. The isolated population of Vγ9Vδ2 T cells of any one of embodiments B1 to B34, wherein the bisphosphonate is selected from the group consisting of zoledronic acid, risedronic acid, ibandronic acid, alendronic acid, pamidronic acid, tiludronic acid, etidronic acid, and clodronic acid.
B36. The isolated population of Vγ9Vδ2 T cells of any one of embodiments B1 to B34, wherein the mevalonate pathway intermediate is selected from the group consisting of HMBPP, BrHPP, and isopentenyl pyrophosphate.
B37. The isolated population of Vγ9Vδ2 T cells of any one of embodiments B1 to B36, wherein the method increases the total percentage of Vγ9Vδ2 T cells in the population of cells comprising T cells to more than 10%, more than 15%, more than 20%, more than 25%, more than 30%, more than 35%, more than 40%, more than 45%, more than 50%, more than 55%, or more than 60%.
B38. The isolated population of Vγ9Vδ2 T cells of embodiment B37, wherein the method increases the total percentage of Vγ9Vδ2 T cells in the population of cells comprising T cells to 10%-99%, 20%-95%, 30%-95%, 35%-95%, 40%-95%, 45%-95%, 50%-95%, 55%-95%, 60%-95%, or 65%-95%.
B39. The isolated population of Vγ9Vδ2 T cells of any one of embodiments B1 to B38, wherein the method increases the total number of Vγ9Vδ2 T cells in the population of cells comprising T cells by at least 10-fold, at least 30-fold, at least 50-fold, at least 100-fold, at least 150-fold, at least 200-fold, at least 250-fold, at least 300-fold, at least 350-fold, at least 400-fold, at least 450-fold, at least 500-fold, at least 550-fold, or at least 600-fold as compared to the total number of Vγ9Vδ2 T cells in the population of cells comprising T cells before the expansion.
B40. The isolated population of Vγ9Vδ2 T cells of embodiment B39, wherein the method increases the total number of Vγ9Vδ2 T cells in the population of cells comprising T cells by 10-700-fold, 30-650-fold, 50-600-fold, 100-600-fold, 150-600-fold, 200-600-fold, 250-600-fold, 300-600-fold, 350-600-fold, 400-600-fold, 450-600-fold, or 500-600-fold as compared to the total number of Vγ9Vδ2 T cells in the population of cells comprising T cells before the expansion.
B41. The isolated population of Vγ9Vδ2 T cells of any one of embodiments B1 to B40, wherein the method further comprises enriching the ex vivo expanded Vγ9Vδ2 T cells from the population of cells comprising T cells after step b).
B42. An isolated population of cells, wherein the percentage of Vγ9Vδ2 T cells in the isolated population of cells is more than 10%, more than 15%, more than 20%, more than 25%, more than 30%, more than 35%, more than 40%, more than 45%, more than 50%, more than 55%, or more than 60%.
B43. An isolated population of cells, wherein the percentage of Vγ9Vδ2 T cells in the isolated population of cells is 10%-99%, 20%-95%, 30%-95%, 35%-95%, 40%-95%, 45%-95%, 50%-95%, 55%-95%, 60%-95%, or 65%-95%.
B44. A pharmaceutical composition comprising the isolated population of Vγ9Vδ2 T cells of any one of embodiments B1 to B41 or the isolated population of cells of embodiments B42 to B43, and a pharmaceutically acceptable excipient.
B45. A method for treating a disease or disorder in a subject, comprising administering to the subject: (i) a therapeutically effective amount of the pharmaceutical composition of embodiment B44, and (ii) a therapeutically effective amount of one or more multispecific antibodies.
B46. The method of embodiment B45, wherein each of the multispecific antibodies comprises:
   1) a first binding domain that binds to an antigen expressed on a Vγ9Vδ2 T cell; and
   2) a second binding domain that binds to an antigen expressed on an unhealthy cell.
B47. The method of embodiment B46, wherein the antigen expressed on the Vγ9Vδ2 T cell is T Cell Receptor Gamma Variable 9 (TRGV9) or CD3.
B48. The method of embodiment B46, wherein the unhealthy cell is a cancer cell, and wherein the antigen expressed on the unhealthy cell is a tumor-associated antigen (TAA).
B49. The method of embodiment B48, wherein the cancer cell is a blood cancer cell or a solid tumor cancer cell.
B50. The method of any one of embodiments B45 to B49, wherein the method comprises administering to the subject: (i) a therapeutically effective amount of the pharmaceutical composition of embodiment B44, and (ii) a therapeutically effective amount of Vγ9×TAA and/or CD3×TAA bispecific antibodies.
B51. The method of any one of embodiments B45 to B50, wherein the disease or disorder is cancer.
B52. The method of embodiment B51, wherein the cancer is a blood cancer.
B53. The method of embodiment B51, wherein the cancer is a solid tumor cancer.
B54. The method of any one of embodiments B45 to B53, wherein the subject is a human subject in need thereof.
B55. A process for making a chimeric antigen receptor (CAR) T cell product, comprising: (i) a step of performing a function of obtaining the isolated population of Vγ9Vδ2 T cells of any one of embodiments B1 to B41 or the isolated population of cells of embodiments B42 to B43; and (ii) a step of performing a function of expressing a CAR in the Vγ9Vδ2 T cells.
B56. The process of embodiment B55, wherein the CAR comprises an extracellular domain, a transmembrane domain, and an intracellular domain.
B57. The process of embodiment B56, wherein the extracellular domain binds to an antigen expressed on an unhealthy cell.
B58. The process of embodiment B57, wherein the unhealthy cell is a cancer cell.
B59. The process of embodiment B58, wherein the cancer cell is a blood cancer cell or a solid tumor cancer cell.

In another set of embodiments (embodiment set C), provided are:
C1. A method for making a chimeric antigen receptor (CAR) T cell product, comprising: (i) obtaining a population of cells comprising Vγ9Vδ2 T cells; and (ii) introducing nucleic acids encoding a CAR into the population of cells.
C2. The method of embodiment C1, wherein step (i) comprises:
   a) contacting a population of cells comprising T cells with a culture system; wherein the activation and expansion conditions comprise IL-2, IL-15, and a bisphosphonate or a mevalonate pathway intermediate; and
   b) culturing the population of cells comprising T cells ex vivo in the culture system under a hypoxic condition for enhanced ex vivo activation and expansion of Vγ9Vδ2 T cells in the population of cells comprising T cells.
C3. The method of embodiment C2, wherein the method further comprises obtaining the population of cells comprising T cells from a subject.
C4. The method of embodiment C3, wherein the subject is healthy.
C5. The method of embodiment C3, wherein the subject is unhealthy.
C6. The method of any one of embodiments C2 to C5, wherein the population of cells comprising T cells are mammalian cells.
C7. The method of embodiment C6, wherein the mammalian cells are human cells.
C8. The method of embodiment C7, wherein the human cells are engineered cells.
C9. The method of embodiment C7, wherein the human cells are non-engineered cells.
C10. The method of any one of embodiments C2 to C9, wherein the population of cells comprising T cells are peripheral blood mononuclear cells (PBMCs).
C11. The method of embodiment C10, wherein the PBMCs are freshly obtained PBMCs.
C12. The method of embodiment C10, wherein the PBMCs are frozen PBMCs.
C13. The method of any one of embodiments C2 to C9, wherein the population of cells comprising T cells are derived from a human tissue.
C14. The method of embodiment C13, wherein the human tissue is fresh.
C15. The method of embodiment C13, wherein the human tissue is frozen.
C16. The method of any one of embodiments C2 to C9, wherein the population of cells comprising T cells are tumor-infiltrating lymphocytes (TILs).
C17. The method of embodiment C16, wherein the TILs are freshly obtained TILs.
C18. The method of embodiment C16, wherein the TILs are frozen TILs.
C19. The method of any one of embodiments C2 to C18, wherein the population of cells comprising T cells are ex vivo activated and expanded in the culture system under the hypoxic condition for at least 3 days, at least 5 days, at least 7 days, at least 9 days, at least 11 days, at least 13 days, at least 15 days, at least 17 days, at least 19 days, or at least 21 days.
C20. The method of embodiment C19, wherein the population of cells comprising T cells are ex vivo activated and expanded in the culture system under the hypoxic condition for 3-25, 4-23, 5-21, 6-19, 7-17, 8-15, 9-14, 10-14, 11-14, or 12-14 days.
C21. The method of embodiment C20, wherein the population of cells comprising T cells are ex vivo activated and expanded in the culture system under the hypoxic condition for about 14 days.
C22. The method of any one of embodiments C2 to C21, wherein the oxygen concentration of the hypoxic condition is less than 15%, less than 13%, less than 11%, less than 9%, less than 7%, less than 5%, less than 3%, less than 1%, or less than 0.5%.
C23. The method of embodiment C22, wherein the oxygen concentration of the hypoxic condition is 0.1%-15%, 0.5%-13%, 1%-13%, 1%-11%, 1%-9%, 1%-7%, or 2%-5%.
C24. The method of embodiment C22, wherein the oxygen concentration of the hypoxic condition is or is about 2%, 5%, or 12%.
C25. The method of embodiment C22, wherein the oxygen concentration of the hypoxic condition is or is about 5%.
C26. The method of any one of embodiments C2 to C25, wherein the method further comprises culturing the population of cells comprising T cells ex vivo in the culture system under a normoxic condition to activate and expand Vγ9Vδ2 T cells in the population of cells comprising T cells prior to step b).
C27. The method of embodiment C26, wherein the population of cells comprising T cells are ex vivo activated and expanded under the normoxic condition for at least 1 hour prior to step (b) of embodiment C2.
C28. The method of embodiment C27, wherein the population of cells comprising T cells are ex vivo activated and expanded under the normoxic condition for 0.5-7, 1-6, 1-5, 1-4, 1-3, or 1-2 days prior to step b).
C29. The method of any one of embodiments C26 to C28, wherein the oxygen concentration of the normoxic condition is or is about 18.2% or 18.6%.
C30. The method of any one of embodiments C2 to C29, wherein the IL-2 concentration within the culture system is 10 IU/mL to 1200 IU/mL.
C31. The method of embodiment C30, wherein the IL-2 concentration within the culture system is adjusted to gradually decrease along the culturing process.
C32. The method of embodiment C31, wherein the IL-2 concentration within the culture system is 1000 IU/mL on days 0 and 1, 800 IU/mL on days 2, 3 and 4, and 100 IU/mL on day and thereafter.
C33. The method of any one of embodiments C2 to C32, wherein the IL-15 concentration within the culture system is 5 ng/ml to 25 ng/ml.
C34. The method of embodiment C33, wherein the IL-15 concentration within the culture system is adjusted along the culturing process.
C35. The method of embodiment C34, wherein the IL-15 concentration within the culture system is 10 ng/mL on days 0 and 1, 20 ng/mL on days 2, 3, and 4, and 10 ng/ml on day 5 and thereafter.
C36. The method of any one of embodiments C1 to C35, wherein the bisphosphonate is selected from the group consisting of zoledronic acid, risedronic acid, ibandronic acid, alendronic acid, pamidronic acid, tiludronic acid, etidronic acid, and clodronic acid.
C37. The method of any one of embodiments C1 to C35, wherein the mevalonate pathway intermediate is selected from the group consisting of HMBPP, BrHPP, and isopentenyl pyrophosphate.
C38. The method of any one of embodiments C2 to C37, wherein the method increases the total percentage of Vγ9Vδ2 T cells in the population of cells comprising T cells to more than 10%, more than 15%, more than 20%, more than 25%, more than 30%, more than 35%, more than 40%, more than 45%, more than 50%, more than 55%, or more than 60%.
C39. The method of embodiment C38, wherein the method increases the total percentage of Vγ9Vδ2 T cells in the population of cells comprising T cells to 10%-99%, 20%-95%, 30%-95%, 35%-95%, 40%-95%, 45%-95%, 50%-95%, 55%-95%, 60%-95%, or 65%-95%.
C40. The method of any one of embodiments C2 to C39, wherein the method increases the total number of Vγ9Vδ2 T cells in the population of cells comprising T cells by at least 10-fold, at least 30-fold, at least 50-fold, at least 100-fold, at least 150-fold, at least 200-fold, at least 250-fold, at least 300-fold, at least 350-fold, at least 400-fold, at least 450-fold, at least 500-fold, at least 550-fold, or at least 600-fold as compared to the total number of Vγ9Vδ2 T cells in the population of cells comprising T cells before the expansion.
C41. The method of embodiment C40, wherein the method increases the total number of Vγ9Vδ2 T cells in the population of cells comprising T cells by 10-700-fold, 30-650-fold, 50-600-fold, 100-600-fold, 150-600-fold, 200-600-fold, 250-600-fold, 300-600-fold, 350-600-fold, 400-600-fold, 450-600-fold, or 500-600-fold as compared to the total number of Vγ9Vδ2 T cells in the population of cells comprising T cells before the expansion.
C42. The method of any one of embodiments C2 to C41, wherein the method further comprises enriching the ex vivo expanded Vγ9Vδ2 T cells from the population of cells comprising T cells after step b).
C43. The method of any one of embodiments C1 to C42, wherein the population of cells comprising Vγ9Vδ2 T cells comprises more than 10%, more than 15%, more than 20%, more than 25%, more than 30%, more than 35%, more than 40%, more than 45%, more than 50%, more than 55%, or more than 60% Vγ9Vδ2 T cells in the population of cells.
C44. The method of embodiment C43, wherein the population of cells comprising Vγ9Vδ2 T cells comprises 10%-99%, 20%-95%, 30%-95%, 35%-95%, 40%-95%, 45%-95%, 50%-95%, 55%-95%, 60%-95%, or 65%-95% Vγ9Vδ2 T cells in the population of cells.
C45. The method of any one of embodiments C1 to C44, wherein the CAR comprises an extracellular domain, a transmembrane domain, and an intracellular domain.
C46. The method of embodiment C45, wherein the extracellular domain binds to an antigen expressed on an unhealthy cell.
C47. The method of embodiment C46, wherein the unhealthy cell is a cancer cell.
C48. The method of embodiment C47, wherein the cancer cell is a blood cancer cell or a solid tumor cancer cell.

In another set of embodiments (embodiment set D), provided are:
D1. A CAR T cell product produced by a method comprising: (i) obtaining a population of cells comprising Vγ9Vδ2 T cells; and (ii) introducing a nucleic acid encoding a chimeric antigen receptor (CAR) into the population of cells.
D2. The CAR T cell product of embodiment D1, wherein step (i) in the method of embodiment D1 comprising:
   a) contacting a population of cells comprising T cells with a culture system; wherein the activation and expansion conditions comprise IL-2, IL-15, and a bisphosphonate or a mevalonate pathway intermediate; and
   b) culturing the population of cells comprising T cells ex vivo in the culture system under a hypoxic condition for enhanced ex vivo activation and expansion of Vγ9Vδ2 T cells in the population of cells comprising T cells.
D3. The CAR T cell product of embodiment D2, wherein the method further comprises obtaining the population of cells comprising T cells from a subject.
D4. The CAR T cell product of embodiment D3, wherein the subject is healthy.
D5. The CAR T cell product of embodiment D3, wherein the subject is unhealthy.
D6. The CAR T cell product of any one of embodiments D2 to D5, wherein the population of cells comprising T cells are mammalian cells.
D7. The CAR T cell product of embodiment D6, wherein the mammalian cells are human cells.
D8. The CAR T cell product of embodiment D7, wherein the human cells are engineered cells.
D9. The CAR T cell product of embodiment D7, wherein the human cells are non-engineered cells.
D10. The CAR T cell product of any one of embodiments D2 to D9, wherein the population of cells comprising T cells are peripheral blood mononuclear cells (PBMCs).
D11. The CAR T cell product of embodiment D10, wherein the PBMCs are freshly obtained PBMCs.
D12. The CAR T cell product of embodiment D10, wherein the PBMCs are frozen PBMCs.
D13. The CAR T cell product of any one of embodiments D2 to D9, wherein the population of cells comprising T cells are derived from a human tissue.
D14. The CAR T cell product of embodiment D13, wherein the human tissue is fresh.
D15. The CAR T cell product of embodiment D13, wherein the human tissue is frozen.
D16. The CAR T cell product of any one of embodiments D2 to D9, wherein the population of cells comprising T cells are tumor-infiltrating lymphocytes (TILs).
D17. The CAR T cell product of embodiment D16, wherein the TILs are freshly obtained TILs.
D18. The CAR T cell product of embodiment D16, wherein the TILs are frozen TILs.
D19. The CAR T cell product of any one of embodiments D2 to D18, wherein the population of cells comprising T cells are ex vivo activated and expanded in the culture system under the hypoxic condition for at least 3 days, at least 5 days, at least 7 days, at least 9 days, at least 11 days, at least 13 days, at least 15 days, at least 17 days, at least 19 days, or at least 21 days.
D20. The CAR T cell product of embodiment D19, wherein the population of cells comprising T cells are ex vivo activated and expanded in the culture system under the hypoxic condition for 3-25, 4-23, 5-21, 6-19, 7-17, 8-15, 9-14, 10-14, 11-14, or 12-14 days.
D21. The CAR T cell product of embodiment D20, wherein the population of cells comprising T cells are ex vivo activated and expanded in the culture system under the hypoxic condition for about 14 days.
D22. The CAR T cell product of any one of embodiments D2 to D21, wherein the oxygen concentration of the hypoxic condition is less than 15%, less than 13%, less than 11%, less than 9%, less than 7%, less than 5%, less than 3%, less than 1%, or less than 0.5%.
D23. The CAR T cell product of embodiment D22, wherein the oxygen concentration of the hypoxic condition is 0.1%-15%, 0.5%-13%, 1%-13%, 1%-11%, 1%-9%, 1%-7%, or 2%-5%.
D24. The CAR T cell product of embodiment D22, wherein the oxygen concentration of the hypoxic condition is or is about 2%, 5%, or 12%.
D25. The CAR T cell product of embodiment D22, wherein the oxygen concentration of the hypoxic condition is or is about 5%.
D26. The CAR T cell product of any one of embodiments D2 to D25, wherein the method further comprises culturing the population of cells comprising T cells ex vivo in the culture system under a normoxic condition to activate and expand Vγ9Vδ2 T cells in the population of cells comprising T cells prior to step b).
D27. The CAR T cell product of embodiment D26, wherein the population of cells comprising T cells are ex vivo activated and expanded under the normoxic condition for at least 1 hour prior to step b).
D28. The CAR T cell product of embodiment D27, wherein the population of cells comprising T cells are ex vivo activated and expanded under the normoxic condition for 0.5-7, 1-6, 1-5, 1-4, 1-3, or 1-2 days prior to step b).
D29. The CAR T cell product of any one of embodiments D26 to D28, wherein the oxygen concentration of the normoxic condition is or is about 18.2% or 18.6%.
D30. The CAR T cell product of any one of embodiments D2 to D29, wherein the IL-2 concentration within the culture system is 10 IU/mL to 1200 IU/mL.
D31. The CAR T cell product of embodiment D30, wherein the IL-2 concentration within the culture system is adjusted to gradually decrease along the culturing process.
D32. The CAR T cell product of embodiment D31, wherein the IL-2 concentration within the culture system is 1000 IU/mL on days 0 and 1, 800 IU/mL on days 2, 3 and 4, and 100 IU/mL on day 5 and thereafter.
D33. The CAR T cell product of any one of embodiments D2 to D32, wherein the IL-15 concentration within the culture system is 5 ng/ml to 25 ng/ml.
D34. The CAR T cell product of embodiment D33, wherein the IL-15 concentration within the culture system is adjusted along the culturing process.
D35. The CAR T cell product of embodiment D34, wherein the IL-15 concentration within the culture system is 10 ng/ml on days 0 and 1, 20 ng/mL on days 2, 3, and 4, and 10 ng/mL on day 5 and thereafter.
D36. The CAR T cell product of any one of embodiments D2 to D35, wherein the bisphosphonate is selected from the group consisting of zoledronic acid, risedronic acid, ibandronic acid, alendronic acid, pamidronic acid, tiludronic acid, etidronic acid, and clodronic acid.
D37. The CAR T cell product of any one of embodiments D2 to D35, wherein the mevalonate pathway intermediate is selected from the group consisting of HMBPP, BrHPP, and isopentenyl pyrophosphate.
D38. The CAR T cell product of any one of embodiments D2 to D37, wherein the method increases the total percentage of Vγ9Vδ2 T cells in the population of cells comprising T cells to more than 10%, more than 15%, more than 20%, more than 25%, more than 30%, more than 35%, more than 40%, more than 45%, more than 50%, more than 55%, or more than 60%.
D39. The CAR T cell product of embodiment D38, wherein the method increases the total percentage of Vγ9Vδ2 T cells in the population of cells comprising T cells to 10%-99%, 20%-95%, 30%-95%, 35%-95%, 40%-95%, 45%-95%, 50%-95%, 55%-95%, 60%-95%, or 65%-95%.
D40. The CAR T cell product of any one of embodiments D2 to D39, wherein the method increases the total number of Vγ9Vδ2 T cells in the population of cells comprising T cells by at least 10-fold, at least 30-fold, at least 50-fold, at least 100-fold, at least 150-fold, at least 200-fold, at least 250-fold, at least 300-fold, at least 350-fold, at least 400-fold, at least 450-fold, at least 500-fold, at least 550-fold, or at least 600-fold as compared to the total number of Vγ9Vδ2 T cells in the population of cells comprising T cells before the expansion.
D41. The CAR T cell product of embodiment D40, wherein the method increases the total number of Vγ9Vδ2 T cells in the population of cells comprising T cells by 10-700-fold, 30-650-fold, 50-600-fold, 100-600-fold, 150-600-fold, 200-600-fold, 250-600-fold, 300-600-fold, 350-600-fold, 400-600-fold, 450-600-fold, or 500-600-fold as compared to the total number of Vγ9Vδ2 T cells in the population of cells comprising T cells before the expansion.
D42. The CAR T cell product of any one of embodiments D2 to D41, wherein the method further comprises enriching the ex vivo expanded Vγ9Vδ2 T cells from the population of cells comprising T cells after step b).
D43. The CAR T cell product of any one of embodiments DI to D42, wherein the population of cells comprising Vγ9Vδ2 T cells comprises more than 10%, more than 15%, more than 20%, more than 25%, more than 30%, more than 35%, more than 40%, more than 45%, more than 50%, more than 55%, or more than 60% Vγ9Vδ2 T cells in the population of cells.
D44. The CAR T cell product of embodiment D43, wherein the population of cells comprising Vγ9Vδ2 T cells comprises 10%-99%, 20%-95%, 30%-95%, 35%-95%, 40%-95%, 45%-95%, 50%-95%, 55%-95%, 60%-95%, or 65%-95% Vγ9Vδ2 T cells in the population of cells.
D45. The CAR T cell product of any one of embodiments D1 to D44, wherein the CAR comprises an extracellular domain, a transmembrane domain, and an intracellular domain.
D46. The CAR T cell product of embodiment D45, wherein the extracellular domain binds to an antigen expressed on an unhealthy cell.
D47. The CAR T cell product of embodiment D46, wherein the unhealthy cell is a cancer cell.
D48. The CAR T cell product of embodiment D47, wherein the cancer cell is a blood cancer cell or a solid tumor cancer cell.
D49. A CAR T cell comprising a CAR comprising an extracellular domain, a transmembrane domain, and an intracellular domain, wherein the CAR T cell is a Vγ9Vδ2 T cell.
D50. The CAR T cell of embodiment D49, wherein the extracellular domain binds to an antigen expressed on an unhealthy cell.
D51. The CAR T cell of embodiment D50, wherein the unhealthy cell is a cancer cell.
D52. The CAR T cell of embodiment D50, wherein the cancer cell is a blood cancer cell or a solid tumor cancer cell.
D53. A pharmaceutical composition comprising the CAR T cell product of any one of embodiments D1 to D48 or the CAR T cell of any one of embodiments 49 to 52, and a pharmaceutically acceptable excipient.

In yet another set of embodiments (embodiment set E), provided are:
E1. A method for treating a disease or disorder in a subject, comprising administering to the subject a therapeutically effective amount of CAR T cells, wherein the CAR T cells are produced by a method comprising: (i) obtaining a population of cells comprising Vγ9Vδ2 T cells; and (ii) introducing a nucleic acid encoding a chimeric antigen receptor (CAR) into the population of cells.
E2. The method of embodiment E1, wherein step (i) in the method of embodiment E1 comprises:
   a) contacting a population of cells comprising T cells with a culture system; wherein the activation and expansion conditions comprise IL-2, IL-15, and a bisphosphonate or a mevalonate pathway intermediate; and
   b) culturing the population of cells comprising T cells ex vivo in the culture system under a hypoxic condition for enhanced ex vivo activation and expansion of Vγ9Vδ2 T cells in the population of cells comprising T cells.
E3. The method of embodiment E2, wherein the method further comprises obtaining the population of cells comprising T cells from a subject.
E4. The method of embodiment E3, wherein the subject is healthy.
E5. The method of embodiment E3, wherein the subject is unhealthy.
E6. The method of any one of embodiments E2 to E5, wherein the population of cells comprising T cells are mammalian cells.
E7. The method of embodiment E6, wherein the mammalian cells are human cells.
E8. The method of embodiment E7, wherein the human cells are engineered cells.
E9. The method of embodiment E7, wherein the human cells are non-engineered cells.
E10. The method of any one of embodiments E2 to E9, wherein the population of cells comprising T cells are peripheral blood mononuclear cells (PBMCs).
E11. The method of embodiment E10, wherein the PBMCs are freshly obtained PBMCs.
E12. The method of embodiment E10, wherein the PBMCs are frozen PBMCs.
E13. The method of any one of embodiments E2 to E9, wherein the population of cells comprising T cells are derived from a human tissue.
E14. The method of embodiment E13, wherein the human tissue is fresh.
E15. The method of embodiment E13, wherein the human tissue is frozen.
E16. The method of any one of embodiments E2 to E9, wherein the population of cells comprising T cells are tumor-infiltrating lymphocytes (TILs).
E17. The method of embodiment E16, wherein the TILs are freshly obtained TILs.
E18. The method of embodiment E16, wherein the TILs are frozen TILs.
E19. The method of any one of embodiments E2 to E18, wherein the population of cells comprising T cells are ex vivo activated and expanded in the culture system under the hypoxic condition for at least 3 days, at least 5 days, at least 7 days, at least 9 days, at least 11 days, at least 13 days, at least 15 days, at least 17 days, at least 19 days, or at least 21 days.
E20. The method of embodiment E19, wherein the population of cells comprising T cells are ex vivo activated and expanded in the culture system under the hypoxic condition for 3-25, 4-23, 5-21, 6-19, 7-17, 8-15, 9-14, 10-14, 11-14, or 12-14 days.
E21. The method of embodiment E20, wherein the population of cells comprising T cells are ex vivo activated and expanded in the culture system under the hypoxic condition for about 14 days.
E22. The method of any one of embodiments E2 to E21, wherein the oxygen concentration of the hypoxic condition is less than 15%, less than 13%, less than 11%, less than 9%, less than 7%, less than 5%, less than 3%, less than 1%, or less than 0.5%.
E23. The method of embodiment E22, wherein the oxygen concentration of the hypoxic condition is 0.1%-15%, 0.5%-13%, 1%-13%, 1%-11%, 1%-9%, 1%-7%, or 2%-5%.
E24. The method of embodiment E22, wherein the oxygen concentration of the hypoxic condition is or is about 2%, 5%, or 12%.
E25. The method of embodiment E22, wherein the oxygen concentration of the hypoxic condition is or is about 5%.
E26. The method of any one of embodiments E2 to E25, wherein the method further comprises culturing the population of cells comprising T cells ex vivo in the culture system under a normoxic condition to activate and expand Vγ9Vδ2 T cells in the population of cells comprising T cells prior to step b).
E27. The method of embodiment E26, wherein the population of cells comprising T cells are ex vivo activated and expanded under the normoxic condition for at least 1 hour prior to step b).
E28. The method of embodiment E27, wherein the population of cells comprising T cells are ex vivo activated and expanded under the normoxic condition for 0.5-7, 1-6, 1-5, 1-4, 1-3, or 1-2 days prior to step b).
E29. The method of any one of embodiments E26 to E28, wherein the oxygen concentration of the normoxic condition is or is about 18.2% or 18.6%.
E30. The method of any one of embodiments E2 to E29, wherein the IL-2 concentration within the culture system is 10 IU/mL to 1200 IU/mL.
E31. The method of embodiment E30, wherein the IL-2 concentration within the culture system is adjusted to gradually decrease during the culturing process.
E32. The method of embodiment E31, wherein the IL-2 concentration within the culture system is 1000 IU/mL on days 0 and 1, 800 IU/mL on days 2, 3 and 4, and 100 IU/mL on day and thereafter.
E33. The method of any one of embodiments E2 to E32, wherein the IL-15 concentration within the culture system is 5 ng/ml to 25 ng/ml.
E34. The method of embodiment E33, wherein the IL-15 concentration within the culture system is adjusted during the culturing process.
E35. The method of embodiment E34, wherein the IL-15 concentration within the culture system is 10 ng/mL on days 0 and 1, 20 ng/ml on days 2, 3, and 4, and 10 ng/mL on day 5 and thereafter.
E36. The method of any one of embodiments E1 to E35, wherein the bisphosphonate is selected from the group consisting of zoledronic acid, risedronic acid, ibandronic acid, alendronic acid, pamidronic acid, tiludronic acid, etidronic acid, and clodronic acid.
E37. The method of any one of embodiments E1 to E35, wherein the mevalonate pathway intermediate is selected from the group consisting of HMBPP, BrHPP, and isopentenyl pyrophosphate.
E38. The method of any one of embodiments E2 to E37, wherein the method increases the total percentage of Vγ9Vδ2 T cells in the population of cells comprising T cells to more than 10%, more than 15%, more than 20%, more than 25%, more than 30%, more than 35%, more than 40%, more than 45%, more than 50%, more than 55%, or more than 60%.
E39. The method of embodiment E38, wherein the method increases the total percentage of Vγ9Vδ2 T cells in the population of cells comprising T cells to 10%-99%, 20%-95%, 30%-95%, 35%-95%, 40%-95%, 45%-95%, 50%-95%, 55%-95%, 60%-95%, or 65%-95%.
E40. The method of any one of embodiments E2 to E39, wherein the method increases the total number of Vγ9Vδ2 T cells in the population of cells comprising T cells by at least 10-fold, at least 30-fold, at least 50-fold, at least 100-fold, at least 150-fold, at least 200-fold, at least 250-fold, at least 300-fold, at least 350-fold, at least 400-fold, at least 450-fold, at least 500-fold, at least 550-fold, or at least 600-fold as compared to the total number of Vγ9Vδ2 T cells in the population of cells comprising T cells before the expansion.
E41. The method of embodiment E40, wherein the method increases the total number of Vγ9Vδ2 T cells in the population of cells comprising T cells by 10-700-fold, 30-650-fold, 50-600-fold, 100-600-fold, 150-600-fold, 200-600-fold, 250-600-fold, 300-600-fold, 350-600-fold, 400-600-fold, 450-600-fold, or 500-600-fold as compared to the total number of Vγ9Vδ2 T cells in the population of cells comprising T cells before the expansion.
E42. The method of any one of embodiments E2 to E41, wherein the method further comprises enriching the ex vivo expanded Vγ9Vδ2 T cells from the population of cells comprising T cells after step b).
E43. The method of any one of embodiments E1 to E42, wherein the population of cells comprising Vγ9Vδ2 T cells comprises more than 10%, more than 15%, more than 20%, more than 25%, more than 30%, more than 35%, more than 40%, more than 45%, more than 50%, more than 55%, or more than 60% Vγ9Vδ2 T cells in the population of cells.
E44. The method of embodiment E43, wherein the population of cells comprising Vγ9Vδ2 T cells comprises 10%-99%, 20%-95%, 30%-95%, 35%-95%, 40%-95%, 45%-95%, 50%-95%, 55%-95%, 60%-95%, or 65%-95% Vγ9Vδ2 T cells in the population of cells.
E45. The method of any one of embodiments E1 to E44, wherein the CAR comprises an extracellular domain, a transmembrane domain, and an intracellular domain.
E46. The method of embodiment E45, wherein the extracellular domain binds to an antigen expressed on an unhealthy cell.
E47. The method of embodiment E46, wherein the unhealthy cell is cancer cell.
E48. The method of embodiment E47, wherein the cancer cell is a blood cancer cell or a solid tumor cancer cell.
E49. A method for treating a disease or disorder in a subject comprising administering to the subject a therapeutically effective amount of CAR T cells, wherein the CAR T cells comprise a CAR comprising an extracellular domain, a transmembrane domain, and an intracellular domain, and wherein the CAR T cell is a Vγ9Vδ2 T cell.
E50. The method of embodiment E49, wherein the extracellular domain binds to an antigen expressed on an unhealthy cell.
E51. The method of embodiment E50, wherein the unhealthy cell is a cancer cell.
E52. The method of embodiment E51, wherein the cancer cell is a blood cancer cell or a solid tumor cancer cell.
E53. The method of any one of embodiments E1 to E52, wherein the disease or disorder is cancer.
E54. The method of embodiment E53, wherein the cancer is a blood cancer.
E55. The method of embodiment E53, wherein the cancer is a solid tumor cancer.
E56. The method of any one of embodiments E1 to E55, wherein the subject is a human subject in need thereof.

In yet another set of embodiments (embodiment set F), provided are:
F1. A method for treating a disease or disorder in a subject, comprising administering to the subject: (i) a therapeutically effective amount of a population of Vγ9Vδ2 T cells, and (ii) a therapeutically effective amount of one or more multispecific antibodies.
F2 The method of embodiment F1, wherein each of the multispecific antibodies comprises:
   1) a first binding domain that binds to an antigen expressed on a Vγ9Vδ2 T cell; and
   2) a second binding domain that binds to an antigen expressed on an unhealthy cell.
F3. The method of embodiment F2, wherein the antigen expressed on the Vγ9Vδ2 T cell is T Cell Receptor Gamma Variable 9 (TRGV9) or CD3.
F4. The method of embodiment F2, wherein the unhealthy cell is a cancer cell, and wherein the antigen expressed on the unhealthy cell is a tumor-associated antigen (TAA).
F5 The method of embodiment F4, wherein the cancer cell is a blood cancer cell or a solid tumor cancer cell.
F6. The method of any one of embodiments F1-F5, wherein the method comprises administering to the subject: (i) a therapeutically effective amount of the population of Vγ9Vδ2 T cells, and (ii) a therapeutically effective amount of Vγ9×TAA and/or CD3×TAA bispecific antibodies.
F7. The method of any one of embodiments F1-F6, wherein the disease or disorder is cancer.
F8 The method of embodiment F7, wherein the cancer is a blood cancer.
F9. The method of embodiment F7, wherein the cancer is a solid tumor cancer.
F10. The method of any one of embodiments F1-F9, wherein the subject is a human subject in need thereof.

In yet another set of embodiments (embodiment set G), provided are:
G1. A method for ex vivo activation and expansion of Vγ9Vδ2 T cells, comprising:
   a) contacting a population of cells comprising T cells with a culture system comprising IL-2, IL-15, and a bisphosphonate or a mevalonate pathway intermediate;
and
b) culturing the population of cells ex vivo in the culture system under a hypoxic condition to activate and expand Vγ9Vδ2 T cells.
G2. The method of embodiment G1, wherein the method further comprises obtaining the population of cells from a subject.
G3. The method of embodiment G2, wherein the subject is healthy.
G4. The method of embodiment G2, wherein the subject is unhealthy.
G5. The method of any one of embodiments G1 to G4, wherein the population of cells is a population of mammalian cells.
G6. The method of embodiment G5, wherein the mammalian cells are human cells.
G7. The method of embodiment G6, wherein the human cells are engineered cells.
G8. The method of embodiment G6, wherein the human cells are non-engineered cells.
G9. The method of any one of embodiments G1 to G8, wherein the population of cells is a population of peripheral blood mononuclear cells (PBMCs).
G10. The method of embodiment G9, wherein the PBMCs are freshly obtained PBMCs.
G11. The method of embodiment G9, wherein the PBMCs are frozen PBMCs.
G12. The method of any one of embodiments G1 to 8, wherein the population of cells is derived from a human tissue.
G13. The method of embodiment G12, wherein the human tissue is fresh.
G14. The method of embodiment G12, wherein the human tissue is frozen.
G15. The method of any one of embodiments G1 to G8, wherein the population of cells comprises tumor-infiltrating lymphocytes (TILs).
G16. The method of embodiment G15, wherein the TILs are freshly obtained TILs.
G17. The method of embodiment G15, wherein the TILs are frozen TILs.
G18. The method of any one of embodiments G1 to G17, wherein the population of cells is cultured in the culture system under the hypoxic condition for at least 3 days, or at least 5 days, or at least 7 days, or at least 9 days, or at least 11 days, or at least 13 days, or at least 15 days, or at least 17 days, or at least 19 days, or at least 21 days.
G19. The method of embodiment G18, wherein the population of cells is cultured in the culture system under the hypoxic condition for from 3 days to 25 days, or from 4 days to 23 days, or from 5 days to 21 days, or from 6 days to 19 days, or from 7 days to 17 days, or from 8 days to 15 days, or from 9 days to 14 days, or from 10 days to 14 days, or from 11 days to 14 days, or from 12 days to 14 days.
G20. The method of embodiment G19, wherein the population of cells is cultured in the culture system under the hypoxic condition for about 14 days.
G21. The method of any one of embodiments G1 to G20, wherein the oxygen concentration of the hypoxic condition is less than 15%, or less than 13%, or less than 11%, or less than 9%, or less than 7%, or less than 5%, or less than 3%, or less than 1%, or less than 0.5%.
G22. The method of embodiment G21, wherein the oxygen concentration of the hypoxic condition is from 0.1% to 15%, or from 0.5% to 13%, or from 1% to 13%, or from 1% to 11%, or from 1% to 9%, or from 1% to 7%, or from 2% to 5%.
G23. The method of embodiment G21, wherein the oxygen concentration of the hypoxic condition is or is about 2%, or 5%, or 12%.
G24. The method of embodiment G21, wherein the oxygen concentration of the hypoxic condition is or is about 5%.
G25. The method of any one of embodiments G1 to G24, wherein the method further comprises culturing the population of cells in the culture system under a normoxic condition to activate and expand Vγ9Vδ2 T cells.
G26. The method of embodiment G25, wherein the population of cells is cultured under the normoxic condition for at least 1 hour prior to being cultured under the hypoxic condition.
G27. The method of embodiment G26, wherein the population of cells is cultured under the normoxic condition for from 0.5 days to 7 days, or from 1 days to 6 days, or from 1 days to 5 days, or from 1 days to 4 days, or from 1 days to 3 days, or from 1 days to 2 days prior to being cultured under the hypoxic condition.
G28. The method of any one of embodiments G25 to G27, wherein the oxygen concentration of the normoxic condition is or is about 18.2% or 18.6%.
G29. The method of any one of embodiments G1 to G28, wherein the IL-2 concentration within the culture system is from 10 IU/mL to 1200 IU/mL, or is or is about 10 IU/ml.
G30. The method of embodiment G29, wherein the IL-2 concentration within the culture system is adjusted to decrease during the culturing.
G31. The method of embodiment G30, wherein the IL-2 concentration within the culture system is 1000 IU/mL or no more than 1000 IU/mL on days 0 and 1, 800 IU/mL or no more than 800 IU/mL on days 2, 3 and 4, and 100 IU/mL or no more than 100 IU/mL on day 5 and thereafter.
G32. The method of any one of embodiments G1 to G31, wherein the IL-15 concentration within the culture system is from 5 ng/mL to 25 ng/mL, or from 50 ng/mL to 300 ng/ml, or is or is about 100 ng/mL, or is or is about 200 ng/ml.
G33. The method of embodiment G32, wherein the IL-15 concentration within the culture system is adjusted during the culturing.
G34. The method of embodiment G33, wherein the IL-15 concentration within the culture system is 10 ng/ml or no more than 10 ng/mL on days 0 and 1, 20 ng/ml or no more than 20 ng/mL on days 2, 3, and 4, and 10 ng/ml or no more than 10 ng/ml on day 5 and thereafter.
G35. The method of any one of embodiments G1 to G34, wherein the bisphosphonate is selected from the group consisting of zoledronic acid, risedronic acid, ibandronic acid, alendronic acid, pamidronic acid, tiludronic acid, etidronic acid, and clodronic acid.
G36. The method of any one of embodiments G1 to G34, wherein the mevalonate pathway intermediate is selected from the group consisting of HMBPP, BrHPP, and isopentenyl pyrophosphate.
G37. The method of any one of embodiments G1 to G36, wherein the method increases the total percentage of Vγ9Vδ2 T cells in the population of cells to more than 10%, or more than 15%, or more than 20%, or more than 25%, or more than 30%, or more than 35%, or more than 40%, or more than 45%, or more than 50%, or more than 55%, or more than 60%.
G38. The method of embodiment G37, wherein the method increases the total percentage of Vγ9Vδ2 T cells in the population of cells to from 10% to 99%, or from 20% to 95%, or from 30% to 95%, or from 35% to 95%, or from 40% to 95%, or from 45% to 95%, or from 50% to 95%, or from 55% to 95%, or from 60% to 95%, or from 65% to 95%.
G39. The method of any one of embodiments G1 to G38, wherein the method increases the total number of Vγ9Vδ2 T cells in the population of cells by at least 10-fold, or at least 30-fold, or at least 50-fold, or at least 100-fold, or at least 150-fold, or at least 200-fold, or at least 250-fold, or at least 300-fold, or at least 350-fold, or at least 400-fold, or at least 450-fold, or at least 500-fold, or at least 550-fold, or at least 600-fold as compared to the total number of Vγ9Vδ2 T cells in the population of cells before the contacting.
G40. The method of embodiment G39, wherein the method increases the total number of Vγ9Vδ2 T cells in the population of cells by from 10-fold to 700-fold, or from 30-fold to 650-fold to fold, or from 50-fold to 600-fold, or from 100-fold to 600-fold, or from 150-fold to 600-fold, or from 200-fold to 600-fold, or from 250-fold to 600-fold, or from 300-fold to 600-fold, or from 350-fold to 600-fold, or from 400-fold to 600-fold, or from 450-fold to 600-fold, or from 500-fold to 600-fold as compared to the total number of Vγ9Vδ2 T cells in the population of cells before the contacting.
G41. The method of any one of embodiments G1 to G40, further comprising enriching the Vγ9Vδ2 T cells in the population of cells.
G42. The method of embodiment G41, wherein the enriching results in the percentage of Vγ9Vδ2 T cells in the population of cells is more than 95%.
G43. A method for ex vivo activation and expansion of Vγ9Vδ2 T cells, comprising:
   a) contacting a population of cells comprising T cells with a culture system comprising IL-2, IL-15, and zoledronic acid,
   b) culturing the population of cells ex vivo in the culture system under a hypoxic condition for about 14 days to activate and expand Vγ9Vδ2 T cells, and wherein (i) the IL-2 concentration in the culture system is (i) 1000 IU/mL or no more than 1000 IU/mL on days 0 and 1, 800 IU/mL or no more than 800 IU/mL on days 2, 3 and 4, and 100 IU/mL or no more than 100 IU/mL on day 5 and thereafter, or (2) is or is about 10; (ii) the IL-15 concentration within the culture system is (1) 10 ng/ml or no more than 10 ng/mL on days 0 and 1, 20 ng/ml or no more than 20 ng/mL on days 2, 3, and 4, and 10 ng/mL or no more than 10 ng/ml on day 5 and thereafter, or (2) is or is about 100 ng/ml, or is or is about 200 ng/ml; (iii) the oxygen concentration of the hypoxic condition is or is about 5%; optionally wherein (iv) the zoledronic acid concentration in the culture system is or is about 350 nM.
G44. A method for ex vivo activation and expansion of Vγ9Vδ2 T cells, comprising:
   a) contacting a population of cells comprising T cells with a culture system comprising IL-2, IL-15, and zoledronic acid,
   b) culturing the population of cells under a normoxic condition to activate and expand Vγ9Vδ2 T cells for at least 1 hour prior to being cultured under a hypoxic condition; and
   c) culturing the population of cells under the hypoxic condition for about 15 days (e.g., 14 days) to further activate and expand Vγ9Vδ2 T cells, wherein (i) the IL-2 concentration in the culture system is (i) 1000 IU/mL or no more than 1000 IU/mL on days 0 and 1, 800 IU/mL or no more than 800 IU/mL on days 2, 3 and 4, and 100 IU/mL or no more than 100 IU/mL on day 5 and thereafter, or (2) is or is about 10; (ii) the IL-15 concentration within the culture system is (1) 10 ng/ml or no more than 10 ng/mL on days 0 and 1, 20 ng/ml or no more than 20 ng/mL on days 2, 3, and 4, and 10 ng/ml or no more than 10 ng/ml on day 5 and thereafter, or (2) is or is about 100 ng/ml, or is or is about 200 ng/ml; (iii) the oxygen concentration of the hypoxic condition is or is about 5%; (iv) the oxygen concentration of the normoxic condition is or is about 18.2% or 18.6%; optionally wherein (v) the zoledronic acid concentration in the culture system is or is about 350 nM.
G45. An isolated population of Vγ9Vδ2 T cells produced by the method of any one of embodiments G1 to G44.
G46. An isolated population of cells, wherein the percentage of Vγ9Vδ2 T cells in the isolated population of cells is (a) more than 10%, or more than 15%, or more than 20%, or more than 25%, or more than 30%, or more than 35%, or more than 40%, or more than 45%, or more than 50%, or more than 55%, or more than 60%; or (b) from 10% to 99%, or from 20% to 95%, or from 30% to 95%, or from 35% to 95%, or from 40% to 95%, or from 45% to 95%, or from 50% to 95%, or from 55% to 95%, or from 60% to 95%, or from 65% to 95%.
G47. A pharmaceutical composition comprising the isolated population of Vγ9Vδ2 T cells of embodiment G45 or the isolated population of cells of embodiment G46, and optionally a pharmaceutically acceptable excipient.
G48. A method for treating a disease or disorder in a subject comprising administering to the subject: (i) a therapeutically effective amount of the isolated population of Vγ9Vδ2 T cells of embodiment G45, the isolated population of cells of embodiment G46, or the pharmaceutical composition of embodiment G47, and (ii) a therapeutically effective amount of at least one multispecific antibody;
optionally wherein the at least one multispecific antibody comprises (a) a first binding domain that binds to an antigen expressed on a Vγ9Vδ2 T cell; and (b) a second binding domain that binds to an antigen expressed on an unhealthy cell;

further optionally wherein (a) the antigen expressed on the Vγ9Vδ2 T cell is T Cell Receptor Gamma Variable 9 (TRGV9) or CD3; or (b) the unhealthy cell is a cancer cell, and wherein the antigen expressed on the unhealthy cell is a tumor-associated antigen (TAA); further optionally wherein the cancer cell is a blood cancer cell or a solid tumor cancer cell.
G49. The method of embodiment G48, wherein the at least one multispecific antibody comprises a Vγ9×TAA and/or CD3×TAA bispecific antibody.
G50. The method of embodiment G48 or G49, wherein the disease or disorder is cancer, optionally wherein the cancer is a blood cancer or a solid tumor cancer; further optionally wherein the subject is a human subject in need thereof.
G51. A process for making a chimeric antigen receptor (CAR) T cell, comprising: (i) a step of performing a function of obtaining the isolated population of Vγ9Vδ2 T cells of embodiment G45 or the isolated population of cells of embodiment G46; and
(ii) a step of performing a function of expressing a CAR in the Vγ9Vδ2 T cells;
optionally wherein the CAR comprises an extracellular domain, a transmembrane domain, and an intracellular domain; further optionally wherein the extracellular domain binds to an antigen expressed on an unhealthy cell; further optionally wherein the unhealthy cell is a cancer cell; further optionally wherein the cancer cell is a blood cancer cell or a solid tumor cancer cell.
G52. A method for making a chimeric antigen receptor (CAR) T cell, comprising: (i) obtaining the isolated population of Vγ9Vδ2 T cells of embodiment G45 or the isolated population of cells of embodiment G46; and (ii) introducing nucleic acids encoding a CAR into the population of cells, optionally wherein the CAR comprises an extracellular domain, a transmembrane domain, and an intracellular domain; further optionally wherein the extracellular domain binds to an antigen expressed on an unhealthy cell; further optionally wherein the unhealthy cell is a cancer cell; further optionally wherein the cancer cell is a blood cancer cell or a solid tumor cancer cell.
G53. A CAR T cell produced by the method of embodiment G52.
G54. A pharmaceutical composition comprising the CAR T cell of embodiment G53, and optionally a pharmaceutically acceptable excipient.
G55. A method for treating a disease or disorder in a subject comprising administering to the subject a therapeutically effective amount of the CAR T cell of embodiment G53 or the pharmaceutical composition of embodiment G54.
G56. A method for establishing an in vivo engraftment of Vγ9Vδ2 T cells in a receiving subject, the method comprising (i) obtaining the isolated population of Vγ9Vδ2 T cells of embodiment G45 or the isolated population of cells of embodiment G46; and (ii) adoptively transferring the population of cells to the receiving subject;

optionally wherein the population of cells comprising Vγ9Vδ2 T cells is produced from the population of cells comprising T cells obtained from the receiving subject;

optionally wherein the population of cells comprising Vγ9Vδ2 T cells is a purified population of Vγ9Vδ2 T cells;

optionally wherein the population of cells comprising Vγ9Vδ2 T cells is enriched to comprise more than 95% Vγ9Vδ2 T cells one day before the adoptively transferring the population of cells to the receiving subject;
optionally wherein the population of cells comprising Vγ9Vδ2 T cells comprises more than 10%, or more than 15%, or more than 20%, or more than 25%, or more than 30%, or more than 35%, or more than 40%, or more than 45%, or more than 50%, or more than 55%, or more than 60% Vγ9Vδ2 T cells in the population of cells;

optionally wherein the population of cells comprising Vγ9Vδ2 T cells comprises from 10% to 99%, or from 20% to 95%, or from 30% to 95%, or from 35% to 95%, or from 40% to 95%, or from 45% to 95%, or from 50% to 95%, or from 55% to 95%, or from 60% to 95%, or from 65% to 95% Vγ9Vδ2 T cells in the population of cells;

optionally wherein the population of cells comprising Vγ9Vδ2 T cells comprises less than 95%, or less than 90%, or less than 85%, or less than 80%, or less than 75%, or less than 70%, or less than 65%, or less than 60%, or less than 55%, or less than 50%, or less than 45%, or less than 40%, or less than 35%, or less than 30%, or less than 25%, or less than 20%, or less than 15%, or less than 10%, or less than 5%, or less than 1%, or is devoid of αβ T cells in the population of cells;

optionally wherein the population of cells comprises at least about or about 5×10⁶ cells, at least about or about 1×10⁷ cells, at least about or about 10×10⁷ cells, at least about or about 20×10⁷ cells, at least about or about 30×10⁷ cells, at least about or about 40×10⁷ cells, at least about or about 50×10⁷ cells, at least about or about 60×10⁷ cells, at least about or about 70×10⁷ cells, at least about or about 80×10⁷ cells, at least about or about 90×10⁷ cells, at least about or about 1×10⁸ cells, at least about or about 10×10⁸ cells, at least about or about 20×10⁸ cells, at least about or about 30×10⁸ cells, at least about or about 40×10⁸ cells, at least about or about 50×10⁸ cells, at least about or about 60×10⁸ cells, at least about or about 70×10⁸ cells, at least about or about 80×10⁸ cells, at least about or about 90×10⁸ cells, or at least about or about 1×10⁹ cells.
G57. The method of embodiment G56, wherein the adoptively transferring comprises administering the population of cells to the receiving subject;
optionally wherein the method further comprises (iii) administering, concurrently or sequentially with the adoptively transferring, an effective amount of a composition comprising IL-2, IL-15, a bisphosphonate or a mevalonate pathway intermediate, or a combination thereof;
optionally wherein the bisphosphonate is selected from the group consisting of zoledronic acid, risedronic acid, ibandronic acid, alendronic acid, pamidronic acid, tiludronic acid, etidronic acid, and clodronic acid;
optionally wherein the mevalonate pathway intermediate is selected from the group consisting of HMBPP, BrHPP, and isopentenyl pyrophosphate;
optionally wherein the IL-2 is administered at the dosage of 2×10⁴ IU/kg of body weight;
optionally wherein the bisphosphonate is zoledronic acid administered at the dosage of 2.5 mg/kg of body weight; and
optionally wherein the administration of the population of the cells and/or administration of the effective amount of the composition is intravenous administration or intraperitoneal administration.
G58. The method of embodiment G56 or G57, wherein the adoptively transferred Vγ9Vδ2

T cells produce progeny cells in the subject;
optionally wherein the progeny cells are CD45+ cells, CD56+ cells, or CD69+ cells;

optionally wherein the adoptively transferred Vγ9Vδ2 T cells are present in the blood of the receiving subject at least 7 days, at least 14 days, at least 21 days, or at least 28 days after the adoptive transfer of the population of cells to the receiving subject;

optionally wherein the adoptively transferred Vγ9Vδ2 T cells infiltrate into an tissue in the receiving subject;

optionally wherein the tissue is a spleen tissue, a liver tissue, a lung tissue, an intestine tissue, a skin tissue, or a combination thereof; optionally wherein the tissue comprises an unhealthy cell;

optionally wherein the unhealthy cell is a cancer cell; optionally wherein the cancer cell is a blood cancer cell or a solid tumor cancer cell;

optionally wherein the receiving subject does not develop any symptom of a Graft Versus Host Disease (GvHD) at least 7 days, at least 14 days, at least 21 days, or at least 28 days after the adoptive transfer of the population of cells to the receiving subject.
G59. The method of anyone of embodiments G56 to G58, wherein the Vγ9Vδ2 T cells are chimeric antigen receptor (CAR) T cells comprising an extracellular domain, a transmembrane domain, and an intracellular domain, optionally wherein the extracellular domain binds to an antigen expressed on an unhealthy cell; further optionally wherein the unhealthy cell is a cancer cell; further optionally wherein the cancer cell is a blood cancer cell or a solid tumor cancer cell.
G60. A method for treating a disease or disorder in a subject comprising administering to the subject: (i) a therapeutically effective amount of a population of Vγ9Vδ2 T cells, and (ii) a therapeutically effective amount of at least one multispecific antibody; optionally wherein the at least one multispecific antibody comprises: (a) a first binding domain that binds to an antigen expressed on a Vγ9Vδ2 T cell; and (b) a second binding domain that binds to an antigen expressed on an unhealthy cell;

further optionally wherein (a) the antigen expressed on the Vγ9Vδ2 T cell is T Cell Receptor Gamma Variable 9 (TRGV9) or CD3; or (b) the unhealthy cell is a cancer cell, and wherein the antigen expressed on the unhealthy cell is a tumor-associated antigen (TAA); optionally wherein the cancer cell is a blood cancer cell or a solid tumor cancer cell.
G61. The method of embodiment G60, wherein the method comprises administering to the subject: (i) a therapeutically effective amount of the population of Vγ9Vδ2 T cells, and (ii) a therapeutically effective amount of Vγ9×TAA and/or CD3×TAA bispecific antibodies.
G62. The method of embodiment G60 or G61, wherein the disease or disorder is cancer, optionally wherein the cancer is a blood cancer, or optionally wherein the cancer is a solid tumor cancer; optionally wherein the subject is a human subject in need thereof.
G63. The pharmaceutical composition according to embodiment G47, the isolated population of Vγ9Vδ2 T cells of embodiment G45, or the isolated population of cells of embodiment G46, for use in the treatment of a disease or disorder in a subject, the treatment comprising administering to the subject: (i) a therapeutically effective amount of the isolated population of Vγ9Vδ2 T cells, the isolated population of cells, or the pharmaceutical composition, and (ii) a therapeutically effective amount of at least one multispecific antibody;
   optionally wherein the at least one multispecific antibody comprises (a) a first binding domain that binds to an antigen expressed on a Vγ9Vδ2 T cell; and (b) a second binding domain that binds to an antigen expressed on an unhealthy cell;
   further optionally wherein (a) the antigen expressed on the Vγ9Vδ2 T cell is T Cell Receptor Gamma Variable 9 (TRGV9) or CD3; or (b) the unhealthy cell is a cancer cell, and wherein the antigen expressed on the unhealthy cell is a tumor-associated antigen (TAA); further optionally wherein the cancer cell is a blood cancer cell or a solid tumor cancer cell.
G64. The pharmaceutical composition, the isolated population of Vγ9Vδ2 T cells, or the isolated population of cells for use according to embodiment G63, wherein the at least one multispecific antibody comprises a Vγ9×TAA and/or CD3×TAA bispecific antibody.
G65. The pharmaceutical composition, the isolated population of Vγ9Vδ2 T cells, or the isolated population of cells for use according to embodiment G63 or embodiment G64, wherein the disease or disorder is cancer, optionally wherein the cancer is a blood cancer or a solid tumor cancer; further optionally wherein the subject is a human subject in need thereof.

### 6. EXAMPLES

The following is a description of various methods and materials of the present disclosure and provide a person of ordinary skill in the art with information to make and use the present invention. The teachings in the present specification are representative and are not intended to limit the scope of what the inventors regard as their invention. Efforts have been made to ensure accuracy with respect to numbers used (e.g., amounts, percentages, etc.), but some experimental errors and deviations are to be taken into consideration, as appropriate.

### 6.1. Example 1-Methods and Materials

### 6.1.1. Isolation of Fresh PBMC from Human Blood

On the day of blood isolation, each donor's blood was collected into heparinized collection tubes and was processed immediately following the steps below. All reagents used were pre-warmed to the room temperature. Each donor's blood was transferred into a separate sterile bottle. An equal volume of 1× DPBS with 2% heat inactivated fetal bovine serum (HI FBS) was added to the blood sample and was mixed with the blood sample by gently swirling the bottle. A Stem Cell SepMate tubes was filled with 15 mL of Lymphoprep and was slowly top up with ˜25 mL of blood-DPBS mixture. The SepMate tube was centrifuged at 1200 g for 10 min. Supernatant containing a ring of PBMC was poured into a new 50 ml conical tube and the SepMate tube was kept in inverted position for less than 2 sec during the process. Volume of supernatant with PBMC was brought up to 50 mL by adding 1× DPBS with 2% HI FBS. The conical tube was centrifuged at 300 g for 5 minutes, and the supernatant was gently discarded. The PBMC pellet was resuspended in 50 mL 1× DPBS with 2% HI FBS. The number of cells were counted and their viability was determined based on propidium iodide (PI) exclusion. PI exclusion was determined by taking 10 μl of the cell mixture, adding 10 μl of PI dye solution (Orflo) and 80 μl of DPBS, 2% HI FBS. The cells were counted on Orflo. The cell gating on Orflo was set to 6 to 20 μm, lower cell viability gating was based on live/dead population separation. Typically, for freshly isolated PBMCs, the viability should be ˜95-100%. T conical tube with PBMC was centrifuged at 300 g for 5 minutes. Cell concentration was adjusted to 1×10⁶ cells/mL in complete Day 0 γδ T cell growth medium and 1×10⁶ cells were analyzed using flow-based analysis to determine the starting percentage of Vγ9Vδ2 T Cells. 6.1.2. Reviving Frozen PBMC

T cells were expanded from frozen PBMC (obtained from HemaCare, 100×10⁶ PBMC/vial) following the steps below. A frozen vial with PBMC was transferred to the 37° C. water bath and was incubated until medium was completely thawed (maximum 2-3 minutes). It was important that the sample was not warmed up above the thawing temperature (0-10° C.). The thawed cells were quickly transferred to pre-warmed complete medium and was centrifuged at 300 g for 5 min. The supernatant was gently discarded and the cells were resuspended in 50 mL of complete medium. The number of cells were counted and their viability was determined based on PI exclusion. PI exclusion was determined by taking 10 μl of the cell mixture, adding 10 μl of PI dye solution (Orflo) and 80 μl of DPBS, 2% HI FBS. The cells were counted on Orflo. The cell gating on Orflo was set to 6 to 20 μm, lower cell viability gating was based on live/dead population separation. Typically, for freshly isolated PBMCs, the viability should be ˜75-95%. The tube with PBMC was centrifuged at 300 g for 5 minutes. Cell concentration was adjusted to 1×10⁶ cells/mL in complete Day 0 γδ T cell growth medium and 1 ×10⁶ cells were analyzed using flow-based analysis to determine the starting percentage of Vγ9Vδ2 T Cells.

### 6.1.3. Selective Expansion of Vγ9Vδ2 T Cells

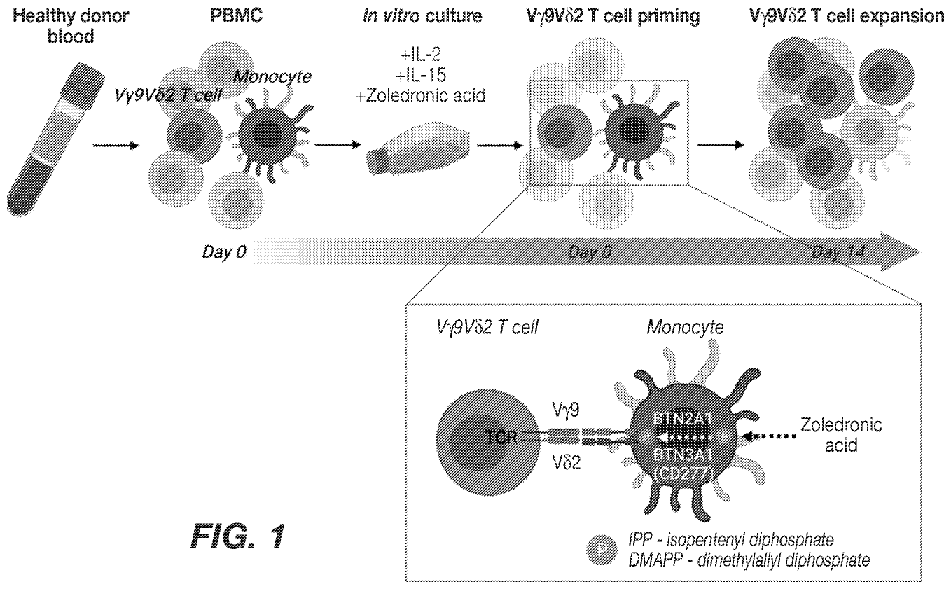

An overall process of the present disclosure is illustrated in FIG. 1. On day 0, PBMC was plated in Day 0 Vγ9Vδ2 T cell medium at cell density 1×10⁶ cells/mL in tissue culture flask. On day 2, the medium was topped up with an equal volume of Day 2 Vγ9Vδ2 T cell medium. On day 5, the entire volume of flask was collected and cells were centrifuged at 300 g for 5 min. The supernatant was carefully aspirated. The cells were resuspended in Day 5-14 Vγ9Vδ2 T cell medium. Cell count and viability were determined, and cell concentration was adjusted to 1×10⁶ cells/ml. The cells were transfer to the new flask for continued culturing. By day 5, most donors exhibited blasting which was observed via microscope. Blasting might start as early as at day 3. Early blasting, however, did not always correlate with efficient expansion.

The cells were examined under a microscope every other day. If the medium became acidic and changed color to yellow, more medium was added or the cells were split. Typically, cells needed to be split every 2-3 days post day 5 split. If media was pink, no additional medium was added to avoid diluting the cell density. When robust expansion was observed, medium would become acidic rapidly and it was necessary to spin cells to re-adjust cell density to 1×10⁶ cells/mL. Tissue culture incubators were used throughout the process for a normoxic condition, which contained 18.2% or 18.6% 02.

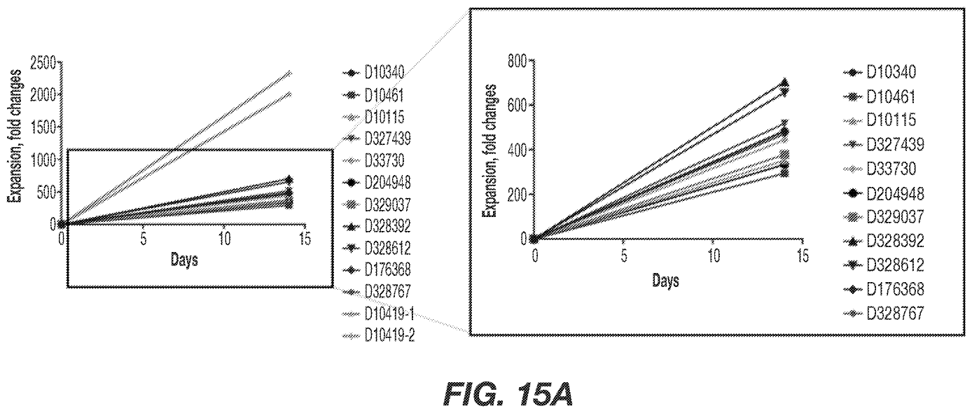

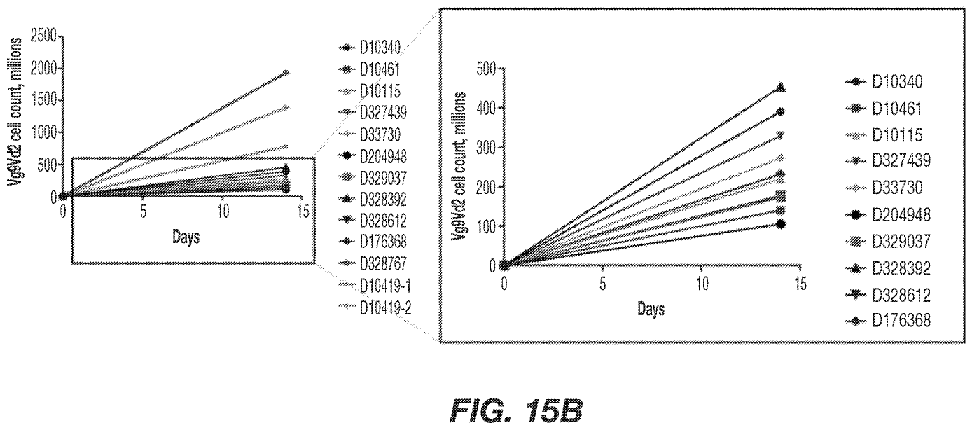

FIG. 15A shows the expansion of Vγ9Vδ2 T cells among different donors (donors 1-3, 12, 13-expanded from fresh PBMC, 4-11 expanded from frozen PBMC). The right panel shows higher resolution for majority of samples on the left. The total number of

Vγ9Vδ2 T cells varies from donor to donor. Some donors resulted in total number of 1-2×10° of Vγ9Vδ2 T cells at day 14 post-expansion. The majority of donors provided 100-500×10⁶ of Vγ9Vδ2 T cells at day 14 post-expansion. FIG. 15B shows the total number of Vγ9Vδ2 cells obtained post-expansion at day 14. No enrichment was performed. The cell number was determined based on percentage of Vγ9⁺ CD3⁺ cells in total cell suspension. The right panel shows higher resolution for majority of samples on the left.

### 6.1.4. Expansion of Vγ9Vδ2 T cells under Hypoxic Conditions

To expand Vγ9Vδ2 T cells under hypoxic conditions, a standardized Vγ9Vδ2 T cells expansion protocol as described in Section 6.1.3 was used. To achieve desired O₂ concentrations, Avatar System incubators (xCellbio) (i.e. hypoxia chambers) were used. Avatar hypoxia chambers were set up to operate under 37° C., 5% CO₂, 0 psi, and range of O₂% (0.1% to 15%).

### 6.1.5. Enrichment of γδ T Cells

Negative selection was performed to obtain pure population of Vγ9Vδ2 T cells. TCRγ/δ⁺ T Cell isolation (Miltenyi Biotec, 130-092-892) was performed according to manufacturer's instruction. Typical yields averaged in 90-98% pure population of γδ T cells. Removal of non-γδ T cells results in population containing pan γδ T cells. Although ˜95-99% of cells represent Vγ9Vδ2 T cells subset, presence of other γδ T cells cannot be ruled out in enriched cell mixture.

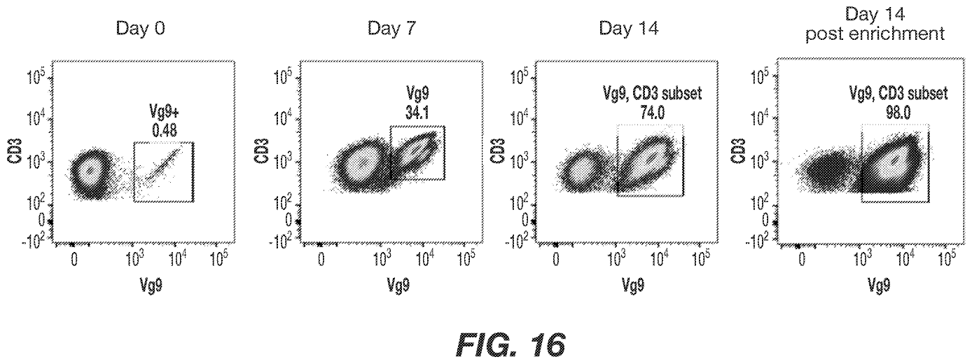

FIG. 16 shows the percentage of Vγ9⁺ CD3⁺ cells gradually increased from day 0 to day 14 reaching ˜75% at day 14. The cells were further enriched with a negative selection kit (panel 4). Data or donor 10419 was shown in FIGS. 15A and 15B. PBMCs were from fresh specimen.

### 6.1.6. Cryopreservation of Vγ9Vδ2 T Cells

Expanded/enriched Vγ9Vδ2 T cells were stored as a frozen stock following the steps below. Vγ9Vδ2 T cells were washed in calcium and magnesium free 1λDPBS and spined at 300 g for 5 min. CryoStor CS10 freezing medium was added to resuspend cell pellet completely to reach a cell concentration of 0.5-10×10⁶ cells/mL (higher concentration was preferred). The CryoStor CS10 cell suspension was incubated at 4° C. for 10 minutes. The cell suspension was transferred into cryovials and freeze down to −80° C. using slow freezing containers. After the sample was frozen for 24 hours, it was transferred to liquid nitrogen storage. 6.1.7. Revival of Vγ9Vδ2 T Cells

Frozen Vγ9Vδ2 T Cells were revived following the steps below. The frozen vial with Vγ9Vδ2 T cells was transferred to the 37° C. water bath and was incubated until the medium was completely thawed. It was important that the sample was not warmed up above the thawing temperature (0-10° C.). The thawed cells were quickly transferred to pre-warmed complete medium and was centrifuged at 300 g for 5 min. The supernatant was gently discarded and the cells were resuspended in 50 mL of complete medium. The number of cells were counted and their viability was determined based on PI exclusion. PI exclusion was determined by taking 10 μl of the cell mixture, adding 10 μl of PI dye solution (Orflo) and 80 μl of DPBS, 2% HI FBS. The cells were counted on Orflo. The cell gating on Orflo was set to 6 to 20 μm, lower cell viability gating was based on live/dead population separation. Typically, the thawed Vγ9Vδ2 T cells viability ranged from ˜75-90%. The tube with Vγ9Vδ2 T cells was centrifuged at 300 g for 5 minutes. The cell concentration was adjusted to 1×10⁶ cells/mL in complete Day 5-14 γδ T cell growth medium.

### 6.1.8. Flow Cytometry Assay

To determine the number and phenotype of Vγ9Vδ2T cells, cells were examined by flow-cytometry at any point of expansion following the steps below. At least 1×10⁶ cells were used for each staining condition. Cells were washed with BD Pharmigen staining buffer (BSA) and centrifuged at 3000 rpm for 1 min. The supernatant was discarded and a staining mix containing antibodies (1 μL per reaction) and FcX blocking solution (5 μL per reaction) was added. The cells were incubated for 30 min at 4° C. in the dark, and were then washed with BD Pharmigen staining buffer (BSA) twice and were fixed for 7-10 min with CytoFix fixation solution. The cells were washed with BD Pharmigen staining buffer (BSA) twice and were resuspended in 100 μL of BD Pharmigen staining buffer (BSA). Events were recorded at the rate 1 μL/sec. Compensation beads staining was performed in parallel with cell staining and same incubation and washing procedures were applied to both.

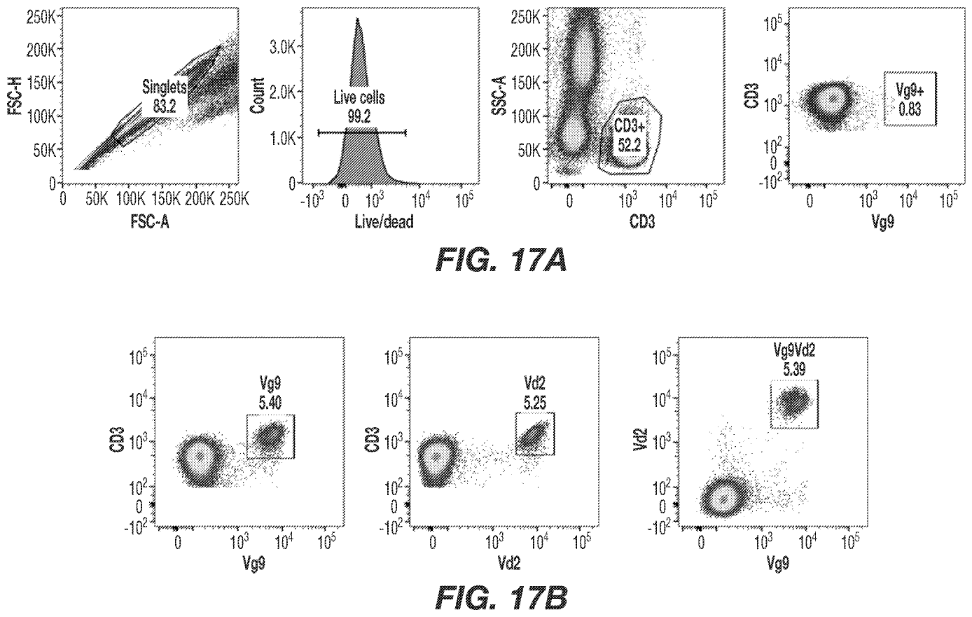

FIG. 17A shows the gating strategy for determining the number of Vγ9Vδ2 T cells by gating singlets, live cells, CD3⁺ cells, and Vγ9+ T cells. Alternatively, both Vγ9 and Vδ2 stains were used for determining the number of Vγ9Vδ2 T cells. FIG. 17B shows that either single Vγ9 or Vδ2 markers or both Vγ9 and Vδ2 were used to identify the Vγ9Vδ2 T cell populations.

### 6.1.9. scRNA-Seg Data Processing, Integration, and Cell Typing

The obtained 3′ single-cell RNA sequencing (scRNA-seq) data was mapped to a mixed reference genome using the Cellranger 3.1.0 software. Data was mapped using the GRcH38 reference genome. For the construction of the count matrices the count tool was run with standard parameters.

Count data was processed using the approaches as described (Luecken and Theis, 2019) by the Scanpy framework (Wolf et al., 2018). Data was filtered by removing cells that have less than 1100 genes for the different samples. Human count data was separated from custom reference count data before further data processing. The data was normalized based on the protocol defined by (Weinreb et al., 2018), log normalize and scaled. PCA dimensionality reduction was performed, and data was projected using the Uniform Manifold Approximation and Projection for Dimension Reduction (UMAP) algorithm (McInnes, Healy, & Melville, 2018).

scRNA-seq data was integrated using the Harmony tool (Korsunsky et al., 2019) for batch correction from scRNA-seq data with parameters theta 0. Data was projected and manually assigned cell types. Based on the annotated dataset, a reference was constructed to extrapolate the identified cell types with SingleR (Aran et al., 2019).

### 6.1.10. Impedance-Based Killing Assay with Adherent Cell Lines (xCELLigence)

A specialized instrument (xCELLige-ce RTCA-Real-Time Cell Analysis) was used to measure real-time impedance data from each well at specified time intervals. Impedance was used to describe a disturbance in the electrical signal passing across a well due to the shape, confluency, and strength of attachment of adherent cell lines. The greater the confluency and strength of attachment of the cells to the assay plate (e-plate), the greater the impedance value. Impedance was expressed in a value termed a cell index (CI), a unitless value.

The assay was performed following the steps below. A vial of target tumor cell line (JIMT1) was thawed following the determination of cell counts and viability. The thawed cells were cultured in complete growth medium for at least 2 passages prior to the assay. Vγ9Vδ2 T cells from selected donors were expanded for 12-14 days. Negative enrichment of Vγ9Vδ2 T cells was performed to remove irrelevant cell types from the cell mixture (αβ T cells, NK cells) one day prior to the assay. Flow-cytometry-based analysis of enriched samples was run to determine purity of Vγ9Vδ2 T cell populations. The cells were rested in complete growth medium overnight. On the day of the assay, target cells (JIMT1) were dislodged by adding 0.25% Trypsin-EDTA. The reaction was quenched with complete growth medium and the target cells were centrifuged down at 300 g for 5 min. Cell number and viability was determined by using trypan blue exclusion on Vi-CELL XR Cell Viability Analyzer. Cell concentration was adjusted to 2.5×10⁵/mL with complete growth medium. Fourth, concentration of test article was adjusted to 400 nM and serial dilutions for test articles were prepared according to Table 1 below. Volume of test articles needed for the assay was adjusted accordingly.

TABLE 1
Serial Dilutions of Test Articles

| Final Concentration | 4x Concentration | Stock | Complete Medium |
|---|---|---|---|
| nM | nM | μL | μL |
| 33.33 | 133.32 | 33.33 | 66.67 |
| 11.11 | 44.44 | 33.33 | 66.67 |
| 3.7 | 14.8 | 33.3 | 66.7 |
| 1.23 | 4.92 | 33.24 | 66.76 |
| 0.41 | 1.64 | 33.33 | 66.67 |
| 0.14 | 0.56 | 34.14 | 65.86 |
| 0.046 | 0.184 | 33.33 | 66.67 |

Instrument for the assay was prepared by setting up the plate maps in software (RTCA). Water bath was filled with water at 37° C. prior to the assay. Since xCELLigence platform was sensitive to temperature changes as that directly affects cell morphology and adherence to the plate, it was critical to allow all reagent to be warmed up to 37° C. and it was recommended to allow the plate to sit at 37° C. prior to the start of the assay. 50 μL of complete growth medium was added to each well on the assay plate and RTCA blank was performed. 4× test article and target cell line were added. The assay was initiated for 24 hrs until target cells completely adhered to the plate and reached cell index value equal to 1. The enriched and rested Vγ9Vδ2 T cells were collected from cell culture and were centrifuged down at 300 g for 5 min. Cell number and viability was determined. Cells were suspended in warm complete growth medium at desired concentration (8.4×10⁴ for E: T 1:3). Once cell suspensions were ready, they were moved to the 37° C. CO2 incubator on shaker to ensure correct temperature for effector cell population. RTCA instrument was paused and the plate from the cradle was removed. The effector cells were added according to the experimental layout and the assay was continued after plated incubate in the RTCA lock cradle for 20-30 min. The data was analyzed using software RTCA.

### 6.1.11. Materials Used

TABLE 2
Culture Medium Compositions

| Reagent | Composition | Reagent | Composition |
|---|---|---|---|
| Complete | RPMI | Day 2 Vγ9Vδ2 | RPMI |
| growth medium | 10% HI FBS | T cells | 10% HI FBS |
| for Vγ9Vδ2T | 1% pen strep | growth medium | 1% pen strep |
| cells | | | 350 nM zol |
| | | | 800 U/mL rhIL-2 |
| | | | 20 ng/mL rhIL-15 |
| Base Media | RPMI1640 + | Day 5 Vγ9Vδ2 | RPMI |
| | Glutamax | T cells | 10% HI FBS |
| | 10% HI FBS | growth medium | 1% pen strep |
| | 1% Anti-Anti | | 350 nM zol |
| | 1x NEAA | | 100 U/mL rhIL-2 |
| | 1x Sodium Pyruvate | | 10 ng/mL rhIL-15 |
| Day 0 Vγ9Vδ2 | RPMI | Complete | DMEM |
| T cells | 10% HI FBS | growth | 10% HI FBS |
| growth medium | 1% pen strep | medium for | 1% pen strep |
| | 350 nM zol | JIMT1 cells | |
| | 1000 U/mL rhIL-2 | | |
| | 10 ng/mL rhIL-15 | | |

### 6.2. Example 2-Vγ9Vδ2 T Cells Expanded under Normoxic Condition

### 6.2.1. Characterize the Starting Vγ9Vδ2 T Cell Populations in PBMC

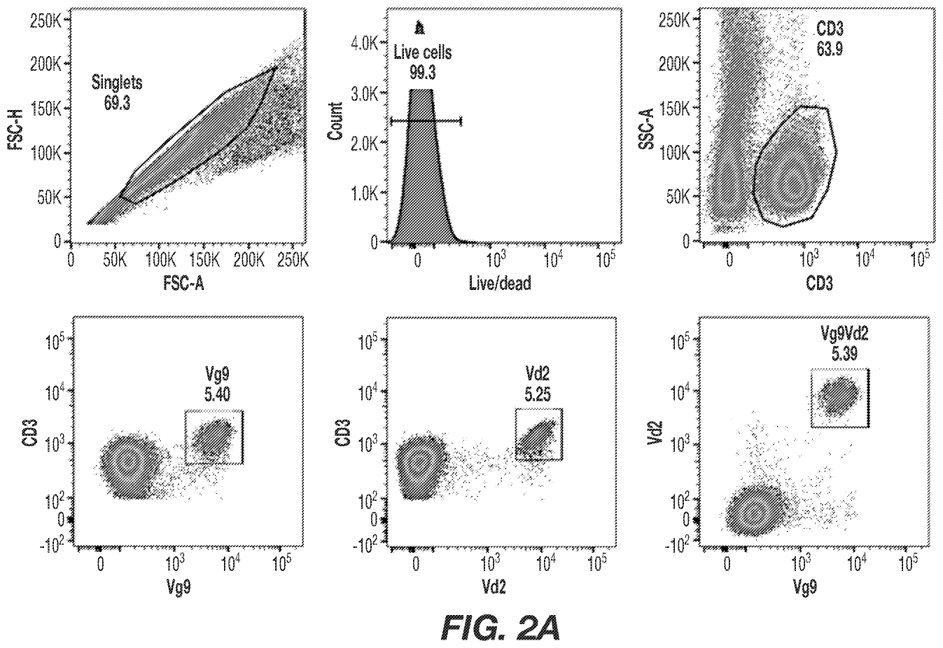

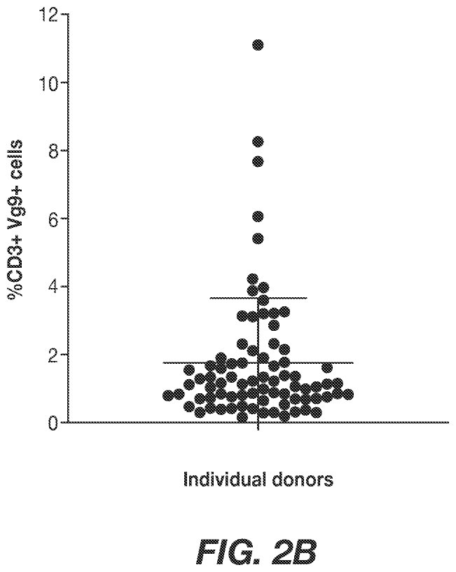

To understand the differences of Vγ9Vδ2 T cell expansion profile across a cohort of 68 healthy donors, the starting Vγ9Vδ2 T cell populations in PBMC of each donor (freshly isolated or freshly thawed) was measured by flow cytometry gating on single viable CD3*Vγ9Vδ2 subset (see FIG. 2A). As shown in FIG. 2B, the starting population of Vγ9Vδ2 T cells varied from 0.1% to 11% of the CD3+ population. However, the majority of donors were found to have ˜0.1 to 2% of CD3+Vγ9Vδ2 T cells. These data demonstrated that Vγ9Vδ2 T cell subset represented a minor population of human lymphocytes.

### 6.2.2. Identify Potential Factors Affecting Vγ9Vδ2 T cell Expansion

To assess expansion capacity of Vγ9Vδ2 T cells, PBMC-based in vitro expansion under the normoxic condition (18.2% oxygen) was performed following the steps described in Section 6.1.3. To evaluate Vγ9Vδ2 T cell expansion in each individual donor, a fold expansion value was determined according to the formula below:

$$Expansion , fold changes = ( total cell count / CD {3}^{+} / V γ 9 V δ 2 at day 14 ) / \\ ( total cell count / CD {3}^{+} / V γ 9 V δ 2 at day 0 )$$

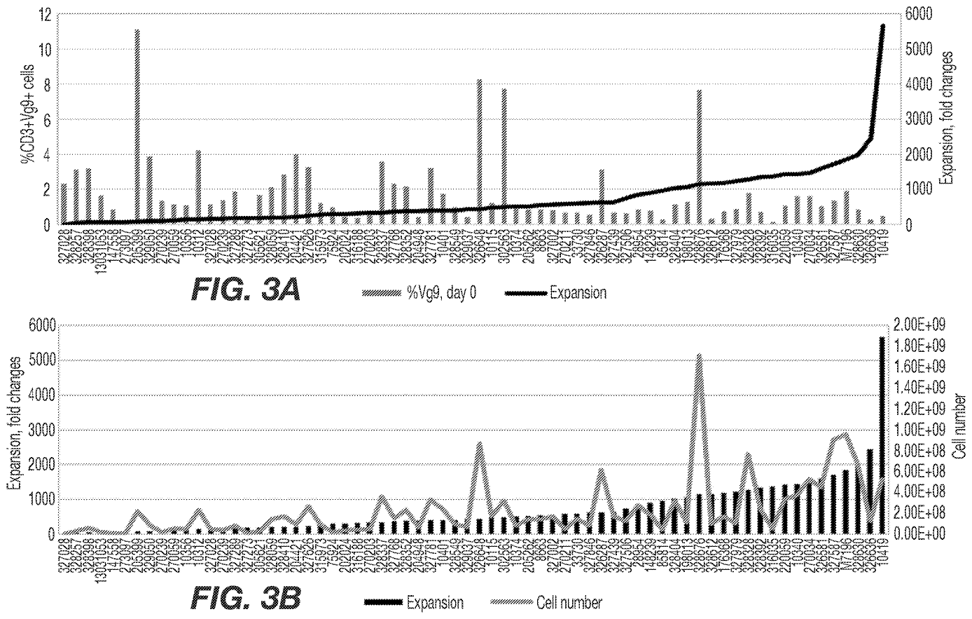

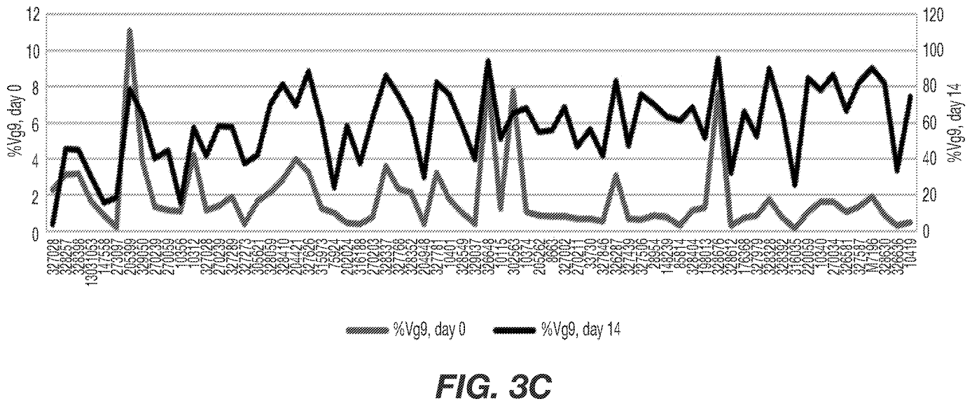

Analysis of expansion values across different donors revealed a diverse and donor-dependent range of expansion from 0 to ˜6000-fold (see FIGS. 3A to 3C). To assess whether expansion efficiency was determined by starting population, Vγ9Vδ2 T cell expansion value was plotted against frequency of starting CD3*Vγ9Vδ2 T cell populations. As shown in FIG. 3A, there was no correlation between the expansion efficiency and the frequency of starting CD3*Vγ9Vδ2 T cell populations. To analyze the relationship between the expansion efficiency and total obtained number of Vγ9Vδ2 T cells after the expansion, Vγ9Vδ2 T cell expansion value was plotted against total number of obtained Vγ9Vδ2 T cells (see FIG. 3B). It was noted that some donors with the largest starting Vγ9Vδ2 T cell populations yielded higher total Vγ9Vδ2 T cell numbers and purity (frequency) of Vγ9Vδ2 T cells at the end of the expansion period, which was expected. Frequencies of initial and final Vγ9Vδ2 T cell populations were compared in FIG. 3C, indicating that donors resembling largest starting Vγ9Vδ2 T cell populations also demonstrated the most Vγ9Vδ2 T cell enriched cultures. Taken together, these data suggested that by using IL-2, IL-15, and zol cocktail, Vγ9Vδ2 T cells were able to be expanded from the cohort of 68 healthy donors. Surprisingly, the Vγ9Vδ2 T cell expansion efficiency was not dependent on the frequency of starting Vγ9Vδ2 T cell populations.

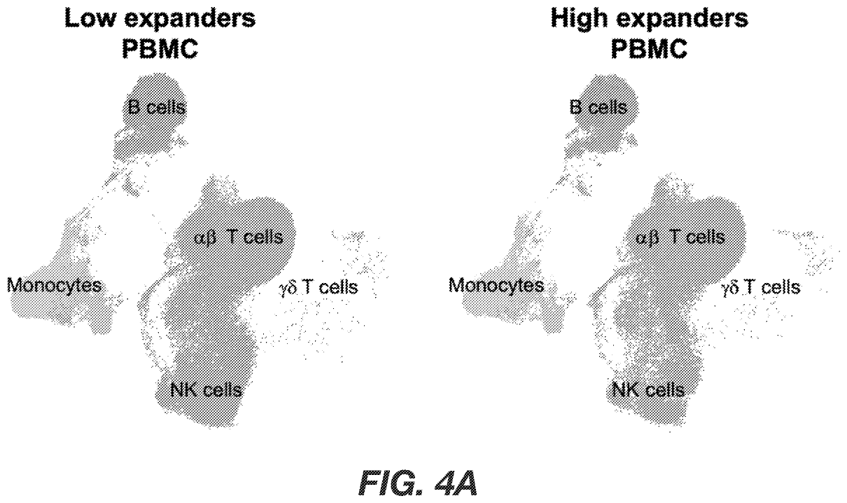

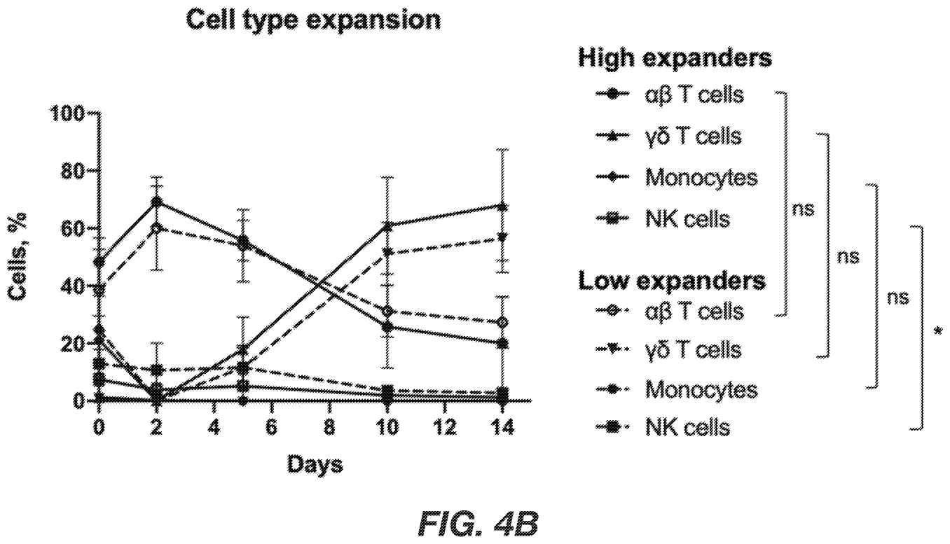

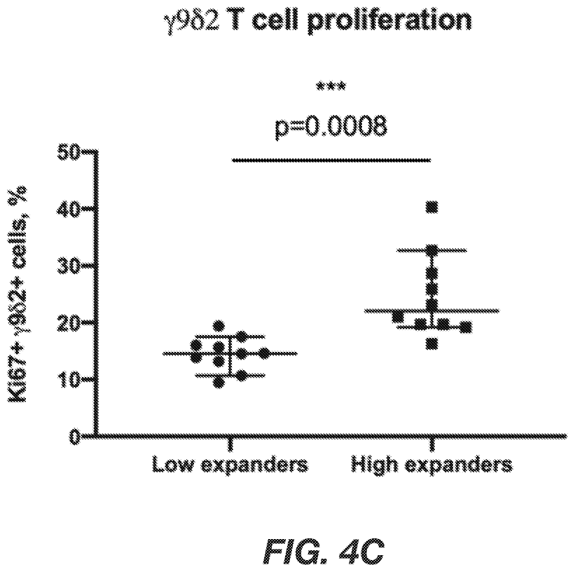

To understand the differences underlying differential Vγ9Vδ2 T cell expansion, donors demonstrating Vγ9Vδ2 T cell expansion above 1000-fold were ranked as high expanders, and donors showing Vγ9Vδ2 T cell expansion below 300-fold were ranked as low expanders. Immune cell clusters within PBMC from 5 low expanders and 5 high expanders were compared. As shown in FIG. 4A, there was minimal presence of γδ T cells in PBMC at day 0 of both low and high expanders. However, the low expanders demonstrated a denser NK cell cluster as compared to the high expanders.

The presence of αβ T cells, Vγ9Vδ2 T cells, NK cells and monocytes throughout the 14-day expansion period between 10 high expanders and 10 low expanders were also compared. As shown in FIG. 4B, both high and low expanders demonstrated similar expansion kinetics. Re αβ T cells, there were high counts of αβ T cells in the beginning of expansion and was a decline from average 60% to 30%. While overall values did not show a significant difference between high and low expanding groups, higher frequency of αβ T cells were typically observed in low expanders by the end of expansion. Re NK cells, NK cells accounted for a minor presence in expansion cultures and showed a decline from around 15% at day 0 to less than 5% by day 14. Interestingly, NK cells demonstrated significantly higher presence in low expanders throughout the entire expansion period (day 0 p=0.005, day 2 p=0.038, day 5 p=0.030, day 10 p=0.035, day 14 p=0.017, multiple t tests). Re monocytes, monocytes did not appear to show differences between high and low expanders, and their presence in the culture declined by day 2. Conversely, Vγ9Vδ2 T cells expanded rapidly starting with <1% at day 0, reaching ˜20% at day 5, and accounting for average of 60% by day 14. It was noted that high expanders tended to have higher frequencies of Vγ9Vδ2 T cells, although this trend was not significant.

Since there was no sticking difference in composition of different immune subsets in Vγ9Vδ2 T cell expansion cultures, the proliferative capacities of 10 high expanders and 10 low expanders were comp by measuring the frequency of Vγ9Vδ2 T cells expressing the proliferation marker Ki-67 at day 14. As shown in FIG. 4C, Vγ9Vδ2 T cells in high expanders were significantly more proliferative (p=0.0008, unpaired t test).

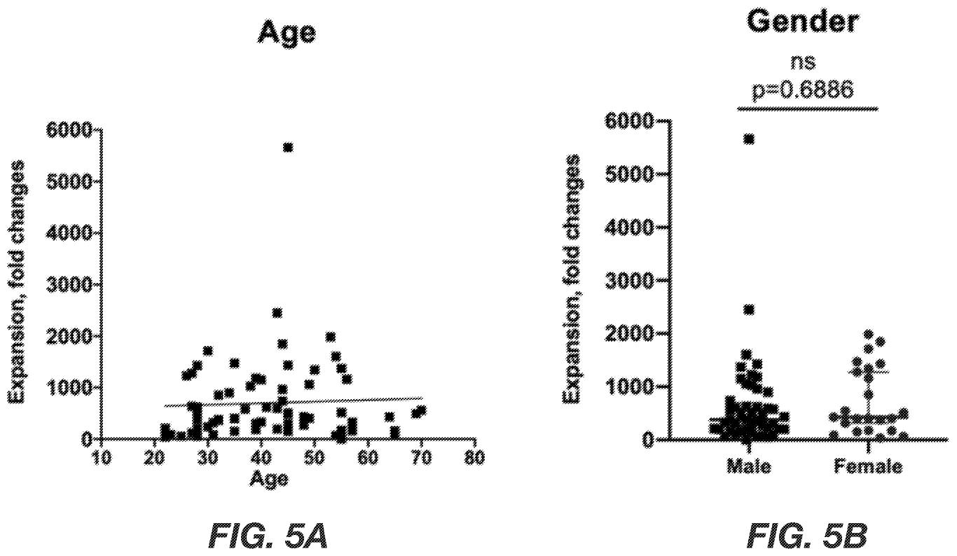

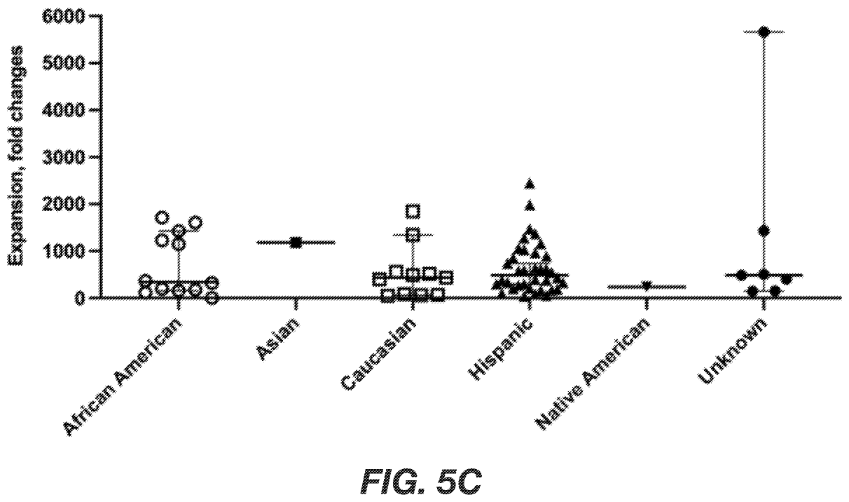

To understand whether any demographic parameters determine differences in Vγ9Vδ2 T cell expansion, expansion efficiency was plotted by age, gender, and ethnicity of 68 healthy donors enrolled into the study. As shown in FIG. 5A, surveyed donors (age 23 to 70 years old) did not show differences in Vγ9Vδ2 T cell expansion efficiency relatively to age. As shown in FIG. 5B, while the majority of surveyed donors were male, there were no differences in Vγ9Vδ2 T cell expansion efficiency between genders (unpaired t test, p=0.6886). As shown in FIG. 5C, high and low expanders were distributed equally across the groups (one-way Anova, p=0.4979). Some ethnical groups were underrepresented in the present study (e.g., Asian, Native American), while some donors were overrepresented (e.g., Hispanic) due to sample availability. Altogether, the above data demonstrated that Vγ9Vδ2 T cell expansion was donor dependent but was not linked to any demographic property.

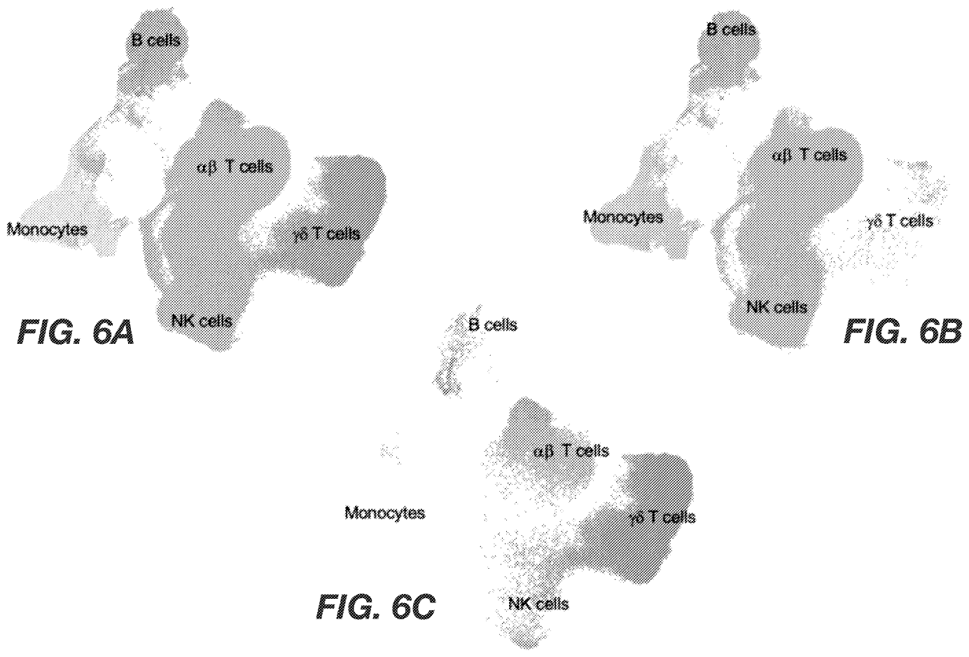

To determine the key biological differences driving enhanced Vγ9Vδ2 T cell expansion and proliferation, single-cell RNA sequencing (scRNA-seq) was performed on 5 high and 5 low expanders' whole blood PBMC at day 0, expanded non-enriched Vγ9Vδ2 T cell at day 14, and non-expanded enriched γδ T cells at day 0. Data from 358370 cells obtained from 10 donors (5 high expanders and 5 low expanders) across 3 experimental conditions was further used to map immune cell identities. FIG. 6A shows UMAP plot of immune cell clusters representing all cells used in the present study. Major cellular clusters included B cells, monocytes, αβ T cells, NK cells and γδ T cells. FIG. 6B shows UMAP plots of immune cell clusters representing compiled sequenced cells from 10 donors' PBMC at day 0. There was minimal presence of γδ T cells in this cohort. FIG. 6C shows UMAP plots of immune cell clusters representing compiled sequenced cells from 10 donors' expanded non-enriched Vγ9Vδ2 T cell at day 14. Majority of cells collected from this cohort represented γδ T cell identity. Additional non-γδ T cell subsets were detected in this cohort and represented αβ T cells and NK cells. The result was consistent with previous flow cytometric analysis shown in FIG. 4B. Since PBMC subset had minimal numbers of γδ T cells (see FIG. 4A), the following experiments focused on expanded non-enriched Vγ9Vδ2 T cell at day 14 (“expanded cohort”), and non-expanded enriched γδ T cells at day 0 (“enriched cohort”).

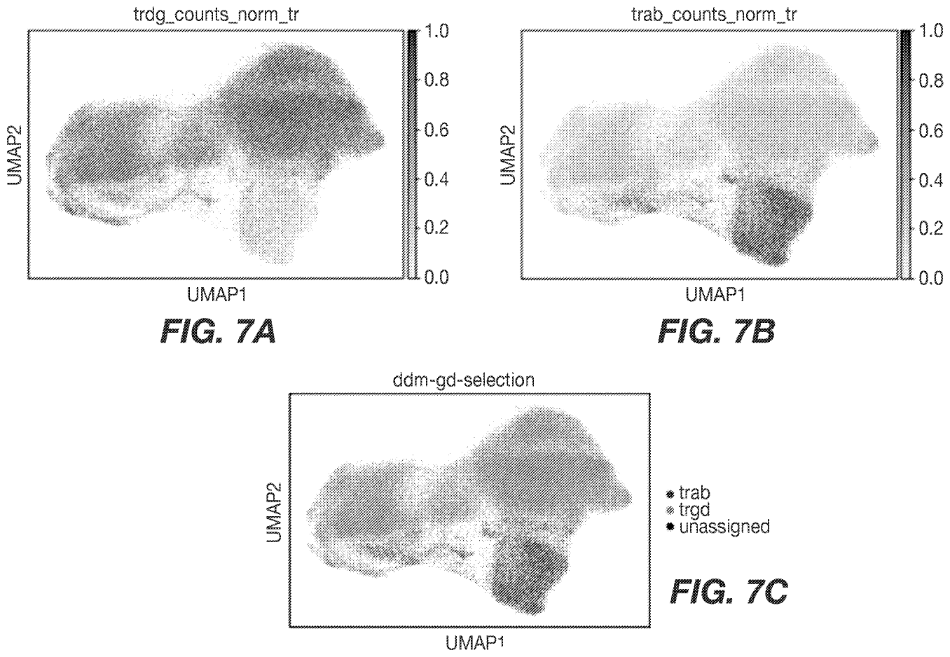

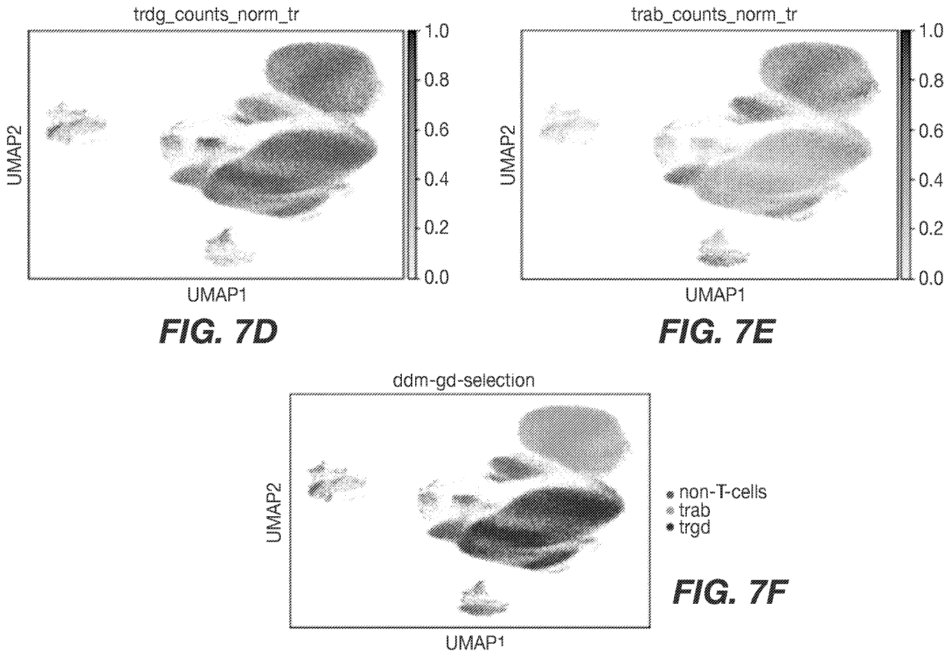

To rule out contamination of γδ T cell cluster with αβ T cells, lineage specific signatures for αβ T cells TRAB (compiled TCR alpha beta signatures) and γδ T cells TRDG (compiled TCR gamma delta signatures) were mapped across the expanded and the enriched cohort. In the expanded cohort, the majority of cells corresponded to TRDG signature (see FIGS. 7A and 7C). Other non-γδ T cells corresponding with TRAB signatures were identified and highlighted in FIG. 7B. In the enriched cohort, most of the cells represented TRDG signature (see FIGS. 7D and 7F). However, a significant portion of non-γδ T cells was present (see FIG. 7F). To eliminate interference of further analyses by non-γδ T cells, non-γδ T cells were removed from the cluster.

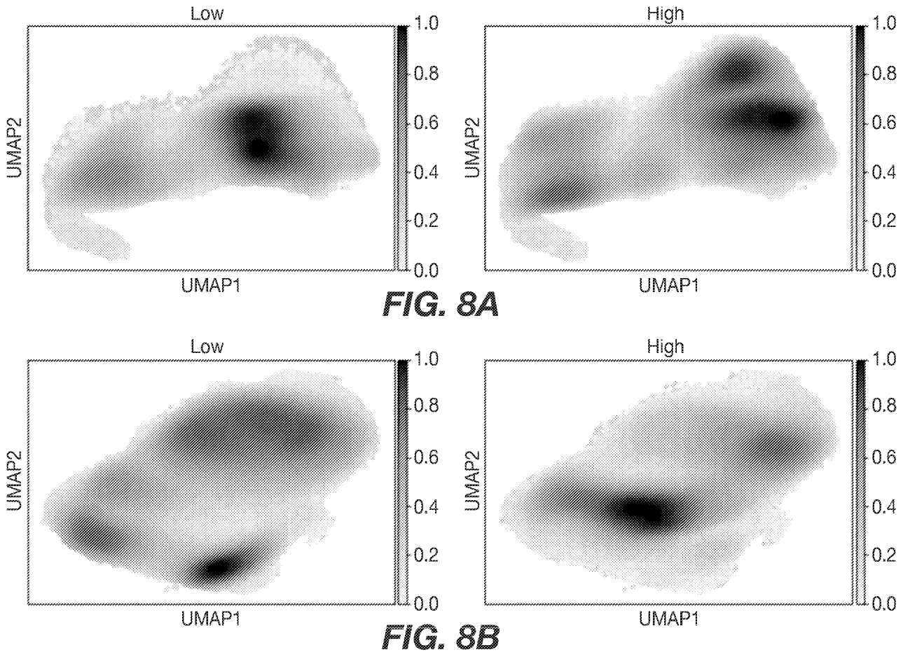

Further analysis of cell densities across high and low expanders revealed presence of a unique clusters of Vγ9Vδ2 T cells in the expanded and the enriched cohorts. As shown in FIG. 8A, high expanders (right panel) showed a presence of high-density subset which was absent in low expanders. Similarly, as shown in FIG. 8B, γδ density plots varied between high and low expanders in the enriched cohort. Detection of specific clusters indicated a presence of unique population of γδ T cells that drives differences between high and low expanders.

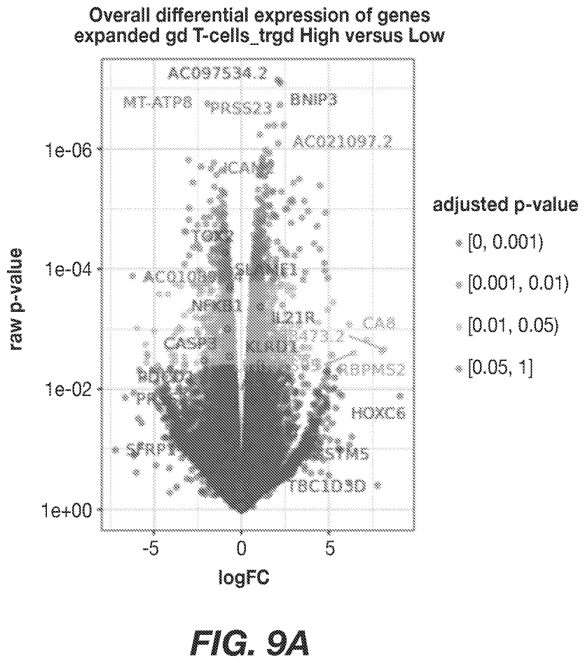

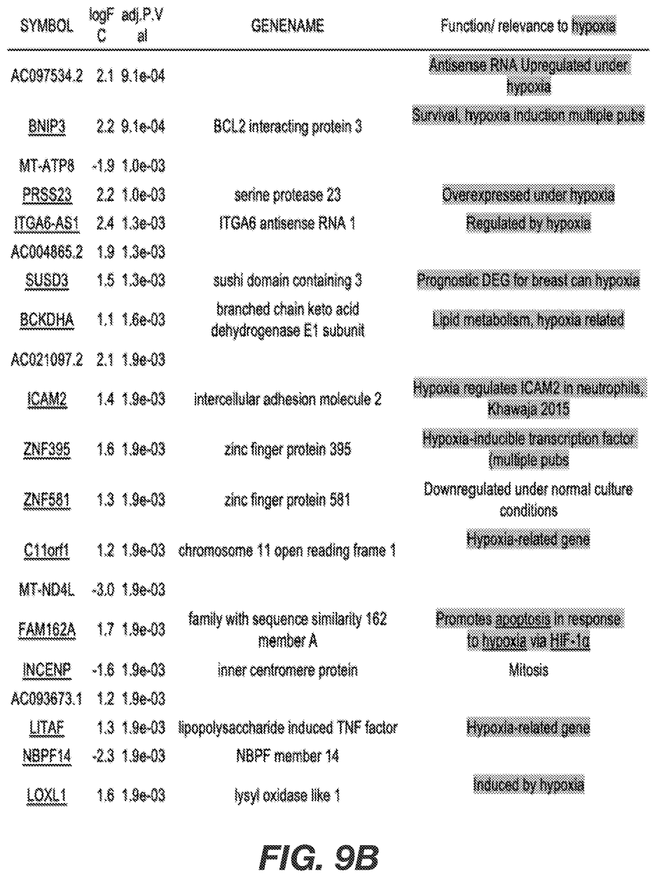

To determine quantitative differences in expression levels between high and low expanders, differentially expressed gene (DEG) analysis was further performed within the expanded cohort. Strikingly, among 20 DEGs identified, 13 were associated or linked to hypoxia (see FIGS. 9A and 9B).

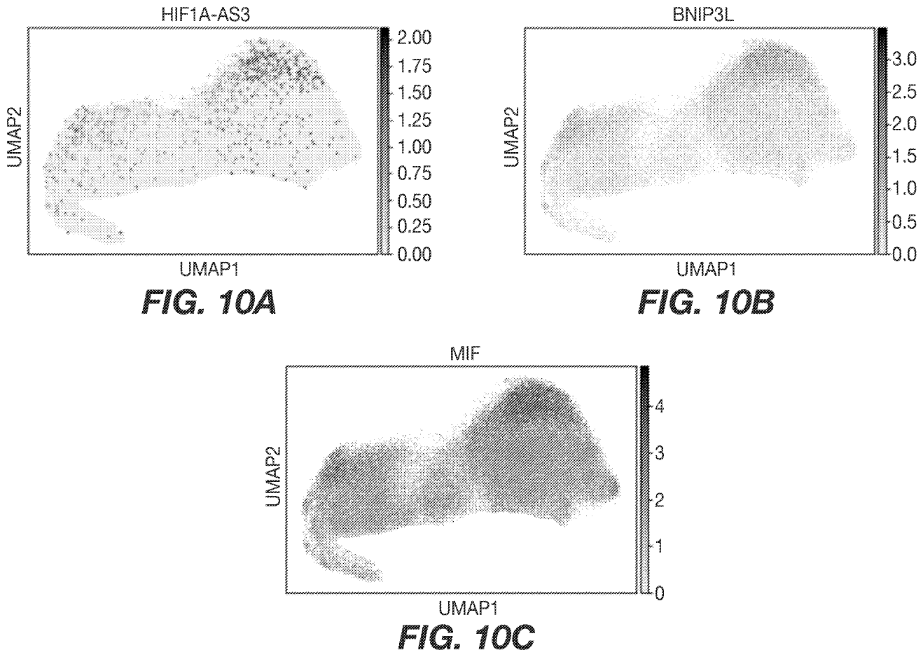

To further assess differential expression of genes associated with hypoxia, expression of 3 established hypoxia-related genes HIF1A-AS3, BNIP3L, and MIF were plotted on cell density clusters of high expanders described in FIG. 8A. Intriguingly, the selected genes were mapped to the unique γδ T cell cluster in high expanders (see FIGS. 10A to 10C), suggesting that hypoxia might have a role in driving expansion of Vγ9Vδ2 T cells.

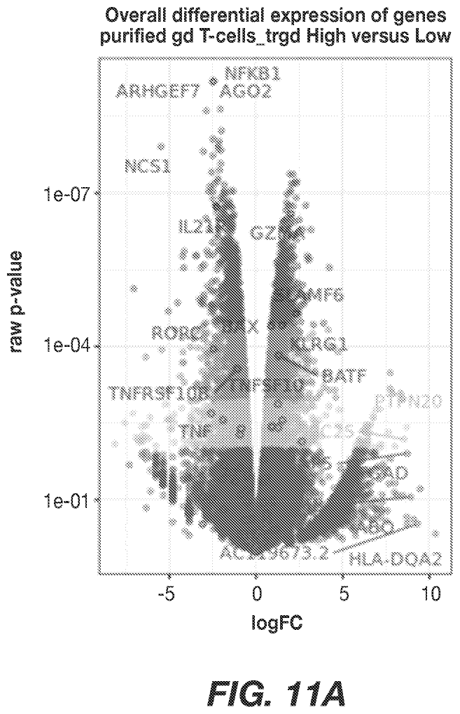

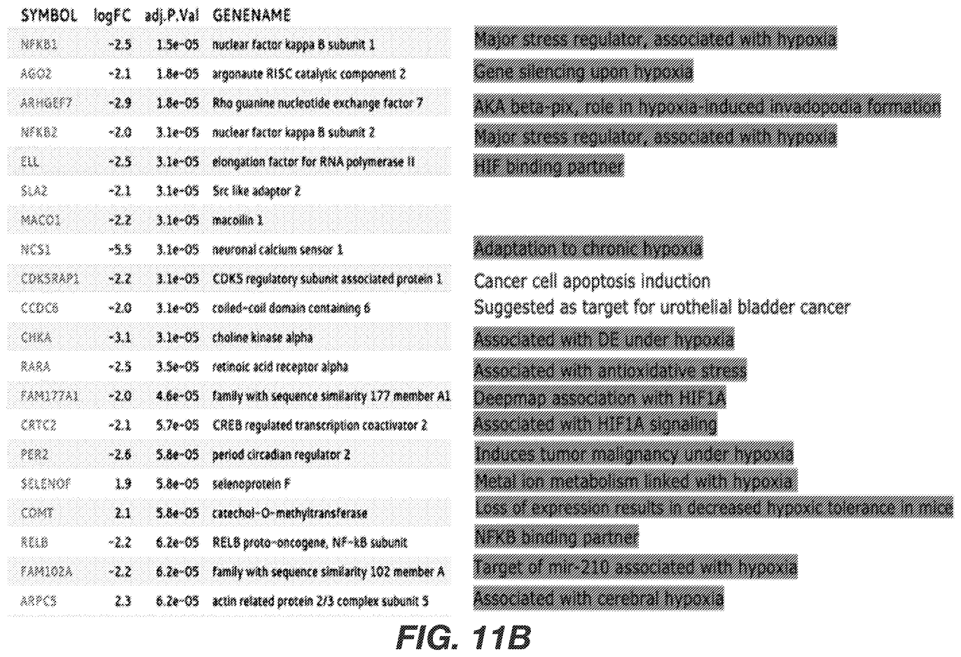

To determine whether similar hypoxia signatures were driving transcriptional differences in the enriched cohort, DEG analysis was performed between high and low expanders within the enriched cohort. Consistent with the previous findings, 16 out 20 identified genes were linked to hypoxia (see FIGS. 11A and 11B). These findings indicated that hypoxia-related gene signatures were critical for Vγ9Vδ2 T cells expansion and highlighted the relevance of hypoxia pathway to the intrinsic biology of Vγ9Vδ2 T cells.

### 6.3. Example 3-Vγ9Vδ2 T Cells Expanded under Hypoxic Conditions

### 6.3.1. Expand Vγ9Vδ2 T Cells under Hypoxic Conditions

Since hypoxia-related genes were found to be among top DEGs between Vγ9Vδ2

T cells obtained from low and high expanders, the advantages of culturing cells under hypoxic conditions were explored. Vγ9Vδ2 T cells expansion was performed under low oxygen conditions inside controlled environment hypoxia chamber. Tissue culture incubator was used as a control environment (18.2% oxygen) and was compared to low oxygen culture conditions by culturing Vγ9Vδ2 T cells at 12%, 5%, and 2% oxygen for an entire duration of experiment (14 days).

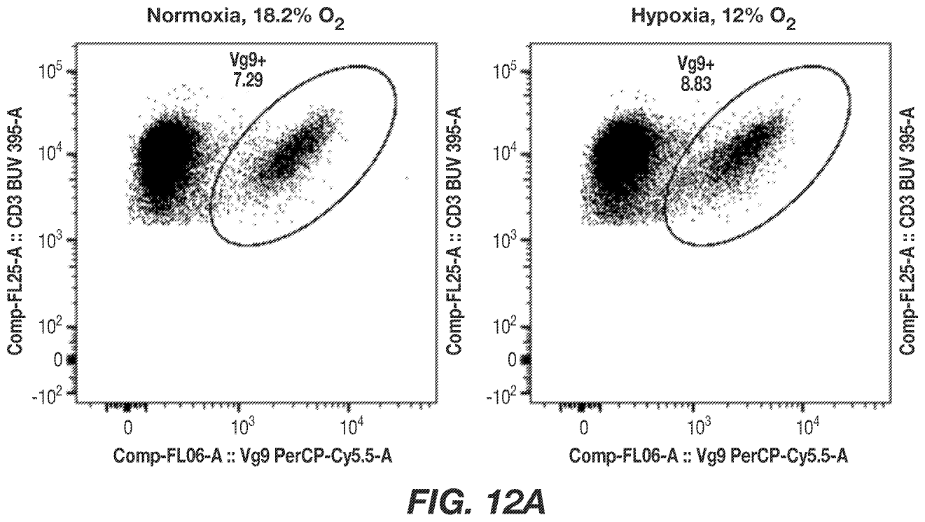

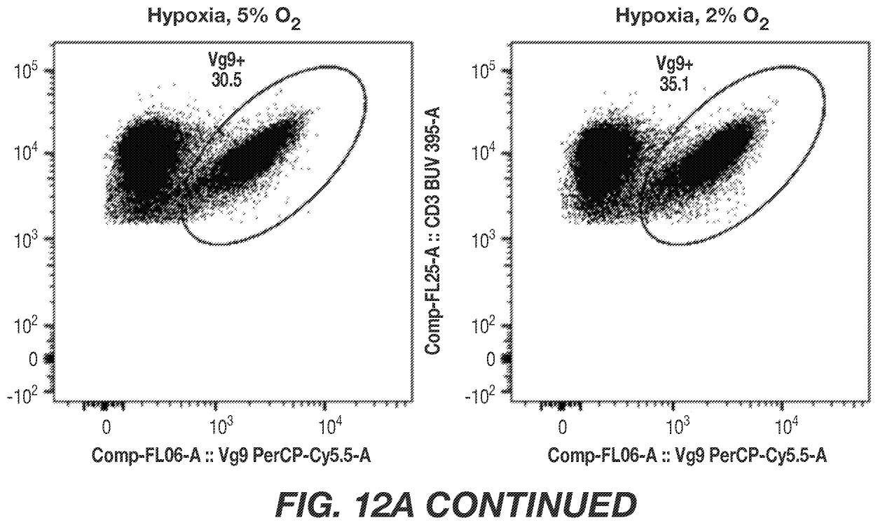

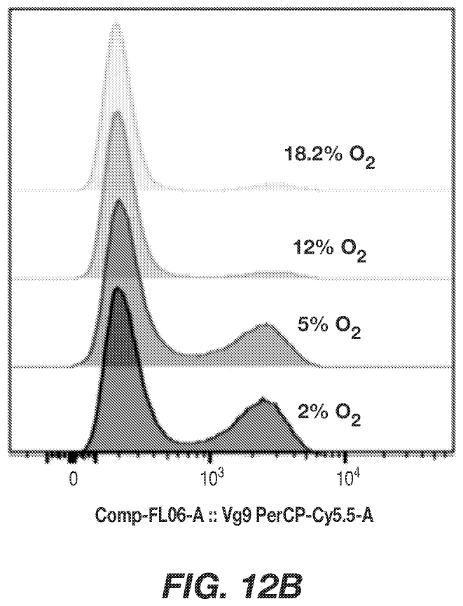

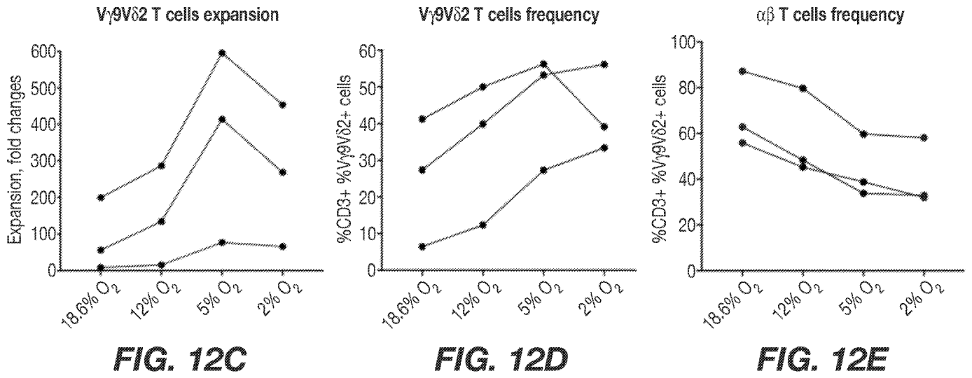

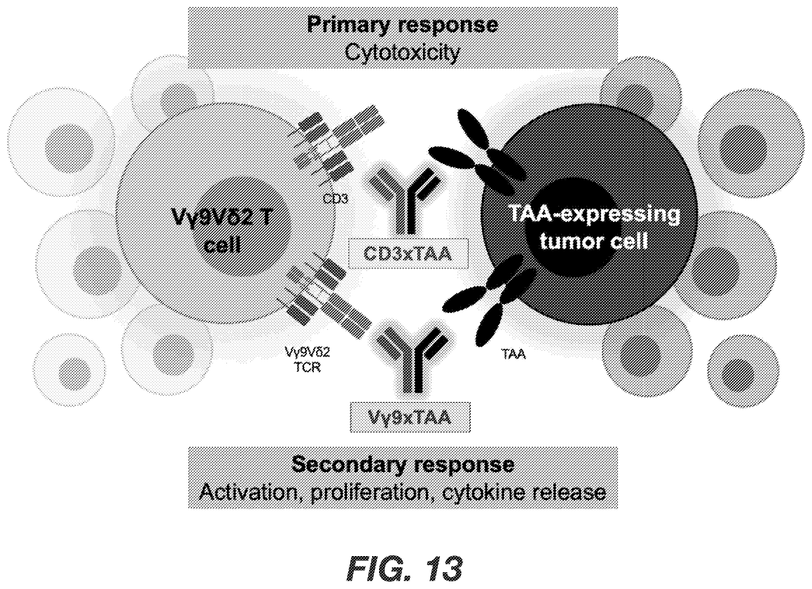

Indeed, expansion of Vγ9Vδ2 T cells under low oxygen culture conditions was more efficient and resulted in higher cell yields. Frequency of Vγ9Vδ2 T cell populations under normoxic and hypoxic conditions was first analyzed by flow cytometry. As shown in FIGS. 12A and 12B, there was an increase of Vγ9Vδ2 T cell frequency upon the decrease of oxygen concentration. The Vγ9Vδ2 T cell expansion across 3 healthy donors was analyzed and found that low oxygen improved Vγ9Vδ2 T cell purity in fold changes (see FIG. 12C) and Vγ9Vδ2 T cell frequency (see FIG. 12D). Interestingly, even the lowest oxygen concentration used to perform the expansion (2% oxygen) yielded better purity and frequency than the normal expansion (18.2% oxygen). Nonetheless, it was noted that 5% oxygen used for Vγ9Vδ2 T cell expansion yielded the highest purity as compared among the three healthy donors. This condition was used in following studies.

Immune effector cells such as αβ T cells and NK cells are unable to proliferate and maintain sufficient cytotoxicity under hypoxic environment (Kosti et al., 2021a; Kosti et al., 2021b; Sarkar et al., 2013). Negative effects of hypoxia on αβ T cells and NK cells within tumor microenvironment is one of the major roadblocks preventing development of effective cell therapy strategy for solid malignancies. Since αβ T cells represent a major cellular subset contaminating Vγ9Vδ2 T cell cultures, the frequency of αβ T cells were analyzed under hypoxic and normoxic conditions. As shown in FIG. 12D and as expected, presence of αβ T cells declined with a decrease of oxygen concentration. The above data supported that Vγ9Vδ2 T cell expansion relied on intrinsic gene signatures corresponding to hypoxia, and that low concentrations of oxygen were associated with improved Vγ9Vδ2 T cell frequency and purity.

### 6.3.2. In Vitro Cytotoxicity of Vγ9Vδ2 T Cells Expanded under Hypoxic Conditions

To further assess functionality of Vγ9Vδ2 T cells expanded under hypoxic conditions, a cytotoxicity assay with bispecific antibodies redirecting Vγ9Vδ2 T cells to HER2+JIMT1 cells was performed following the steps described in Section 6.1.10. Experiment design is shown in Table 3 below. Vγ9Vδ2 T cells were expanded under normoxic (18.2% oxygen) or hypoxic (5% oxygen) condition.

TABLE 3
Experimental Design

| | |
|---|---|
| Plate format | 96 wells (exclude edge wells) |
| Assay duration | up to 48 hrs, data collection every 15 min |
| Effector:Tumor ratios | 1:3 |
| (E:T ratios) | |
| Volume per well | 200 μL total = 50 μL target cells (12 500 cells/well) + 50 μL |
| | effector cells (adjust based on E:T) + 50 μL 4x test article + 50 |
| | μL complete growth medium (without cytokines) for blanking |
| Controls | Target only |
| | Target + Effector only (baseline killing control) |
| | Target + Test article only (cytostatic effects control) |
| Test articles | Isotype control (UKNB1.001, 1.06 mg/mL) |
| | Vγ9 × Null (Vγ9-7A5-17-Fab RF (Hole) × Null MSCD334- |
| | scFv (Knob), 0.75 mg/mL) |
| | HER2 × Null (Null-LH-MSCD334-scFv × HER2-HC-RF × |
| | HER2-LC, 1.1 mg/mL) |
| | Vγ9 × HER2 (Vγ9-7A5-17-RF - PBD000091606 (Hole) × |
| | HER2-LH-MSCD331-scFv (Knob), 1.16 mg/mL) |
| | CD3 × HER2 (ZWA: HER2- Fab (ERBH7); ZWB: CD3-ScFV |
| | (CD3B219), 1.6 mg/mL) |

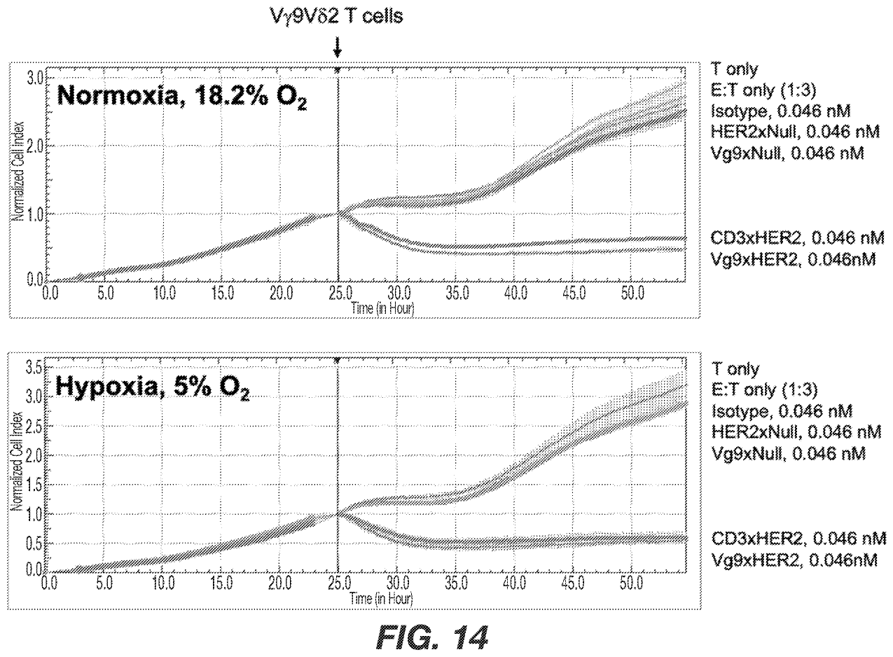

As shown in FIG. 14, both Vγ9×HER2 and CD3×HER2 conditions resulted in robust killing of tumor line within 4-8 hours after Vγ9Vδ2 T cells expanded under hypoxic or normoxic condition were added, indicating that Vγ9Vδ2 T cells expanding under hypoxic conditions retained their functional profile and demonstrated robust cytotoxicity in vitro.

### 6.4. Example 4-In Vivo Engraftment of Vγ9v82 T Cells Using Various Mouse Models

### 6.4.1. Model Development of In Vivo Engraftment Study

The following studies in Example 4 sought to determine and optimize (a) the mouse strain for adoptively transfer of Vγ9Vδ2 T cells; (b) the dose of adoptively transferred Vγ9Vδ2 T cells, and (c) conditions and procedures needed to achieve successful engraftment of adoptively transferred Vγ9Vδ2 T cells, including enrichment of Vγ9Vδ2 T cells before transfer and the benefits of IL-2 and zoledronic acid (pAg) treatment.

Particularly, to evaluate the ability of adoptively transferred Vγ9Vδ2 T cells to be engrafted and persist across various mouse strains, the mouse strains used in this study were NOD.Cg-Prkdc^{scid} Il2rg^{tm1Wjl} (“NSG”), NOD.Cg-Prkdc^{scid} Il2rg^{tm1Wljl}Tg(IL15)1Sz/SzJ, (“NSG-IL15”), and NOD.Cg-Prkdc^{scid} Il2rg^{tm1Sug} Tg(CMV-IL2/IL15) 1-1Jic/JicTac (“NOG-IL15”). NSG mice were B and T cell deficient and were null for the IL-2 receptor common gamma chain. NSG-IL15 mice combined the features of NSG mice and also expressed human IL-15. NOG-IL15 mice were B, T, and NK cell deficient, had reduced complement activity, dysfunctional macrophages, and expressed human IL15.

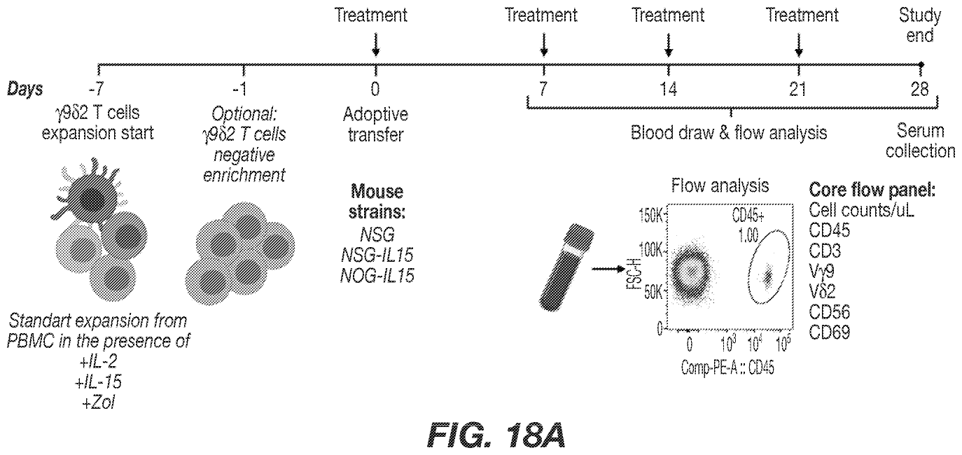

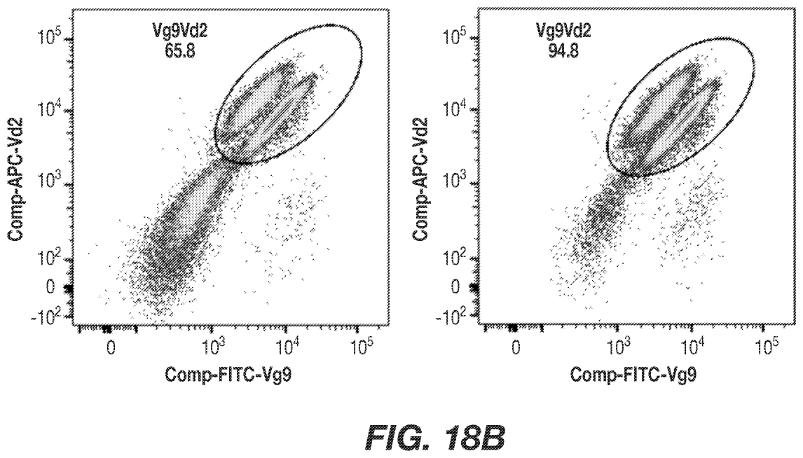

The overall process is depicted in FIG. 18A. Briefly, 7 days before the adoptive transfer into a given mouse strain (day −7), Vγ9Vδ2 T cell expansion was started. Expansion of Vγ9Vδ2 T cells was performed according to a standardized Vγ9Vδ2 T cell expansion procedure as described herein. Cells were cultured in RPMI, 10% FBS, 1% anti-anti supplemented with IL-2, IL-15 and zoledronic acid. Cells were cultured for 7 days or until day 0 (cells were enriched at Day-1 and rested overnight for ˜16 hours).

For one of the groups, negative selection was performed on Vγ9Vδ2 T cells 1 day before the adoptive transfer (day −1) to create a population of cells enriched for TCRγ/δ+ T Cells or (γδ T Cells) (this process was referred to as enrichment). TCRγ/δ+ T Cell Isolation (Miltenyi Biotec, 130-092-892) was performed according to manufacturer's instruction. The cell pellet was resuspended in 80 μl of buffer per 107 total cells. 20 μl biotin-antibody cocktail was then added per 107 total cells. The cell suspension was mixed well and refrigerated for 10 minutes at 4-8° C. Working on ice may require increased incubation times as higher temperatures and/or longer incubation times led to non-specific cell labeling. Fourth, the cells were then washed by adding 1-2 mL of buffer per 10⁷ cells and the cells were then centrifuged at 300×g for 10 minutes. The supernatant was then completely aspirated. 80 μL buffer was added per 107 total cells. 20 μL anti-biotin microbeads were added per 107 total cells. The cell suspension was mixed well and refrigerated for an additional 15 minutes at 4-8° C. The cells were then washed by adding 1-2 mL of buffer per 10⁷ cells, and centrifuged at 300×g for 10 minutes and the supernatant was completely aspirated. The cells were then resuspended up to 10⁸ cells in 500 μL of buffer. For higher cell numbers, the buffer volume should be scaled up accordingly. The cells then undergo a magnetic separation with MS and LS columns. A column was placed in the magnetic field of a suitable MACS Separator. For details, see the manufactures MACS column data sheet. The column was then prepared by rinsing with an appropriate amount of buffer: 500 μl for an MS column and 3 mL for an LS column. The cell suspension was then applied onto the column. The cells were then allowed to pass through and the effluent was collected as a fraction with unlabeled cells, which represented the enriched γδ T cells.

Upon completion of the expansion and negative selection process, Vγ9Vδ2 T cells or γδ T cells were provided for an adoptive transfer into NSG, NSG-IL15, and NOG-IL15 mice (day 0). NSG and NSG-IL15 were obtained from The Jackson Laboratory. NOG-IL15 mice were obtained from Taconic. The mice were 24-28 weeks of age and housed in irradiated filtered top plastic cages. Mice was maintained on autoclaved Laboratory Rodent Chow (PMI). On day 0, the respective groups received an IV injection of either 5×10⁶or 20×10⁶Vγ9Vδ2 T cells or γδ T cells in 0.100 mL of PBS. FIG. 18B shows the Vγ9Vδ2 T cell purity at the day of the adoptive transfer. For the non-enriched groups (left panel), the total cells consisted of 65% Vγ9Vδ2 T cells. For the enriched groups (right panel), the total cells consisted of 95% Vγ9Vδ2 T cells. All groups were weighed and bled on days post Vγ9Vδ2 T cell adoptive transfer (days 7, 14, 21, and 28) to assess Vγ9Vδ2 T cell engraftment and persistence. For the baseline bleed at day 0 prior to the adoptive transfer, 100-150 μl blood was collected from 7 randomly selected mice (1 animal from 1 group) in purple cap BD microtainers, maintained as a whole blood at room temperature. For the bleed on days 7, 14, and 21 (post adoptive transfer), 100-150 μl of blood (RO bleed from isoflurane anesthetized mice) from all mice in purple cap BD microtainers, maintained as a whole blood at room temperature. For the bleed on day 28 (post adoptive transfer and the end of the study), whole blood was collected by cardiac puncture in EDTA tubes and maintained at room temperature as whole blood. All groups were weighed weekly. Individual mice or groups of mice were euthanized if body weight loss of more than 20% was observed or if severe symptoms of Graft Versus Host Disease (GvHD) or animal discomfort were observed as described in Janssen's Animal Care and Use Policy on Euthanasia of Laboratory.

On days 0, 7, 14, 21, and 28, the mice were treated with either PBS, IL2, or IL2 and Zometa (Zoledronic acid) as outlined in Table 4. For each treatment group, there were 5 mice for the NSG, NSG-IL15, and NOG-IL 15 groups. Also, on days 7, 14, 21, and 28 post adoptive transfer, blood was drawn from the respective mice for flow analysis. Once blood was drawn, staining was performed immediately. A master mix was prepared containing antibodies with human and mouse Fc blocks. Each antibody was used at a concentration of 1 μl per reaction. Human Fc block contained 5 μl/reaction, the mouse Fc block contained 5 μl/reaction, and Brilliant stain buffer contained 10 μl/reaction. The total volume of each reaction was 37 μl/test. Aliquot 37 μl of mastermix into the 50 mL conical tube and then added 100 μl of mouse blood and pipetted up and down to mix (5×). It was ensured to use exactly 100 μl of blood per reaction. The mix was then incubated at room temperature for 30 minutes in the dark. RBC lysis buffer was prepared by mixing stock RBC solution with sterile distilled water at a ratio of 1:9 making sure the solution was at room temperature upon use. Once the incubation with the antibodies was complete, 20 mL lysis buffer was added to the mix and the mix was vortexed for 5 seconds. The mixture was then incubated at room temperature for 15 minutes while protected from light. After the incubation, 30 mL room temperature PBS was added to the mix and the mix was centrifuged at 1500 rpm for 5 minutes at room temperature. The supernatant was carefully aspirated making sure to not disturb the cell pellet. The liquid on the bottom of the tube was completely removed to avoid contamination with red blood cells. The cell pellet was resuspended in 1 mL of PBS, mix, and then added an additional PBS to reach a volume of 50 mL of PBS. The mix was then centrifuged at 1500 rpm for 5 minutes at room temperature. The supernatant was carefully aspirated making sure not to disturb the cell pellet. The cell pellet was resuspended in 250 μl of BD BSA stain buffer for flow acquisition. The sample was transferred to BD TrueCount tubes containing counting beads and the sample was vortexed well. Data was then recorded on the cytometer. The flow panel consisted of the following: cell counts/μl, CD45, CD3, Vγ9, Vγ2, CD56, and CD69. For the controls, the analysis included gating for the fluorescence minus one (FMO) and the isotype controls CD69+ and CD56+. The cell number was determined and rested overnight at the concentration 1×10*6/mL in complete growth media.

TABLE 4
Dose of Vγ9Vδ2 T cells and Treatment

| Dose of adoptively | | |
|---|---|---|
| transferred | | |
| Vγ9Vδ2 T cells | Treatment | Enrichment |
| 5 × 10⁶ cells/mouse | PBS | — |
| 20 × 10⁶ cells/mouse | PBS | — |
| 20 × 10⁶ cells/mouse | PBS | Miltenyi MACS Negative |
| | | Selection Kit |
| 20 × 10⁶ cells/mouse | IL-2, | — |
| | 25 × 10⁴ IU/kg, ip | |
| 20 × 10⁶ cells/mouse | IL-2, | — |
| | 25 × 10⁴ IU/kg, ip | |
| | Zol, 2.5 mg/kg, iv | |

### 6.4.2. Engraftment analysis using Vγ9Vδ2 T cells and γδ T Cells

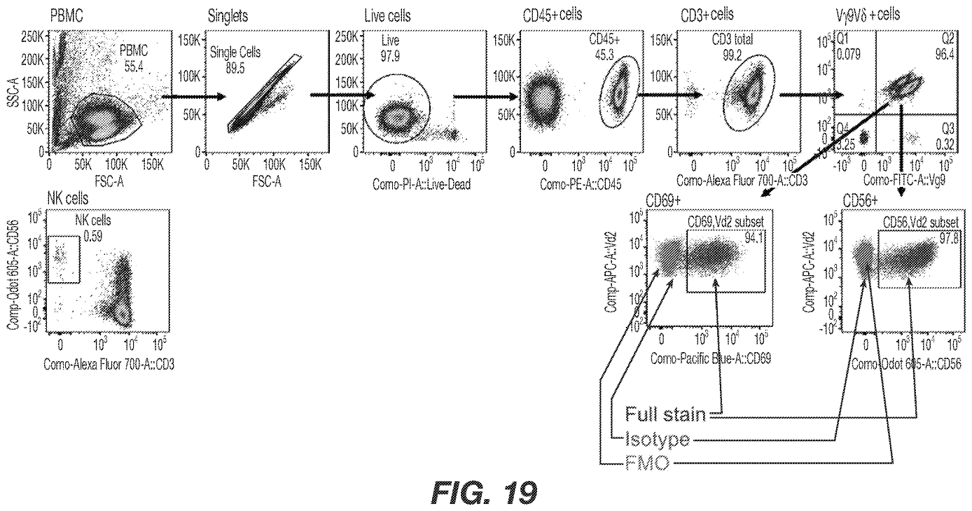

To determine whether Vγ9Vδ2 T cells can be detected in mouse peripheral blood, cell populations were measured using flow cytometry 7 days post adoptive transfer. The protocol for flow cytometry was performed as described herein. FIG. 19 shows the gating strategy as cells were identified by PBMCs, singlets, live cells, CD45+ cells, CD3+ cells, Vγ9Vδ2 T cells, and natural killer (NK) cells. These data demonstrated that Vγ9Vδ2 T cells were present in mouse peripheral blood confirming the adoptive transfer of the Vγ9Vδ2 T cells into the immunocompromised mice.

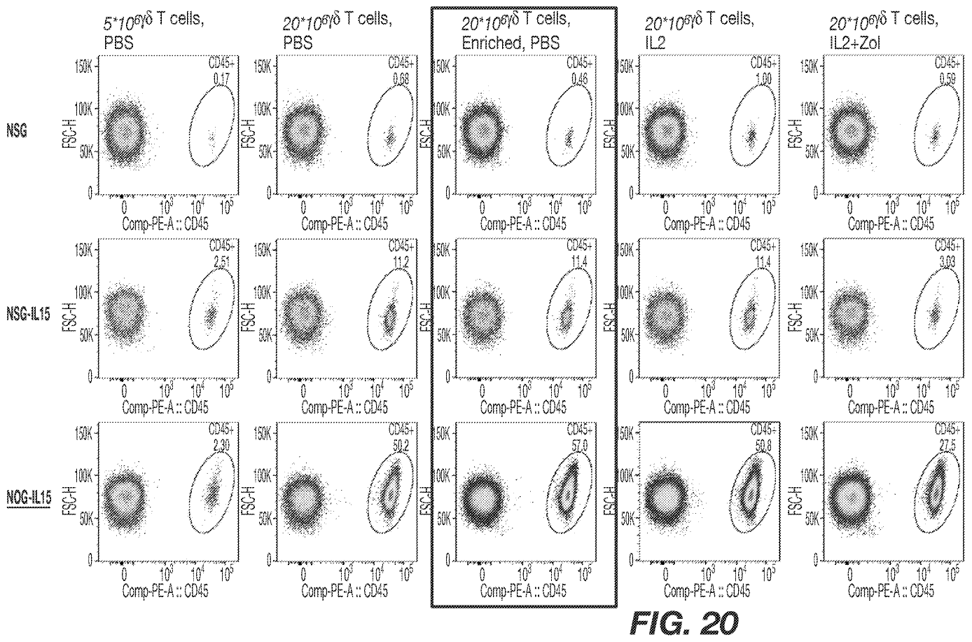

To determine whether the Vγ9Vδ2 T cells were engrafted in the NSG, NSG-IL15, and NOG-IL 15 mouse strains among the various treatment groups, flow cytometry analysis was performed on blood samples collected 7 days post adoptive transfer (gated for CD45⁺). The protocol for flow cytometry was performed as described herein. As shown in FIG. 20, Vγ9Vδ2 T cell purity in highlighted populations was about 85-95% across all groups, and the NOG-IL 15 strain showed the most efficient Vγ9Vδ2 T cell engraftment across all treatment groups due to the strong signal of human CD45⁺ cells. These data demonstrated that the NOG-IL15 mouse strains was a suitable model for Vγ9Vδ2 T cell engraftment.

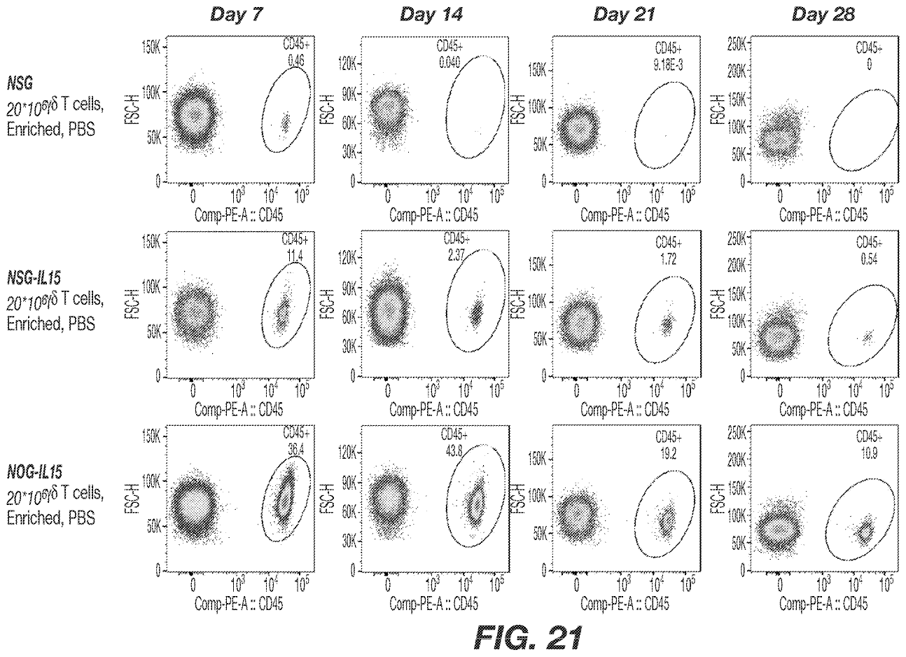

To determine whether the engraftment of enriched Vγ9Vδ2 T cells (enriched group shown in FIG. 21, >95% of CD45⁺ cells were Vγ9Vδ2+) persisted in the NSG, NSG-IL15, and NOG-IL 15 mouse strains treated with PBS, flow cytometry was performed on blood samples collected at days 7, 14, 21, and 28 post adoptive transfer (gated for CD45⁺). The protocol for flow cytometry was performed as described herein. As shown in FIG. 21, by day 28, the percentage of engrafted human cells declined in the enriched Vγ9Vδ2 T cell group as there was a decreased cell population for CD45⁺ human cells among the NSG and NSG-IL 15 mouse strains. In contrast, the enriched Vγ9Vδ2 T cells engrafted in NOG-IL 15 mice showed a strong population of CD45⁺ human cells on day 28. These data show that enriched Vγ9Vδ2 T cells in NOG-IL15 mice demonstrated optimal persistence.

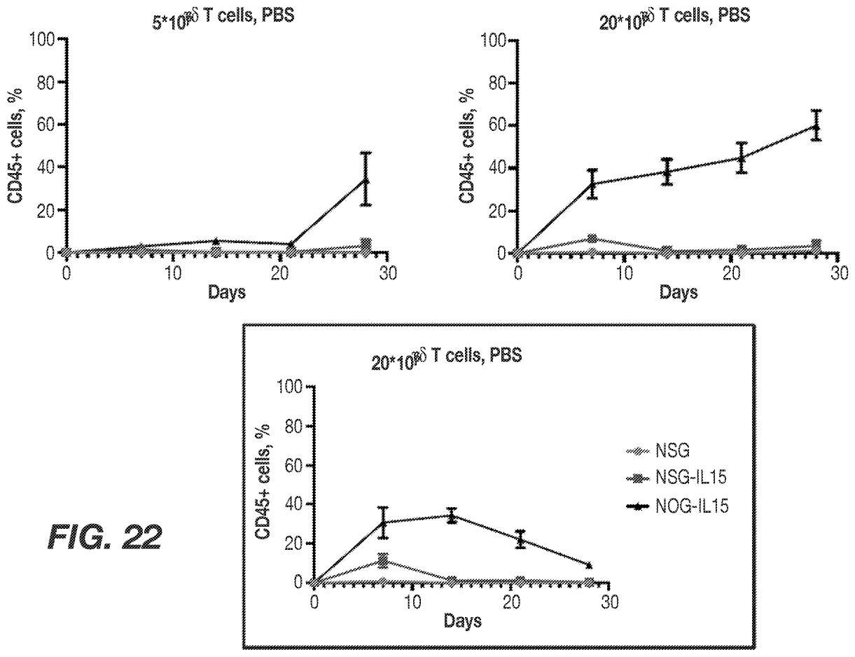

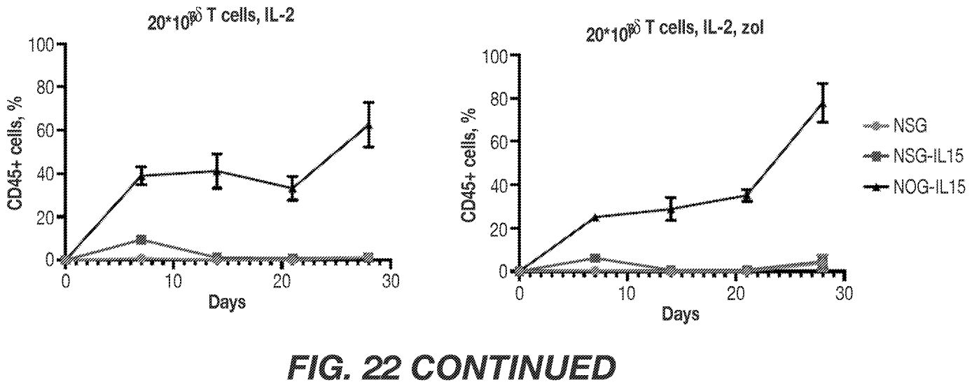

It was then determined whether non-enriched Vγ9Vδ2 T cells treated with PBS, IL-2 or IL-2 and zoledronic acid results in persistent engraftment of CD45⁺ human cells in the NSG, NSG-IL15, and NOG-IL15 mouse strains. Further, the enriched Vγ9Vδ2 T cell group treated with PBS was also analyzed for persistent engraftment of CD45⁺ human cells. Flow cytometry analysis was performed on mouse peripheral blood samples collected on days 7, 14, 21, and 28 post adoptive transfer from the respective mouse strains. The protocol for flow cytometry was performed as described herein. FIG. 22 shows that NOG-IL 15 mice received 20×10⁶Vγ9Vδ2 T cells demonstrated the most efficient engraftment, as CD45⁺ human cells remained present on day 28 while NSG-IL 15 mice showed modest engraftment with CD45⁺ human cells present on day 7. However, the CD45⁺ human cells rapidly expand in the NOG-IL 15 mice resulting in GvHD by day 21 except in the group where the NOG-IL15 received enriched γδ T cells. These data demonstrated that the enriched γδ T cells had the most efficient engraftment in NOG-IL15 cells without leading to GvHD.

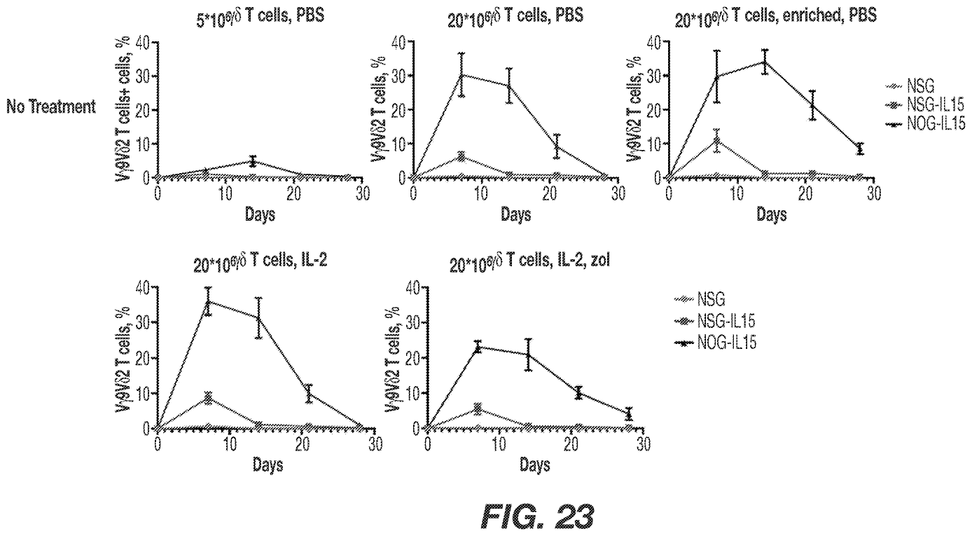

To determine the percentage of Vγ9Vδ2 T cells the NSG, NSG-IL 15, and NOG-IL15 mouse strains treated with PBS, IL-2 (ProleukinR), and IL-2 (ProleukinR) and zoledronic acid ZometaR), flow cytometry was used. The protocol for flow cytometry was performed as described herein. The percentage of Vγ9Vδ2 T cells in circulation was normalized to the percentage of CD45 CD3⁺ cells. FIG. 23 shows increased engraftment in NOG-IL15 mice that were given 20×10⁶ Vγ9Vδ2 T cells and treated with either PBS or IL-2. However, in the IL-2 and zoledronic acid treatment group, there was a decrease in engraftment, which was likely related to zoledronic acid associated toxicity. No adverse events were observed in the animals. These data show that Vγ9Vδ2 T cells do not persist for longer than 3 weeks due to a high engraftment of Vγ9Vδ2 T cells in NOG-IL15 mice.

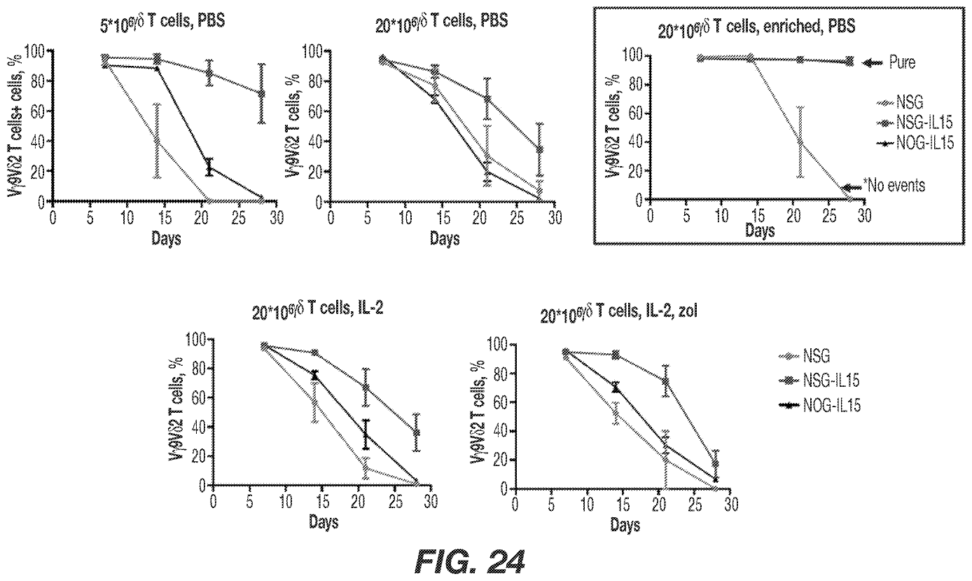

To determine whether Vγ9Vδ2 T cells could maintain their purity during engraftment, flow cytometry was performed on blood samples on days 7, 14, 21, and 28 post adoptive transfer from the respective mouse strains. The protocol for flow cytometry was performed as described herein. As shown in FIG. 24, the purity of non-enriched Vγ9Vδ2 T cells decreases in NSG, NSG-IL15, and NOG-IL15 mice across the PBS, IL-2, and IL2 and zoledronic acid treatment groups. However, purity was maintained in the enriched γδ cells in the NSG-IL15 and NOG-IL 15 mouse strains treated with PBS. A decline in the NSG group was observed due to the absence of any Vγ9Vδ2 cellular events. These data demonstrated that groups engrafted with enriched γδ cells maintained a high purity of the Vγ9Vδ2 T cell populations.

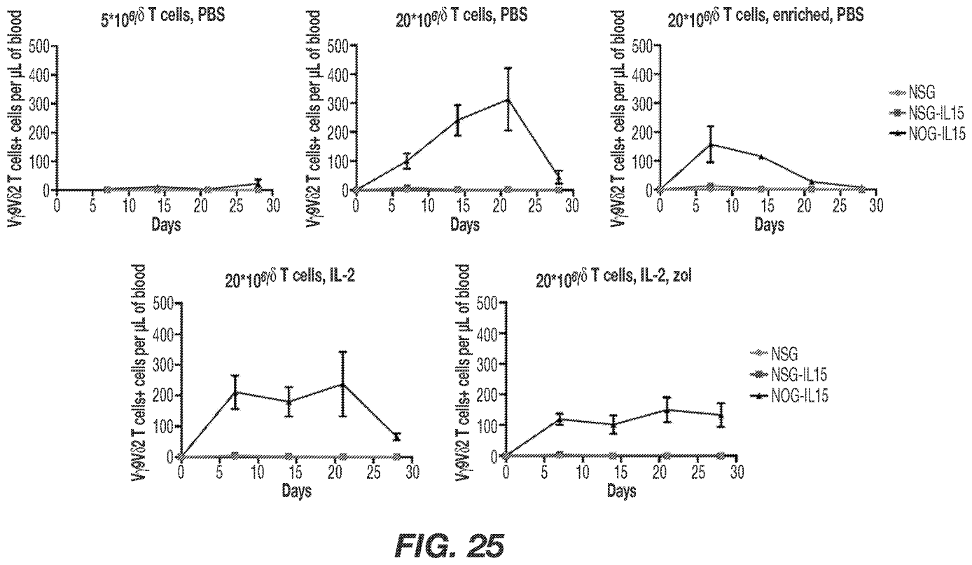

To determine the number of Vγ9Vδ2 T cells in the NSG, NSG-IL15, and NOG-IL15 mouse strains among the treatment groups, cell counts per microliter of mouse blood were measured on days 7, 14, 21, and 28 post adoptive transfer. FIG. 25 demonstrates that Vγ9Vδ2 T cell counts decreased by day 28 in NOG-IL 15 mice treated with PBS or IL-2 and also decreased in enriched Vγ9Vδ2 T cells by day 28 in NOG-IL15 mice treated with PBS. NOG-IL15 mice treated with IL-2 and zoledronic acid did not decrease by day 28. No changes were observed in the NSG or NSG-IL 15 mouse strains. These data show the number of engrafted cells per ul of mouse blood.

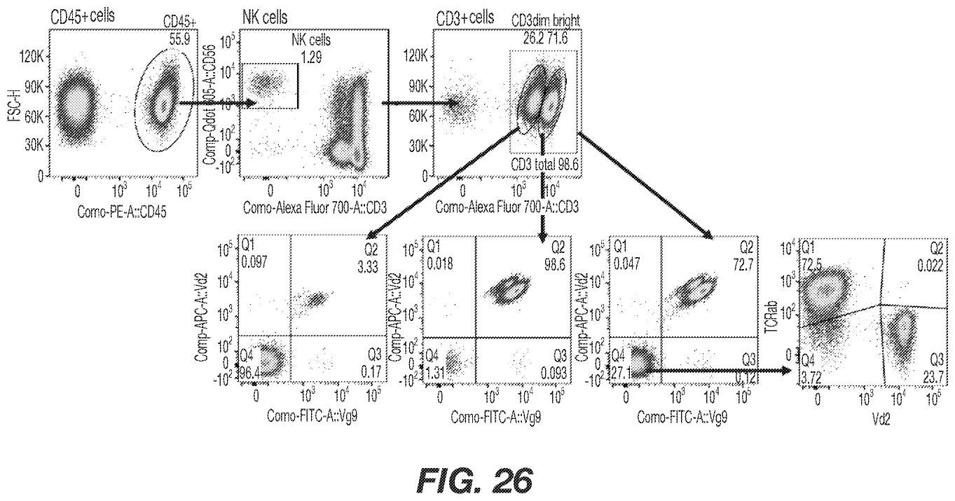

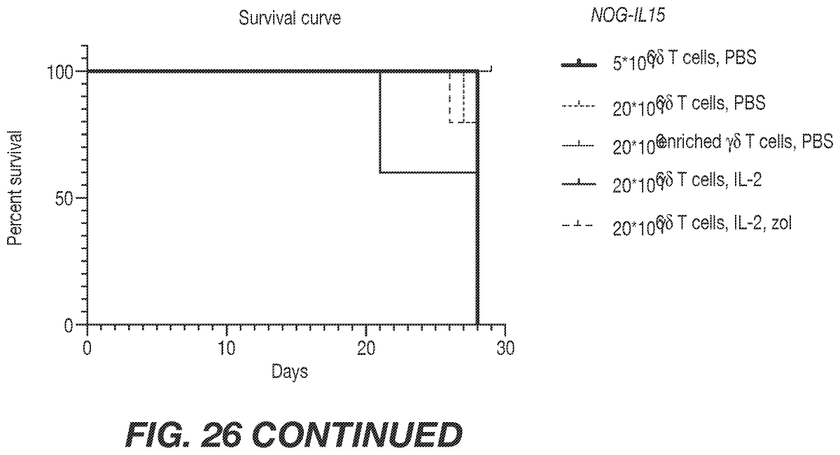

To better understand why NOG-IL15 mice developed GvHD, additional flow cytometry analysis was performed for gating analysis of CD3⁺ cells. The protocol for flow cytometry was performed as described herein. As shown in FIG. 26, the flow cytometry analysis showed CD3⁺ cell population resembling β T cells. This expansion in αβ T cells was the likely cause of GvHD in the NOG-IL 15 mice that received the non-enriched Vγ9Vδ2 T cells. Additionally, the natural killer cells expanded up to 2%. Further, FIG. 26 shows a survival curve where NOG-IL 15 mice that received enriched Vγ9Vδ2 T cells had 100% survival as these mice did not contract GvHD. These data demonstrated that non-enriched Vγ9Vδ2 T cells results in the death of a subset of NOG-IL 15 mice due to GvHD.

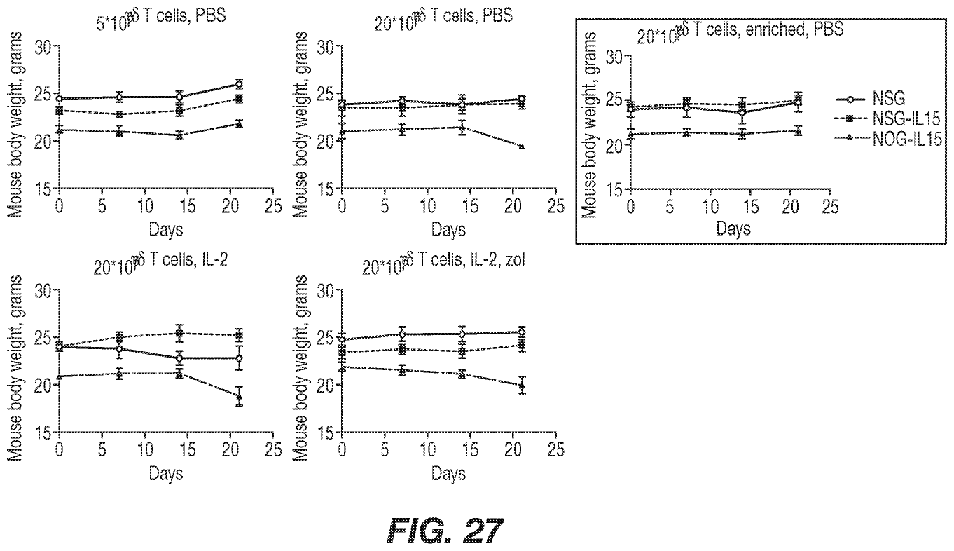

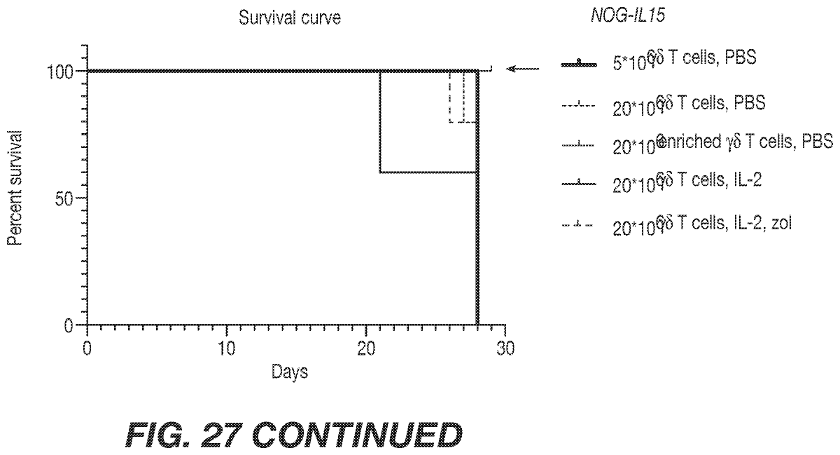

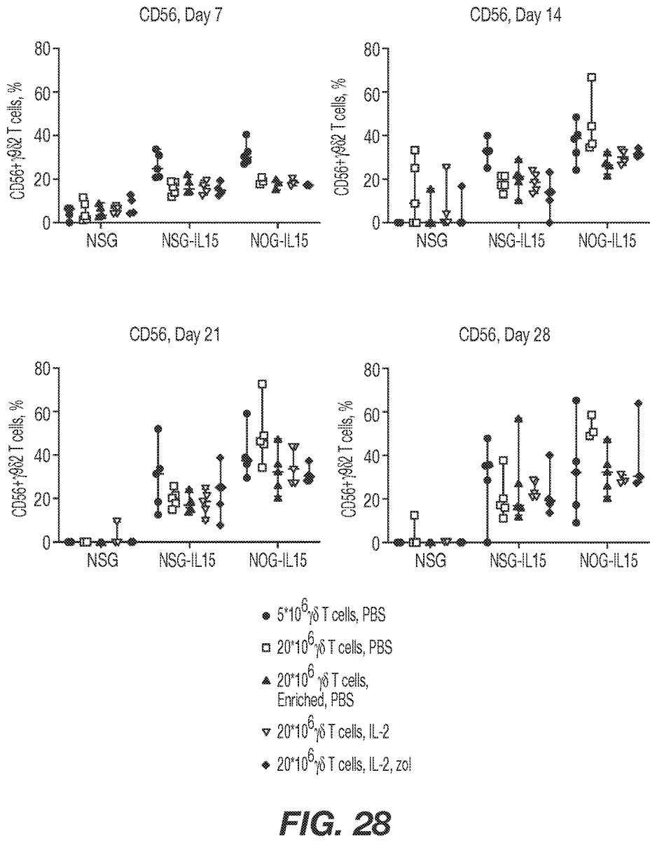

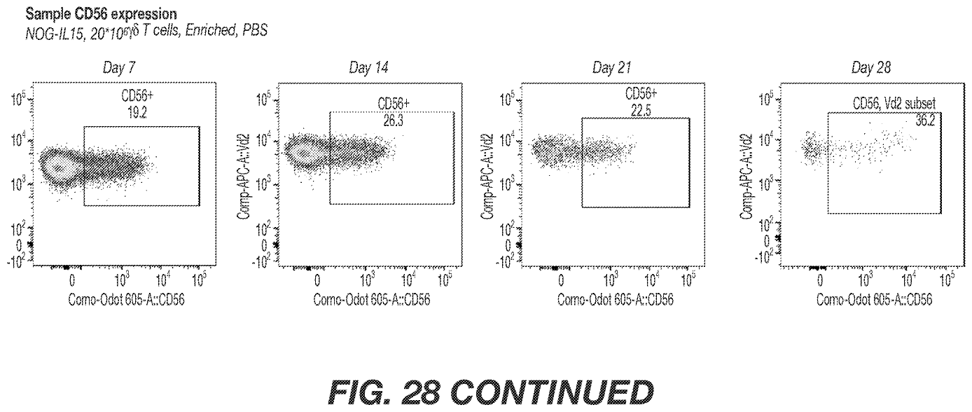

To determine whether the adoptive transfer of purified Vγ9Vδ2 T cells causes GvHD in mice, the NSG, NSG-IL15, and NOG-IL 15 strains were administered 5×10⁶ or 20×10⁶ purified Vγ9Vδ2 T cells following the protocol outlined in FIG. 18A. Mouse body weight, in grams, was determined at days 7, 14, and 21 post adoptive transfer. FIG. 27 shows NSG, NSG-IL15, and NOG-IL15 mice that received purified non-enriched Vγ9Vδ2 T cells had a similar weight on day 21 post adoptive transfer as compared to day 0 (the day of the adoptive transfer). Further, NSG, NSG-IL15, and NOG-IL 15 mice that received purified enriched Vγ9Vδ2 T cells also had a similar weight on day 21 post adoptive transfer as compared to day 0 (the day of the adoptive transfer). Further, a survival curve where NOG-IL15 mice that received enriched Vγ9Vδ2 T cells had 100% survival as these mice did not contract GvHD. These data demonstrated that NSG, NSG-IL15, and NOG-IL 15 mice receiving purified Vγ9Vδ2 T cells for the adoptive transfer did not contract GvHD. CD56 expression on adoptively transferred Vγ9Vδ2 T cells was measured using flow cytometry in NSG, NSG-IL15, and NOG-IL15 mice treated with PBS, IL-2, and IL-2 and zoledronic acid. The analysis was performed on blood from the respective strains collected on days 7, 14, 21, and 28. The protocol for flow cytometry was performed as described herein. FIG. 28 shows that Vγ9Vδ2 T cells expressed higher levels of CD56 in the NSG-IL 15 and NOG-IL 15 strains following the adoptive transfer. This higher expression of CD56 in these strains was because the strains expressed human IL 15, whereas the NSG strain did not. The CD56 expression increased post adoptive transfer and reaches a maximum level on Vγ9Vδ2 T cells on day 21 in NOG-IL15 mice. FIG. 28 also shows a sample CD56 expression flow cytometry plot of NOG-IL15 mice that received enriched Vγ9Vδ2 T cells and treated with PBS. These data demonstrated CD56 was expressed on Vγ9Vδ2 T cells on days after the adoptive transfer.

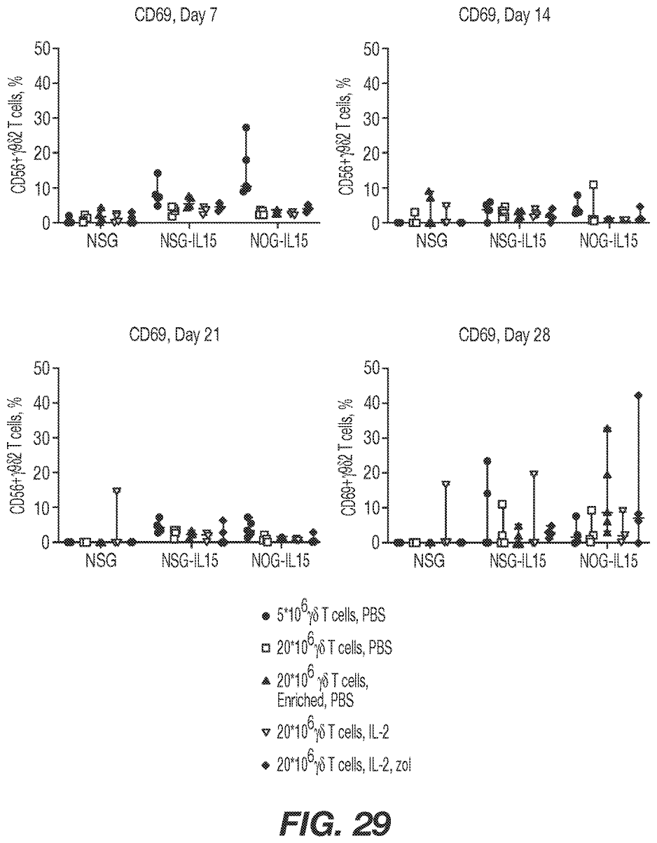

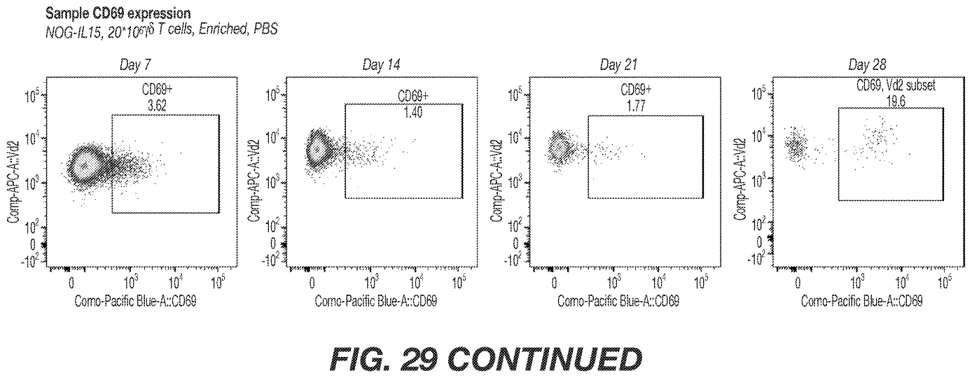

CD69 expression on adoptively transferred Vγ9Vδ2 T cells was measured using flow cytometry in NSG, NSG-IL15, and NOG-IL15 mice treated with PBS, IL-2, and IL-2 and zoledronic acid. The analysis was performed on blood from the respective strains collected on days 7, 14, 21, and 28. The protocol for flow cytometry was performed as described herein. FIG. 29 shows that Vγ9Vδ2 T cells expressed low levels of CD69 in the NSG, NSG-IL15, and NOG-IL15 strains following the adoptive transfer. The CD69 expression increased post adoptive transfer and reached a maximum level on Vγ9Vδ2 T cells on day 28. FIG. 28 also shows a sample CD69 expression flow cytometry plot of NOG-IL15 mice that received enriched Vγ9Vδ2 T cells and treated with PBS. These data demonstrated CD69 was mildly expressed on Vγ9Vδ2 T cells on day 28 after the adoptive transfer.

The studies described in Example 4 surveyed engraftment and persistence of adoptively transferred Vγ9Vδ2 T cells across 3 mouse strains (NSG, NSG-IL15 and NOG-IL15) and determined that NOG-IL 15 strain demonstrated the most efficient engraftment and persistence. As observed in these studies, engrafted Vγ9Vδ2 T cells persisted in mouse periphery for about weeks post injection. Expansion of αβ T cells in NOG-IL 15 animals that received non-enriched for γδ injections caused GvHD in these animals. Animals that received enriched for γδ T cell injections did not show any signs of GvHD. Therefore, enrichment for γδ T cells was required for future adoptive transfer studies. Injections of IL-2 and IL-2 in combination with zoledronic acid did not improve engraftment or persistence and had negative effect on total Vγ9Vδ2 T cell counts. Expression of CD56 and CD69 did not show drastic change throughout the study.

### 6.5. Example 5-In vivo engraftment of fresh and frozen Vγ9Vδ2 T cells using NOG-IL15 mice

### 6.5.1. Model Development

Studies of Example 5 sought to optimize and determine the (a) the appropriate donor of Vγ9Vδ2 T cells, (b) dose of adoptively transferred Vγ9Vδ2 T cells, and (c) the option between using fresh vs. frozen Vγ9Vδ2 T cells for the in vivo engraftment.

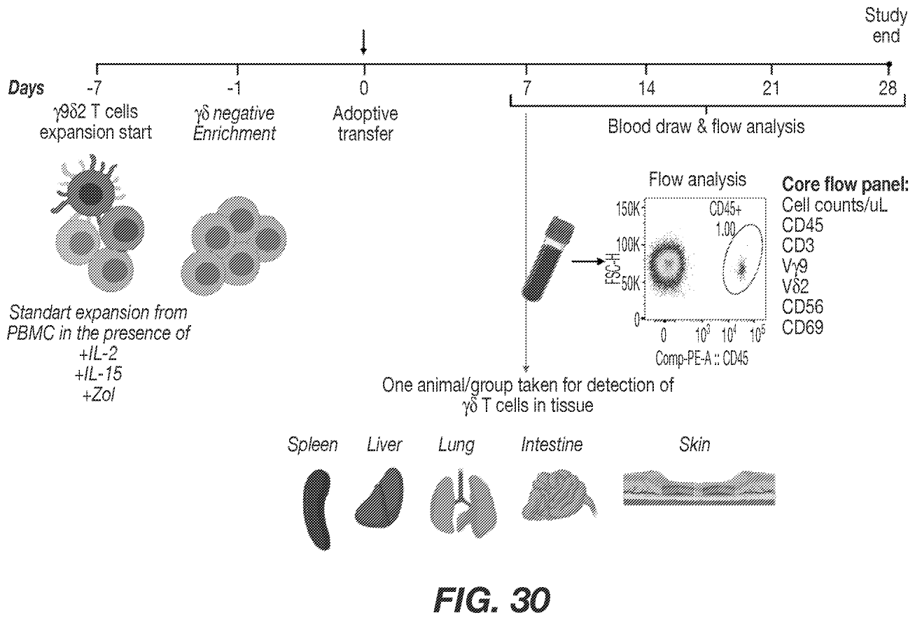

The overall process of the study is depicted in FIG. 30. Briefly, 7 days before the adoptive transfer (day −7) into NOG-IL 15 mice, Vγ9Vδ2 T cell expansion was started.

Expansion of Vγ9Vδ2 T cells was performed according to the in vitro Vγ9Vδ2 T cell expansion procedure as described herein. Briefly, cells were cultured in RPMI, 10% FBS, 1% anti-anti supplemented with IL-2, IL-15, and zoledronic acid. Cells were cultured for 7 days or until day 0 (cells were enriched at Day-1 and rested overnight for ˜16 hours).

Enrichment by negative selection was performed 1 day before the adoptive transfer (day −1) to create a population of cells enriched for γδ T Cells according to the procedure described in Example 4.

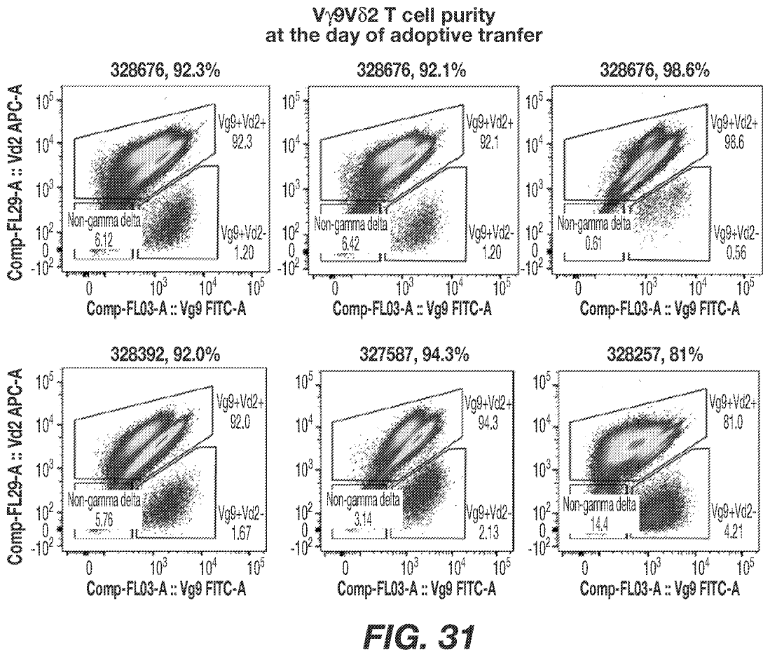

Upon completion of the expansion and negative selection process, γδ T cells were provided for an adoptive transfer into NOG-IL 15 mice (day 0). NOG-IL 15 mice were obtained from Taconic. The mice were 24-28 weeks of age and housed in irradiated filtered top plastic cages. Mice were maintained on autoclaved Laboratory Rodent Chow (PMI). On the day of the adoptive transfer (day 0) and as shown in Table 5, the respective groups received an IV injection of one of the respective donor cell populations containing either 10×10⁶ or 20×10⁶ fresh or frozen enriched γδ T cells in 0.100 mL of PBS. The purity of the Vγ9Vδ2 T cells from the various donors is shown in Table 5 and in FIG. 31. All mice were weighed and bled on days post γδ T cell adoptive transfer (days 7, 14, 21, and 28) to assess yo T cell engraftment and persistence. For the baseline bleed at day 0 prior to the adoptive transfer, 100-150 mL blood was collected from 7 randomly selected mice (1 animal from 1 group) in purple cap BD microtainers, maintained as a whole blood at room temperature. For the bleed on days 7, 14, and 21 (post adoptive transfer), 100-150 mL of blood (RO bleed from isoflurane anesthetized mice) from all mice in purple cap BD microtainers, maintained as a whole blood at room temperature. For the bleed on day 28 (post adoptive transfer and the end of the study), whole blood was collected by cardiac puncture in EDTA tubes and maintained at room temperature as whole blood. All groups were weighed weekly. Individual mice or groups of mice were euthanized if body weight loss of more than 20% was observed or if severe symptoms of Graft Versus Host Disease (GvHD) or animal discomfort were observed as described in Janssen's Animal Care and Use Policy on Euthanasia of Laboratory.

TABLE 5
Purity of the Vγ9Vδ2 T Cells from the Various Donors

| | | | | | Enrichment |
|---|---|---|---|---|---|
| | Donor | Cell | Fresh or | Expansion | (negative |
| Group | ID | number/mouse | Frozen | Status | selection) |
| 1 | 328676 | 20 × 10⁶/mouse | Fresh | High | 92.3 |
| | | | | expander | |
| 2 | 328676 | 10 × 10⁶/mouse | Fresh | High | 92.1 |
| | | | | expander | |
| 3 | 328676 | 10 × 10⁶/mouse | Frozen | High | 98.6 |
| | | | | expander | |
| 4 | 328392 | 10 × 10⁶/mouse | Fresh | High | 92.0 |
| | | | | expander | |
| 5 | 327587 | 10 × 10⁶/mouse | Fresh | High | 94.3 |
| | | | | expander | |
| 6 | 328257 | 10 × 10⁶/mouse | Fresh | Low | 81.0 |
| | | | | expander | |

On day 7 post adoptive transfer, one animal per group was utilized for the detection of γδ T cells in the following tissues: spleen, liver, lung, intestine and skin. On days 7, 14, 21, and 28 post adoptive transfer, blood was drawn from the respective mice for flow analysis. Once blood was drawn, staining was performed immediately following the same protocol as described in Example 4. The flow panel consisted of the following: cell counts/μl, CD45, CD3, Vγ9, Vδ2, CD56, and CD69. The cell number was determined and rested overnight at the concentration 1×10*6/mL in complete growth media.

### 6.5.2. Engraftment Analysis Using Vγ9Vδ2 T Cells from the Various Donors and Fresh or Frozen Cells

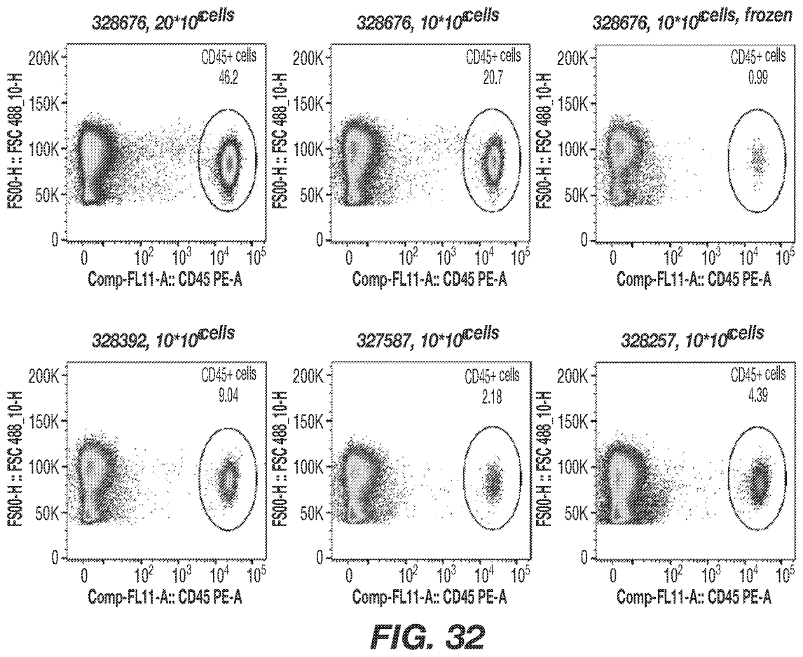

To determine whether the various donor Vγ9Vδ2 T cells were engrafted in the NOG-IL15 mouse strain, flow cytometry analysis was performed on blood samples collected 7 days post adoptive transfer (gated for CD45⁺). The protocol for flow cytometry was performed as described herein. FIG. 32 shows engraftment among the various donor Vγ9Vδ2 T cells. Specifically, donor 328676 showed the most efficient engraftment as shown by the strong signal of human CD45⁺ cells. Donors 328392, 327587, and 328257 showed modest engraftment as shown by the modest signal of CD45⁺ cells. There was donor-to-donor variability in engraftment efficiency, which was not correlated to donor expansion status (e.g. high vs low). Also, frozen Vγ9Vδ2 T cells demonstrated poor engraftment as shown in the 328676 frozen donor cells. These data demonstrated that fresh Vγ9Vδ2 T cells were able to engraft more efficiently in the NOG-IL15 mouse strain.

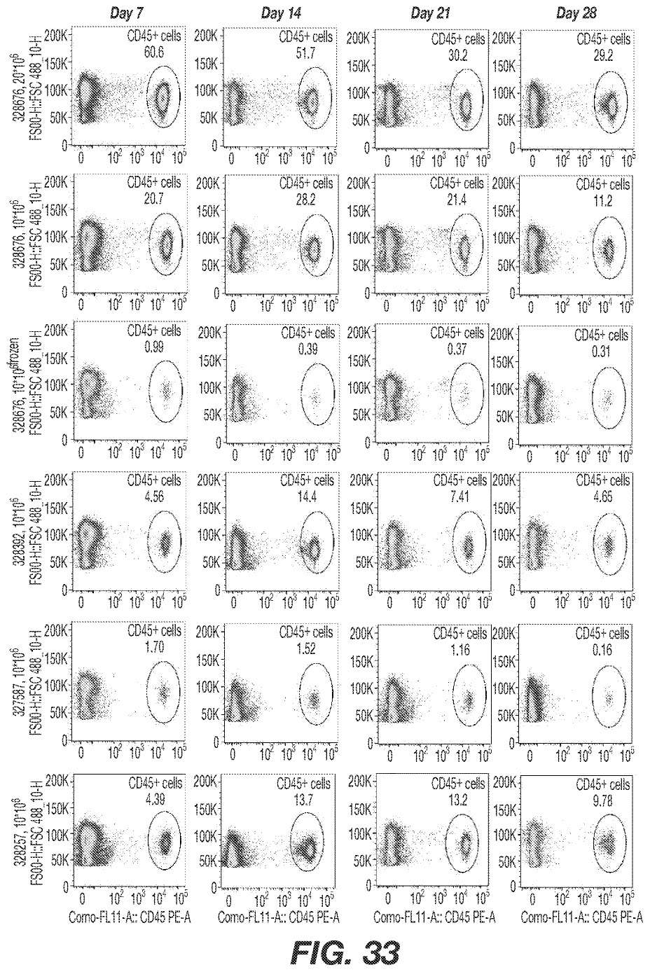

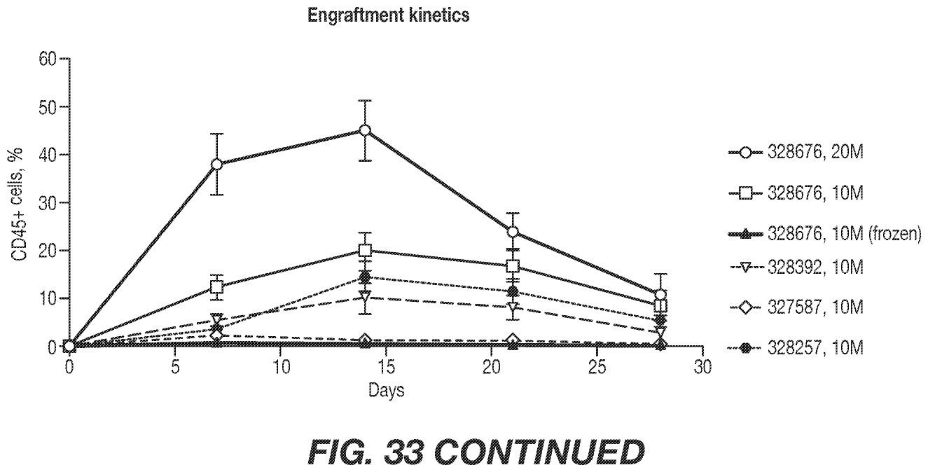

To determine whether engraftment of Vγ9Vδ2 T cells persisted in the NOG-IL15 mouse strain, flow cytometry was performed on blood samples collected at days 7, 14, 21, and 28 post adoptive transfer (gated for CD45⁺). The protocol for flow cytometry was performed as described herein. As shown in FIG. 33, similar engraftment kinetics was observed in Vγ9Vδ2 T cells reconstitution as compared to results observed in Example 4. Particularly, the peak of percentage human cells present in mouse circulation was observed on Day 14, and subsequently declined. Specifically, the group received 20×10⁶ of fresh Vγ9Vδ2 T cells from donor 328676 showed the highest percentage of human CD45⁺ cells comparing to other groups receiving 10×10⁶ Vγ9Vδ2 T cells. There was some variability in the fresh donor Vγ9Vδ2 T cell groups as donor 327587 showed poor engraftment and thus a small percentage of CD45⁺ cells. Further, the frozen donor Vγ9Vδ2 T cells also showed poor engraftment and thus a small percentage of CD45⁺ cells.

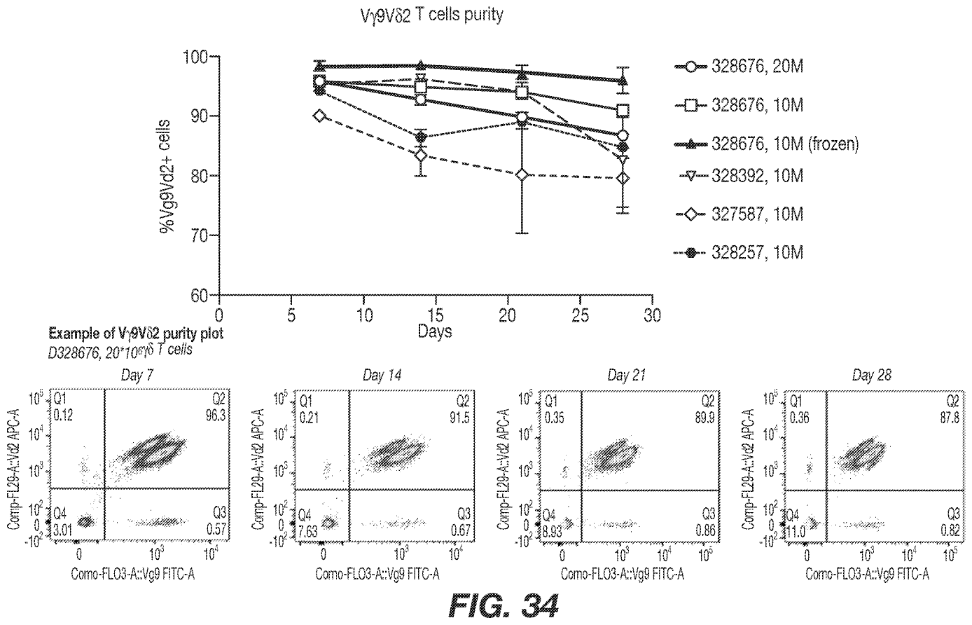

To determine whether negatively enriched donor fresh and frozen Vγ9Vδ2 T cells can maintain their purity during engraftment, flow cytometry was performed on blood samples on days 7, 14, 21, and 28 post adoptive transfer from the NOG-IL 15 mouse strain. The protocol for flow cytometry was performed as described herein. As shown in FIG. 34, high purity was observed in donors 328676 (both fresh and frozen) and 328392 Vγ9Vδ2 T cells in NOG-IL 15 mice. In contrast, donors 327587 (poor engraftment) and 328257 (low expander) Vγ9Vδ2 T cells showed lower purity in NOG-IL15 mice. Donor 327587 cells had poor engraftment, and Donor 328257 cells had low expansion before the adoptive transfer. These data demonstrated that donors 328676 and 328392 Vγ9Vδ2 T cells maintained high purity post adoptive transfer.

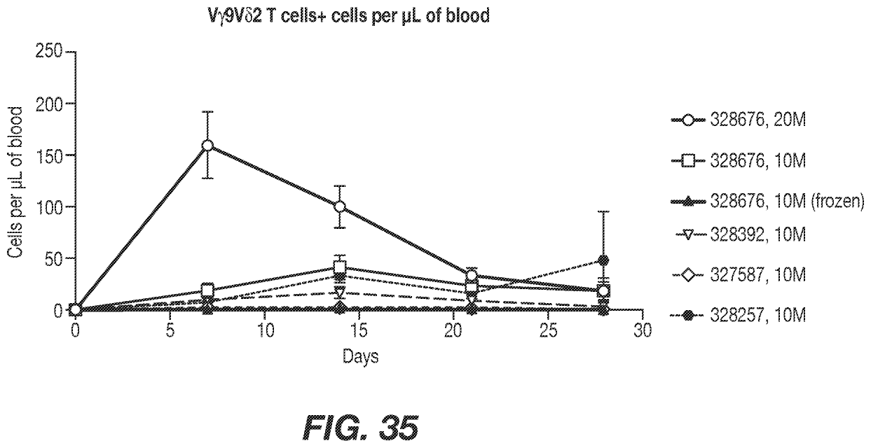

To determine the number of donor Vγ9Vδ2 T cells in the NOG-IL15 mouse strain, cell counts per microliter of mouse blood were measured on days 7, 14, 21, and 28 post adoptive transfer. FIG. 35 demonstrates that the group received 20×10⁶ fresh Vγ9Vδ2 T cells from donor 328676 had the highest cell count per microliter of blood on day 7 and then decreased on days 14, 21, and 28 in NOG-IL 15 mice. The other donor groups showed modest cell counts per microliter of blood in NOG-IL 15 mice on days 7, 14, 21, and 28.

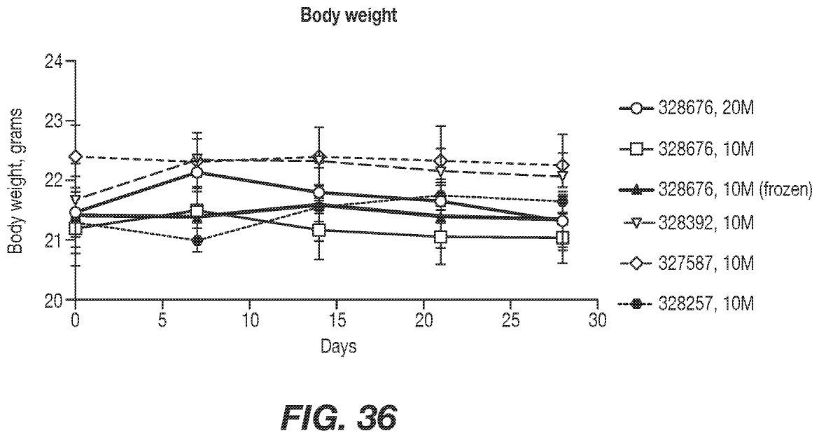

To determine whether the adoptive transfer of donor Vγ9Vδ2 T cells causes GvHD, NOG-IL 15 mice were administered purified donor Vγ9Vδ2 T cells and were weighed on days 0, 7, 14, 21, and 28. FIG. 36 shows NOG-IL15 mice that received the purified donor Vγ9Vδ2 T cells had a similar weight on day 28 post adoptive transfer compared to day 0 (the day of the adoptive transfer). These data demonstrated that NOG-IL 15 mice receiving the purified donor Vγ9Vδ2 T cells for the adoptive transfer did not contract GvHD, consistent with the results of Example 4.

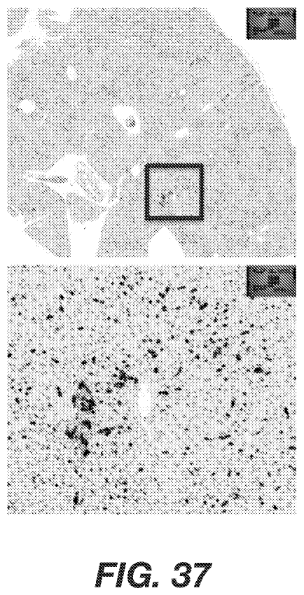

To determine whether the enriched Vγ9Vδ2 T cells were able to infiltrate tissue in NOG-IL15 mice harboring the JIMT1-GFP/fluc containing tumor, tissue was collected on day 28 post adoptive transfer. Using in situ hybridization (ISH), FIG. 37 shows enriched Vγ9Vδ2 T cells (γδ T cells) (dark spots) were able to infiltrate mouse liver tissues. These data demonstrated enriched Vγ9Vδ2 T cells were capable of infiltrating tissue in the subject.

The studies of Example 5 surveyed engraftment and persistence of adoptively transferred Vγ9Vδ2 T cells from 4 healthy donors in the NOG-IL15 strain. The donor-to-donor variation was observed, however, was not correlated to expansion status of the donor. Engraftment of frozen cells were not successful and therefore it is recommended to use fresh cultures for adoptive transfer of Vγ9Vδ2 T cells in future studies. Injection of 20×10⁶ cells per animal appears to show best engraftment efficiency and is recommended for the future studies. Similar trends in term of Vγ9Vδ2 T cells engraftment, persistence, purity, cell count was observed in the studies of Example 5 as compared to studies described in Example 4, highlighting reproducibility of the model.

### 6.6. Example 6-In Vivo Vγ9Vδ2 T Cells Redirection in NOG-IL15 Mice Engrafted with Tumor Antigen-Targeted Therapy-Resistant Tumor Cells

Studies of Example 6 sought to monitor the growth of an in vivo engrafted tumor cell line following the adoptive transfer and redirection of expanded and enriched Vγ9Vδ2 T cells in NOG-IL15 mice, which received a bispecific antibody treatment (designated as A1×A2 treatment). The A1×A2 bispecific antibody comprised two antigen-binding domains, where one antigen-binding domain binds to A1, which was expressed by the Vγ9Vδ2 T cells, and the other binding domain binds to A2, which was expressed by the engrafted tumor cell line. In addition, the engrafted tumor cell line was an established cell line that was resistant to treatments targeting A2 (e.g., the A1×A2 bispecific antibody). Two arms were included in the studies, where one cohort received Vγ9Vδ2 T cells through adoptive transfer (intravenous injection), and the other cohort received a mixture of Vγ9Vδ2 T cells and tumor cells upon engraftment (subcutaneous injection).

Expanded and enriched Vγ9Vδ2 T cells (under normoxic condition as disclosed in the present disclosure) from a healthy donor were provided in PBS. Twenty (20) female NOD.Cg-Prkdc^{scid} 112rg^{tm1Sug} Tg(CMV-IL2/IL15) 1-1Jic/JicTac (“NOG-IL15”) mice with 24-28 weeks of age were used. NOG-IL 15 mice are B cell, T cell, and NK cell deficient, have reduced complement activity, dysfunctional macrophages, and expressed human IL15.

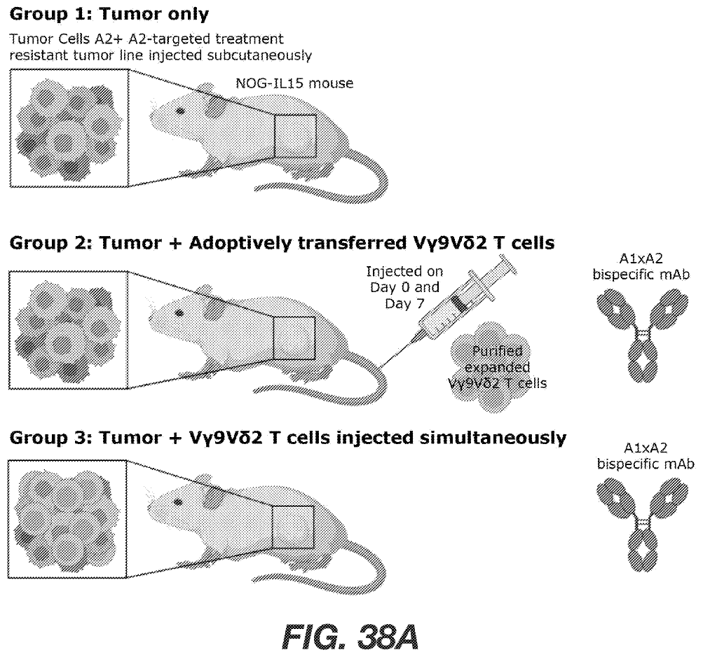

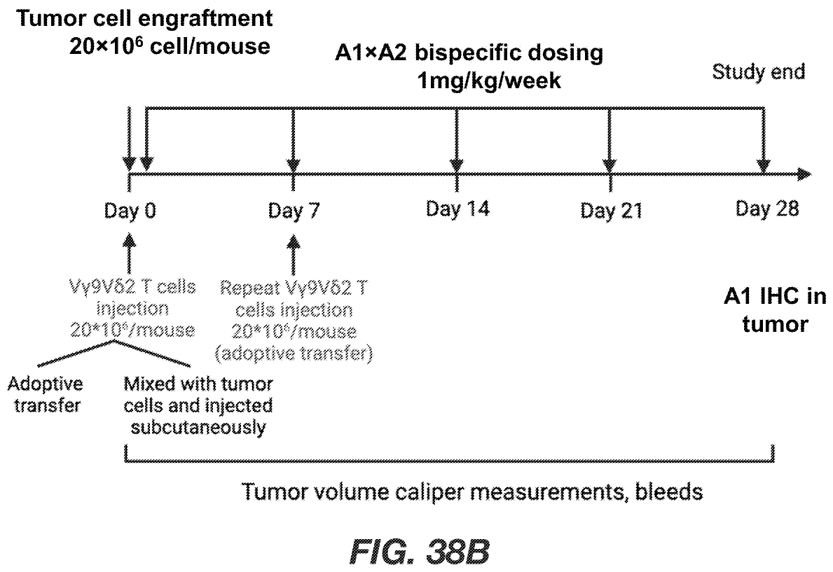

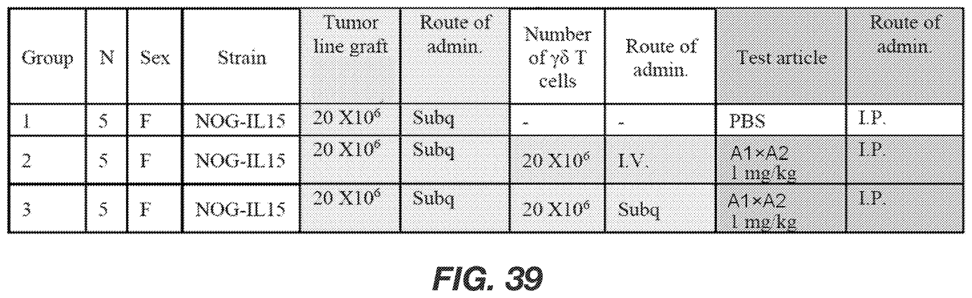

Study design of Example 6 is depicted in FIGS. 38A & 38B. Briefly, at the initiation of the study (day 0), all animals were weighed and divided into three groups (see FIG. 39 for a brief summary of the study groups). Group 1 (control group) received tumor cells with no A1×A2 bispecific antibody or Vγ9Vδ2 T cell treatment. Group 2 received tumor cells subcutaneously once and adoptively transferred Vγ9Vδ2 T cells twice (day 0 and day 7) with weekly bispecific treatments. Group 3 received a mixture of tumor cells and Vγ9Vδ2 T cells subcutaneously once with weekly bispecific treatments.

In particular, on day 0, Group 1 and Group 2 received an injection of the tumor cells in Cultrex ECM (20×10⁶ cell/mouse) via subcutaneous injection in the area of the right hip. Group 3 received an injection containing a mixture of the tumor cells (20×10⁶ cell/mouse) and expanded and enriched Vγ9Vδ2 T cells (20×10⁶ cell/mouse) in 0.1 mL of Cultrex ECM via subcutaneous injection in the area of the right hip. Additionally, Group 2 received an injection of expanded and enriched Vγ9Vδ2 T cells in 0.1 mL PBS via intravenous injection (20×10⁶ cell/mouse) on day 0 and day 7.

Animals were weighed weekly. Tumor measurements by caliper were performed twice a week. Group 2 and Group 3 were dosed with A1×A2 bispecific antibody (1 mg/kg in 100 μL, intraperitoneal injection) weekly (day 0, day 7, day 14, and day 21). Group 1 received 100 μL of PBS weekly (day 0, day 7, day 14, and day 21). All groups were weighed and bled on days post Vγ9Vδ2 T cell adoptive transfer (day 7, day 14, day 21, and day 28) to assess γδ T cell engraftment and persistence.

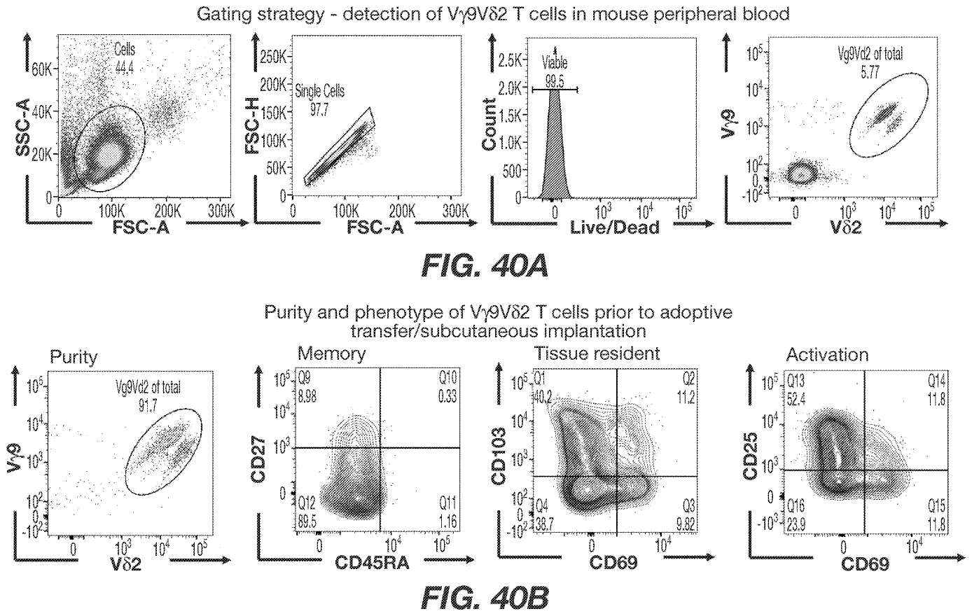

Prior to administering the Vγ9Vδ2 T cells to the animals, the purity and phenotype of the Vγ9Vδ2 T cells were measured (FIGS. 40A & 40B). The purity of Vγ9Vδ2 T cells prior to adoptive transfer and subcutaneous implantation was about 91% (FIG. 40B). Moreover, the majority of the Vγ9Vδ2 T cells resembled effector memory phenotype, having moderate expression of CD103, CD25 and CD69 (FIG. 40B).

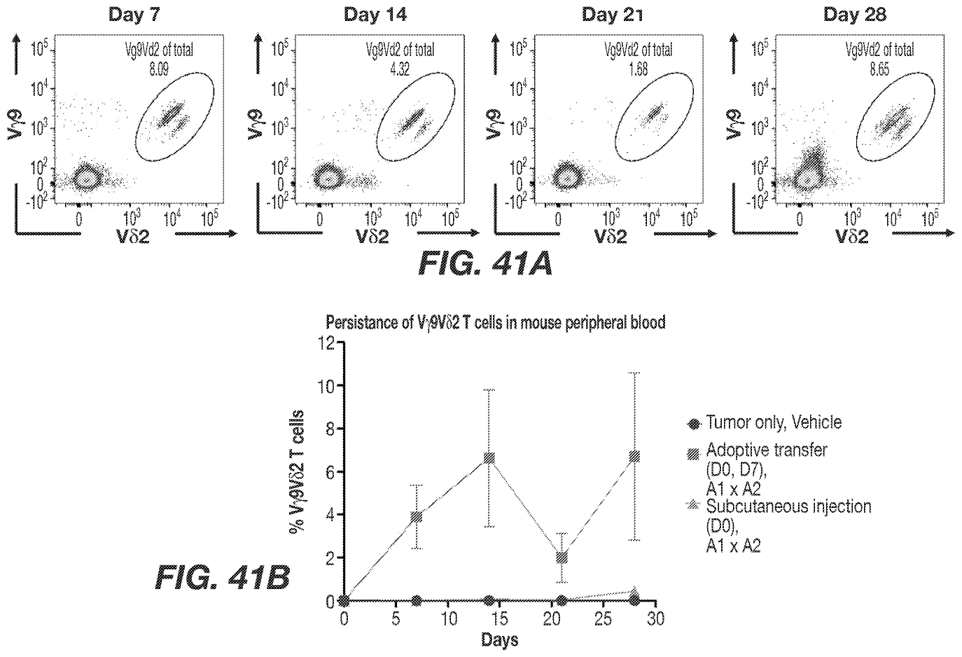

The adoptively transferred Vγ9Vδ2 T cells persisted in mouse peripheral blood for the entire duration of the study and were represented by 2-8% of total cells (FIGS. 41A & 41B). Notably, there was a lag of Vγ9Vδ2 T cells in mouse periphery at day 21, however, higher overall numbers of Vγ9Vδ2 T cells were observed on mouse blood on day 28 (FIGS. 41A & 41B).

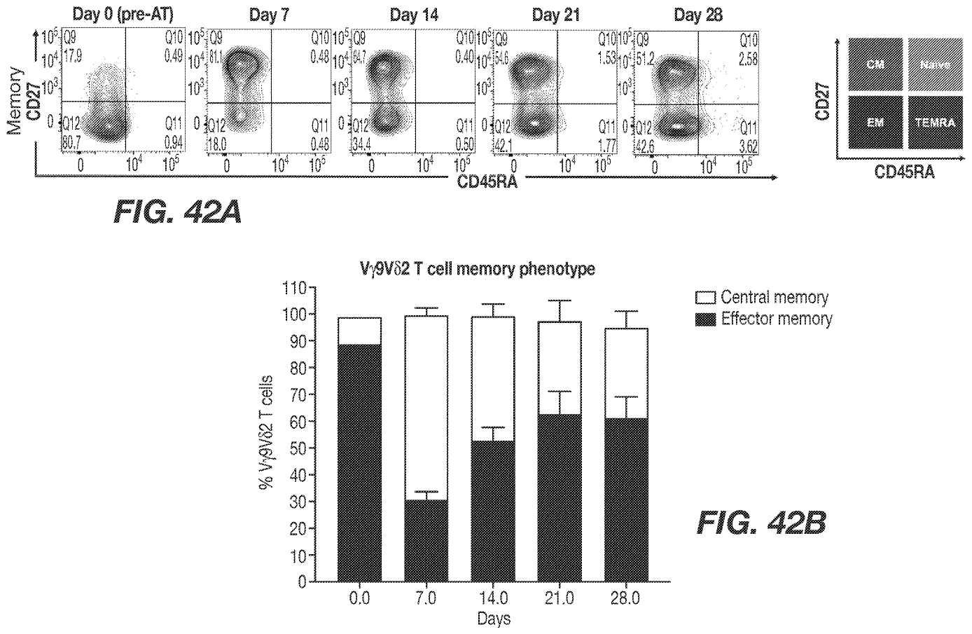

Kinetic changes in Vγ9Vδ2 T cell memory phenotype were evaluated (FIGS. 42A & 42B). The majority of Vγ9Vδ2 T cells resembled effector memory phenotype upon adoptive transfer and subcutaneous implantation. However, 7 days post injection, the majority of cells resembled central memory phenotype. Further analysis in the rest of the study period revealed equal distribution between effector and memory phenotype of Vγ9Vδ2 T cells in mouse periphery. These differences could be due to differential migration pattern of central memory and effector memory cells.

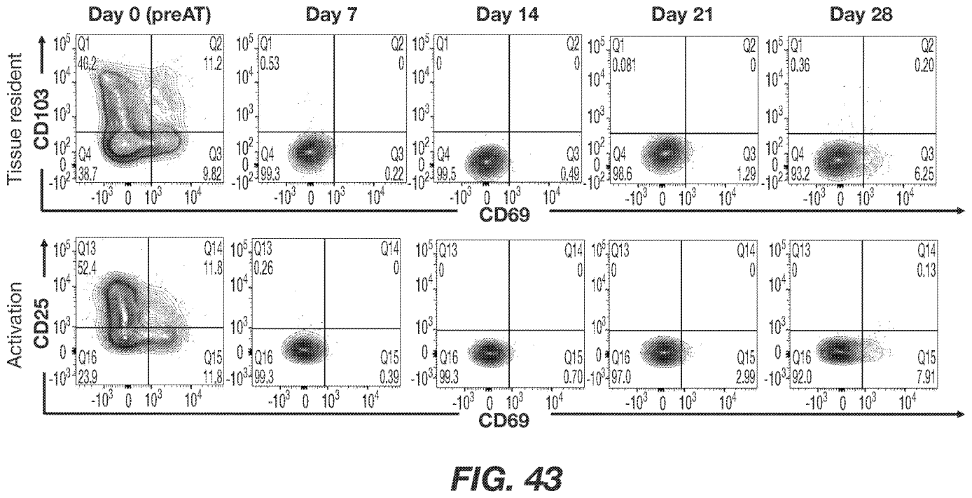

Additional phenotype of Vγ9Vδ2 T cells was further evaluated (FIG. 43). The majority of Vγ9Vδ2 T cells resembled moderate expression of tissue-resident T cell markers CD103 and CD69, and activation markers CD25 and CD69. However, upon adoptive transfer, Vγ9Vδ2 T cells lost expression of CD103, CD25 and CD69. In addition, there was a mild increase of CD69 on the 28th day of the study.

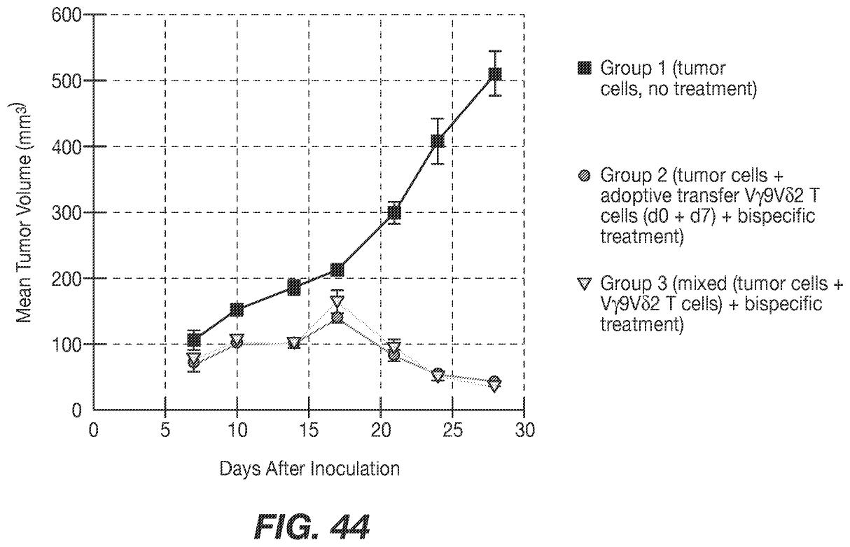

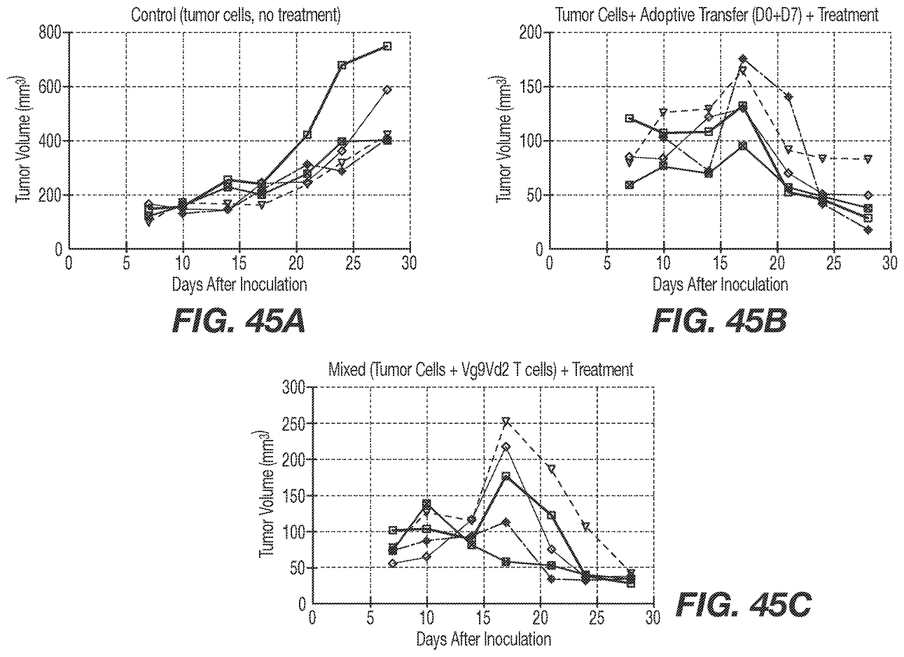

As shown in FIGS. 44 and 45A-45C, Vγ9Vδ2 T cells, injected either via adoptive transfer (twice) or transplanted subcutaneously, and administered the bispecific antibody, provided strong anti-tumor response. Tumor growth kinetics of Group 2 (adoptively transferred (twice)) and Group 3 (transplanted subcutaneously) demonstrated comparable pattern, suggesting adoptively transferred Vγ9Vδ2 T cells can migrate to the tumor and exhibit strong cytotoxicity. Additionally, when transferred adoptively, the Vγ9Vδ2 T cells migrated to the hypoxic solid tumor site and exhibited efficient response against solid tumor. In a parallel study arm, subcutaneously injected Vγ9Vδ2 T cells also did very well in hypoxic tumor environment.

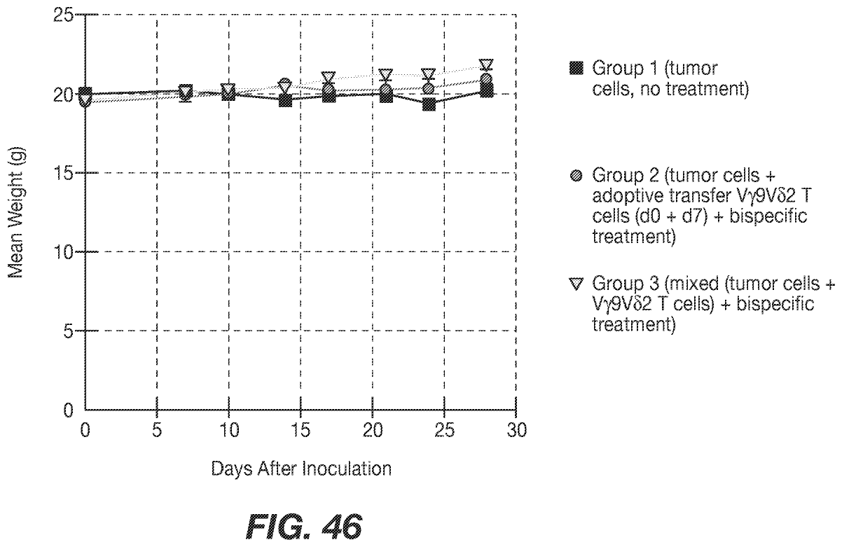

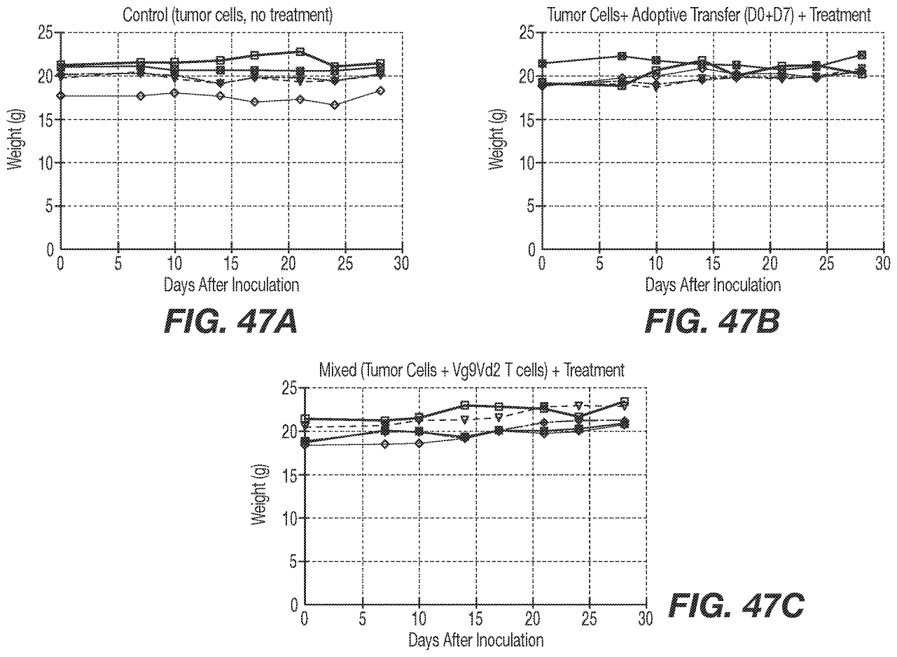

Body weight was not significantly impacted by the Vγ9Vδ2 T cell and A1×A2 bispecific antibody treatments (FIGS. 46 and 47A-47C).

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

1. A method for ex vivo activation and expansion of Vγ9Vδ2 T cells, comprising:
a) contacting a population of cells comprising T cells with a culture system comprising IL-2, IL-15, and a bisphosphonate or a mevalonate pathway intermediate; and
b) culturing the population of cells ex vivo in the culture system under a hypoxic condition to activate and expand Vγ9Vδ2 T cells.

2. The method of claim 1,
wherein the method further comprises obtaining the population of cells from a subject, wherein the subject is healthy or unhealthy or wherein the population of the cells is derived from a human tissue wherein the human tissue is fresh or frozen; and
wherein the population of cells is a population of mammalian cells, human cells, peripheral blood mononuclear cells (PBMCs) or tumor-infiltrating lymphocytes (TILs);
wherein the human cells are engineered cells or non-engineered cells, wherein the PBMCs are freshly obtained PBMCs or frozen PBMCs, or wherein the TILs are freshly obtained TILs or frozen TILs.

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. The method of claim 1, wherein the population of cells is cultured in the culture system under the hypoxic condition for at least 3 days, or at least 5 days, or at least 7 days, or at least 9 days, or at least 11 days, or at least 13 days, or at least 15 days, or at least 17 days, or at least 19 days, or at least 21 days; or
wherein the population of cells is cultured in the culture system under the hypoxic condition for from 3 days to 25 days, or from 4 days to 23 days, or from 5 days to 21 days, or from 6 days to 19 days, or from 7 days to 17 days, or from 8 days to 15 days, or from 9 days to 14 days, or from 10 days to 14 days, or from 11 days to 14 days, or from 12 days to 14 days; or
wherein the population of cells is cultured in the culture system under the hypoxic condition for about 15 days, optionally wherein the population of cells is cultured in the culture system under the hypoxic condition for 14 days.

19. (canceled)

20. (canceled)

21. The method of claim 1, wherein the oxygen concentration of the hypoxic condition is less than 15%, or less than 13%, or less than 11%, or less than 9%, or less than 7%, or less than 5%, or less than 3%, or less than 1%, or less than 0.5%;
wherein the oxygen concentration of the hypoxic condition is from 0.1% to 15%, or from 0.5% to 13%, or from 1% to 13%, or from 1% to 11%, or from 1% to 9%, or from 1% to 7%, or from 2% to 5%; or
wherein the oxygen concentration of the hypoxic condition is or is about 2%, or 5%, or 12%.

22. (canceled)

23. (canceled)

24. (canceled)

25. The method of claim 1, wherein the method further comprises culturing the population of cells in the culture system under a normoxic condition to activate and expand Vγ9Vδ2 T cells;
wherein the population of cells is cultured under the normoxic condition for at least 1 hour prior to being cultured under the hypoxic condition;
wherein the population of cells is cultured under the normoxic condition for from 0.5 days to 7 days, or from 1 days to 6 days, or from 1 days to 5 days, or from 1 days to 4 days, or from 1 days to 3 days, or from 1 days to 2 days prior to being cultured under the hypoxic condition; and
wherein the oxygen concentration of the normoxic condition is or is about 18.2% or 18.6%.

26. (canceled)

27. (canceled)

28. (canceled)

29. The method of claim 1, wherein the IL-2 concentration within the culture system is from 10 IU/mL to 1200 IU/mL, or is or is about 10 IU/ml, wherein the IL-2 concentration within the culture system is adjusted to decrease during the culturing, wherein the IL-2 concentration within the culture system is 1000 IU/mL or no more than 1000 IU/mL on days 0 and 1, 800 IU/mL or no more than 800 IU/mL on days 2, 3 and 4, and 100 IU/mL or no more than 100 IU/mL on day 5 and thereafter; and
wherein the IL-15 concentration within the culture system is from 5 ng/ml to 25 ng/ml, or from 50 ng/ml to 300 ng/ml, or is or is about 100 ng/mL, or is or is about 200 ng/ml, and wherein the IL-15 concentration within the culture system is adjusted during the culturing, wherein the IL-15 concentration within the culture system is 10 ng/ml or no more than 10 ng/ml on days 0 and 1, 20 ng/ml or no more than 20 ng/mL on days 2, 3, and 4, and 10 ng/ml or no more than 10 ng/mL on day 5 and thereafter.

30. (canceled)

31. (canceled)

32. (canceled)

33. (canceled)

34. (canceled)

35. The method of claim 1, wherein the bisphosphonate is selected from the group consisting of zoledronic acid, risedronic acid, ibandronic acid, alendronic acid, pamidronic acid, tiludronic acid, etidronic acid, and clodronic acid, and wherein the mevalonate pathway intermediate is selected from the group consisting of HMBPP, BrHPP, and isopentenyl pyrophosphate.

36. (canceled)

37. The method of claim 1, wherein the method increases the total percentage of Vγ9Vδ2 T cells in the population of cells to more than 10%, or more than 15%, or more than 20%, or more than 25%, or more than 30%, or more than 35%, or more than 40%, or more than 45%, or more than 50%, or more than 55%, or more than 60%; or
wherein the method increases the total percentage of Vγ9Vδ2 T cells in the population of cells to from 10% to 99%, or from 20% to 95%, or from 30% to 95%, or from 35% to 95%, or from 40% to 95%, or from 45% to 95%, or from 50% to 95%, or from 55% to 95%, or from 60% to 95%, or from 65% to 95%.

38. (canceled)

39. The method of claim 1, wherein the method increases the total number of Vγ9Vδ2 T cells in the population of cells by at least 10-fold, or at least 30-fold, or at least 50-fold, or at least 100-fold, or at least 150-fold, or at least 200-fold, or at least 250-fold, or at least 300-fold, or at least 350-fold, or at least 400-fold, or at least 450-fold, or at least 500-fold, or at least 550-fold, or at least 600-fold as compared to the total number of Vγ9Vδ2 T cells in the population of cells before the contacting; or
wherein the method increases the total number of Vγ9Vδ2 T cells in the population of cells by from 10-fold to 700-fold, or from 30-fold to 650-fold to fold, or from 50-fold to 600-fold, or from 100-fold to 600-fold, or from 150-fold to 600-fold, or from 200-fold to 600-fold, or from 250-fold to 600-fold, or from 300-fold to 600-fold, or from 350-fold to 600-fold, or from 400-fold to 600-fold, or from 450-fold to 600-fold, or from 500-fold to 600-fold as compared to the total number of Vγ9Vδ2 T cells in the population of cells before the contacting.

40. (canceled)

41. The method of claim 1, further comprising enriching the Vγ9Vδ2 T cells in the population of cells, wherein the enriching results in the percentage of Vγ9Vδ2 T cells in the population of cells is more than 95%.

42. (canceled)

43. A method for ex vivo activation and expansion of Vγ9Vδ2 T cells, comprising:
1a) contacting a population of cells comprising T cells with a culture system comprising IL-2, IL-15, and zoledronic acid, and
1b) culturing the population of cells ex vivo in the culture system under a hypoxic condition for about 14 days to activate and expand Vγ9Vδ2 T cells, and
wherein (i) the IL-2 concentration in the culture system is (i) 1000 IU/mL or no more than 1000 IU/mL on days 0 and 1, 800 IU/mL or no more than 800 IU/mL on days 2, 3 and 4, and 100 IU/mL or no more than 100 IU/mL on day and thereafter, or (2) is or is about 10; (ii) the IL-15 concentration within the culture system is (1) 10 ng/mL or no more than 10 ng/mL on days 0 and 1, 20 ng/mL or no more than 20 ng/mL on days 2, 3, and 4, and 10 ng/mL or no more than 10 ng/mL on day 5 and thereafter, or (2) is or is about 100 ng/ml, or is or is about 200 ng/ml; (iii) the oxygen concentration of the hypoxic condition is or is about 5%; optionally wherein (iv) the zoledronic acid concentration in the culture system is or is about 350 nM; or
2a) contacting a population of cells comprising T cells with a culture system comprising IL-2, IL-15, and zoledronic acid,
2b) culturing the population of cells under a normoxic condition to activate and expand Vγ9Vδ2 T cells for at least 1 hour prior to being cultured under a hypoxic condition; and
2c) culturing the population of cells under the hypoxic condition for about 14 days to further activate and expand Vγ9Vδ2 T cells,
wherein (i) the IL-2 concentration in the culture system is (i) 1000 IU/mL or no more than 1000 IU/mL on days 0 and 1, 800 IU/mL or no more than 800 IU/mL on days 2, 3 and 4, and 100 IU/mL or no more than 100 IU/mL on day 5 and thereafter, or (2) is or is about 10; (ii) the IL-15 concentration within the culture system is (1) 10 ng/ml or no more than 10 ng/ml on days 0 and 1, 20 ng/ml or no more than 20 ng/mL on days 2, 3, and 4, and 10 ng/ml or no more than 10 ng/mL on day 5 and thereafter, or (2) is or is about 100 ng/ml, or is or is about 200 ng/ml; (iii) the oxygen concentration of the hypoxic condition is or is about 5%; (iv) the oxygen concentration of the normoxic condition is or is about 18.2% or 18.6%; and optionally wherein (iv) the zoledronic acid concentration in the culture system is or is about 350 nM.

44. (canceled)

45. An isolated population of Vγ9Vδ2 T cells produced by the method of claim 1.

46. An isolated population of cells, wherein the percentage of Vγ9Vδ2 T cells in the isolated population of cells is (a) more than 10%, or more than 15%, or more than 20%, or more than 25%, or more than 30%, or more than 35%, or more than 40%, or more than 45%, or more than 50%, or more than 55%, or more than 60%; or (b) from 10% to 99%, or from 20% to 95%, or from 30% to 95%, or from 35% to 95%, or from 40% to 95%, or from 45% to 95%, or from 50% to 95%, or from 55% to 95%, or from 60% to 95%, or from 65% to 95%.

47. A pharmaceutical composition comprising the isolated population of Vγ9Vδ2 T cells of claim 45, and optionally a pharmaceutically acceptable excipient.

48. A method for treating a disease or disorder in a subject, comprising administering to the subject: (i) a therapeutically effective amount of the isolated population of Vγ9Vδ2 T cells of claim 45, and (ii) a therapeutically effective amount of at least one multispecific antibody;
optionally wherein the at least one multispecific antibody comprises (a) a first binding domain that binds to an antigen expressed on a Vγ9Vδ2 T cell; and (b) a second binding domain that binds to an antigen expressed on an unhealthy cell;
further optionally wherein (a) the antigen expressed on the Vγ9Vδ2 T cell is T Cell Receptor Gamma Variable 9 (TRGV9) or CD3; or (b) the unhealthy cell is a cancer cell, and wherein the antigen expressed on the unhealthy cell is a tumor-associated antigen (TAA); wherein the disease or disorder is cancer, further optionally wherein the cancer cell is a blood cancer cell or a solid tumor cancer cell, further optionally wherein the subject is a human subject in need thereof.

49. The method of claim 48, wherein the at least one multispecific antibody comprises a Vγ9×TAA and/or CD3×TAA bispecific antibody.

50. (canceled)

51. A process for making a chimeric antigen receptor (CAR) T cell, comprising:
(i) a step of performing a function of obtaining the isolated population of Vγ9Vδ2 T cells of claim 45; and
(ii) a step of performing a function of expressing a CAR in the Vγ9Vδ2 T cells;
optionally wherein the CAR comprises an extracellular domain, a transmembrane domain, and an intracellular domain; further optionally wherein the extracellular domain binds to an antigen expressed on an unhealthy cell;
further optionally wherein the unhealthy cell is a cancer cell; further optionally wherein the cancer cell is a blood cancer cell or a solid tumor cancer cell.

52. A method for making a chimeric antigen receptor (CAR) T cell, comprising: (i) obtaining the isolated population of Vγ9Vδ2 T cells of claim 45; and (ii) introducing a nucleic acid encoding a CAR into the Vγ9Vδ2 T cells,
optionally wherein the CAR comprises an extracellular domain, a transmembrane domain, and an intracellular domain; further optionally wherein the extracellular domain binds to an antigen expressed on an unhealthy cell; further optionally wherein the unhealthy cell is a cancer cell; further optionally wherein the cancer cell is a blood cancer cell or a solid tumor cancer cell.

53. A CAR T cell produced by the method of claim 52.

54. A pharmaceutical composition comprising the CAR T cell of claim 53, and optionally a pharmaceutically acceptable excipient.

55. A method for treating a disease or disorder in a subject, comprising administering to the subject a therapeutically effective amount of the CAR T cell of claim 53.

56. A method for establishing an in vivo engraftment of Vγ9Vδ2 T cells in a receiving subject, the method comprising (i) obtaining the isolated population of Vγ9Vδ2 T cells of claim 45; and (ii) adoptively transferring the population of cells to the receiving subject;
optionally wherein the population of cells comprising Vγ9Vδ2 T cells is produced from the population of cells comprising T cells obtained from the receiving subject;
optionally wherein the population of cells comprising Vγ9Vδ2 T cells is a purified population of Vγ9Vδ2 T cells;
optionally wherein the population of cells comprising Vγ9Vδ2 T cells is enriched to comprise more than 95% Vγ9Vδ2 T cells one day before the adoptive transfer of the population of cells to the receiving subject;
optionally wherein the population of cells comprising Vγ9Vδ2 T cells comprises more than 10%, or more than 15%, or more than 20%, or more than 25%, or more than 30%, or more than 35%, or more than 40%, or more than 45%, or more than 50%, or more than 55%, or more than 60% Vγ9Vδ2 T cells in the population of cells;
optionally wherein the population of cells comprising Vγ9Vδ2 T cells comprises from 10% to 99%, or from 20% to 95%, or from 30% to 95%, or from 35% to 95%, or from 40% to 95%, or from 45% to 95%, or from 50% to 95%, or from 55% to 95%, or from 60% to 95%, or from 65% to 95% Vγ9Vδ2 T cells in the population of cells;
optionally wherein the population of cells comprising Vγ9Vδ2 T cells comprises less than 95%, or less than 90%, or less than 85%, or less than 80%, or less than 75%, or less than 70%, or less than 65%, or less than 60%, or less than 55%, or less than 50%, or less than 45%, or less than 40%, or less than 35%, or less than 30%, or less than 25%, or less than 20%, or less than 15%, or less than 10%, or less than 5%, or less than 1%, or is devoid of αβ T cells in the population of cells;
optionally wherein the population of cells comprises at least about or about 5×10⁶ cells, at least about or about 1×10⁷ cells, at least about or about 10×10⁷ cells, at least about or about 20×10⁷ cells, at least about or about 30×10⁷ cells, at least about or about 40×10⁷ cells, at least about or about 50×10⁷ cells, at least about or about 60×10⁷ cells, at least about or about 70×10⁷ cells, at least about or about 80×10⁷ cells, at least about or about 90×10⁷ cells, at least about or about 1×10⁸ cells, at least about or about 10×10⁸ cells, at least about or about 20×10⁸ cells, at least about or about 30×10⁸ cells, at least about or about 40×10⁸ cells, at least about or about 50×10⁸ cells, at least about or about 60×10⁸ cells, at least about or about 70×10⁸ cells, at least about or about 80×10⁸ cells, at least about or about 90×10⁸ cells, or at least about or about 1×10⁹ cells; and
wherein the adoptively transferring comprises administering the population of cells to the receiving subject;
optionally wherein the method further comprises (iii) administering, concurrently or sequentially with the adoptively transferring, an effective amount of a composition comprising IL-2, IL-15, a bisphosphonate or a mevalonate pathway intermediate, or a combination thereof;
optionally wherein the bisphosphonate is selected from the group consisting of zoledronic acid, risedronic acid, ibandronic acid, alendronic acid, pamidronic acid, tiludronic acid, etidronic acid, and clodronic acid;
optionally wherein the mevalonate pathway intermediate is selected from the group consisting of HMBPP, BrHPP, and isopentenyl pyrophosphate;
optionally wherein the IL-2 is administered at the dosage of 2×104 IU/kg of body weight;
optionally wherein the bisphosphonate is zoledronic acid administered at the dosage of 2.5 mg/kg of body weight; and
optionally wherein the administration of the population of the cells and/or administration of the effective amount of the composition are intravenous administration or intraperitoneal administration; and
wherein the adoptively transferred Vγ9Vδ2 T cells produce progeny cells in the subject;
optionally wherein the progeny cells are CD45+ cells, CD56+ cells, or CD69+ cells;
optionally wherein the adoptively transferred Vγ9Vδ2 T cells are present in the blood of the receiving subject at least 7 days, at least 14 days, at least 21 days, or at least 28 days after the adoptive transfer of the population of cells to the receiving subject;
optionally wherein the adoptively transferred Vγ9Vδ2 T cells infiltrate into a tissue in the receiving subject;
optionally wherein the tissue is a spleen tissue, a liver tissue, a lung tissue, an intestine tissue, a skin tissue, or a combination thereof; optionally wherein the tissue comprises an unhealthy cell;
optionally wherein the unhealthy cell is a cancer cell; optionally wherein the cancer cell is a blood cancer cell or a solid tumor cancer cell;
optionally wherein the receiving subject does not develop any symptom of a Graft Versus Host Disease (GvHD) at least 7 days, at least 14 days, at least 21 days, or at least 28 days after the adoptive transfer of the population of cells to the receiving subject; and
wherein the Vγ9Vδ2 T cells are chimeric antigen receptor (CAR) T cells comprising an extracellular domain, a transmembrane domain, and an intracellular domain,
optionally wherein the extracellular domain binds to an antigen expressed on an unhealthy cell; further optionally wherein the unhealthy cell is a cancer cell;
further optionally wherein the cancer cell is a blood cancer cell or a solid tumor cancer cell.

57. (canceled)

58. (canceled)

59. (canceled)

60. A method for treating a disease or disorder in a subject, comprising administering to the subject: (i) a therapeutically effective amount of a population of Vγ9Vδ2 T cells, and (ii) a therapeutically effective amount of at least one multispecific antibody; optionally wherein the at least one multispecific antibody comprises: (a) a first binding domain that binds to an antigen expressed on a Vγ9Vδ2 T cell; and (b) a second binding domain that binds to an antigen expressed on an unhealthy cell;
further optionally wherein (a) the antigen expressed on the Vγ9Vδ2 T cell is T Cell Receptor Gamma Variable 9 (TRGV9) or CD3; or (b) the unhealthy cell is a cancer cell, and wherein the antigen expressed on the unhealthy cell is a tumor-associated antigen (TAA); wherein the disease or disorder is cancer, optionally wherein the cancer cell is a blood cancer cell or a solid tumor cancer cell, optionally wherein the subject is a human subject in need thereof;
wherein the method comprises administering to the subject: (i) a therapeutically effective amount of the population of Vγ9Vδ2 T cells, and (ii) a therapeutically effective amount of Vγ9×TAA and/or CD3×TAA bispecific antibodies.

61. (canceled)

62. (canceled)

63. The pharmaceutical composition of claim 47, for use in the treatment of a disease or disorder in a subject, the treatment comprising administering to the subject: (i) a therapeutically effective amount of the isolated population of Vγ9Vδ2 T cells, the isolated population of cells, or the pharmaceutical composition, and (ii) a therapeutically effective amount of at least one multispecific antibody;
optionally wherein the at least one multispecific antibody comprises (a) a first binding domain that binds to an antigen expressed on a Vγ9Vδ2 T cell; and (b) a second binding domain that binds to an antigen expressed on an unhealthy cell;
further optionally wherein (a) the antigen expressed on the Vγ9Vδ2 T cell is T Cell Receptor Gamma Variable 9 (TRGV9) or CD3; or (b) the unhealthy cell is a cancer cell, and wherein the antigen expressed on the unhealthy cell is a tumor-associated antigen (TAA); wherein the disease or disorder is cancer, further optionally wherein the cancer cell is a blood cancer cell or a solid tumor cancer cell, further optionally wherein the subject is a human subject in need thereof.

64. The pharmaceutical composition for use according to claim 63, wherein the at least one multispecific antibody comprises a Vγ9×TAA and/or CD3×TAA bispecific antibody.

65. (canceled)

66. A CAR-T cell for use in the treatment of a disease or disorder in a subject, the treatment comprising administering to the subject a therapeutically effective amount of the CAR T cell of claim 53, optionally wherein the disease or disorder is cancer, optionally wherein the cancer is a blood cancer or a solid tumor cancer, optionally wherein the subject is a human subject in need thereof.
